(19) United States
(12) Patent Application Publication
(10) Pub. No.: US 2012/0157439 A1
(43) Pub. Date: Jun. 21, 2012

(54) RAF INHIBITOR COMPOUNDS AND METHODS OF USE THEREOF

(75) Inventors: Ignacio Aliagas, South San Francisco, CA (US); Stefan Gradl, South San Francisco, CA (US); Janet Gunzner, South San Francisco, CA (US); Simon Mathieu, South San Francisco, CA (US); Rebecca Pulk, South San Francisco, CA (US); Joachim Rudolph, South San Francisco, CA (US); Zhaoyang Wen, South San Francisco, CA (US); Jonas Grina, Boulder, CO (US); Joshua D. Hansen, Boulder, CO (US); Ellen Laird, Boulder, CO (US); David Moreno, Boulder, CO (US); Li Ren, Boulder, CO (US); Steven Mark Wenglowsky, Boulder, CO (US)

(73) Assignees: GENENTECH, INC., South San Francisco, CA (US); ARRAY BIOPHARMA INC., Boulder, CO (US)

(21) Appl. No.: 13/393,163

(22) PCT Filed: Aug. 27, 2010
(86) PCT No.: PCT/US10/46955

§ 371 Date: Feb. 28, 2012

Related U.S. Application Data
(60) Provisional application No. 61/238,109, filed on Aug. 28, 2009.
(60) Provisional application No. 61/314,528, filed on Mar. 16, 2010.

(51) Int. Cl. A61K 31/519; C07D 495/04; A61P 35/00; A61K 31/5377; A61K 31/53; C07D 487/04; C07D 513/04

(52) U.S. Cl. 514/210.21; 544/280; 544/278; 544/255; 544/117; 544/183; 514/265.1; 514/260.1; 514/234.2; 514/243

(57) ABSTRACT

Compounds of Formula I are useful for inhibition of Raf kinases. Methods of using compounds of Formula I and stereoisomers, tautomers, prodrugs and pharmaceutically acceptable salts thereof, for in vitro, in situ, and in vivo diagnosis, prevention or treatment of such disorders in mammalian cells, or associated pathological conditions are disclosed.

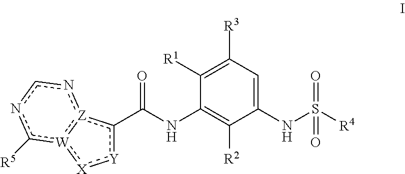

### PRIORITY OF INVENTION

This application claims priority under 35 U.S.C. 119(e) from U.S. Provisional Patent Application No. 61/238,109, filed 28 Aug. 2009 and U.S. Provisional Patent Application No. 61/314,528, filed 16 Mar. 2010, the contents of which are incorporated herein in their entirety.

### BACKGROUND OF THE INVENTION

1. Field of the Invention

The present invention relates to novel compounds, to pharmaceutical compositions comprising the compounds, to a process for making the compounds and to the use of the compounds in therapy. More particularly, it relates to certain substituted compounds useful for inhibiting Raf kinase and for treating disorders mediated thereby.

2. Description of the State of the Art

The Raf/MEK/ERK pathway is critical for cell survival, growth, proliferation and tumorigenesis. Li, Nanxin, et al. “B-Raf kinase inhibitors for cancer treatment.” Current Opinion in Investigational Drugs. Vol. 8, No. 6 (2007): 452-456. Raf kinases exist as three isoforms, A-Raf, B-Raf and C-Raf. Among the three isoforms, studies have shown that B-Raf functions as the primary MEK activator. B-Raf is one of the most frequently mutated genes in human cancers. B-Raf kinase represents an excellent target for anticancer therapy based on preclinical target validation, epidemiology and drugability.

Small molecule inhibitors of B-Raf are being developed for anticancer therapy. Nexavar® (sorafenib tosylate) is a multikinase inhibitor, which includes inhibition of B-Raf, and is approved for the treatment of patients with advanced renal cell carcinoma and unresectable hepatocellular carcinoma. Other Raf inhibitors have also been disclosed or have entered clinical trials, for example RAF-265, PLX-4032, PLX-3603, XL-281, or GSK-2118436.

Other B-Raf inhibitors are also known, see for example, U.S. Patent Application Publication 2006/0189627, U.S. Patent Application Publication 2006/0281751, U.S. Patent Application Publication 2007/0049603, U.S. Patent Application Publication 2009/0176809, International Patent Application Publication WO 2007/002325, International Patent Application Publication WO 2007/002433, International Patent Application Publication WO 2008/028141, International Patent Application Publication WO 2008/079903, International Patent Application Publication WO 2008/079906 and International Patent Application Publication WO 2009/012283.

International Patent Application Publication WO 2006/066913, International Patent Application Publication WO 2008/028617 and International Patent Application Publication WO 2008/079909 also disclose kinase inhibitors.

### SUMMARY OF THE INVENTION

In one aspect, the invention relates to compounds that are inhibitors of Raf kinases, particularly B-Raf inhibitors. Certain hyperproliferative disorders are characterized by the overactivation of Raf kinase function, for example by mutations or overexpression of the protein. Accordingly, the compounds of the invention are useful in the treatment of hyperproliferative disorders, such as cancer.

More specifically, one aspect of the present invention provides compounds of Formula I:

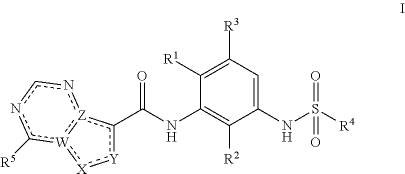

and stereoisomers, tautomers and pharmaceutically acceptable salts thereof, wherein R¹, R², R³, R⁴, R⁵, R⁶, W, X, Y and Z are as defined herein.

Another aspect of the present invention provides methods of preventing or treating a disease or disorder modulated by B-Raf, comprising administering to a mammal in need of such treatment an effective amount of a compound of this invention or a stereoisomer, tautomer, prodrug or pharmaceutically acceptable salt thereof. Examples of such diseases and disorders include, but are not limited to, hyperproliferative disorders (such as cancer, including melanoma and other cancers of the skin), neurodegeneration, cardiac hypertrophy, pain, migraine and neurotraumatic disease.

Another aspect of the present invention provides methods of preventing or treating a disease or disorder modulated by B-Raf, comprising administering to a mammal in need of such treatment an effective amount of a compound of this invention or a stereoisomer, tautomer, or pharmaceutically acceptable salt thereof. Examples of such diseases and disorders include, but are not limited to, hyperproliferative disorders (such as cancer, including melanoma and other cancers of the skin), neurodegeneration, cardiac hypertrophy, pain, migraine and neurotraumatic disease.

Another aspect of the present invention provides methods of preventing or treating cancer, comprising administering to a mammal in need of such treatment an effective amount of a compound of this invention, or a stereoisomer, tautomer, prodrug or pharmaceutically acceptable salt thereof, alone or in combination with one or more additional compounds having anti-cancer properties.

Another aspect of the present invention provides methods of preventing or treating cancer, comprising administering to a mammal in need of such treatment an effective amount of a compound of this invention, or a stereoisomer, tautomer, or pharmaceutically acceptable salt thereof, alone or in combination with one or more additional compounds having anti-cancer properties.

Another aspect of the present invention provides a method of treating a hyperproliferative disease in a mammal comprising administering a therapeutically effective amount of a compound of this invention to the mammal.

Another aspect of the present invention provides methods of preventing or treating kidney disease, comprising administering to a mammal in need of such treatment an effective amount of a compound of this invention, or a stereoisomer, tautomer, prodrug or pharmaceutically acceptable salt thereof, alone or in combination with one or more additional compounds. Another aspect of the present invention provides methods of preventing or treating polycystic kidney disease, comprising administering to a mammal in need of such treatment an effective amount of a compound of this invention, or a stereoisomer, tautomer, prodrug or pharmaceutically acceptable salt thereof, alone or in combination with one or more additional compounds.

Another aspect of the present invention provides the compounds of the present invention for use in therapy.

Another aspect of the present invention provides the compounds of the present invention for use in the treatment of a hyperproliferative disease. In a further embodiment, the hyperproliferative disease may be cancer (or still further, a specific cancer as defined herein).

Another aspect of the present invention provides the compounds of the present invention for use in the treatment of a kidney disease. In a further embodiment, the kidney disease may be polycystic kidney disease.

Another aspect of the present invention provides the use of a compound of this invention in the manufacture of a medicament for the treatment of a hyperproliferative disease. In a further embodiment, the hyperproliferative disease may be cancer (or still further, a specific cancer as defined herein).

Another aspect of the present invention provides the use of a compound of this invention in the manufacture of a medicament for the treatment of a kidney disease. In a further embodiment, the kidney disease may be polycystic kidney disease.

Another aspect of the present invention provides the use of a compound of the present invention in the manufacture of a medicament, for use as a B-Raf inhibitor in the treatment of a patient undergoing cancer therapy.

Another aspect of the present invention provides the use of a compound of the present invention in the manufacture of a medicament, for use as a B-Raf inhibitor in the treatment of a patient undergoing polycystic kidney disease therapy.

Another aspect of the present invention provides a pharmaceutical composition comprising a compound of the present invention for use in the treatment of a hyperproliferative disease.

Another aspect of the present invention provides a pharmaceutical composition comprising a compound of the present invention for use in the treatment of cancer.

Another aspect of the present invention provides a pharmaceutical composition comprising a compound of the present invention for use in the treatment of polycystic kidney disease.

Another aspect of the present invention provides a pharmaceutical composition comprising a compound of this invention, a stereoisomer, tautomer, prodrug or a pharmaceutically acceptable salt thereof, and a pharmaceutically acceptable carrier or excipient.

Another aspect of the present invention provides a pharmaceutical composition comprising a compound of this invention or a pharmaceutically acceptable salt thereof, and a pharmaceutically acceptable carrier or excipient.

Another aspect of the present invention provides intermediates for preparing compounds of Formulas I-XXV. Certain compounds of Formulas I-XXV may be used as intermediates for other compounds of Formulas I-XXV.

Another aspect of the present invention includes methods of preparing, methods of separation, and methods of purification of the compounds of this invention.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

### DEFINITIONS

The term “alkyl” includes linear or branched-chain radicals of carbon atoms. In one example, the alkyl radical is one to six carbon atoms (C₁-C₆). In other examples, the alkyl radical is C₁-C₅, C₁-C₄ or C₁-C₃. C₀ refers to a bond. Some alkyl moieties have been abbreviated, for example, methyl (“Me”), ethyl (“Et”), propyl (“Pr”) and butyl (“Bu”), and further abbreviations are used to designate specific isomers of compounds, for example, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”) and the like. Other examples of alkyl groups include 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂) and 3,3-dimethyl-2-butyl (—CH(CH₃)C(CH₃)₃. The abbreviations are sometimes used in conjunction with elemental abbreviations and chemical structures, for example, methanol (“MeOH”) or ethanol (“EtOH”).

Additional abbreviations used throughout the application include, for example, benzyl (“Bn”), phenyl (“Ph”) and acetyl (“Ac”).

The following terms are abbreviated: dimethylsulfoxide (“DMSO”), dimethylformamide (“DMF”), dichloromethane (“DCM”), ethylacetate (“EtOAc”) and tetrahydrofuran (“THF”).

The term “alkenyl” refers to linear or branched-chain monovalent hydrocarbon radical with at least one site of unsaturation, i.e., a carbon-carbon double bond, wherein the alkenyl radical may be optionally substituted independently with one or more substituents described herein, and includes radicals having “cis” and “trans” orientations, or alternatively, “E” and “Z” orientations. In one example, the alkenyl radical is two to six carbon atoms (C₂-C₆). In other examples, the alkenyl radical is C₂-C₅, C₂-C₄ or C₂-C₃. Examples include, but are not limited to, ethenyl or vinyl (—CH═CH₂), prop-1-enyl (—CH═CHCH₃), prop-2-enyl (—CH₂CH═CH₂), 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, hexa-1,3-dienyl.

The term “alkynyl” refers to a linear or branched monovalent hydrocarbon radical with at least one site of unsaturation, i.e., a carbon-carbon, triple bond, wherein the alkynyl radical may be optionally substituted independently with one or more substituents described herein. In one example, the alkynyl radical is two to six carbon atoms (C₂-C₆). In other examples, the alkynyl radical is C₂-C₅, C₂-C₄ or C₂-C₃. Examples include, but are not limited to, ethynyl (—C≡CH), prop-1-ynyl (—C≡CCH₃), prop-2-ynyl (propargyl, CH₂C≡CH), but-1-ynyl, but-2-ynyl and but-3-ynyl.

The term “alkoxy” refers to a linear or branched monovalent radical represented by the formula —OR in which R is alkyl, alkenyl, alkynyl or cycloalkyl, which can be further optionally substituted as defined herein. Alkoxy groups include methoxy, ethoxy, 2-methoxyethoxy, propoxy, isopropoxy, mono-, di- and tri-fluoromethoxy and cyclopropoxy.

“Cycloalkyl” refers to a non-aromatic, saturated or partially unsaturated hydrocarbon ring group wherein the cycloalkyl group may be optionally substituted independently with one or more substituents described herein. In one example, the cycloalkyl group is 3 to 6 carbon atoms (C₃-C₆). In other examples, cycloalkyl is C₃-C₄ or C₃-C₅. In other examples, the cycloalkyl group, as a monocycle, is C₃-C₆ or C₅-C₆. In another example, the cycloalkyl group, as a bicycle, is C₇-C₁₂. Examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl. Exemplary arrangements of bicyclic cycloalkyls having 7 to 12 ring atoms include, but are not limited to, [4,4], [4,5], [5,5], [5,6] or [6,6] ring systems. Exemplary bridged bicyclic cycloalkyls include, but are not limited to, bicyclo[2.2.1]heptane, bicyclo[2.2.2]octane, and bicyclo[3.2.2]nonane.

The terms “heterocyclic” or “heterocycle” or “heterocyclyl” refers to a saturated or a partially unsaturated (i.e., having one or more double and/or triple bonds within the ring) cyclic group in which at least one ring atom is a heteroatom independently selected from nitrogen, oxygen, and sulfur, the remaining ring atoms being carbon. In one embodiment, heterocyclyl includes saturated or partially unsaturated 4-6 membered heterocyclyl groups, another embodiment includes 5-6 membered heterocyclyl groups. The heterocyclyl group may be optionally substituted with one or more substituents described herein. Exemplary heterocyclyl groups include, but are not limited to, oxiranyl, aziridinyl, thiiranyl, azetidinyl, oxetanyl, thietanyl, 1,2-dithietanyl, 1,3-dithietanyl, pyrrolidinyl, piperidinyl, dihydropyridinyl, tetrahydropyridinyl, morpholinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, imidazolidinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Heterocycles include 4 to 6 membered rings containing one or two heteroatoms selected from oxygen, nitrogen and sulfur.

The term “heteroaryl” refers to an aromatic cyclic group in which at least one ring atom is a heteroatom independently selected from nitrogen, oxygen and sulfur, the remaining ring atoms being carbon. Heteroaryl groups may be optionally substituted with one or more substituents described herein. In one example, heteroaryl includes 5-6 membered heteroaryl groups. Other examples of heteroaryl groups include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, and furopyridinyl. Heteroaryls includes 5 to 6 membered aromatic rings containing one, two or three heteroatoms selected from oxygen, nitrogen and sulfur.

“Halogen” refers to F, Cl, Br or I.

The abbreviation “TLC” stands for thin layer chromatography.

The terms “treat” or “treatment” refer to therapeutic, prophylactic, palliative or preventative measures. In one example, treatment includes therapeutic and palliative treatment. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be prevented.

The phrases “therapeutically effective amount” or “effective amount” mean an amount of a compound of the present invention that, when administered to a mammal in need of such treatment, sufficient to (i) treat or prevent the particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound that will correspond to such an amount will vary depending upon factors such as the particular compound, disease condition and its severity, the identity (e.g., weight) of the mammal in need of treatment, but can nevertheless be routinely determined by one skilled in the art.

The terms “cancer” and “cancerous” refer to or describe the physiological condition in mammals that is typically characterized by abnormal or unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, as well as head and neck cancer.

The term cancer may be used generically to include various types of cancer or specifically (as listed above).

The phrase “pharmaceutically acceptable” indicates that the substance or composition is compatible chemically and/or toxicologically, with the other ingredients comprising a formulation, and/or the mammal being treated therewith.

The phrase “pharmaceutically acceptable salt,” as used herein, refers to pharmaceutically acceptable organic or inorganic salts of a compound of the invention.

The compounds of this invention also include other salts of such compounds which are not necessarily pharmaceutically acceptable salts, and which may be useful as intermediates for preparing and/or purifying compounds of this invention and/or for separating enantiomers of compounds of this invention.

The term “mammal” means a warm-blooded animal that has or is at risk of developing a disease described herein and includes, but is not limited to, guinea pigs, dogs, cats, rats, mice, hamsters, and primates, including humans.

The terms “compound of this invention,” and “compounds of the present invention”, unless otherwise indicated, include compounds of Formulas I-XXV, stereoisomers, tautomers, solvates, metabolites, salts (e.g., pharmaceutically acceptable salts) and prodrugs thereof. Unless otherwise stated, structures depicted herein are also meant to include compounds that differ only in the presence of one or more isotopically enriched atoms. For example, compounds of Formulas I-XXV, wherein one or more hydrogen atoms are replaced deuterium or tritium, or one or more carbon atoms are replaced by a ¹³C- or ¹⁴C-enriched carbon are within the scope of this invention.

### B-RAF Inhibitor Compounds

The present invention provides compounds, and pharmaceutical formulations thereof, that are potentially useful in the treatment of diseases, conditions and/or disorders modulated by B-Raf.

One embodiment of this invention provides compounds of Formula I:

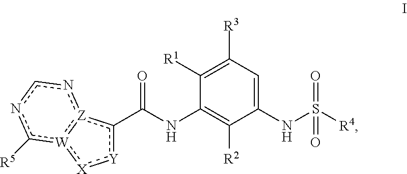

stereoisomers, tautomers, prodrugs and pharmaceutically acceptable salts thereof, wherein:

the dashed lines represent optional double bonds such that the bicycle containing the double bonds is aromatic;

W and Z are independently C or N;

X is O, S, NR⁶ or CR⁶, and Y is NR⁷ or CR⁷; or X is NR⁶ or CR⁶, and Y is O, S, NR⁷ or CR⁷; provided at least one of W, X, Y and Z is other than C, CR⁶ and CR⁷;

R¹ and R² are independently selected from hydrogen, halogen, CN, C₁-C₃ alkyl, C₁-C₃ alkoxy, C₁-C₃ alkenyl and C₁-C₃ alkynyl;

R³ is hydrogen, halogen or C₁-C₃ alkyl;

R⁴ is C₃-C₆ cycloalkyl, C₁-C₆ alkyl, C₂-C₆ alkenyl, C₂-C₆ alkynyl, phenyl, 3-6 membered heterocyclyl, a 5-6 membered heteroaryl, or NR⁸R⁹, wherein the cycloalkyl, alkyl, alkenyl, alkynyl, phenyl, heterocyclyl and heteroaryl are optionally substituted with OR²⁰, halogen, phenyl, C₃-C₆ cycloalkyl, or C₁-C₆ alkyl optionally substituted with halogen;

R⁵ is hydrogen, C₁-C₃ alkyl optionally substituted by halogen, or NR¹⁰R¹¹;

R⁶ is hydrogen, C₁-C₆ alkoxy or C₁-C₆ alkyl, wherein each C₁-C₆ alkoxy and C₁-C₆ alkyl is optionally substituted with halogen, OR²⁰, SR²⁰, NR¹⁴R¹⁵, C₃-C₆ cycloalkyl, 4-6 membered heterocyclyl, 5-6 membered heteroaryl or phenyl; provided when X is NR⁶ and is double bonded to an adjacent atom in formula I then R⁶ is absent;

R⁷ is hydrogen, C₁-C₆ alkoxy or C₁-C₆ alkyl, wherein each C₁-C₆ alkoxy and C₁-C₆ alkyl is optionally substituted with halogen, OR²⁰, SR²⁰, NR¹⁶R¹⁷, C₃-C₆ cycloalkyl, 4-6 membered heterocyclyl, 5-6 membered heteroaryl or phenyl; provided when Y is NR⁷ and is double bonded to an adjacent atom in formula I then R⁷ is absent;

R⁸ and R⁹ are each independently hydrogen or C₁-C₆ alkyl optionally substituted by halogen; or R⁸ and R⁹ are independently taken together with the atom to which they are attached to form a 3-6 membered heterocyclyl, optionally substituted by halogen, oxo or C₁-C₃ alkyl;

R¹⁰ is hydrogen;

R¹¹ is hydrogen, —(C₀-C₃ alkyl)CN, (C₀-C₃ alkyl)NR¹²R¹³, (C₀-C₃ alkyl)OR²⁰, (C₁-C₃ alkyl)SR²⁰, C₁-C₆ alkyl, C₂-C₆ alkenyl, C₂-C₆ alkynyl, (C₀-C₃ alkyl)C₃-C₆ cycloalkyl, (C₀-C₃ alkyl)phenyl, (C₀-C₃ alkyl) 3-6-membered heterocyclyl or (C₀-C₃ alkyl) 5-6-membered heteroaryl, wherein said alkyl, alkenyl, alkynyl, cycloalkyl, heterocyclyl, heteroaryl and phenyl are optionally substituted by halogen, oxo, OR²¹, NR¹⁸R¹⁹ or C₁-C₃ alkyl;

R¹² and R¹³ are independently hydrogen or C₁-C₆ alkyl optionally substituted by halogen; or R¹² and R¹³ are taken together with the atom to which they attached to form a 3-6 membered heterocyclyl, optionally substituted by halogen, oxo or C₁-C₃ alkyl;

R¹⁴ and R¹⁵ are independently hydrogen or C₁-C₆ alkyl optionally substituted by halogen; or R¹⁴ and R¹⁵ are taken together with the atom to which they attached to form a 3-6 membered heterocyclyl optionally substituted by halogen, oxo or C₁-C₃ alkyl;

R¹⁶ and R¹⁷ are independently hydrogen or C₁-C₆ alkyl optionally substituted by halogen; or R¹⁶ and R¹⁷ are taken together with the atom to which they attached to form a 3-6 membered heterocyclyl optionally substituted by halogen, oxo or C₁-C₃ alkyl;

R¹⁸ and R¹⁹ are independently hydrogen or C₁-C₆ alkyl optionally substituted by halogen; or R¹⁸ and R¹⁹ are taken together with the atom to which they attached to form a 3-6 membered heterocyclyl optionally substituted by halogen, oxo or C₁-C₃ alkyl;

each R²⁰ is independently hydrogen or C₁-C₆ alkyl optionally substituted by halogen; and

each R²¹ is independently hydrogen or C₁-C₆ alkyl optionally substituted by halogen.

Another embodiment includes compounds of Formula I, stereoisomers, tautomers, prodrugs and pharmaceutically acceptable salts thereof, wherein:

the dashed lines represent optional double bonds such that the bicycle containing the double bonds is aromatic;

W and Z are independently C or N;

X is O, S, NR⁶ or CR⁶, and Y is NR⁷ or CR⁷; or X is NR⁶ or CR⁶, and Y is O, S, NR⁷ or CR⁷; provided at least one of W, X, Y and Z is other than C, CR⁶ and CR⁷;

R¹ and R² are independently selected from hydrogen, halogen, CN, C₁-C₃ alkyl, C₁-C₃ alkoxy, C₁-C₃ alkenyl and C₁-C₃ alkynyl;

R³ is hydrogen, halogen or C₁-C₃ alkyl;

R⁴ is C₃-C₅ cycloalkyl, C₁-C₆ alkyl, C₂-C₆ alkenyl, C₂-C₆ alkynyl, phenyl, a 5-6 membered heteroaryl, or NR⁸R⁹, wherein the cycloalkyl, alkyl, alkenyl, alkynyl, phenyl and heteroaryl are optionally substituted with OR²⁰, halogen, phenyl, C₃-C₄ cycloalkyl, or C₁-C₄ alkyl optionally substituted with halogen;

R⁵ is hydrogen, C₁-C₃ alkyl, or NR¹⁰R¹¹;

R⁶ is hydrogen, C₁-C₆ alkoxy or C₁-C₆ alkyl, wherein each C₁-C₆ alkoxy and C₁-C₆ alkyl is optionally substituted with halogen, OR²⁰, SR²⁰, NR¹⁴R¹⁵, C₃-C₆ cycloalkyl, 4-6 membered heterocyclyl, 5-6 membered heteroaryl or phenyl; provided when X is NR⁶ and is double bonded to an adjacent atom in formula I then R⁶ is absent;

R⁷ is hydrogen, C₁-C₆ alkoxy or C₁-C₆ alkyl, wherein each C₁-C₆ alkoxy and C₁-C₆ alkyl is optionally substituted with halogen, OR²⁰, SR²⁰, NR¹⁶R¹⁷, C₃-C₆ cycloalkyl, 4-6 membered heterocyclyl, 5-6 membered heteroaryl or phenyl; provided when Y is NR⁷ and is double bonded to an adjacent atom in formula I then R⁷ is absent;

R⁸ and R⁹ are each independently hydrogen or C₁-C₆ alkyl optionally substituted by halogen; or R⁸ and R⁹ are independently taken together with the atom to which they are attached to form a 3-6 membered heterocyclyl, optionally substituted by halogen, oxo or C₁-C₃ alkyl;

R¹⁰ is hydrogen;

R¹¹ is hydrogen, —(C₀-C₃ alkyl)CN, (C₀-C₃ alkyl)NR¹²R¹³, (C₀-C₃ alkyl)OR²⁰, (C₁-C₃ alkyl)SR²⁰, C₁-C₆ alkyl, C₂-C₆ alkenyl, C₂-C₆ alkynyl, (C₀-C₃ alkyl)C₃-C₆ cycloalkyl, (C₀-C₃ alkyl)phenyl, (C₀-C₃ alkyl) 3-6-membered heterocyclyl or (C₀-C₃ alkyl) 5-6-membered heteroaryl, wherein said alkyl, alkenyl, alkynyl, cycloalkyl, heterocyclyl, heteroaryl and phenyl are optionally substituted by halogen, oxo, OR²¹, NR¹⁸R¹⁹ or C₁-C₃ alkyl;

R¹² and R¹³ are independently hydrogen or C₁-C₆ alkyl optionally substituted by halogen; or R¹² and R¹³ are taken together with the atom to which they attached to form a 3-6 membered heterocyclyl, optionally substituted by halogen, oxo or C₁-C₃ alkyl;

R¹⁴ and R¹⁵ are independently hydrogen or C₁-C₆ alkyl optionally substituted by halogen; or R¹⁴ and R¹⁵ are taken together with the atom to which they attached to form a 3-6 membered heterocyclyl optionally substituted by halogen, oxo or C₁-C₃ alkyl;

R¹⁶ and R¹⁷ are independently hydrogen or C₁-C₆ alkyl optionally substituted by halogen; or R¹⁶ and R¹⁷ are taken together with the atom to which they attached to form a 3-6 membered heterocyclyl optionally substituted by halogen, oxo or C₁-C₃ alkyl;

R¹⁸ and R¹⁹ are independently hydrogen or C₁-C₆ alkyl optionally substituted by halogen; or R¹⁸ and R¹⁹ are taken together with the atom to which they attached to form a 3-6 membered heterocyclyl optionally substituted by halogen, oxo or C₁-C₃ alkyl;

each R²⁰ is independently hydrogen or C₁-C₆ alkyl optionally substituted by halogen; and

each R²¹ is independently hydrogen or C₁-C₆ alkyl optionally substituted by halogen.

Another embodiment includes compounds of Formula I, stereoisomers, tautomers, prodrugs and pharmaceutically acceptable salts thereof, wherein:

the dashed lines represent optional double bonds such that the bicycle containing the double bonds is aromatic;

W and Z are independently C or N;

X is O, S, NR⁶ or CR⁶, and Y is NR⁷ or CR⁷; or X is NR⁶ or CR⁶, and Y is O, S, NR⁷ or CR⁷; provided at least one of W, X, Y and Z is other than C, CR⁶ and CR⁷;

R¹ and R² are independently selected from hydrogen, halogen, CN, C₁-C₃ alkyl and C₁-C₃ alkoxy;

R³ is hydrogen, halogen or C₁-C₃ alkyl;

R⁴ is C₃-C₅ cycloalkyl, C₁-C₆ alkyl, C₂-C₆ alkenyl, C₂-C₆ alkynyl, phenyl, a 5-6 membered heteroaryl, or NR⁸R⁹, wherein the cycloalkyl, alkyl, alkenyl, alkynyl, phenyl and heteroaryl are optionally substituted with OR²⁰, halogen, phenyl, C₃-C₄ cycloalkyl, or C₁-C₄ alkyl optionally substituted with halogen;

R⁵ is hydrogen or NR¹⁰R¹¹;

R⁶ is hydrogen, C₁-C₆ alkoxy or C₁-C₆ alkyl, wherein each C₁-C₆ alkoxy and C₁-C₆ alkyl is optionally substituted with halogen, OR²⁰, SR²⁰, NR¹⁴R¹⁵, C₃-C₆ cycloalkyl, 4-6 membered heterocyclyl, 5-6 membered heteroaryl or phenyl; provided when X is NR⁶ and is double bonded to an adjacent atom in formula I then R⁶ is absent;

R⁷ is hydrogen, C₁-C₆ alkoxy or C₁-C₆ alkyl, wherein each C₁-C₆ alkoxy and C₁-C₆ alkyl is optionally substituted with halogen, OR²⁰, SR²⁰, NR¹⁶R¹⁷, C₃-C₆ cycloalkyl, 4-6 membered heterocyclyl, 5-6 membered heteroaryl or phenyl; provided when Y is NR⁷ and is double bonded to an adjacent atom in formula I then R⁷ is absent;

R⁸ and R⁹ are each independently hydrogen or C₁-C₆ alkyl optionally substituted by halogen; or R⁸ and R⁹ are independently taken together with the atom to which they are attached to form a 3-6 membered heterocyclyl, optionally substituted by halogen, oxo or C₁-C₃ alkyl;

R¹⁰ is hydrogen;

R¹¹ is hydrogen, (C₀-C₃ alkyl)NR¹²R¹³, (C₀-C₃ alkyl)OR²⁰, (C₁-C₃ alkyl)SR²⁰, C₁-C₆ alkyl, C₂-C₆ alkenyl, C₂-C₆ alkynyl, (C₀-C₃ alkyl)C₃-C₆ cycloalkyl, (C₀-C₃ alkyl)phenyl, (C₀-C₃ alkyl) 3-6-membered heterocyclyl or (C₀-C₃ alkyl) 5-6-membered heteroaryl, wherein said alkyl, alkenyl, alkynyl, cycloalkyl, heterocyclyl, heteroaryl and phenyl are optionally substituted by halogen, oxo, OR²¹, NR¹⁸R¹⁹ or C₁-C₃ alkyl;

R¹² and R¹³ are independently hydrogen or C₁-C₆ alkyl optionally substituted by halogen; or R¹² and R¹³ are taken together with the atom to which they attached to form a 3-6 membered heterocyclyl, optionally substituted by halogen, oxo or C₁-C₃ alkyl;

R¹⁴ and R¹⁵ are independently hydrogen or C₁-C₆ alkyl optionally substituted by halogen; or R¹⁴ and R¹⁵ are taken together with the atom to which they attached to form a 3-6 membered heterocyclyl optionally substituted by halogen, oxo or C₁-C₃ alkyl;

R¹⁶ and R¹⁷ are independently hydrogen or C₁-C₆ alkyl optionally substituted by halogen; or R¹⁶ and R¹⁷ are taken together with the atom to which they attached to form a 3-6 membered heterocyclyl optionally substituted by halogen, oxo or C₁-C₃ alkyl;

R¹⁸ and R¹⁹ are independently hydrogen or C₁-C₆ alkyl optionally substituted by halogen; or R¹⁸ and R¹⁹ are taken together with the atom to which they attached to form a 3-6 membered heterocyclyl optionally substituted by halogen, oxo or C₁-C₃ alkyl;

each R²⁰ is independently hydrogen or C₁-C₆ alkyl optionally substituted by halogen; and

each R²¹ is independently hydrogen or C₁-C₆ alkyl optionally substituted by halogen.

One embodiment of this invention provides compounds of Formula I, stereoisomers, tautomers and pharmaceutically acceptable salts thereof.

In certain embodiments, W is C.

In certain embodiments, W and Z are C.

In certain embodiments, X is CR⁶ and Y is S.

In certain embodiments, X is NR⁶ and Y is S.

In certain embodiments, X is S and Y is N.

In certain embodiments, X is CR⁶ and Y is CR⁷.

In certain embodiments, X is O, NR⁶ or S; and Y is CR⁷.

In certain embodiments, X is S and Y is CR⁷. In one embodiment, R⁷ is hydrogen or C₁-C₆ alkyl. In another embodiment, R⁷ is hydrogen.

In certain embodiments, X is NR⁶ and Y is CR⁷. In one embodiment, R⁶ and R⁷ are independently hydrogen or C₁-C₆ alkyl. In another embodiment, R⁶ is methyl or ethyl; and R⁷ is hydrogen.

In certain embodiments, X is O and Y is CR⁷. In one embodiment, R⁷ is hydrogen or C₁-C₆ alkyl. In another embodiment, R⁷ is hydrogen.

In certain embodiments, X is NR⁶ and Y is N.

In certain embodiments, X is S, Y is CR⁷ and W and Z are C.

In certain embodiments, X is S, Y is N or CR⁷ and W and Z are C.

In certain embodiments, X is S, Y is N or CR⁷, W and Z are C, and R² is hydrogen, halogen other than F, CN, C₁-C₃ alkyl, C₁-C₃ alkoxy, C₁-C₃ alkenyl or C₁-C₃ alkynyl.

In certain embodiments, R¹ and R² are independently selected from hydrogen, halogen, CN, C₁-C₃ alkyl, C₁-C₃ alkoxy or C₁-C₃ alkynyl.

In certain embodiments, R¹ and R² are independently selected from hydrogen, halogen, CN, C₁-C₃ alkyl or C₁-C₃ alkoxy.

In certain embodiments, R¹, R² and R³ are independently selected from hydrogen, halogen or C₁-C₃ alkyl.

In certain embodiments, R¹, R² and R³ are independently selected from hydrogen, F, Cl or methyl.

In certain embodiments, R¹ is hydrogen, halogen, CN, C₁-C₃ alkyl or C₁-C₃ alkoxy.

In certain embodiments, R¹ is hydrogen.

In certain embodiments, R¹ is halogen. In certain embodiments, R¹ is F or Cl.

In certain embodiments, R¹ is C₁-C₃ alkyl. In certain embodiments, R¹ is methyl.

In certain embodiments, R² is hydrogen, halogen, CN, C₁-C₃ alkyl or C₁-C₃ alkoxy.

In certain embodiments, R² is hydrogen.

In certain embodiments, R² is halogen. In certain embodiments, R² is F or Cl.

In certain embodiments, R² is C₁-C₃ alkyl. In certain embodiments, R² is methyl.

In certain embodiments, R² is Cl.

In certain embodiments, R² is hydrogen.

In certain embodiments, R³ is hydrogen, halogen or C₁-C₃ alkyl.

In certain embodiments, R³ is hydrogen.

In certain embodiments, R³ is halogen. In certain embodiments, R³ is F or Cl.

In certain embodiments, R¹ and R² are F and R³ is hydrogen.

In certain embodiments, R¹ is hydrogen and R² and R³ are F.

In certain embodiments, R¹ is hydrogen, R² is F and R³ is Cl.

In certain embodiments, R¹ is F and R² is Cl and R³ is hydrogen.

In certain embodiments, R¹ is Cl and R² is F and R³ is hydrogen.

In certain embodiments, R¹ is F and R² and R³ are hydrogen.

In certain embodiments, R¹ and R³ are hydrogen and R² is F.

In certain embodiments, R² and R³ are F and R¹ is hydrogen.

In certain embodiments, R¹ is Cl and R² and R³ are hydrogen.

In certain embodiments, R¹, R² and R³ are F.

In certain embodiments, R¹ is F and R² is methyl and R³ is hydrogen.

In certain embodiments, R¹ is methyl and R² is F and R³ is hydrogen.

In certain embodiments, R¹ is F and R² and R³ are hydrogen.

In certain embodiments, R¹ is Cl and R² and R³ are hydrogen.

In certain embodiments, R² is F and R¹ and R³ are hydrogen.

In certain embodiments, R¹ is Cl, R² is ethynyl and R³ is hydrogen.

In certain embodiments, R¹ is H, R² is Cl and R³ is F.

In certain embodiments, R¹ and R³ are hydrogen and R² is —CN.

In certain embodiments, the residue:

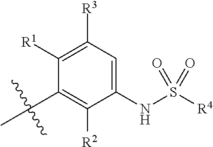

of Formula I, wherein the wavy line represents the point of attachment of the residue in Formula I, is selected from:

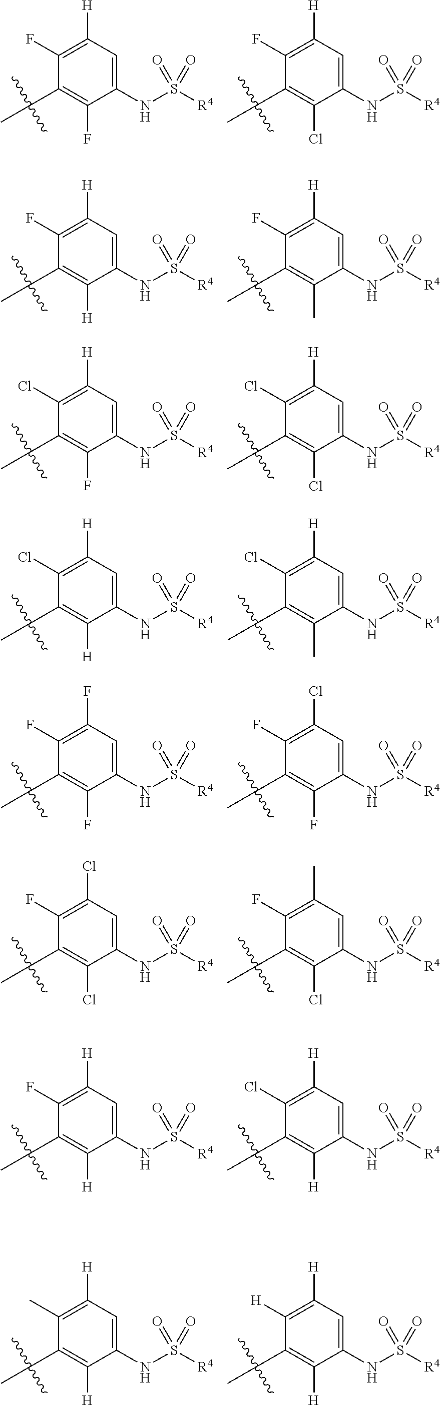

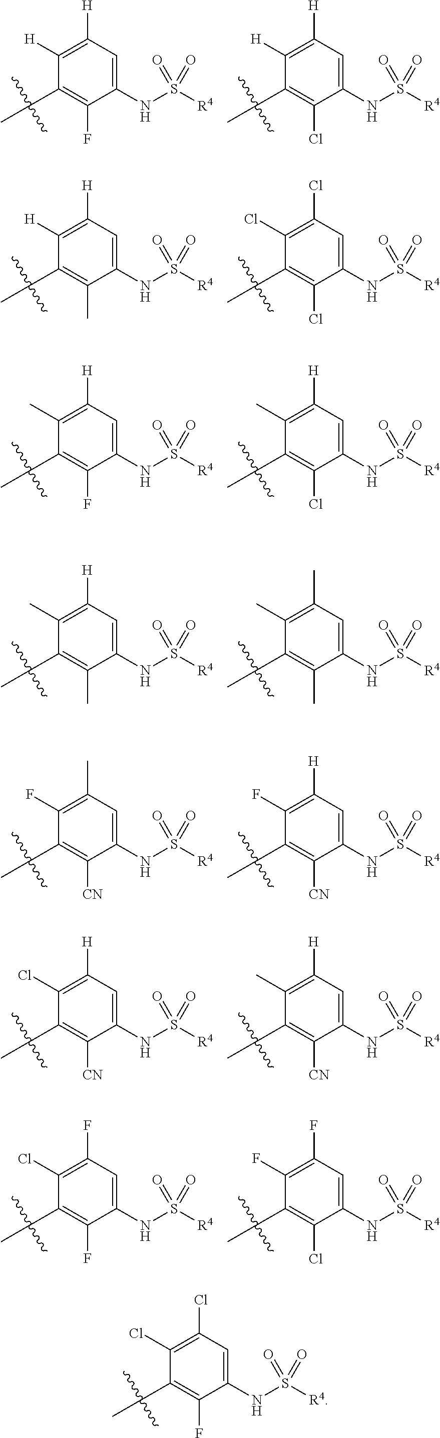

In certain embodiments, the residue:

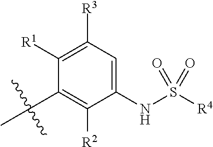

of Formula I, wherein the wavy line represents the point of attachment of the residue in Formula I, is selected from:

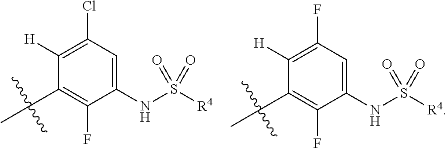

In certain embodiments, the residue:

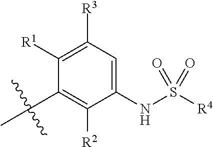

of Formula I, wherein the wavy line represents the point of attachment of the residue in Formula I, is selected from:

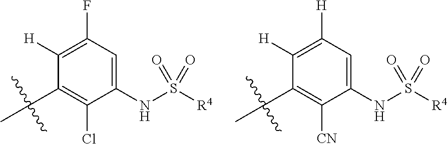

In certain embodiments, R⁴ is C₃-C₅ cycloalkyl, C₁-C₆ alkyl, C₂-C₆ alkenyl, C₂-C₆ alkynyl, phenyl, a 5-6 membered heteroaryl or NR⁸R⁹, wherein the cycloalkyl, alkyl, alkenyl, alkynyl, phenyl and heteroaryl are optionally substituted with OR²⁰, halogen, phenyl, C₃-C₄ cycloalkyl, or C₁-C₄ alkyl optionally substituted with halogen.

In certain embodiments, R⁴ is C₃-C₄ cycloalkyl, C₁-C₆ alkyl optionally substituted with halogen or C₃-C₄ cycloalkyl, or NR⁸R⁹. In certain embodiments, R⁸ and R⁹ are independently selected from hydrogen and C₁-C₅ alkyl.

In certain embodiments, R⁴ is C₃-C₅ cycloalkyl, C₁-C₆ alkyl, C₂-C₆ alkenyl or C₂-C₆ alkynyl, wherein the cycloalkyl, alkyl, alkenyl and alkynyl are optionally substituted with OR²⁰, halogen or C₃-C₄ cycloalkyl.

In certain embodiments, R⁴ is cyclopropyl, ethyl, propyl, butyl, isobutyl, —CH₂Cl, —CH₂CF₃, —CH₂CH₂CH₂F, —CH₂CH₂CF₃, phenylmethyl, cyclopropylmethyl, phenyl, 2-fluorophenyl, 3-fluorophenyl, 4-fluorophenyl, 2,5-difluorophenyl, 4-chloro-3-trifluoromethylphenyl, 1-methyl-1H-imidazol-4-yl, furan-2-yl, pyridin-2-yl, pyridin-3-yl, thiophen-2-yl, —NHCH₂CH₃, —NHCH₂CH₂CH₃, —N(CH₃)CH₂CH₃, —N(CH₃)₂, or pyrrolidine.

In certain embodiments, R⁴ is cyclopropyl, propyl, butyl, isobutyl, —CH₂Cl, —CH₂CF₃, —CH₂CH₂CH₂F, —CH₂CH₂CF₃, cyclopropylmethyl, —NHCH₂CH₂CH₃, —N(CH₃)CH₂CH₃, —N(CH₃)₂, or pyrrolidine.

In certain embodiments, R⁴ is cyclopropyl, propyl, butyl, isobutyl, —CH₂Cl, —CH₂CF₃, —CH₂CH₂CH₂F, —CH₂CH₂CF₃, cyclopropylmethyl or —NHCH₂CH₂CH₃.

In certain embodiments, R⁴ is propyl, butyl, isobutyl, —CH₂CH₂CH₂F, —CH₂CH₂CF₃ or cyclopropylmethyl.

In certain embodiments, R⁴ is C₃-C₅ cycloalkyl or C₁-C₆ alkyl optionally substituted with OH, halogen or C₃-C₄ cycloalkyl.

In certain embodiments, R⁴ is C₃-C₅ cycloalkyl. In certain embodiments, R⁴ is C₃-C₄ cycloalkyl. In certain embodiments, R⁴ is cyclopropyl or cyclobutyl. In certain embodiments, R⁴ is cyclopropyl.

In certain embodiments, R⁴ is C₁-C₆ alkyl. In certain embodiments, R⁴ is ethyl, propyl, butyl or isobutyl. In certain embodiments, R⁴ is propyl.

In certain embodiments, R⁴ is C₁-C₆ alkyl optionally substituted with halogen. In certain embodiments, R⁴ is —CF₃, —CH₂Cl, —CH₂CF₃, —CH₂CH₂CH₂F, —CH₂CH₂CF₃, —CF₂CF₃ or —CF₂CF₂CF₃. In certain embodiments, R⁴ is —CH₂CH₂CH₂F or —CH₂CH₂CF₃.

In certain embodiments, R⁴ is C₁-C₆ alkyl optionally substituted with OH, halogen or C₃-C₄ cycloalkyl. In certain embodiments, R⁴ is cyclopropylmethyl (—CH₂-cyclopropyl) or cyclobutylmethyl (—CH₂-cyclobutyl). In certain embodiments, R⁴ is cyclopropylmethyl (—CH₂-cyclopropyl).

In certain embodiments, R⁴ is C₁-C₆ alkyl optionally substituted with phenyl. In certain embodiments, R⁴ is phenylmethyl.

In certain embodiments, R⁴ is phenyl optionally substituted with OR⁸, halogen, C₃-C₄ cycloalkyl, or C₁-C₄ alkyl optionally substituted with halogen. In certain embodiments, R⁴ is phenyl optionally substituted with halogen. In certain embodiments, R⁴ is phenyl optionally substituted with C₁-C₄ alkyl optionally substituted with halogen. In certain embodiments, R⁴ is phenyl optionally substituted with halogen and C₁-C₄ alkyl optionally substituted with halogen. In certain embodiments, R⁴ is phenyl. In certain embodiments, R⁴ is phenyl, 2-fluorophenyl, 3-fluorophenyl, 4-fluorophenyl, 2,5-difluorophenyl or 4-chloro-3-trifluoromethylphenyl.

In certain embodiments, R⁴ is a 5-6 membered heteroaryl optionally substituted with OR²⁰, halogen, C₃-C₄ cycloalkyl or C₁-C₄ alkyl optionally substituted with halogen. In certain embodiments, R⁴ is a 5-6 membered heteroaryl optionally substituted with C₁-C₄ alkyl. In certain embodiments, R⁴ is a 5-6 membered heteroaryl, wherein the heteroaryl contains one or two heteroatoms selected from the group consisting of oxygen, nitrogen and sulfur. In certain embodiments, R⁴ is a 5-6 membered heteroaryl, wherein the heteroaryl is imidazolyl, furanyl, pyridinyl or thiophenyl. In certain embodiments, R⁴ is 1-methyl-1H-imidazol-4-yl, furan-2-yl, pyridin-2-yl, pyridin-3-yl or thiophen-2-yl.

In certain embodiments, R⁴ is NR⁸R⁹. In certain embodiments, R⁸ and R⁹ are independently selected from hydrogen and C₁-C₆ alkyl. In certain embodiments, R⁸ is hydrogen. In certain embodiments, R⁸ is C₁-C₆ alkyl. In certain embodiments, R⁸ is ethyl or propyl. In certain embodiments, R⁴ is selected from the group consisting of —NHCH₂CH₃, —NHCH₂CH₂CH₃, —N(CH₃)CH₂CH₃ and —N(CH₃)₂.

In certain embodiments, R⁸ and R⁹ together with the nitrogen to which they are attached form a 4 to 6 membered heterocyclic ring. In certain embodiments, R⁸ and R⁹ together with the nitrogen to which they are attached form a 4 to 6 membered heterocyclic ring, wherein the heterocyclic ring contains one nitrogen heteroatom. In certain embodiments, R⁴ is pyrrolidine. In certain embodiments, R⁴ is pyrrolidin-1-yl.

In certain embodiments, R⁴ is selected from propyl, cyclopropylmethyl, —CH₂CH₂CH₂F and phenyl. In a further embodiment, R⁴ is selected from propyl, cyclopropylmethyl and —CH₂CH₂CH₂F.

An embodiment of Formula I provides compounds of Formulas II-XIII and XXIV:

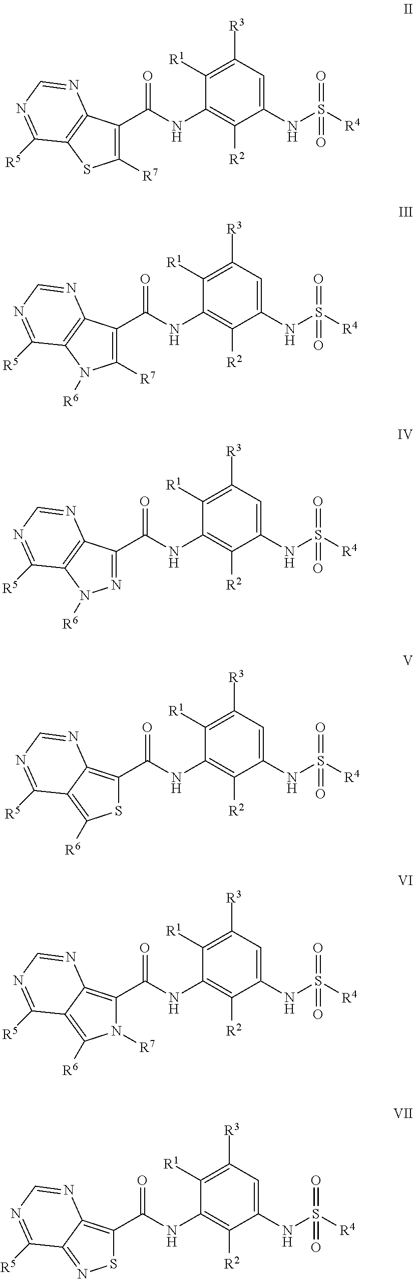

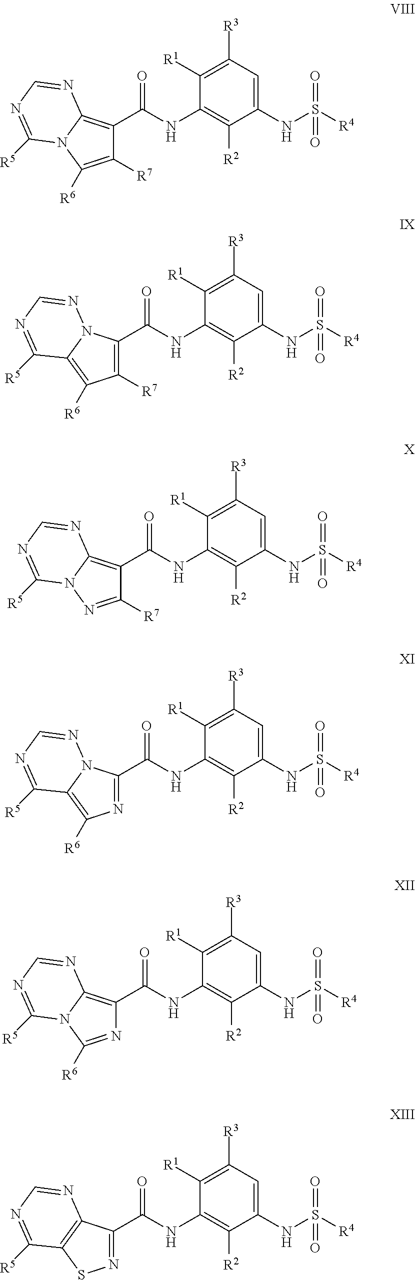

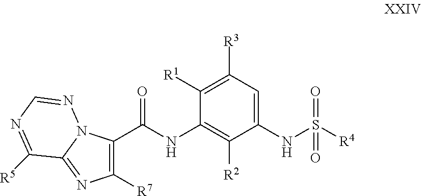

An embodiment of Formula I provides compounds of Formula XXV:

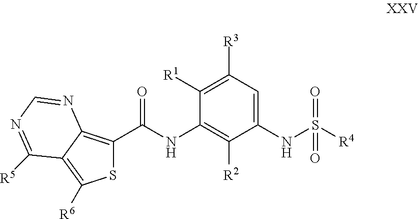

In certain embodiments of Formula I, R¹ and R² are F, R³ is hydrogen and R⁴ is propyl, such that the compounds have the structure of Formulas XIV-XV:

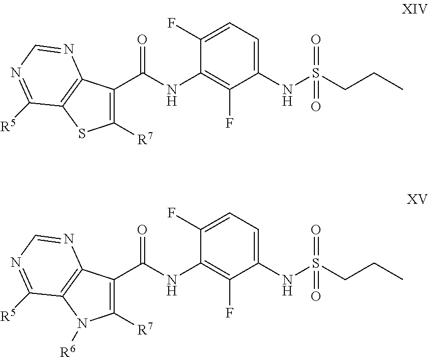

In certain embodiments of Formula I, R¹ and R² are F, R³ is hydrogen and R⁴ is 3-fluororopropyl.

In certain embodiments of Formula I, R¹ is Cl and R² is F, R³ is hydrogen and R⁴ is propyl, such that the compounds have the structure of Formulas XVI-XVII:

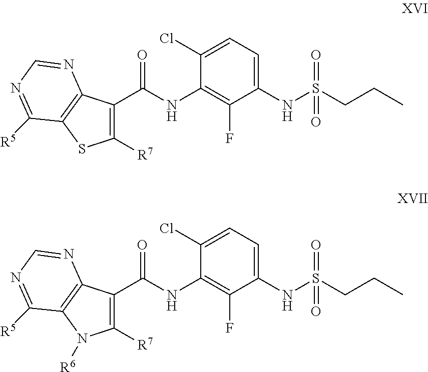

In certain embodiments of Formula I, R¹ is Cl and R² is F, R³ is hydrogen and R⁴ is 3-fluororopropyl.

In certain embodiments of Formula I, R¹ is F and R² is Cl, R³ is hydrogen and R⁴ is propyl, such that the compounds have the structure of Formula XVIII-XIX:

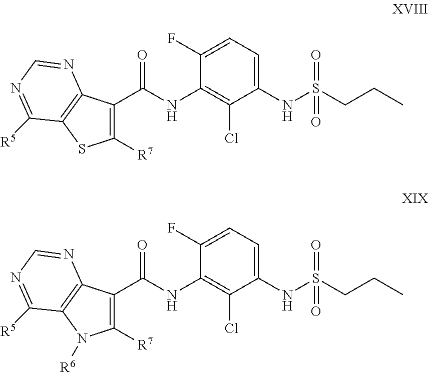

In certain embodiments of Formula I, R¹ is F and R² is Cl, R³ is hydrogen and R⁴ is 3-fluororopropyl.

In certain embodiments, R⁵ is hydrogen.

In certain embodiments, R⁵ is C₁-C₃ alkyl. In one embodiment, R⁵ is methyl.

In certain embodiments, R⁵ is C₁-C₃ alkyl optionally substituted by halogen. In one embodiment, R⁵ is methyl, ethyl, CF₃ or CHF₂.

In certain embodiments, R⁵ is NR¹⁰R¹¹, wherein R¹⁰ is hydrogen and R¹¹ is hydrogen, (C₀-C₃ alkyl)NR(C₀-C₃ alkyl)OR²⁰, (C₁-C₃ alkyl)SR²⁰, C₁-C₆ alkyl, C₂-C₆ alkenyl, C₂-C₆ alkynyl, (C₀-C₃ alkyl)C₃-C₆ cycloalkyl, (C₀-C₃ alkyl)phenyl, (C₀-C₃ alkyl) 3-6-membered heterocyclyl or (C₀-C₃ alkyl) 5-6-membered heteroaryl, wherein said alkyl, alkenyl, alkynyl, cycloalkyl, heterocyclyl, heteroaryl and phenyl are optionally substituted by halogen, oxo, OR²¹, NR¹⁸R¹⁹ or C₁-C₃ alkyl. In certain embodiments, R⁵ is —NH₂ or —NHCH₃. In certain embodiments, R⁵ is —NH₂, —NHCH₃ or —NHCH₂CH₃.

In certain embodiments, R⁵ is NR¹⁰R¹¹, R¹⁰ is hydrogen, and R¹¹ is (C₀-C₃ alkyl)NR¹²R¹³ (C₀-C₃ alkyl)OR²⁰, (C₁-C₃ alkyl)SR²⁰, C₁-C₆ alkyl, C₂-C₆ alkenyl, C₂-C₆ alkynyl, (C₀-C₃ alkyl)C₃-C₆ cycloalkyl, (C₀-C₃ alkyl)phenyl, (C₀-C₃ alkyl) 3-6-membered heterocyclyl or (C₀-C₃ alkyl) 5-6-membered heteroaryl, wherein said alkyl, alkenyl, alkynyl, cycloalkyl, heterocyclyl, heteroaryl and phenyl are optionally substituted by halogen, oxo, OR²¹, NR¹⁸R¹⁹ or C₁-C₃ alkyl.

In certain embodiments, R⁵ is NR¹⁰R¹¹, and R¹⁰ and R¹¹ are hydrogen.

In certain embodiments, R⁵ is NR¹⁰R¹¹, and R¹⁰ is hydrogen and R¹¹ is C₁-C₃ alkyl, optionally substituted by halogen. In one embodiment, R¹¹ is methyl, ethyl, n-propyl or isopropyl. In one embodiment, R¹¹ is methyl. In one embodiment, R¹¹ is methyl, ethyl, 2-fluoroethyl, 2,2-difluoroethyl, 2,2,2-trifluoroethyl, n-propyl, isopropyl, 2,2-dimethylpropyl, butyl, sec-butyl, t-butyl, pentyl or pent-2-yl. In one embodiment, R¹¹ is methyl, ethyl, 2-fluoroethyl, 2,2-difluoroethyl, 2,2,2-trifluoroethyl, n-propyl, isopropyl, 2,2-dimethylpropyl, butyl, iso-butyl, sec-butyl, t-butyl, pentyl or pent-2-yl.

In certain embodiments, R⁵ is NR¹⁰R¹¹, and R¹⁰ is hydrogen and R¹¹ is C₁-C₃ alkyl, optionally substituted by halogen or OR²¹. In one embodiment, R¹¹ is 2-hydroxyethyl or 2-methoxyethyl.

In certain embodiments, R⁵ is NR¹⁰R¹¹, and R¹⁰ is hydrogen and R¹¹ is C₃-C₆ cycloalkyl, optionally substituted by halogen. In one embodiment, R¹¹ is cyclopropyl, cyclobutyl, 3,3-difluorocyclobut-1-yl, cyclopentyl, cyclohexyl or 4,4-difluorocyclohex-1-yl.

In certain embodiments, R⁵ is NR¹⁰R¹¹, and R¹⁰ is hydrogen and R¹¹ is —(C₀-C₃ alkyl)CN. In one embodiment, R⁵ is —NH(CN).

In certain embodiments, R⁵ is NR¹⁰R¹¹, and R¹⁰ is hydrogen and R¹¹ is (C₀-C₃ alkyl)OR²⁰. In certain embodiments, R⁵ is NR¹⁰R¹¹, R¹⁰ is hydrogen, and R¹¹ is OH, OCH₃, CH₂CH₂OH, CH₂CH₂OCH₃. In one embodiment, R⁵ is —NH(OH) or —NH(OCH₃). In one embodiment, R⁵ is —NH(OH), —NHCH₂CH₂OH, —NHCH₂CH₂OCH₃ or —NH(OCH₃).

In certain embodiments, R⁵ is NR¹⁰R¹¹, R¹⁰ is hydrogen, and R¹¹ is (C₀-C₃ alkyl)NR¹²R¹³ and R¹² and R¹³ are hydrogen or C₁-C₃ alkyl. In certain embodiments, R⁵ is CH₂CH₂N(CH₃)₂.

In certain embodiments, R⁵ is NR¹⁰R¹¹, and R¹⁰ is hydrogen and R¹¹ is (C₀-C₃ alkyl) 5-6-membered heteroaryl optionally substituted by halogen, oxo, OR²¹, NR¹⁸R¹⁹ or C₁-C₃ alkyl. In certain embodiments, R⁵ is NR¹⁰R¹¹, and R¹⁰ is hydrogen and R¹¹ is pyrazolyl or pyridinyl optionally substituted by C₁-C₃ alkyl. In certain embodiments, R¹¹ is 1-methylpyrazol-4-yl or pyridin-2-yl.

In certain embodiments, R⁵ is NR¹⁰R¹¹, and R¹⁰ is hydrogen and R¹¹ is (C₀-C₃ alkyl) 3-6-membered heterocyclyl optionally substituted by halogen, oxo, OR²¹, NR¹⁸R¹⁹ or C₁-C₃ alkyl. In certain embodiments, R⁵ is NR¹⁰R¹¹, and R¹⁰ is hydrogen and R¹¹ is azetidinyl, oxetanyl, tetrahydrofuranyl, tetrahydropyranyl, piperidinyl, piperazinyl or morpholinyl, optionally substituted by optionally substituted by C₁-C₃ alkyl. In certain embodiments, R¹¹ is N-methylazetidin-3-yl, tetrahydrofuranyl, tetrahydropyranyl, piperidinyl, piperazinyl or morpholinyl.

In certain embodiments, R⁶ is hydrogen or C₁-C₆ alkyl, optionally substituted by halogen. In certain embodiments, R⁶ is hydrogen or methyl. In certain embodiments, R⁶ is methyl.

In certain embodiments, R⁷ is hydrogen or C₁-C₆ alkyl, optionally substituted by halogen. In certain embodiments, R⁷ is hydrogen or methyl. In certain embodiments, R⁷ is methyl.

Another embodiment includes compounds of Formulas XX-XXIII:

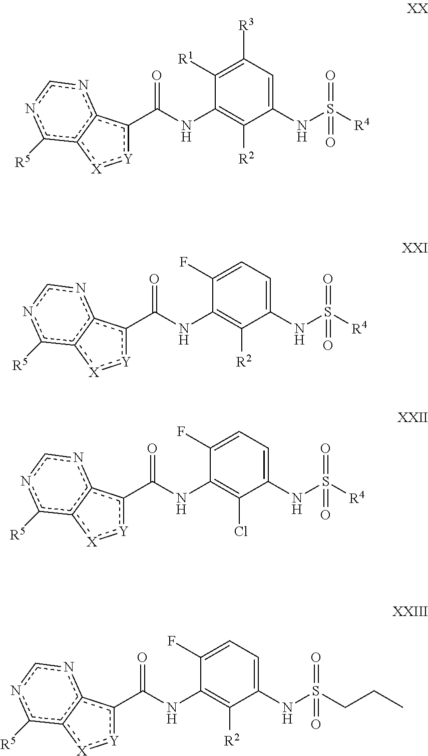

and stereoisomers, tautomers and pharmaceutically acceptable salts thereof, wherein R¹, R², R³, R⁴, R⁵, X and Y are as defined herein.

Another embodiment includes compounds of Formula I, stereoisomers, tautomers and pharmaceutically acceptable salts thereof, wherein W and Z are C, R¹ is Cl, R³ is hydrogen and R⁴ is 3-fluoropropyl.

Another embodiment includes compounds of Formula I, stereoisomers, tautomers and pharmaceutically acceptable salts thereof, wherein W and Z are C, Y is CR⁷, R¹ is Cl, R³ is hydrogen, R⁴ is 3-fluoropropyl and R⁷ is hydrogen.

It will be appreciated that certain compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

It will also be appreciated that compounds of Formulas I-XXV include tautomeric forms. Tautomers are compounds that are interconvertible by tautomerization. This commonly occurs due to the migration of a hydrogen atom or proton, accompanied by the switch of a single bond and adjacent double bond. For instance, 1H-pyrrolo[2,3-b]pyridine is one of the tautomeric forms of 7-azaindole. Another tautomeric form of 7-azaindole is 7H-pyrrolo[2,3-b]pyridine. Other tautomers of Formulas I-XXV may also form at other positions, including, but not limited to, the sulfonamide or R5/R6 position depending on the substitution. The compounds of Formulas I-XXV are intended to include all tautomeric forms.

It will also be appreciated that certain compounds of Formulas I-XXV may be used as intermediates for further compounds of Formulas I-XXV.

It will be further appreciated that the compounds of the present invention may exist in unsolvated, as well as solvated forms with pharmaceutically acceptable solvents, such as water, ethanol, and the like, and it is intended that the invention embrace both solvated and unsolvated forms.

The term “prodrug” as used in this application refers to a precursor or derivative form of a compound of the invention that is less active or inactive compared to the parent compound or drug and is capable of being metabolized in vivo into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, N-methyl prodrugs (including N-methyl sulphonamide prodrugs), phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs, optionally substituted phenylacetamide-containing prodrugs, 5-fluorocytosine and other 5-fluorouridine prodrugs which can be converted into the more active cytotoxic free drug.

Prodrugs of compounds of Formulas I-XXV may not be as active as the compounds of Formulas I-XXV in the assay as described in Example A. However, the prodrugs are capable of being converted in vivo into more active metabolites of compounds of the present invention.

### Synthesis of Compounds

Compounds of the present invention may be synthesized by synthetic routes that include processes analogous to those well-known in the chemical arts, particularly in light of the description contained herein. The starting materials are generally available from commercial sources such as Sigma-Aldrich (St. Louis, Mo.), Alfa Aesar (Ward Hill, Mass.), or TCI (Portland, Oreg.), or are readily prepared using methods well known to those skilled in the art (e.g., prepared by methods generally described in Louis F. Fieser and Mary Fieser, Reagents for Organic Synthesis. v. 1-23, New York: Wiley 1967-2006 ed. (also available via the Wiley InterScience® website), or Beilsteins Handbuch der organischen Chemie, 4, Aufl. ed. Springer-Verlag, Berlin, including supplements (also available via the Beilstein online database)).

In preparing compounds of The present invention, protection of remote functionalities (e.g., primary or secondary amines, etc.) of intermediates may be necessary. The need for such protection will vary depending on the nature of the remote functionality and the conditions of the preparation methods. Suitable amino-protecting groups (NH-Pg) include acetyl, trifluoroacetyl, t-butyloxycarbonyl (“Boc”), benzyloxycarbonyl (“CBz”), p-methoxybenzyl (“PMB”) and 9-fluorenylmethyleneoxycarbonyl (“Fmoc”). The need for such protection is readily determined by one skilled in the art. For a general description of protecting groups and their use, see T. W. Greene, et al. Greene's Protective Groups in Organic Synthesis. New York: Wiley Interscience, 2006.

For illustrative purposes, Schemes 1-33 show general methods for preparing the compounds of the present invention, as well as key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

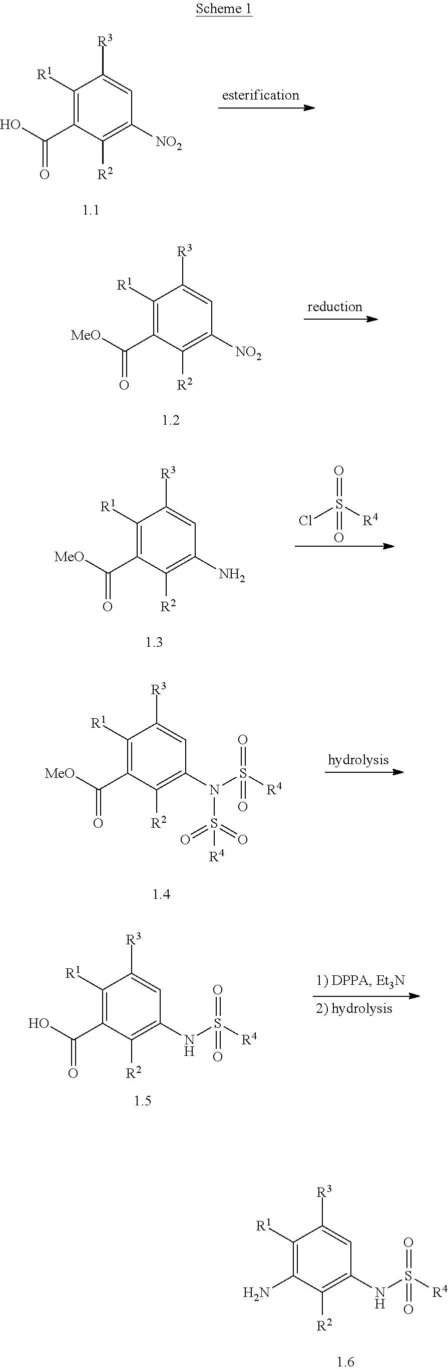

Scheme 1 shows a general method for preparing a compound 1.6, wherein R¹, R², R³ and R⁴ are as defined herein. A benzoic acid 1.1 is esterified to a methyl benzoate 1.2 by treatment with trimethylsilyl diazomethane in MeOH, or via Fischer esterification conditions, such as treatment with trimethylsilyl chloride (“TMSCl”) in MeOH. Reduction of nitro intermediate 1.2 to its amino analog 1.3 is performed using a standard condition, such as treatment with Pd/C and H₂. Bis-sulfonamide 1.4 is obtained by treatment of the aniline 1.3 with a sulfonyl chloride R⁴SO₂Cl in the presence of a base, such as NEt₃, in an organic solvent, such as dichloromethane (“DCM”). Hydrolysis of compound 1.4 is accomplished under basic conditions, such as aqueous NaOH, in the appropriate solvent system such as THF and/or MeOH, to provide a carboxylic acid 1.5. This compound in a suitable solvent, such as THF, is treated with diphenylphosphonic azide (“DPPA”) and a base, such as triethylamine (Curtius rearrangement conditions), and subsequently hydrolyzed to form an amine 1.6.

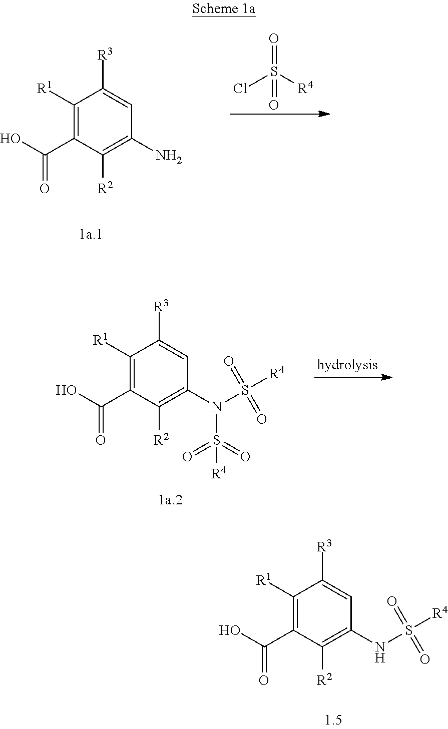

Scheme 1a shows an alternative method for the synthesis of compounds 1.5. Aminobenzoic acid 1a.1 is treated with a sulfonyl chloride R⁴SO₂Cl in the presence of a base, such as NEt₃, in an organic solvent, such as dichloromethane (“DCM”). Hydrolysis of compound 1a.2 is accomplished under basic conditions, such as aqueous NaOH, in the appropriate solvent system, such as THF and/or MeOH, to provide the mono-sulfonamide 1.5.

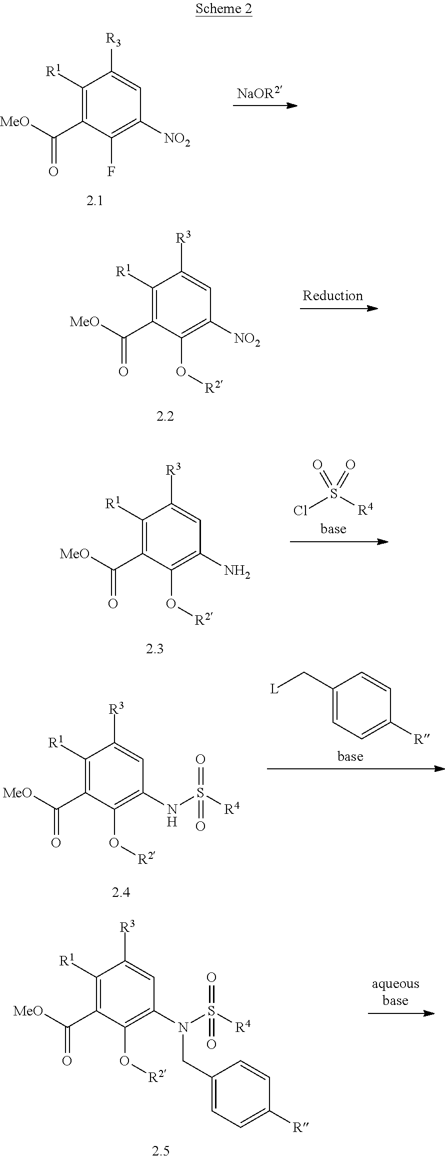

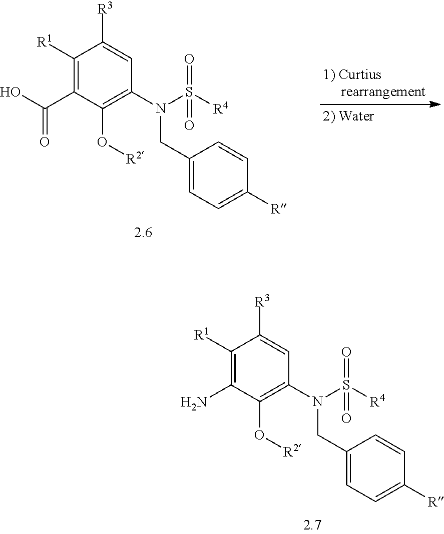

Scheme 2 describes the synthesis of aniline intermediates 2.7, wherein R¹, R^{2′}, R³ and R⁴ and R″ are as defined herein. A benzoic acid ester 2.1 is treated with an alkoxide NaOR²′ (wherein R²′ is C1-C3 alkyl) in an appropriate solvent, such as methanol, to form the ether intermediate 2.2. Reduction of the nitro group affords an aniline 2.3, which is reacted with a sulfonyl chloride

R⁴SO₂Cl in the presence of base, such as pyridine, to give a sulfonamide intermediate 2.4. Benzylation with an optionally substituted benzyl halide, for example p-methoxybenzyl chloride, (wherein L is a leaving group such as chloro, bromo, iodo, triflate, tosylate; and R″ is hydrogen, C₁-C₃ alkyl or C₁-C₆ alkoxy; and in one example, R″ is hydrogen, in another example, R″ is OMe) in the presence of a base, such as sodium hydride, yields the protected sulfonamide ester 2.5, which is hydrolyzed with aqueous base, such as NaOH, to form the acid 2.6. In the last step, application of Curtius rearrangement conditions and subsequent hydrolysis gives the amino intermediate 2.7.

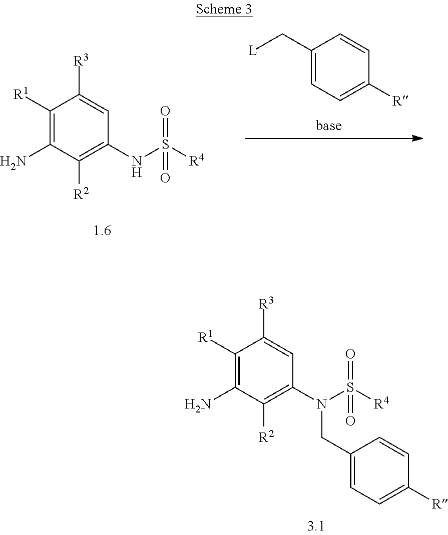

Scheme 3 shows a procedure for generating the aniline intermediate 3.1, wherein R″ and L are defined in Scheme 2 and R¹, R², R³ and R⁴ are as defined herein, through protection of the sulfonamide moiety of aniline 1.6. This transformation can be accomplished by treatment with an optionally substituted benzyl halide (e.g. p-methoxybenzyl chloride) and a base, such as sodium hydride.

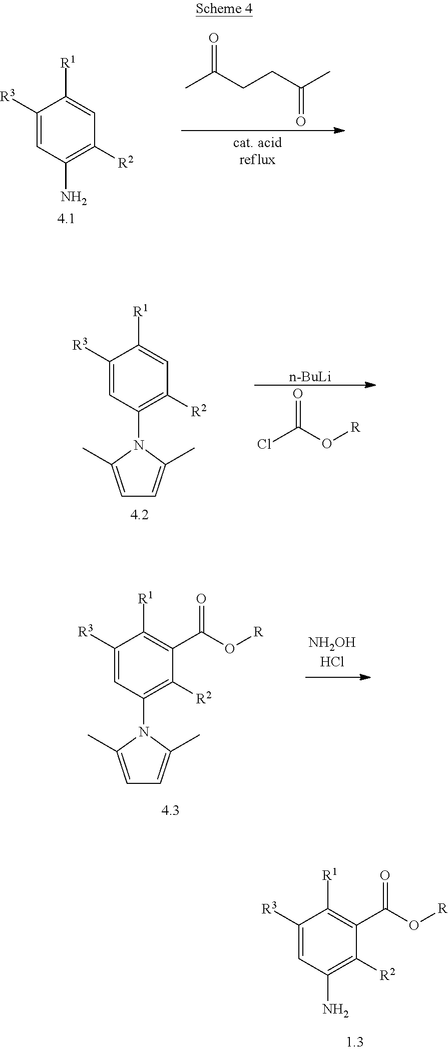

Scheme 4 describes the synthesis of an aniline ester of Formula 1.3, wherein R¹, R², and R³ are defined herein and R is alkyl such as methyl, ethyl or benzyl. The amino group of an aniline 4.1 is protected by reacting with hexane-2,5-dione in the presence of a catalytic amount of an acid, such as p-toluenesulfonic acid, in a solvent, such as toluene, to form the 2,5-dimethylpyrrole derivative 4.2. Reaction with a carbamoyl chloride RO(C═O)Cl in the presence of n-butyllithium or a comparable agent in a suitable solvent such as THF leads to formation of the ester analog 4.3. The amino function of compound 4.3 is deprotected by reaction with hydroxylamine in a suitable solvent, such as ethanol, leading to formation of intermediate 1.3.

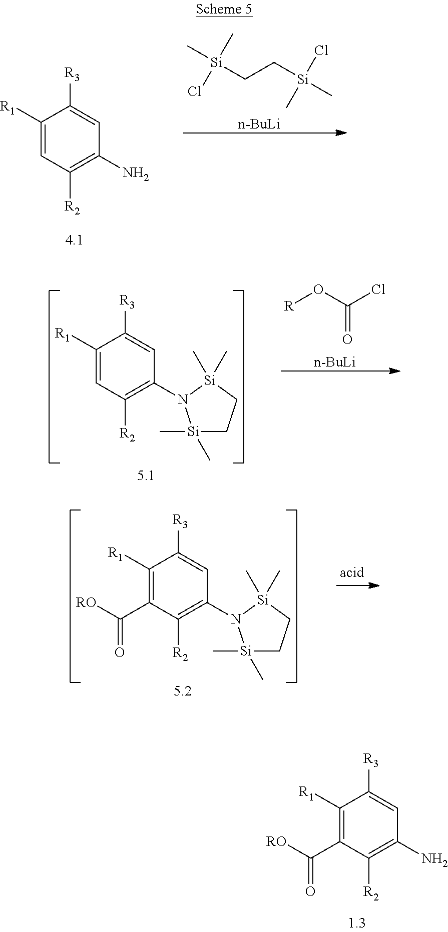

Scheme 5 describes the synthesis of an aniline ester of Formula 1.3, wherein R¹, R², and R³ are defined herein and R is alkyl such as methyl, ethyl or benzyl. The amino group of an aniline 4.1 is protected by reacting with 1,2-bis(chlorodimethylsilyl)ethane in the presence of a strong base such as n-butyllithium in a suitable solvent, such as THF, at low temperatures, e.g. −78° C., to form the 1-aza-2,5-disilacyclopentane intermediate 5.1, which is reacted with a carbamoyl chloride RO(C═O)Cl in the presence of n-butyllithium or a comparable agent in a suitable solvent such as THF leading to formation of the ester analog 5.2. The amino function of compound 5.2 is deprotected by reaction with an acid such as HCl in a suitable solvent, leading to formation of intermediate 1.3.

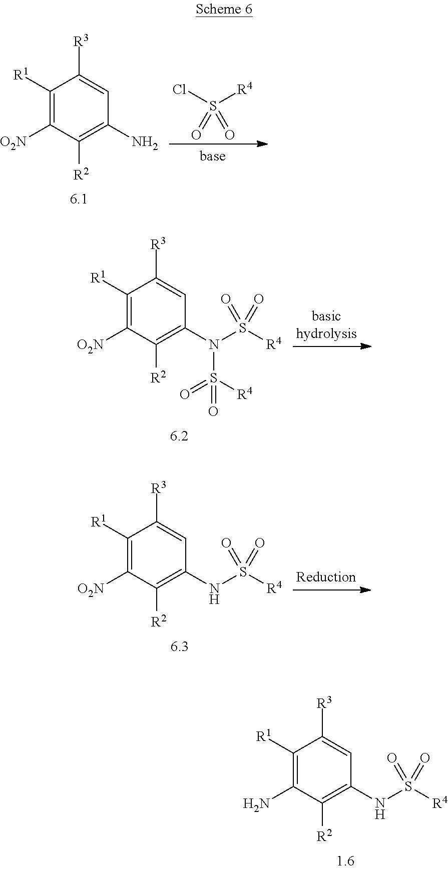

Scheme 6 describes another way of synthesizing an intermediate of Formula 1.6, wherein R¹, R², R³ and R⁴ are as defined herein. Bis-sulfonamide 6.2 is obtained by treatment of the aniline 6.1 with a sulfonyl chloride R⁴SO₂Cl in the presence of a base, such as NEt₃, in an organic solvent, such as dichloromethane. Hydrolysis of compound 6.2 is accomplished under basic conditions, such as aqueous NaOH, in the appropriate solvent system, such as THF and/or MeOH, to provide the mono-sulfonamide 6.3. This compound in a suitable solvent, such as ethanol, is treated with a reducing agent, such as iron and additional reagents, such as ammonium chloride, to form an amine 1.6.

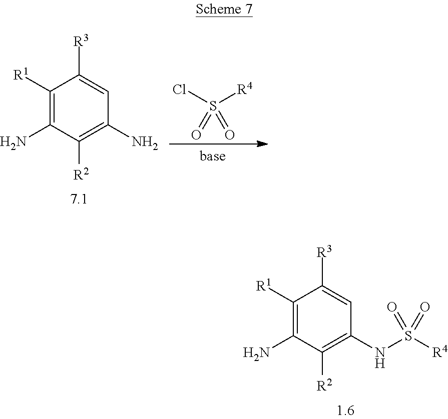

Scheme 7 shows another way of preparing an intermediate of Formula 1.6. This transformation is accomplished by mono-sulfonylation of a diamino derivative 7.1 with a sulfonyl chloride R⁴SO₂Cl in the presence of a base, such as pyridine, in an organic solvent, such as dichloromethane.

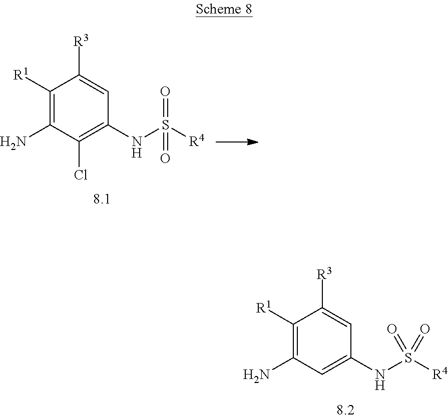

Scheme 8 describes the synthesis of an intermediate of Formula 8.2, wherein R¹, R³ and R⁴ are as defined herein and R² is hydrogen. This transformation is accomplished by using reducing conditions, such as hydrogen in the presence of a palladium catalyst, in a suitable solvent such as ethanol.

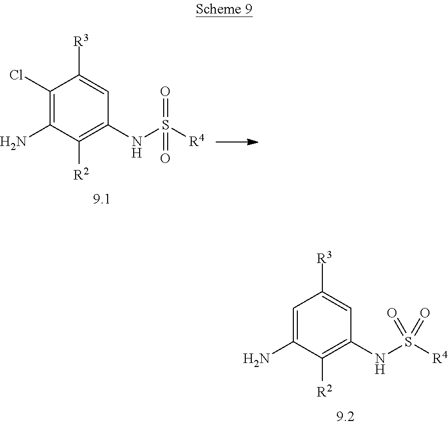

Scheme 9 describes the synthesis of an intermediate of Formula 9.2, wherein R², R³ and R⁴ are as defined herein and R¹ is hydrogen. This transformation is accomplished by using reducing conditions, such as hydrogen in the presence of a palladium catalyst, in a suitable solvent such as ethanol.

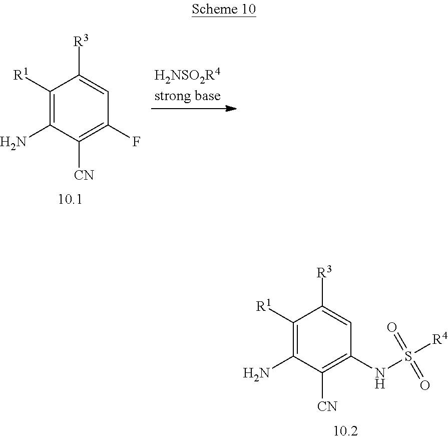

Scheme 10 shows a method for preparing nitrile-substituted aniline intermediates 10.2. Reaction of fluoronitrile 10.1 with the sodium salt of H₂NSO₂R⁴ (generated by a strong base such as sodium hydride) in a suitable solvent, such as DMSO or N-methylpyrrolidone (“NMP”) at elevated temperature, results in the formation of intermediate 10.2.

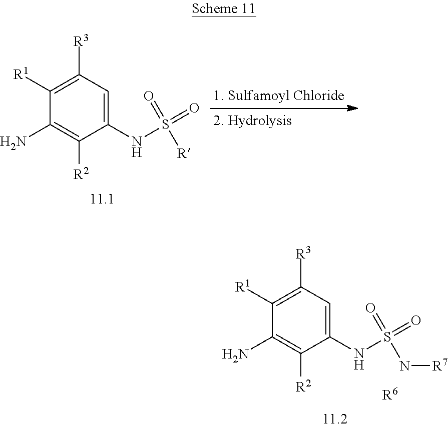

Scheme 11 shows a general method for preparing sulfamides of Formula 11.2, wherein R¹, R², R³, R⁶, and R⁷ are defined herein. A sulfonamide 11.1 (R′ is alkyl) is treated with a sulfamoyl chloride in a solvent such as DMF and subsequently hydrolyzed to a sulfamide 11.2 by addition of a base and water such as sodium hydroxide.

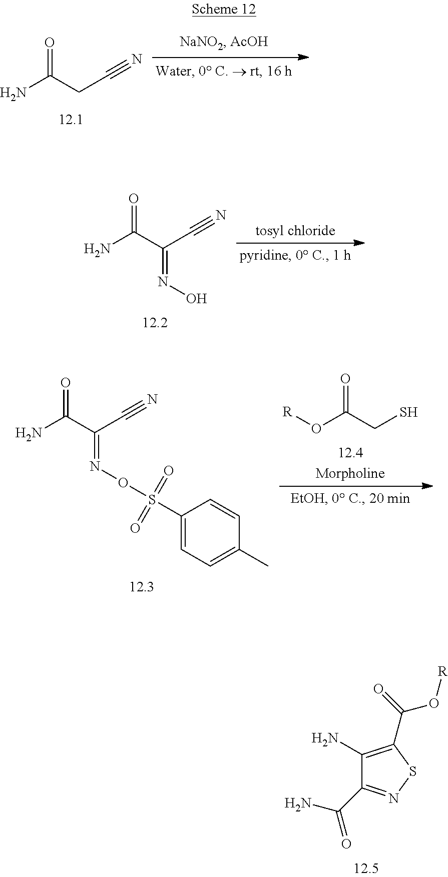

Scheme 12 describes the synthesis of an alkyl ester of 4-amino-3-carbamoylisothiazole-5-carboxylic acid. 2-Cyanoacetamide 12.1 is treated with sodium nitrite and acetic acid in a suitable solvent such as water to afford (E)-2-amino-N-hydroxy-2-oxoacetimidoyl cyanide 12.2. Reaction of 12.2 with tosyl chloride in a suitable solvent, such as pyridine gives (E)-2-amino-2-oxo-N-(tosyloxy)acetimidoyl cyanide 12.3, which is then, in the presence of a base such as morpholine and a solvent such as ethanol, treated with an alkyl 2-mercaptoacetate 12.4 (R is alkyl) to give an alkyl 4-amino-3-carbamoylisothiazole-5-carboxylate 12.5.

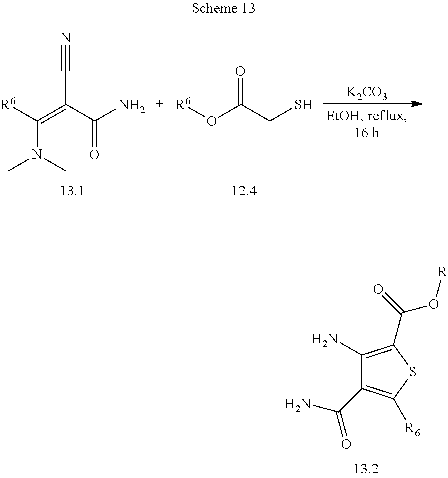

Scheme 13 describes the formation of a substituted thiophene 13.2 through reaction of the acrylamide derivative 13.1 with an alkyl 2-mercaptoacetate 12.4 (R is alkyl) in the presence of an inorganic base such as potassium carbonate and a suitable solvent such as ethanol at elevated temperatures.

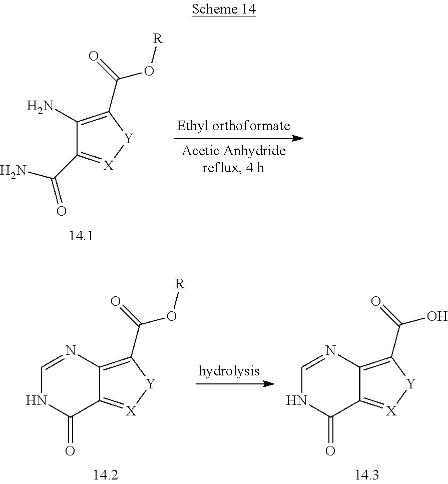

Scheme 14 describes the synthesis of the fused pyrimidone 14.3 where X and Y are as defined herein. The 5-membered heterocycle 14.1 is reacted with ethyl orthoformate in acetic anhydride to form the fused pyrimidone ester 14.2, which is subsequently hydrolyzed to the free acid 14.3 by heating in aqueous acid or base.

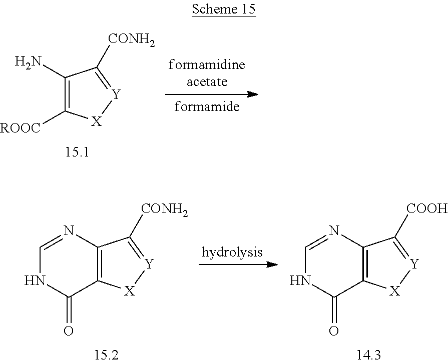

Scheme 15 outlines another method for the preparation of intermediate 14.3 where X and Y are as defined herein. Ester amide 15.1 can be cyclized to the fused pyrimidone 15.2 by heating with formamide, alternatively, with a mixture of formamidine acetate and formamide. Amide 15.2 is hydrolyzed to the corresponding acid 14.3 by heating in aqueous acid or base.

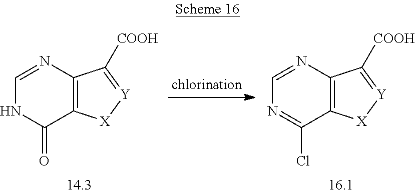

Scheme 16 describes the synthesis of the chlorinated intermediate 16.1 where X and Y are as defined herein. This transformation can be accomplished by treatment with a suitable chlorinating agent such as thionyl chloride or phosphoryl chloride in the presence of N,N-dimethylformamide and/or a base such as 2,6-lutidine using an appropriate solvent, such as acetonitrile.

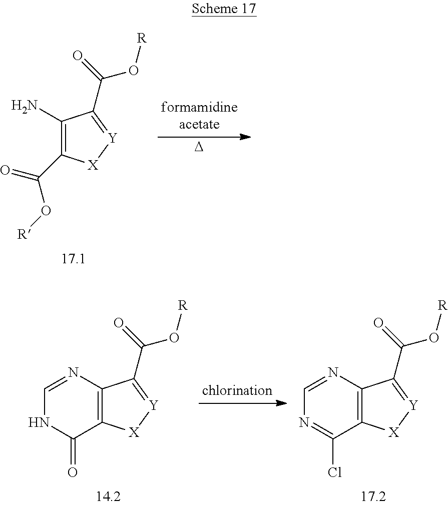

Scheme 17 describes a general method for synthesizing intermediates 17.2, wherein X and Y are as defined herein, containing a chloropyrimidine moiety that is fused to a 5-membered heterocyclic system. Treatment of the di-ester derivative 17.1 (wherein R and R′ are independently C₁-C₃ alkyl) with formamidine acetate in a suitable solvent, such as ethanol, at elevated temperatures gives the fused pyrimidone 14.2, which is further transformed with a chlorinating agent, such as POCl₃, to intermediate 17.2.

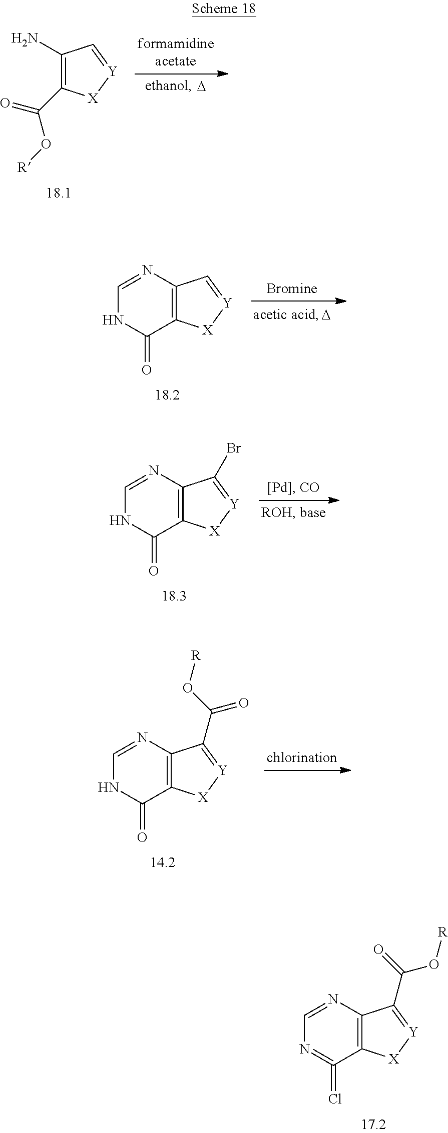

Scheme 18 describes another general method for synthesizing intermediates 17.2 containing a chloropyrimidine moiety that is fused to a 5-membered heterocyclic system, wherein R is C₁-C₃ alkyl. The amino ester derivative 18.1 is reacted with formamidine acetate in a suitable solvent, such as ethanol, at elevated temperatures to form the pyrimidone derivative 18.2. Treatment with bromine in a suitable solvent, such as acetic acid, at elevated temperatures gives the bromo intermediate 18.3. Carbonylation reaction under pressure using carbon monoxide, a suitable palladium catalyst, such as [1,1′-bis(diphenylphosphino)ferrocene]dichloropalladium(II), a base, such as triethylamine, an alcohol, such as methanol, at elevated temperatures furnishes the ester derivative 14.2, which is further transformed with a chlorinating agent such as POCl₃ to intermediate 17.2.

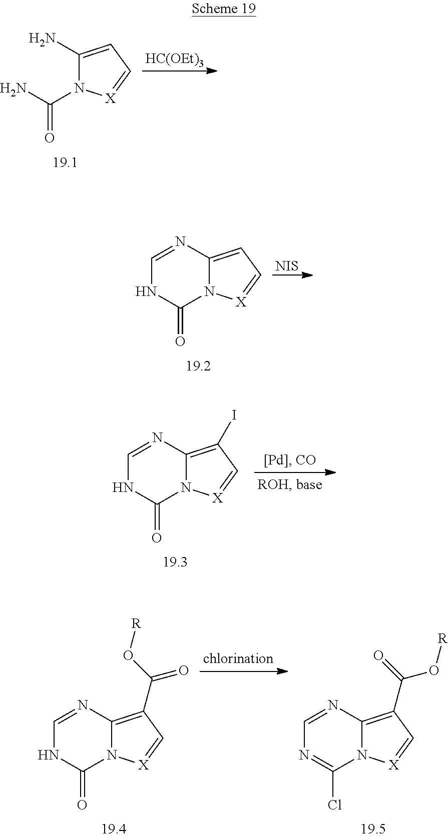

Scheme 19 describes a general method for synthesizing intermediates 19.5 containing a chlorotriazine moiety that is fused to a 5-membered heterocyclic system, wherein R is as defined in Scheme 418. The N-linked urea derivative 19.1 (Chu et al., J Het Chem (1980), 17(7), p. 1435) is reacted with triethylorthoformate in a suitable solvent, such as ethanol, at elevated temperatures to form the triazinone derivative 19.2. Treatment with N-iodosuccinimide in a suitable solvent, such as acetone, gives the iodo intermediate 19.3. Carbonylation reaction under pressure using carbon monoxide, a suitable palladium catalyst, such as [1,1′-bis(diphenylphosphino)ferrocene]dichloropalladium(II), a base, such as triethylamine, an alcohol, such as methanol, at elevated temperatures furnishes the ester derivative 19.4, which is further transformed with a chlorinating agent, such as POCl₃ to intermediate 19.5.

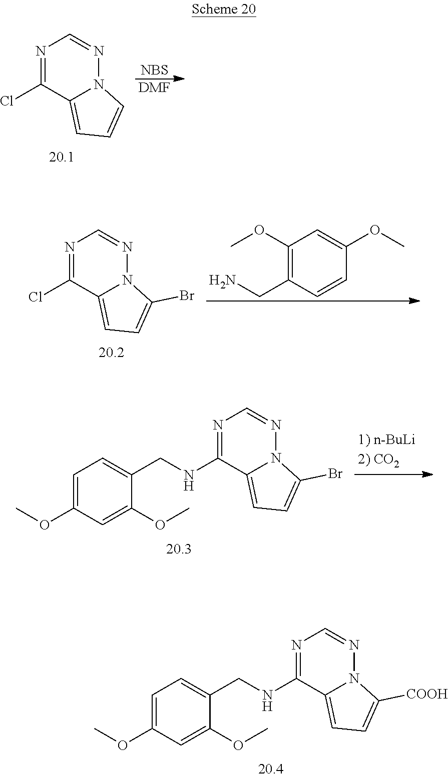

Scheme 20 describes a general method for synthesizing pyrrolo[1,2-f][1,2,4]triazines 20.4. 4-Chloropyrrolo[1,2-f][1,2,4]triazine 20.1 is reacted with N-bromosuccinimide in a suitable solvent, such as chloroform to give the bromo intermediate 20.2. Protection of the amine functionality with 2,4-dimethoxybenzyl bromide furnishes the N-protected intermedediate derivative 20.3. Subsequent treatment of 20.3 with a strong base, such as butyllithium in a suitable solvent, such as THF, and carbon dioxide (gas) gives intermediate 20.4.

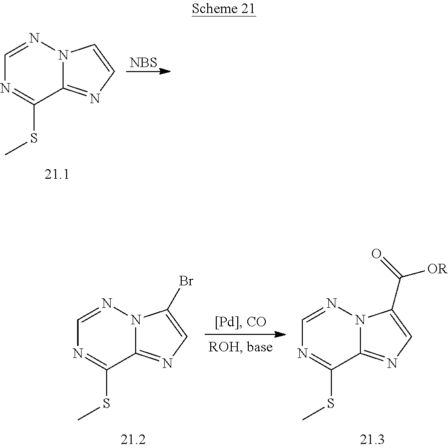

Scheme 21 describes a general method for synthesizing imidazo[1,2-f][1,2,4]triazine 21.3, wherein R is as defined in Scheme 418. 4-(Methylthio)imidazo[1,2-f][1,2,4]triazine 21.1 (Dudfield et al., J. Chem. Soc., Perkin Trans. 1 (1999), p. 2929) is reacted with N-bromosuccinimide in a suitable solvent, such as chloroform, to give the bromo intermediate 21.2. Carbonylation reaction under pressure using carbon monoxide, a suitable palladium catalyst, such as [1,1′-bis(diphenylphosphino)ferrocene]dichloropalladium(II), a base, such as triethylamine, an alcohol, such as methanol, at elevated temperatures furnishes the ester derivative 21.3.

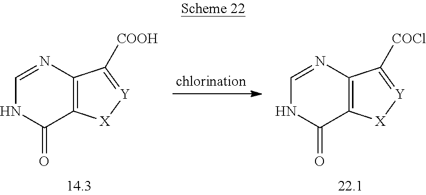

Scheme 22 describes the preparation of another intermediate acid chloride 22.1 which can be obtained from intermediate 14.3 by treatment with thionyl chloride with catalytic N,N,-dimethylformamide, or preferably, with thionyl chloride neat or in a suitable solvent such as chloroform.

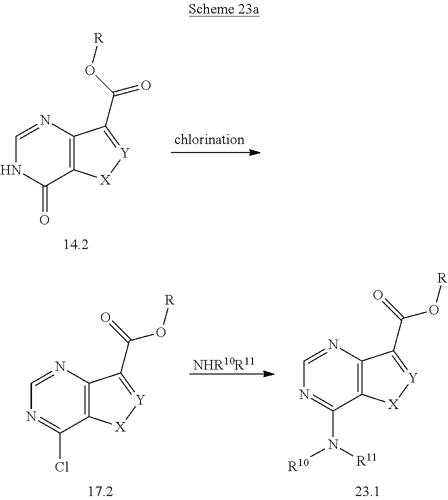

Scheme 23a describes the synthesis of an intermediate 23.1, wherein X, Y, R¹⁰, and R¹¹ are as defined herein and R is as defined in Scheme 18. Chlorination of intermediate 14.2 by treatment with thionyl chloride or phosphoryl chloride with catalytic N,N,-dimethylformamide and/or a base such as 2,6-lutidine gives intermediate 17.2 which is reacted with an amine HNR¹⁰R¹¹ to form 23.1.

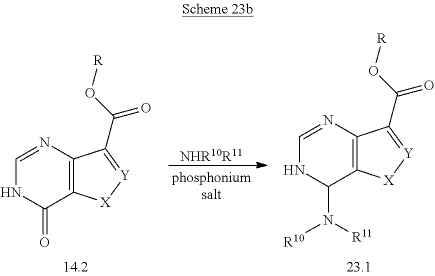

Scheme 23b describes an alternative method of synthesizing an intermediate 23.1. Pyridone 14.2 is reacted with an amine HNR¹⁰R¹¹ via coupling with a suitable phosphonium salt such as 1H-benzotriazol-1-yloxy-tris(dimethylamino)phosphonium hexafluorophosphate (“BOP”) or 1H-benzotriazol-1-yloxytris(pyrrolidino)phosphonium hexafluorophosphate (“PyBOP”) to form 23.1.

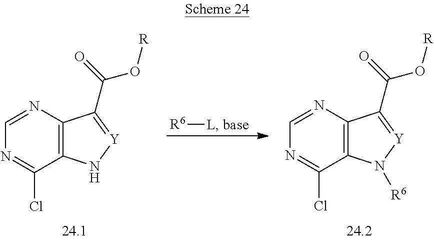

Scheme 24 describes a general method of derivatizing the amino group of the chloropyrimidine derivative 24.1 with a base, such as sodium hydride, in a suitable solvent, such as DMF, to form intermediate

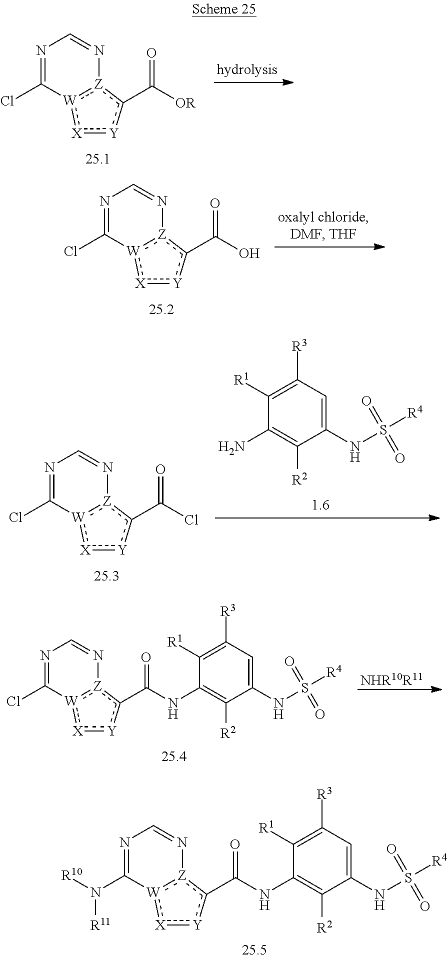

Scheme 25 describes the synthesis of compound 25.5, wherein R¹, R², R³, R⁴, R¹⁰, R¹¹, W, X, Y, and Z are as defined herein. An ester of Formula 25.1, wherein R, W, X, Y, and Z are as defined herein is hydrolyzed to the corresponding carboxylic acid derivative 25.2 using basic or acidic hydrolysis conditions, subsequently converted to its acid chloride derivative 25.3, e.g. by treatment with oxalyl chloride and catalytic DMF in a solvent, such as THF, and further reacted with aniline derivative 1.6, wherein R¹, R², R³, and R⁴ are as defined herein, to form the coupling product 25.4. Compound 25.4 can be isolated, or without isolation, further reacted with an amine HNR¹⁰R¹¹ to give compounds of Formula 25.5.

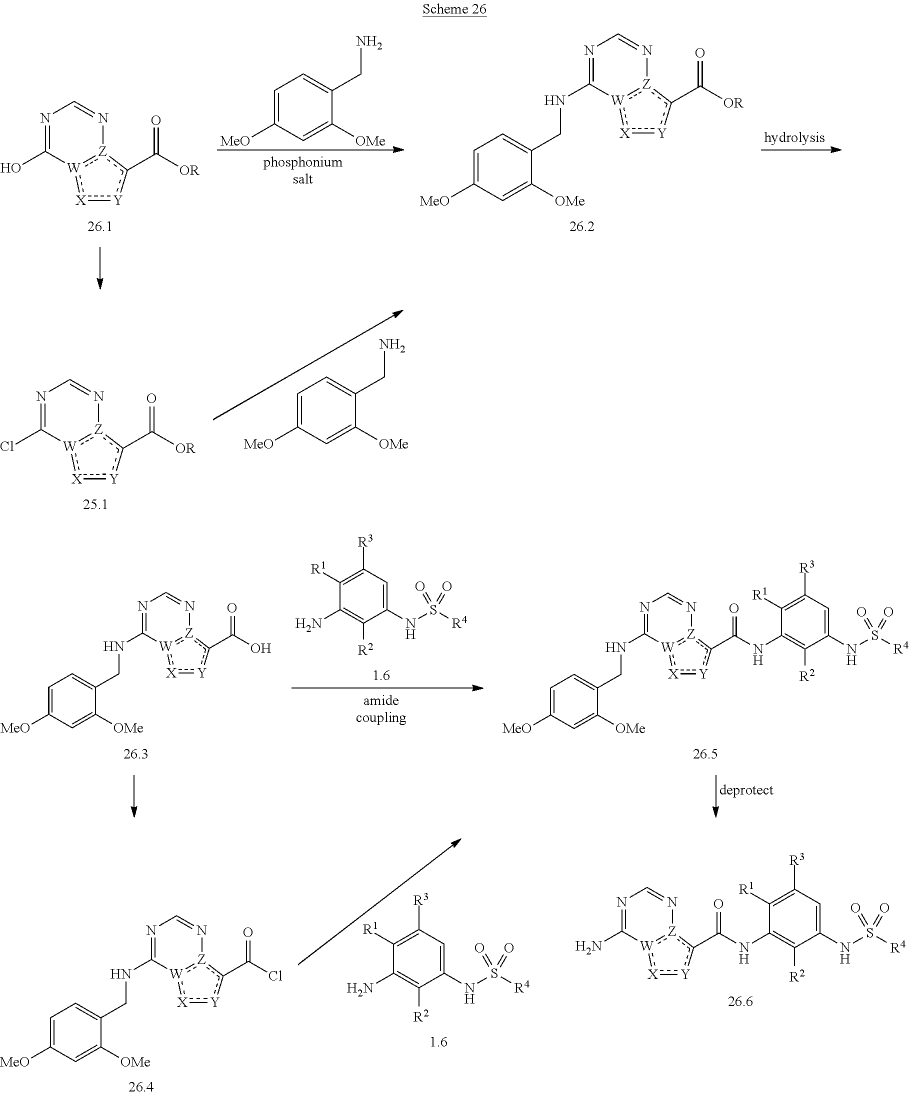

Scheme 26 shows a general procedure for obtaining compound 26.6, wherein R¹, R², R³, R⁴, W, X, Y and Z are defined herein. Ester 26.1 is converted to a dimethoxybenzyl-protected aminopyrimidine 26.2 via coupling with a suitable phosphonium salt such as 1H-benzotriazol-1-yloxy-tris(dimethylamino)phosphonium hexafluorophosphate (“BOP”) or 1H-benzotriazol-1-yloxytris(pyrrolidino)phosphonium hexafluorophosphate (“PyBOP”). Alternatively, compound 26.2 can be prepared via intermediate chloride 25.1, prepared from 26.1 with a chlorinating reagent such as thionyl chloride or phosphorus oxychloride. Hydrolysis of ester 26.2 under aqueous basic conditions to carboxylic acid 26.3 and amide bond coupling with aniline 1.6 with a peptide coupling reagent such as 2-(1H-7-Azabenzotriazol-1-yl)-1,1,3,3-tetramethyl uronium hexafluorophosphate methanaminium (“HATU”) or O-Benzotriazole-N,N,N′,N′-tetramethyl uronium hexafluorophosphate (“HBTU”) gives amide derivative 26.5. Alternatively, compound 26.5 can be prepared via intermediate acid chloride 26.4, prepared from 26.3 using a chlorinating reagent such as thionyl chloride or phosphorus oxychloride, and subsequent coupling with aniline 1.6. Deprotection of the dimethoxybenzyl group under acidic conditions, for example with trifluoroacetic acid under reflux, provides compounds of Formula 26.6.

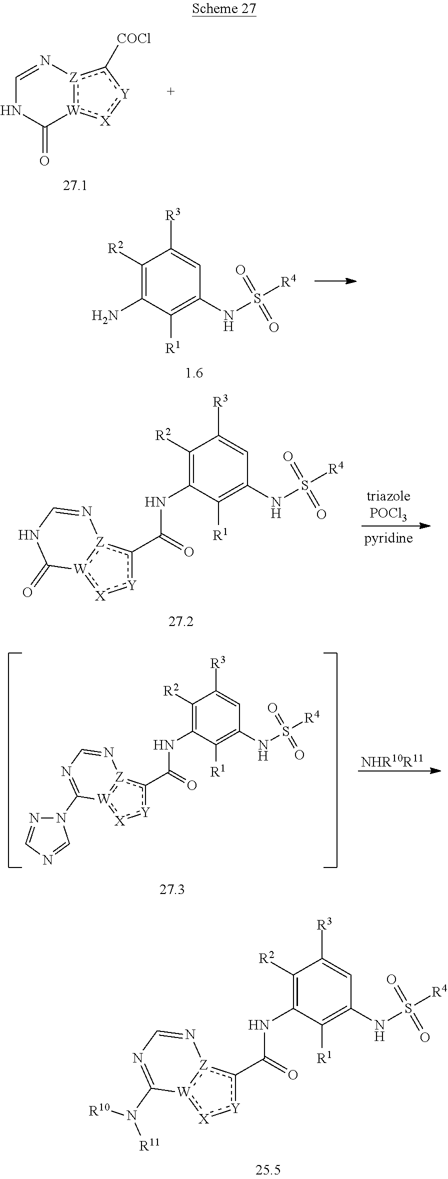

Scheme 27 describes another alternative route to amide derivatives 25.5, where R¹, R², R³, R⁴, R¹⁰, R¹¹, W, X, Y, and Z are defined herein. Reaction of acid chlorides 27.1, with compounds of Formula 22.1, with aniline 1.6 can be accomplished in a suitable solvent, such as chloroform, at ambient or elevated temperature, with or without an added base, such as triethylamine or pyridine. The pyrimidone intermediate 27.2 is reacted with POCl₃ and triazole in pyridine to afford triazole adduct 27.3, which can be isolated or carried on in one pot to the amide derivative 25.5 by treatment with excess amine NHR¹⁰R¹¹ in the original reaction mixture, or, after evaporation, in another solvent such as dioxane or isopropanol.

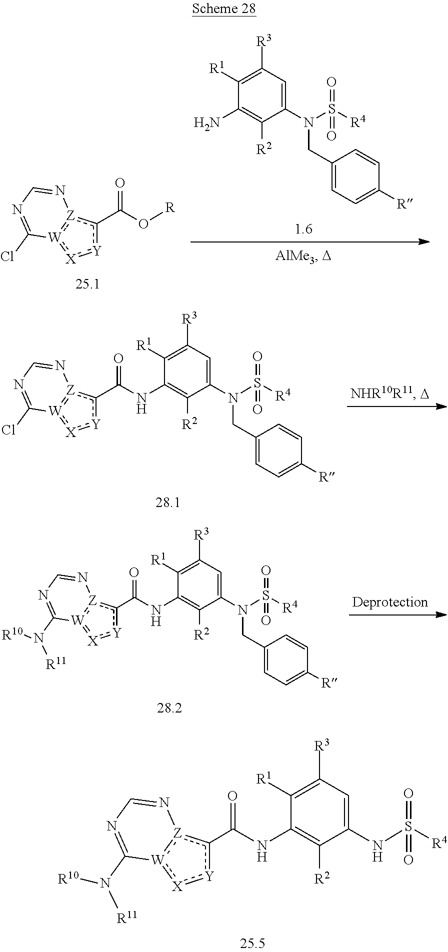

Scheme 28 describes another general method for synthesizing compounds 25.5, wherein R¹, R², R³, R⁴, R″, W, X, Y and Z are as defined herein. Amidation of an ester intermediate of Formula 25.1, with aniline 1.6, using standard Weinreb amidation conditions, affords the amide derivative 28.1. Treatment with an amine NHR¹⁰R¹¹ (e.g. ammonia) in a suitable solvent, such as dioxane or isopropanol, at elevated temperatures forms intermediate 28.2. Optionally a palladium catalyst, such as trisdibenzylidene acetone bispalladium, a ligand, such as Xantphos, and a base, such as cesium carbonate (Buchwald-Hartwig conditions) can be used to facilitate the transformation from 28.1 to 28.2. Subsequent deprotection, using trifluoroacetic acid (if R″═OMe), or Pd/C and hydrogen or ammonium formate (if R″═H), gives target compounds of Formula 25.5.

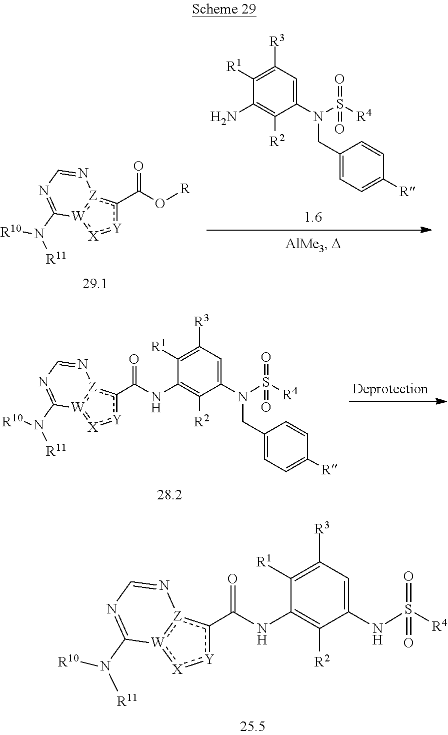

Scheme 29 describes another general method for synthesizing compounds 25.5, wherein R¹, R², R³, R⁴, R″, W, X, Y and Z are as defined herein. Amidation of an ester intermediate of Formula 29.1, with aniline 1.6, using standard Weinreb amidation conditions, affords the amide derivative 28.2 which is subsequently deprotected using trifluoroacetic acid (if R″═OMe) or Pd/C and hydrogen or ammonium formate (if R″═H), to give target compounds of Formula 25.5.

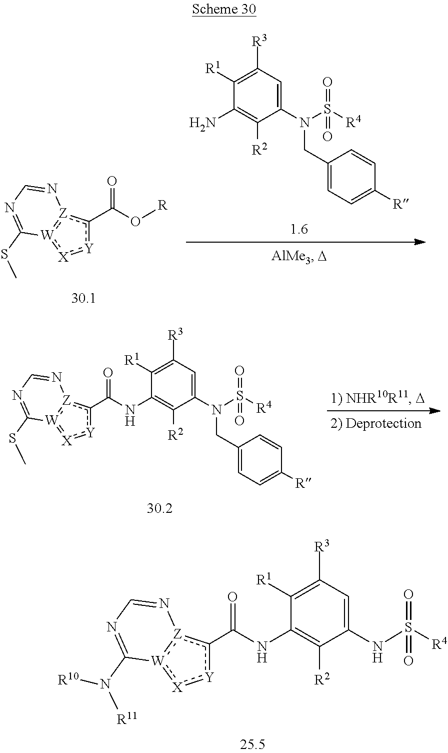

Scheme 30 describes another general method for synthesizing compounds 25.5, wherein R¹, R², R³, R⁴, R¹⁰, R¹¹, W, X, Y and Z are as defined herein. Amidation of ester intermediates 30.1 with aniline 1.6, using standard Weinreb amidation conditions, affords the amide derivative 30.2. Treatment with an amine NHR¹⁰R¹¹ (e.g. ammonia) in a suitable solvent, such as dioxane or isopropanol, at elevated temperatures and subsequent deprotection, using trifluoroacetic acid (if R″═OMe) or Pd/C and hydrogen or ammonium formate (if R″═H), gives target compounds of Formula 25.5.

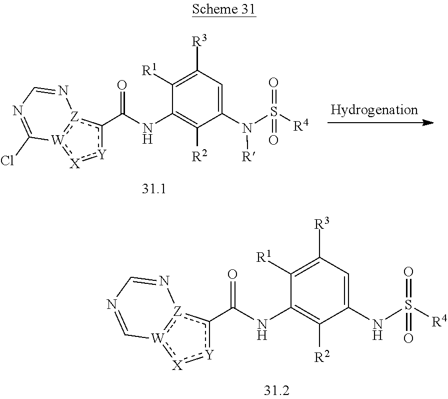

Scheme 31 shows a general method for synthesizing compounds 31.2, wherein R¹, R², R³, R⁴, W, X, Y and Z are as defined herein. This transformation can be accomplished by subjecting chloro intermediate 31.1 (R′ is H, benzyl, or p-methoxybenzyl) to standard hydrogenation conditions, for example using hydrogen and Pd/C catalyst in a suitable solvent such as methanol.

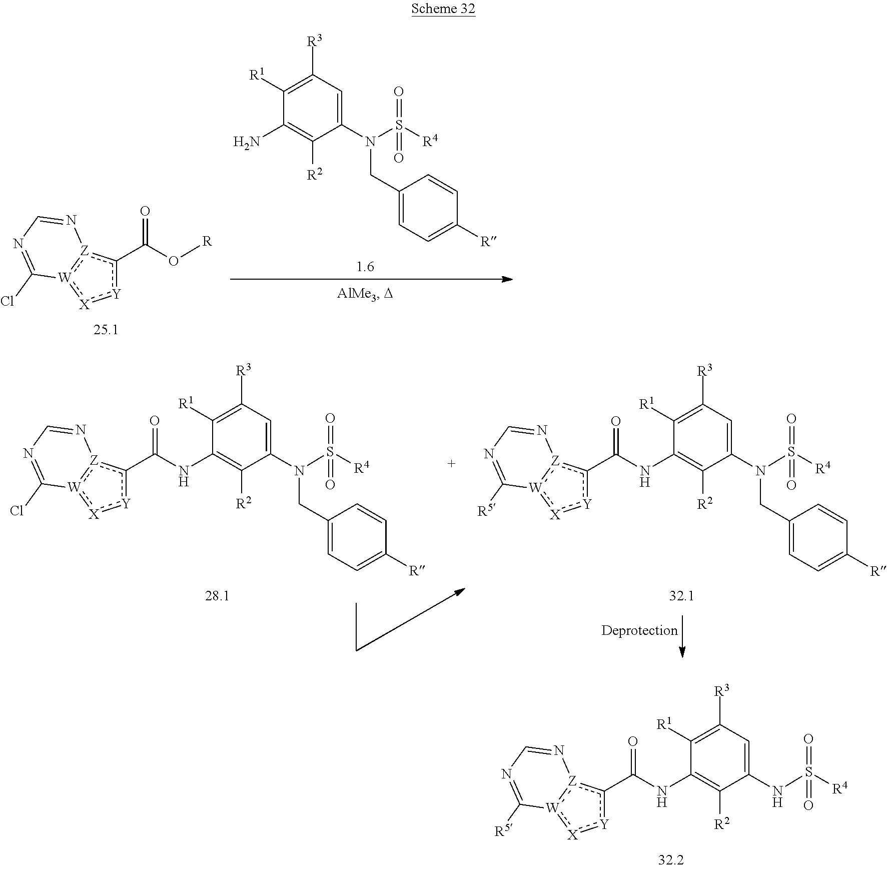

Scheme 32 describes a method for synthesizing compounds of Formula 32.2 wherein R¹, R², R³, R⁴, R″, W, X, Y and Z are as defined herein and R⁵′ is lower alkyl Amidation of an ester intermediate of Formula 25.1, with aniline 1.6, using standard Weinreb amidation conditions, affords the amide derivative 28.1 and compounds of Formula 32.1 as byproduct (with R⁵′=Me). Compound 28.1 can be further transformed as outlined in Scheme 28 or can be transformed to compounds of Formula 32.1 (with R⁵′=lower alkyl) through treatment with an organometallic reagent, such as a methyl magnesium bromide, ethyl magnesium bromide, dimethyl zinc, trimethyl boroxine, triethyl boroxine, methylboronic acid, ethyl boronic acid, potassium trifluoromethylborate, or tetramethyltin, in the presence of a suitable palladium catalyst, such as [1,1′-bis(diphenylphosphino)ferrocene]dichloropalladium(II), and a base, such as cesium carbonate. 32.1 can be deprotected using trifluoroacetic acid (if R″═OMe) or Pd/C and hydrogen or ammonium formate (if R″═H), to form compounds of Formula 32.2.

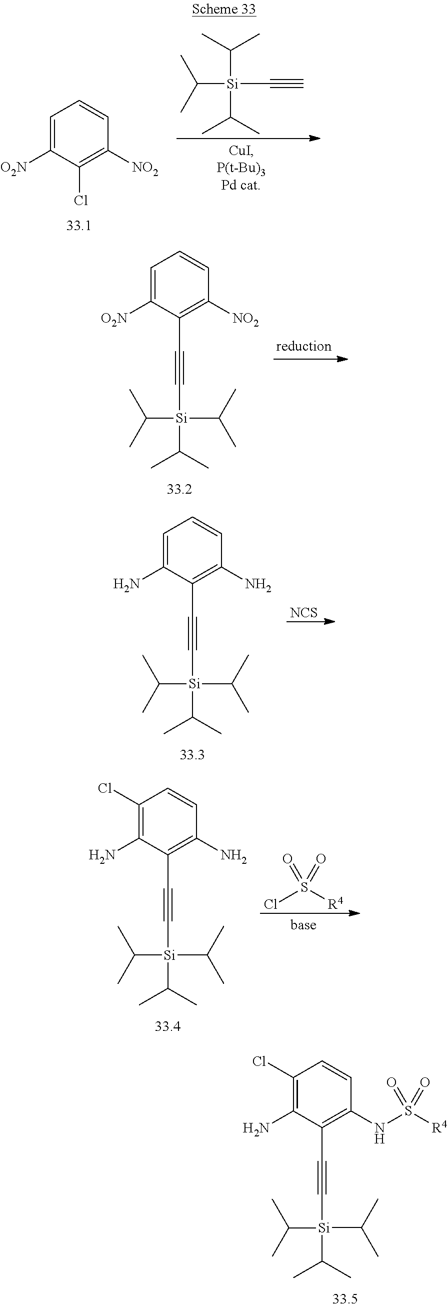

Scheme 33 describes the general synthesis of intermediates of Formula 33.5. 2-Chloro-1,3-dinitrobenzene (33.1), CuI, P(t-Bu)₃, and ethynyltriisopropylsilane and a Pd catalyst, such as PdCl₂(MeCN)₂, in a suitable sovent mixture, such as acetonitrile/triethylamine (5:1), are reacted to form the triisopropylsilane derivative 33.2. Reduction, for example using SnCl₂ in dichloromethane/DMF (1:1), affords the corresponding diamine 33.3. Reaction with n-chlorosuccinimide (“NCS”) in a suitable solvent, such as THF, gived the chlorinated product 33.4, which is further transformed into sulfonamide 33.5 through reaction with a sulfonylchloride R⁴SO₂Cl.

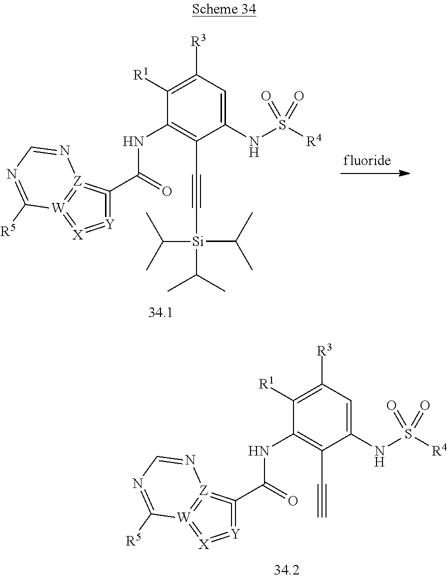

Scheme 34 describes the general synthesis of compounds of Formula 34.2, R² is ethynyl. Trisopropylsilane-protected alkyne 34.1 is treated with a fluoride reagent, such as tetrabutylammonium fluoride (“TBAF”) in a suitable solvent, such as THF, to afford deprotected products of Formula 34.2.

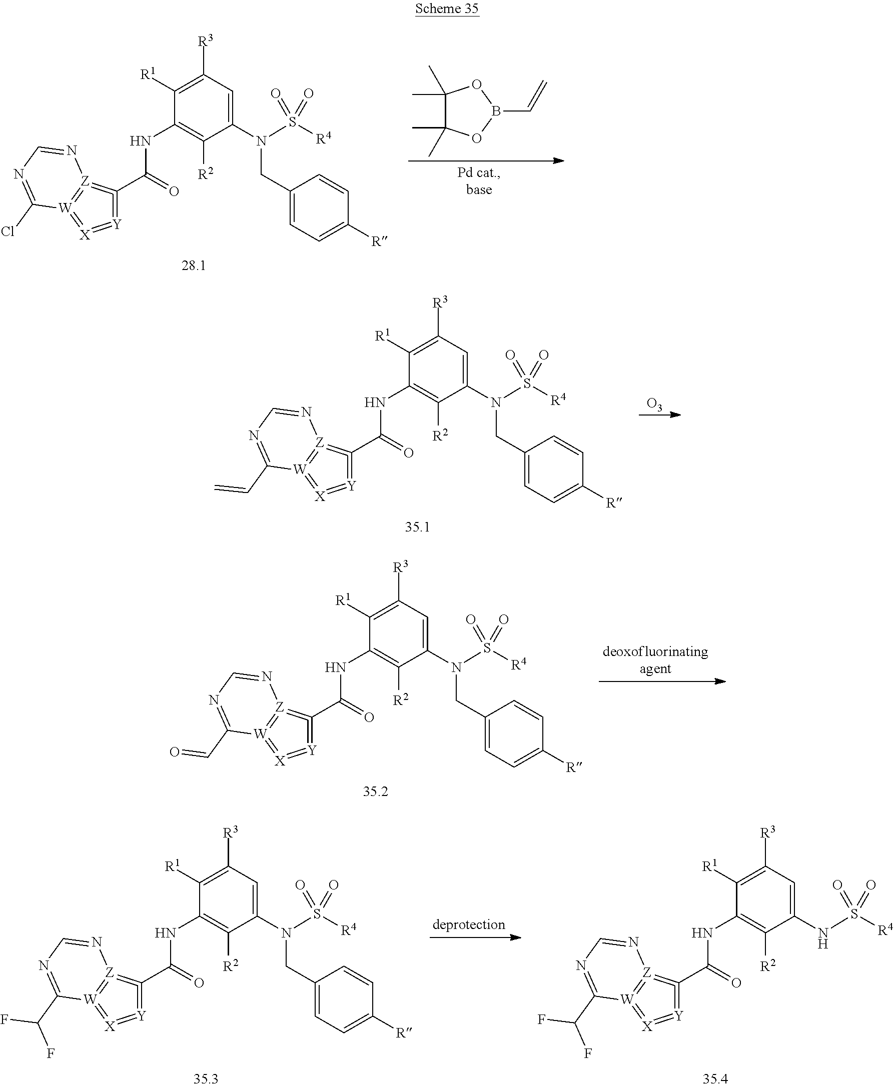

Scheme 35 describes a general method of generating products 35.4, carrying a difluoromethyl group as R⁵. Compounds of Formula 28.1 are reacted at elevated temperatures optionally in a microwave reactor with 4,4,5,5-tetramethyl-2-vinyl-1,3,2-dioxaborolane in the presence of a Pd catalyst, such as bis(triphenylphosphine)palladium (II) chloride, and a base, such as sodium carbonate, in a suitable solvent, such as acetonitrile, to afford vinylated compounds of Formula 35.1. Treatment with ozone under standard ozonolysis conditions yields aldehyde derivatives of Formula 35.2 which are further treated with a deoxofluorinating reagent, such as bis(2-methoxyethyl)aminosulfur trifluoride (“Deoxo-Fluor”) or diethylaminosulfur trifluoride (“DAST”), at low temperatures, for example at −30° C., in a suitable solvent, such as dichloromethane. Deprotection of the 4-methoxybezyl group, for example with a strong acid such as TFA, affords products of Formula 35.4.

### Methods of Separation

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid, can result in formation of the diastereomeric salts.

Alternatively, by method (2), the substrate to be resolved is reacted with one enantiomer of a chiral compound to form a diastereomeric pair (Eliel, E. and Wilen, S. Stereochemistry of Organic Compounds. New York: John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III, Peyton. “Resolution of (±)-5-Bromonornicotine. Synthesis of (R)- and (S)-Nornicotine of High Enantiomeric Purity.” J. Org. Chem. Vol. 47, No. 21 (1982): pp. 4165-4167), of the racemic mixture, and analyzing the 1H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111).

By method (3), a racemic mixture of two enantiomers can be separated by chromatography using a chiral stationary phase (Lough, W. J., Ed. Chiral Liquid Chromatography. New York: Chapman and Hall, 1989; Okamoto, Yoshio, et al. “Optical resolution of dihydropyridine enantiomers by high-performance liquid chromatography using phenylcarbamates of polysaccharides as a chiral stationary phase.” J. Chromatogr. Vol. 513 (1990): pp. 375-378). Enriched or purified enantiomers can be distinguished by methods used to distinguish other chiral molecules with asymmetric carbon atoms, such as optical rotation and circular dichroism.

### Biological Evaluation

B-Raf mutant protein 447-717 (V600E) was co-expressed with the chaperone protein Cdc37, complexed with Hsp90 (Roe, S. Mark, et al. “The Mechanism of Hsp90 Regulation by the Protein Kinase-Specific Cochaperone p50^{cdc37}.” Cell. Vol. 116 (2004): pp. 87-98; Stancato, L F, et al. “Raf exists in a native heterocomplex with Hsp90 and p50 that can be reconstituted in a cell free system.” J. Biol. Chem. 268(29) (1993): pp. 21711-21716).

Determining the activity of Raf in the sample is possible by a number of direct and indirect detection methods (US 2004/0082014). Activity of human recombinant B-Raf protein may be assessed in vitro by assay of the incorporation of radio labeled phosphate to recombinant MAP kinase (MEK), a known physiologic substrate of B-Raf, according to US 2004/0127496 and WO 03/022840. The activity/inhibition of V600E full-length B-Raf was estimated by measuring the incorporation of radio labeled phosphate from [γ-33P]ATP into FSBA-modified wild-type MEK (see Example A).

### Administration and Pharmaceutical Formulations

The compounds of the invention may be administered by any convenient route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal.

The compounds may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents. If parenteral administration is desired, the compositions will be sterile and in a solution or suspension form suitable for injection or infusion.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

One embodiment of the present invention includes a pharmaceutical composition comprising a compound of Formulas I-XXV, or a stereoisomer or pharmaceutically acceptable salt thereof. In a further embodiment, the present invention provides a pharmaceutical composition comprising a compound of Formulas I-XXV, or a stereoisomer or pharmaceutically acceptable salt thereof, together with a pharmaceutically acceptable carrier or excipient.

Another embodiment of the present invention provides a pharmaceutical composition comprising a compound of Formulas I-XXV for use in the treatment of a hyperproliferative disease.

Another embodiment of the present invention provides a pharmaceutical composition comprising a compound of Formulas I-XXV for use in the treatment of cancer.

Another embodiment of the present invention provides a pharmaceutical composition comprising a compound of Formulas I-XXV for use in the treatment of kidney disease. A further embodiment of the present invention provides a pharmaceutical composition comprising a compound of Formulas I-XXV for use in the treatment of polycystic kidney disease.

Methods of Treatment with Compounds of the Invention

The invention includes methods of treating or preventing disease or condition by administering one or more compounds of this invention, or a stereoisomer or pharmaceutically acceptable salt thereof. In one embodiment, a human patient is treated with a compound of Formulas I-XXV, or a stereoisomer, tautomer or pharmaceutically acceptable salt thereof, and a pharmaceutically acceptable carrier, adjuvant, or vehicle in an amount to detectably inhibit B-Raf activity.

In another embodiment, a human patient is treated with a compound of Formulas I-XXV, or a stereoisomer, tautomer, prodrug or pharmaceutically acceptable salt thereof, and a pharmaceutically acceptable carrier, adjuvant, or vehicle in an amount to detectably inhibit B-Raf activity.

In another embodiment of the present invention, a method of treating a hyperproliferative disease in a mammal comprising administering a therapeutically effective amount of the compound of Formulas I-XXV, or a stereoisomer, tautomer, prodrug or pharmaceutically acceptable salt thereof, to the mammal is provided.

In another embodiment of the present invention, a method of treating a hyperproliferative disease in a mammal comprising administering a therapeutically effective amount of the compound of Formulas I-XXV, or a stereoisomer, tautomer or pharmaceutically acceptable salt thereof, to the mammal is provided.

In another embodiment of the present invention, a method of treating kidney disease in a mammal comprising administering a therapeutically effective amount of the compound of Formulas I-XXV, or a stereoisomer, tautomer, prodrug or pharmaceutically acceptable salt thereof, to the mammal is provided. In a further embodiment, the kidney disease is polycystic kidney disease.

In another embodiment, a method of treating or preventing cancer in a mammal in need of such treatment, wherein the method comprises administering to said mammal a therapeutically effective amount of a compound of Formulas I-XXV, or a stereoisomer, tautomer or pharmaceutically acceptable salt thereof. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia. Another embodiment of the present invention provides the use of a compound of Formulas I-XXV, or a stereoisomer, tautomer or pharmaceutically acceptable salt thereof, in the manufacture of a medicament for the treatment of cancer.

In another embodiment, a method of treating or preventing cancer in a mammal in need of such treatment, wherein the method comprises administering to said mammal a therapeutically effective amount of a compound of Formulas I-XXV, or a stereoisomer, tautomer, prodrug or pharmaceutically acceptable salt thereof.

Another embodiment of the present invention provides the use of a compound of Formulas I-XXV, or a stereoisomer, tautomer, prodrug or pharmaceutically acceptable salt thereof, in the manufacture of a medicament for the treatment of cancer.

Another embodiment of the present invention provides the use of a compound of Formulas I-XXV, or a stereoisomer, tautomer, prodrug or pharmaceutically acceptable salt thereof, in the manufacture of a medicament for the treatment of kidney disease. In a further embodiment, the kidney disease is polycystic kidney disease.

In another embodiment, a method of preventing or treating cancer, comprising administering to a mammal in need of such treatment an effective amount of a compound of Formulas I-XXV, or a stereoisomer, tautomer, prodrug or pharmaceutically acceptable salt thereof, alone or in combination with one or more additional compounds having anti-cancer properties.

In another embodiment, a method of preventing or treating cancer, comprising administering to a mammal in need of such treatment an effective amount of a compound of Formulas I-XXV, or a stereoisomer or pharmaceutically acceptable salt thereof, alone or in combination with one or more additional compounds having anti-cancer properties.

In one further embodiment, the cancer is a sarcoma.

In another further embodiment, the cancer is a carcinoma. In one further embodiment, the carcinoma is squamous cell carcinoma. In another further embodiment, the carcinoma is an adenoma or adenocarcinoma.

In another embodiment, a method of treating or preventing a disease or disorder modulated by B-Raf, comprising administering to a mammal in need of such treatment an effective amount of a compound of Formulas I-XXV, or a stereoisomer, tautomer or pharmaceutically acceptable salt thereof. Examples of such diseases and disorders include, but are not limited to, cancer. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

In another embodiment, a method of treating or preventing a disease or disorder modulated by B-Raf, comprising administering to a mammal in need of such treatment an effective amount of a compound of Formulas I-XXV, or a stereoisomer, tautomer, prodrug or pharmaceutically acceptable salt thereof.

In another embodiment of the present invention, a method of preventing or treating kidney disease, comprising administering to a mammal in need of such treatment an effective amount of a compound of Formulas I-XXV, or a stereoisomer, tautomer, prodrug or pharmaceutically acceptable salt thereof, alone or in combination with one or more additional compounds. In another embodiment of the present invention, a method of preventing or treating polycystic kidney disease, comprising administering to a mammal in need of such treatment an effective amount of a compound of Formulas I-XXV, or a stereoisomer, tautomer, prodrug or pharmaceutically acceptable salt thereof, alone or in combination with one or more additional compounds.

Another embodiment of the present invention provides the use of a compound of Formulas I-XXV, or a stereoisomer, tautomer or pharmaceutically acceptable salt thereof, in the manufacture of a medicament for the treatment of cancer. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia. In a further embodiment, the use of a compound of Formulas I-XXV in the manufacture of a medicament, for use as a b-Raf inhibitor in the treatment of a patient undergoing cancer therapy.

Another embodiment of the present invention provides the use of a compound of Formulas I-XXV, or a stereoisomer, tautomer, prodrug or pharmaceutically acceptable salt thereof, in the manufacture of a medicament for the treatment of cancer.

Another embodiment of the present invention provides the use of a compound of Formulas I-XXV, or a stereoisomer, tautomer, prodrug or pharmaceutically acceptable salt thereof, in the manufacture of a medicament for the treatment of polycystic kidney disease. In a further embodiment, the kidney disease is polycystic kidney disease.

Another embodiment of the present invention provides the compounds of Formulas I-XXV for use in therapy.

Another embodiment of the present invention provides the compounds of Formulas I-XXV for use in the treatment of a hyperproliferative disease. In a further embodiment, the hyperproliferative disease is cancer (as further defined and may be individually selected from those above).

Another embodiment of the present invention provides the compounds of Formulas I-XXV for use in the treatment of kidney disease. In a further embodiment, the kidney disease is polycystic kidney disease.

### Combination Therapy

The compounds of this invention and stereoisomers and pharmaceutically acceptable salts thereof may be employed alone or in combination with other therapeutic agents for treatment. The compounds of the present invention can be used in combination with one or more additional drugs, for example an anti-hyperproliferative, anti-cancer, or chemotherapeutic agent. The second compound of the pharmaceutical combination formulation or dosing regimen preferably has complementary activities to the compound of this invention such that they do not adversely affect each other. Such agents are suitably present in combination in amounts that are effective for the purpose intended. The compounds may be administered together in a unitary pharmaceutical composition or separately and, when administered separately this may occur simultaneously or sequentially in any order. Such sequential administration may be close in time or remote in time.

A “chemotherapeutic agent” is a chemical compound useful in the treatment of cancer, regardless of mechanism of action. Chemotherapeutic agents include compounds used in “targeted therapy” and conventional chemotherapy. A number of suitable chemotherapeutic agents to be used as combination therapeutics are contemplated for use in the methods of the present invention. The present invention contemplates, but is not limited to, administration of numerous anticancer agents, such as: agents that induce apoptosis; polynucleotides (e.g., ribozymes); polypeptides (e.g., enzymes); drugs; biological mimetics; alkaloids; alkylating agents; antitumor antibiotics; antimetabolites; hormones; platinum compounds; monoclonal antibodies conjugated with anticancer drugs, toxins, and/or radionuclides; biological response modifiers (e.g., interferons [e.g., IFN-a, etc.] and interleukins [e.g., IL-2, etc.], etc.); adoptive immunotherapy agents; hematopoietic growth factors; agents that induce tumor cell differentiation (e.g., all-trans-retinoic acid, etc.); gene therapy reagents; antisense therapy reagents and nucleotides; tumor vaccines; inhibitors of angiogenesis, and the like.

Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sunitinib (SUTENT®, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (NEXAVAR®, Bayer), Irinotecan (CAMPTOSAR®, Pfizer) and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE™ (Cremophor-free), albumin-engineered nanoparticle formulations of paclitaxel (American Pharmaceutical Partners, Schaumberg, Ill.), and TAXOTERE® (doxetaxel; Rhône-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Raf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); (x) PI3k/AKT/mTOR pathway inhibitors, including GDC-0941 (2-(1H-Indazol-4-yl)-6-(4-methanesulfonyl-piperazin-1-ylmethyl)-4-morpholin-4-yl-thieno[3,2-d]pyrimidine), XL-147, GSK690693 and temsirolimus; (xi) Ras/Raf/MEK/ERK pathway inhibitors; and (xii) pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the Raf inhibitors of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

### EXAMPLES

In order to illustrate the invention, the following Examples are included. However, it is to be understood that these Examples do not limit the invention and are only meant to suggest a method of practicing the invention. Persons skilled in the art will recognize that the chemical reactions described may be readily adapted to prepare a number of other compounds of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

In the Examples described below, unless otherwise indicated all temperatures are set forth in degrees Celsius. Reagents were purchased from commercial suppliers such as Sigma-Aldrich, Alfa Aesar, or TCI, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography purification was done on a Biotage system (Manufacturer: Dyax Corporation) having a silica gel column or on a silica SepPak cartridge (Waters) or on a Teledyne Isco Combiflash purification system using prepacked silica gel cartridges. 1H NMR spectra were recorded on a Bruker AVIII 400 MHz or Bruker AVIII 500 MHz or on a Varian 400 MHz NMR spectrometer.

¹H-NMR spectra were obtained as CDCl₃, CD₂Cl₂, CD₃OD, D₂O, d₆-DMSO, d₆-acetone or CD₃CN solutions (reported in ppm), using tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05 ppm; CD₃CN: 1.94 ppm) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

### Example A

### B-Raf IC₅₀ Assay Protocol

Activity of human recombinant B-Raf protein may be assessed in vitro by assay of the incorporation of radio labeled phosphate to recombinant MAP kinase (MEK), a known physiologic substrate of B-Raf, according to U.S. Patent Appl. Publication No. 2004/0127496 and WO 03/022840. Catalytically active human recombinant B-Raf protein is obtained by purification from sf9 insect cells infected with a human B-Raf recombinant baculovirus expression vector.

The activity/inhibition of V600E full-length B-Raf was estimated by measuring the incorporation of radio labeled phosphate from [γ-33P]ATP into FSBA-modified wild-type MEK. The 30-4 assay mixtures contained 25 mM Na Pipes, pH 7.2, 100 mM KCl, 10 mM MgCl2, 5 mM β-glycerophosphate, 100 μM Na Vanadate, 4 μM ATP, 500 nCi [γ-33P]ATP, 1 μM FSBA-MEK and 20 nM V600E full-length B-Raf. Incubations were carried out at 22° C. in a Costar 3365 plate (Corning). Prior to the assay, the B-Raf and FSBA-MEK were preincubated together in assay buffer at 1.5× (20 μL of 30 nM and 1.5 μM, respectively) for 15 minutes, and the assay was initiated by the addition of 10 μL of 10 μM ATP. Following the 60-minute incubation, the assay mixtures were quenched by the addition of 100 μL of 25% TCA, the plate was mixed on a rotary shaker for 1 minute, and the product was captured on a Perkin-Elmer GF/B filter plate using a Tomtec Mach III Harvester. After sealing the bottom of the plate, 35 μL of Bio-Safe II (Research Products International) scintillation cocktail were added to each well and the plate was top-sealed and counted in a Topcount NXT (Packard).

The compounds of Examples 1-74, 76-95 and 97-113 were tested in the above assay and found to have an IC50 of less than about 1 μM.

The compounds of Examples 1-26, 28-34, 37-40, 42-50, 52, 53, 55-57, 59, 61-74, 76-78, 82-87, 89-92, 95, 97-102, 104, 107 and 109-112 were tested in the above assay and found to have an IC50 of less than 100 nM.

### Example B

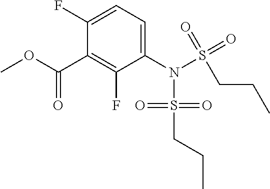

### Methyl 2,6-difluoro-3-(N-(propylsulfonyl)propylsulfonamido)benzoate

Step A: A 1 L flask was charged with 2,6-difluoro-3-nitrobenzoic acid (17.0 g, 83.7 mmol) and MeOH (170 mL, 0.5M). The flask was placed in a cold water bath, and an addition funnel charged with a 2M solution of trimethylsilyl (“TMS”) diazomethane in hexanes (209 mL, 419 mmol) was attached to the flask. The TMS diazomethane solution was added slowly to the reaction flask over the course of 2 hours. A large excess of reagent was required in order for the reaction to reach completion as determined by the ceased evolution of N₂ upon further addition of reagent. The volatiles were removed in vacuo to afford methyl 2,6-difluoro-3-nitrobenzoate as a solid (18.2 g). The material was taken directly onto Step B.

Step B: 10% (wt.) Pd on activated carbon (4.46 g, 4.19 mmol) was added to a 1 L flask charged with methyl 2,6-difluoro-3-nitrobenzoate (18.2 g, 83.8 mmol) under a nitrogen atmosphere. EtOH (350 mL, 0.25 M) was added, and then H₂ was passed through the reaction mixture for 15 minutes. The reaction mixture was stirred under two H₂ balloons overnight. The following day the reaction mixture was re-flushed with fresh H₂ balloons and stirred an additional 4 hours. Upon consumption of the starting material and intermediate hydroxylamine as determined by TLC, N₂ gas was flushed through the reaction mixture. The mixture was then filtered through glass □ulfonamid filter (“GF/F”) paper twice. The volatiles were removed to afford methyl 3-amino-2,6-difluorobenzoate as an oil (15.66 g). The material was taken directly onto the next step.

Step C: Propane-1-sulfonyl chloride (23.46 mL, 209.3 mmol) was slowly added to a solution of methyl 3-amino-2,6-difluorobenzoate (15.66 g, 83.7 mmol) and triethylamine (35.00 mL, 251.1 mmol) in CH₂Cl₂ (175 mL, 0.5 M) maintained in a cool water bath. The reaction mixture was stirred for 1 hour at room temperature. Water (300 mL) was added, and the organic layer was separated, washed with water (2×300 mL), brine (200 mL), then dried (Na₂SO₄), filtered and concentrated to an oil. The crude product was purified by column chromatography, eluting with 15% ethyl acetate (“EtOAc”)/hexane. The isolated fractions were triturated with hexanes to afford methyl 2,6-difluoro-3-(N-(propylsulfonyl)propyl-□ulfonamide)benzoate as a solid (24.4 g, 73% yield for 3 steps). ¹H NMR (400 MHz, CDCl₃) δ 7.52-7.45 (m, 1H), 7.08-7.02 (m, 1H), 3.97 (s, 3H), 3.68-3.59 (m, 2H), 3.53-3.45 (m, 2H), 2.02-1.89 (m, 4H), 1.10 (t, J=7.4 Hz, 6H). m/z (APCI-neg) M-(SO₂Pr)=292.2.

### Example C

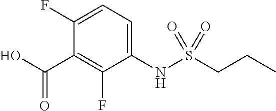

### 2,6-Difluoro-3-(propylsulfonamido)benzoic acid

A 1N aqueous NaOH solution (150 mL, 150 mmol) was added to a solution of methyl 2,6-difluoro-3-(N-(propylsulfonyl)propylsulfonamido)benzoate (20.0 g, 50.1 mmol) in 4:1 THF/MeOH (250 mL, 0.2M). The reaction mixture was stirred at room temperature overnight. The majority of the organic solvents were then removed in vacuo (water bath temperature 35° C.). 1N HCl (150 mL) was slowly added to the mixture, and the resulting solid was filtered and rinsed with water (4×50 mL). The material was then washed with Et₂O (4×15 mL) to give 2,6-difluoro-3-(propylsulfonamido)benzoic acid as a solid (10.7 g, 77% yield). ¹H NMR (400 MHz, DMSO-d₆) δ 9.74 (s, 1H), 7.57-7.50 (m, 1H), 7.23-7.17 (m, 1H), 3.11-3.06 (m, 2H), 1.79-1.69 (m, 2H), 0.98 (t, J=7.4 Hz, 3H). m/z (APCI-neg) M−1=278.0.

### Example D

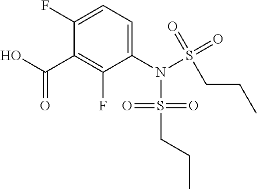

### 2,6-Difluoro-3-(N-(propylsulfonyl)propylsulfonamido)benzoic acid

Propane-1-sulfonyl chloride (1.225 mL, 10.92 mmol) was added to a mixture of 3-amino-2,6-difluorobenzoic acid (0.573 g, 3.310 mmol), triethylamine (2.030 mL, 14.56 mmol) and CH₂Cl₂ (17 mL, 0.2M) cooled to 0° C. The reaction mixture was allowed to warm to room temperature and stirred for 1 hour. The mixture was then partitioned between saturated NaHCO₃ (100 mL) and ethyl acetate (75 mL). The aqueous layer was washed with ethyl acetate (50 mL) and then acidified with concentrated HCl to a pH of about 1. The acidified aqueous layer was extracted with ethyl acetate (2×50 mL), and the combined ethyl acetate extracts were dried (Na₂SO₄), filtered and concentrated. The resulting residue was triturated with hexanes to afford 2,6-difluoro-3-(N-(propylsulfonyl)propyl-sulfonamido)-benzoic acid as a solid (0.948 g, 74% yield). ¹H NMR (400 MHz, DMSO-d₆) δ 7.90-7.84 (m, 1H), 7.39-7.34 (m, 1H), 3.73-3.58 (m, 4H), 1.88-1.74 (m, 4H), 1.01 (t, J=7.5 Hz, 6H). m/z (APCI-neg) M-(SO2Pr)=278.1.

### Example E

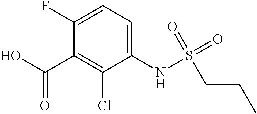

### 2-Chloro-6-fluoro-3-(propylsulfonamido)benzoic acid

Step A: Into a 20-L 4-neck round flask was placed a solution of 2-chloro-4-fluorobenzenamine (1300 g, 8.82 mol, 1.00 equiv, 99%) in toluene (10 L), 4-methylbenzenesulfonic acid (3.1 g, 17.84 mmol, 99%), and hexane-2,5-dione (1222.5 g, 10.62 mol, 1.20 equiv, 99%). The resulting solution was heated to reflux for 1 h in an oil bath and cooled. The pH value of the solution was adjusted to 8 with sodium carbonate (1 mol/L). The resulting mixture was washed with 1×5000 mL of water and concentrated under vacuum. The crude product was purified by distillation and the fraction was collected at 140° C. to afford 1-(2-chloro-4-fluorophenyl)-2,5-dimethyl-1H-pyrrole (1700 g, yield: 85%).

Step B: Into a 5000-mL 4-necked round-bottom flask purged and maintained with an inert atmosphere of nitrogen was placed a solution of 1-(2-chloro-4-fluorophenyl)-2,5-dimethyl-1H-pyrrole (390 g, 1.65 mol, 1.00 equiv, 95%) in tetrahydrofuran (2000 mL). The reaction vessel was cooled to −78° C. To the above reaction vessel was added n-BuLi (800 mL, 1.10 equiv, 2.5%) dropwise with stirring over 80 minutes and methyl carbonochloridate (215.5 g, 2.27 mol, 1.20 equiv, 99%) dropwise with stirring over 90 minutes. The reaction solution was further stirred for 60 minutes at −78° C. and quenched by the addition of 1000 mL of NH₄Cl/water. The resulting solution was extracted with 1500 mL of ethyl acetate. The organic layers were combined, washed with 1×1500 mL of water and 1×1500 mL of sodium chloride(aq), dried over anhydrous magnesium sulfate, and concentrated under vacuum to afford methyl 2-chloro-3-(2,5-dimethyl-1H-pyrrol-1-yl)-6-fluorobenzoate (crude, 566.7 g).

Step C: Into five 5000-mL 4-neck round-bottom flasks was placed a solution of methyl 2-chloro-3-(2,5-dimethyl-1H-pyrrol-1-yl)-6-fluorobenzoate (1500 g, 5.05 mol, 1.00 equiv, 95%) in ethanol/H₂O (7500/2500 mL), NH₂OH—HCl (5520 g, 79.20 mol, 15.00 equiv, 99%), and triethylamine (2140 g, 20.98 mol, 4.00 equiv, 99%). The resulting solution was refluxed for 18 h in an oil bath, cooled to room temperature, concentrated, and extracted with 3×3000 mL of ethyl acetate. The organic layers were combined, dried over anhydrous sodium sulfate, and concentrated under vacuum. The residue was purified using a silica gel column eluting with PE:EA (20:1-10:1) to afford methyl 3-amino-2-chloro-6-fluorobenzoate (980 g, yield: 95%).

Step D: Into four 5000-mL 4-neck round-bottom flasks was placed a solution of methyl 3-amino-2-chloro-6-fluorobenzoate (980 g, 4.76 mol, 1.00 equiv, 99%) in dichloromethane (8000 mL). Triethylamine (1454 g, 14.25 mol, 3.00 equiv, 99%) was added dropwise with stirring at 0° C. over 80 minutes followed by the addition of propane-1-sulfonyl chloride (1725 g, 11.94 mol, 2.50 equiv, 99%). The resulting solution was stirred at room temperature for 2 h, diluted with 1000 mL of water. The organic layer was washed with 1×1000 mL of hydrogen chloride and 1×1000 mL of water, dried over sodium sulfate, and concentrated to afford methyl 2-chloro-6-fluoro-3-(propylsulfonamido)benzoate as a brown solid (1500 g, 97%).

Step E: Into a 10000-mL 4-necked round-bottom flask was placed a solution of methyl 2-chloro-6-fluoro-3-(propylsulfonamido)benzoate (1500 g, 4.61 mol, 1.00 equiv, 95%) in tetrahydrofuran/H₂O (3000/3000 mL) and potassium hydroxide (1000 g, 17.68 mol, 4.50 equiv, 99%). The resulting solution was refluxed for 2 hours, cooled to room temperature and extracted with 3×2000 mL of ethyl acetate. The aqueous layers were combined and the pH was adjusted to 2 with hydrogen chloride (2 mol/L). The resulting solution was extracted with 2×3000 mL of dichloromethane. The organic layers were combined, dried over anhydrous sodium sulfate and concentrated to afford 2-chloro-6-fluoro-3-(propylsulfonamido)benzoic acid (517.5 g, yield: 37%). ¹H NMR (400 MHz, CDCl₃): δ 1.058-1.096 (m, J=15.2 Hz, 3H), 1.856-1.933 (m, 2H), 3.073-3.112 (m, 2H); 6.811 (1H, s), 7.156-7.199 (d, J=17.2 Hz, 1H), 7.827-7.863 (d, J=14.4 Hz, 1H); (ES, m/z): [M+H]⁺ 296.

### Example F

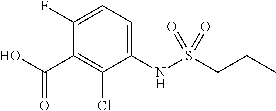

### Benzyl 6-chloro-2-fluoro-3-(propylsulfonamido) benzoic acid

Step A: A flame dried flask equipped with a stir bar and rubber septum was charged with 4-chloro-2-fluoroaniline (5.00 g, 34.35 mmol) and anhydrous THF (170 mL). This solution was chilled to −78° C., and n-BuLi (14.7 mL, 1.07 eq. of 2.5M solution in hexanes) was then added over a 15 minute period. This mixture was stirred at −78° C. for 20 minutes, and then a THF solution (25 mL) of 1,2-bis(chlorodimethylsilyl)ethane (7.76 g, 1.05 eq.) was added slowly (over a 10 minute period) to the reaction mixture. This was stirred for 1 hour, and then 2.5M n-BuLi in hexanes (15.11 mL, 1.1 eq.) was added slowly. After allowing the mixture to warm to room temperature for one hour, the mixture was chilled back to −78° C. A third allotment of n-BuLi (15.66 mL, 1.14 eq.) was added slowly, and the mixture was stirred at −78° C. for 75 minutes. Benzyl chloroformate (7.40 g, 1.2 eq.) was then added slowly, and the mixture was stirred at −78° C. for one hour. The cooling bath was then removed. The mixture was allowed to warm for 30 minutes and then quenched with water (70 mL) and concentrated HCl (25 mL). The mixture was allowed to continue to warm to room temperature. The mixture was then extracted with EtOAc. The extracts were washed twice with a saturated NaHCO₃ solution, once with water, dried over sodium sulfate and concentrated. The resulting residue was purified via silica gel column chromatography (30% ethyl acetate/hexane) to produce benzyl 3-amino-6-chloro-2-fluorobenzoate (4.3 g, 45%) as an oil. ¹H NMR (DMSO-d₆, 400 MHz) δ 7.37-7.48 (m, 5H), 7.07 (dd, J=8 Hz, 2 Hz, 1H), 6.87 (t, J=8 Hz, 1H), 5.61 (br s, 2H), 5.40 (s, 2H).

Step B: Benzyl 3-amino-6-chloro-2-fluorobenzoate (4.3 g, 15.37 mmol) was dissolved in dry dichloromethane (270 mL). Triethylamine (5.36 mL, 2.5 eq.) was added, and the mixture was chilled to 0° C. Propane-1-sulfonyl chloride (3.63 mL, 32.3 mmol, 2.1 eq.) was then added via syringe, and a precipitate resulted. Once the addition was complete, the mixture was allowed to warm to room temperature, and the starting material was consumed as determined by TLC (3:1 hexane:ethyl acetate). The mixture was then diluted with dichloromethane (200 mL), washed with 2M aqueous HCl (2×100 mL), saturated NaHCO₃ solution, dried over sodium sulfate and concentrated. The resulting residue was purified via silica gel column chromatography (40% ethyl acetate/hexane) to produce benzyl 6-chloro-2-fluoro-3-(N-(propylsulfonyl)propylsulfonamido)benzoate (5.5 g, 72%) as an oil that slowly solidified upon standing. ¹H NMR (CDCl₃, 400 MHz) δ 7.28-7.45 (m, 7H), 5.42 (s, 2H), 3.58-3.66 (m, 2H), 3.43-3.52 (m, 2H), 1.08 (t, J=8 Hz, 6H).

Step C: Benzyl 6-chloro-2-fluoro-3-(N-(propylsulfonyl)propylsulfonamido) benzoate (5.4 g, 10.98 mmol) was dissolved in THF (100 mL) and 1M aqueous KOH (100 mL). This mixture was refluxed for 16 hours and then allowed to cool to room temperature. The mixture was then acidified to a pH of 2 with 2M aqueous HCl and extracted with EtOAc (2×). The extracts were washed with water, dried over sodium sulfate and concentrated to a solid that was triturated with hexanes/ether to give 6-chloro-2-fluoro-3-(propylsulfonamido)benzoic acid (2.2 g, 68%) as a solid. ¹H NMR (DMSO-d₆, 400 MHz) δ 9.93 (s, 1H), 7.49 (t, J=8 Hz, 1H), 7.38 (dd, J=8 Hz, 2 Hz, 1H), 3.11-3.16 (m, 2H), 1.68-1.78 (m, 2H), 0.97 (t, J=8 Hz, 3H).

### Example G

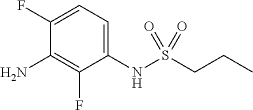

### N-(3-Amino-2,4-difluorophenyl)propane-1-sulfonamide

To a solution of 2,6-difluoro-3-(propylsulfonamido)benzoic acid (4.078 g, 14.6 mmol) in THF (60 mL) was added triethylamine (4.68 mL, 33.59 mmol) and diphenylphosphonic azide (3.73 mL, 16.79 mmol). The reaction mixture was stirred at room temperature for 3 hours and then warmed to 80° C. for 2 hours. Water (10 mL) was added, and the mixture stirred at 80° C. for 15 hours. The reaction mixture was diluted with 300 mL of EtOAc, and the organic layer was washed with saturated aq. NaHCO₃ solution and brine. The solvent was removed under reduced pressure, and the residual purified via silica gel column chromatography eluting with 30/70 EtOAc/hexane to obtain 2.03 g (55%) of the title compound. ¹H NMR (400 MHz, DMSO-d₆) δ 9.32 (s, 1H), 6.90-6.80 (m, 1H), 6.51 (td, J=8.7 Hz, 5.5 Hz, 1H), 5.28 (s, 2H), 3.05-2.96 (m, 2H), 1.82-1.64 (m, 2H), 1.01-0.90 (m, 3H). LC/MS: m/z 251.1 [M+1].

### Example H

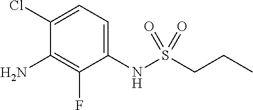

### N-(3-Amino-4-chloro-2-fluorophenyl)propane-1-sulfonamide

The compound was made using the procedure described in Example G using 6-chloro-2-fluoro-3-(propylsulfonamido)benzoic acid instead of 2,6-difluoro-3-(propylsulfonamido)benzoic acid as starting material. ¹H NMR (500 MHz, DMSO-d₆) δ 9.54 (s, 1H), 7.02 (d, 1H), 6.58 (t, 1H), 5.50 (s, 2H), 3.09-2.95 (t, 2H), 1.81-1.64 (sx, 2H), 0.96 (t, 3H). LC/MS: m/z 267.1 [M+1].

### Example I

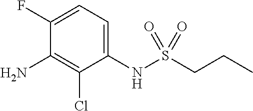

### N-(3-Amino-2-chloro-4-fluorophenyl)propane-1-sulfonamide

The compound was made using the procedure described in Example G using 2-chloro-6-fluoro-3-(propylsulfonamido)benzoic acid instead of 2,6-difluoro-3-(propylsulfonamido)benzoic acid as starting material. ¹H NMR (400 MHz, DMSO-d₆) δ 9.20 (s, 1H), 7.28-6.99 (m, 1H), 6.63 (td, J=8.7 Hz, 5.5 Hz, 1H), 5.45 (s, 2H), 3.07-2.99 (m, 2H), 1.88-1.69 (m, 2H), 1.03-0.95 (m, 3H). LC/MS: m/z 267.1 [M+1].

### Example J

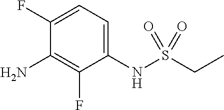

### N-(3-amino-2,4-difluorophenyl)ethanesulfonamide

The compound was made using the procedure described in Example G using 2,6-difluoro-3-(ethylsulfonamido)benzoic acid instead of 2,6-difluoro-3-(propylsulfonamido)benzoic acid as starting material. ¹H NMR (400 MHz, DMSO-d₆) δ 9.37 (d, J=9.6 Hz, 1H), 6.86 (ddd, J=10.7 Hz, 9.1 Hz, 1.9 Hz, 1H), 6.52 (td, J=8.7 Hz, 5.5 Hz, 1H), 5.28 (s, 2H), 3.03 (q, J=7.3 Hz, 2H), 1.25 (td, J=7.3 Hz, 2.5 Hz, 3H). LC/MS: m/z 237.1 [M+1].

### Example K

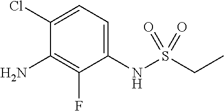

### N-(3-Amino-4-chloro-2-fluorophenyl)ethanesulfonamide

The compound was made using the procedure described in Example G using 6-chloro-3-(ethylsulfonamido)-2-fluorobenzoic acid instead of 2,6-difluoro-3-(propylsulfonamido)benzoic acid as starting material. ¹H NMR (500 MHz, DMSO-d₆) δ 9.48 (s, 1H), 7.02 (dd, J=8.8 Hz, 1.7 Hz, 1H), 6.59 (t, J=8.3 Hz, 1H), 5.44 (s, 2H), 3.07 (q, J=7.3 Hz, 2H), 1.24 (t, J=7.3 Hz, 3H). LC/MS: m/z 253.2 [M+1].

### Example L

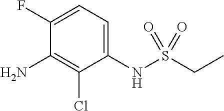

### N-(3-Amino-2-chloro-4-fluorophenyl)ethanesulfonamide

2-Chloro-6-fluoro-3-(ethylsulfonamido)benzoic acid (3.3 g, 12.0 mmol) was treated with thionyl chloride (21.0 mL, 0.29 mmol) and heated at reflux for 15 hours. The reaction mixture was concentrated and then azeotrophed with toluene (2×20 mL). The residue was treated with a solution of sodium azide (3.1 g, 48.0 mmol) dissolved in water (20 mL) and acetone (20 mL). After stirring at room temperature for 1 hour, the intermediate acyl azide was extracted into ethyl acetate (2×25 mL), dried with magnesium sulfate and concentrated. The residue was dissolved in dioxane (40 mL) and water (5 mL) and heated to reflux for 3 hours. After cooling to room temperature, the product was extracted into dichloromethane (2×25 mL), dried with magnesium sulfate and concentrated. The residue was purified by flash silica gel chromatography (2-30% isopropanol in dichloromethane) to afford N-(3-amino-2-chloro-4-fluorophenyl)ethanesulfonamide. (2.0 g, 66%). ¹H NMR (400 MHz, DMSO-d₆) δ 9.15 (s, 1H), 7.02 (dd, J=10.7 Hz, 8.8 Hz, 1H), 6.64 (dd, J=8.8 Hz, 5.1 Hz, 1H), 5.45 (s, 2H), 3.06 (q, J=7.3 Hz, 2H), 0.96 (t, J=7.3 Hz, 3H). LC/MS: m/z 253.0 [M+1].

### Example M

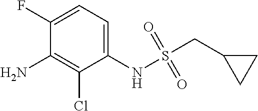

### N-(3-Amino-2-chloro-4-fluorophenyl)-1-cyclopropylmethanesulfonamide

Step A: To a solution of methyl 3-amino-2-chloro-6-fluorobenzoate (2.97 g, 14.6 mmol) in THF (26 mL) and triethylamine (6.10 mL, 43.8 mmol) at 0° C. was added cyclopropylmethanesulfonyl chloride (4.74 g, 30.6 mmol) dropwise. The reaction mixture was stirred at 0° C. for 90 minutes, after which 8N NaOH (18.2 mL, 140 mmol) was added. The reaction mixture was then warmed up at 40° C. and stirred for 16 hours. The volatiles were removed in vacuo and the mixture acidified with concentrated HCl at 0° C. to pH 1. The acidified mixture was extracted with ethyl acetate twice. The organic phases were combined, dried with sodium sulfate, filtered and concentrated in vacuo to obtain crude 2-chloro-3-(cyclopropylmethylsulfonamido)-6-fluorobenzoic acid, which was used directly in the next step without further purification.

Step B: To a solution of 2-chloro-3-(cyclopropylmethylsulfonamido)-6-fluorobenzoic acid (4.11 g, 13.4 mmol) in 1,4-dioxane (30 mL) was added triethylamine (2.05 mL, 14.7 mmol), followed by diphenylphosphonic azide (3.12 mL, 14.0 mmol) at room temperature. The reaction was stirred at room temperature for 4 hours and the resulting mixture added dropwise, via an addition funnel, over 15 minutes in a round-bottom flask containing 1,4-dioxane (16 mL) and water (1.20 mL, 66.8 mmol) at 95° C. The reaction mixture was stirred at this temperature for 16 hours. Half of the reaction mixture was concentrated in vacuo, then the rest of the solution was diluted with ethyl acetate and a saturated solution of NaHCO₃. The layers were separated and the aqueous layer extracted twice with ethyl acetate. The organic phases were combined, dried with sodium sulfate, filtered and concentrated in vacuo. The crude product was purified by flash chromatography to afford N-(3-amino-2-chloro-4-fluorophenyl)-1-cyclopropylmethanesulfonamide (2.05 g, 55%). ¹H NMR (500 MHz, DMSO-d₆) δ 9.07 (s, 1H), 7.01 (dd, J=10.7 Hz, 8.9 Hz, 1H), 6.66 (dd, J=8.8 Hz, 5.1 Hz, 1H), 5.43 (s, 2H), 3.04 (d, J=7.1 Hz, 2H), 1.12-0.99 (m, 1H), 0.59-0.52 (m, 2H), 0.36-0.30 (m, 2H); m/z (ES-MS) M+1=279.2.

### Example N

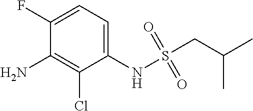

### N-(3-Amino-2-chloro-4-fluorophenyl)-2-methylpropane-1-sulfonamide

Step A: To a solution of methyl 3-amino-2-chloro-6-fluorobenzoate (2.97 g, 14.6 mmol) in THF (20 mL) and triethylamine (6.10 mL, 43.8 mmol) at 0° C. was added 2-methylpropane-1-sulfonyl chloride (4.80 g, 30.6 mmol) dropwise. The reaction mixture was stirred at 0° C. for 90 minutes, after which 8N aqueous NaOH (18.2 mL, 140 mmol) was added. The reaction mixture was warmed up at 40° C. and stirred for 16 hours. The volatiles were then removed in vacuo and the mixture acidified with concentrated HCl at 0° C. to pH 1. The acidified mixture was extracted with ethyl acetate twice. The organic phases were combined, dried with sodium sulfate, filtered and concentrated in vacuo to obtain crude 2-chloro-6-fluoro-3-(2-methylpropylsulfonamido)benzoic acid, which was used directly in the next step without further purification.

Step B: N-(3-Amino-2-chloro-4-fluorophenyl)-2-methylpropane-1-sulfonamide was prepared according to the general procedure for Example M (step B), substituting 2-chloro-6-fluoro-3-(2-methylpropylsulfonamido)benzoic acid for 2-chloro-3-(cyclopropylmethylsulfonamido)-6-fluorobenzoic acid. ¹H NMR (500 MHz, DMSO-d₆) δ 9.14 (s, 1H), 7.02 (dd, J=10.7 Hz, 8.9 Hz, 1H), 6.64 (dd, J=8.8 Hz, 5.1 Hz, 1H), 5.44 (s, 2H), 2.96 (d, J=6.4 Hz, 2H), 2.20-2.10 (m, 1H), 1.01 (d, J=6.7 Hz, 6H); m/z (ES-MS) M+1=281.2.

### Example O

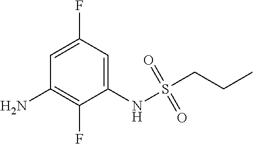

### N-(3-Amino-2,5-difluorophenyl)propane-1-sulfonamide

To a solution of 2,5-difluorobenzene-1,3-diamine (2.00 g, 13.9 mmol) (described in E.P. Pat. Appl. Publication No. 0,415,595) in THF (40 mL) and pyridine (1.571 mL, 19.43 mmol) was added propane-1-sulfonyl chloride (1.867 mL, 16.65 mmol) at 0° C. The reaction mixture was stirred at 50° C. for 90 minutes and dichloromethane and a saturated solution of NaHCO₃ were then added. The layers were separated and the aqueous layer was extracted twice with dichloromethane. The organic layers were combined, dried with sodium sulfate, filtered and concentrated in vacuo. The crude mixture was re-submitted to exact same reaction conditions, and the reaction was stirred at 55° C. for 16 hours, and ethyl acetate and a saturated solution of NaHCO₃ were then added. The layers were separated and the aqueous layer extracted twice with ethyl acetate. The organic layers were combined, dried with sodium sulfate, filtered and concentrated in vacuo. The crude product was purified by flash chromatography to afford N-(3-amino-2,5-difluorophenyl)propane-1-sulfonamide (485 mg, 14%). ¹H NMR (400 MHz, DMSO-d₆) δ 9.57 (s, 1H), 6.39-6.23 (m, 2H), 5.55 (s, 2H), 3.12-2.99 (m, 2H), 1.77-1.66 (m, 2H), 0.96 (t, J=7.3 Hz, 3H); m/z (ES-MS) M+1=251.2.

### Example P

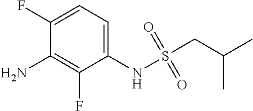

### N-(3-Amino-2,4-difluorophenyl)-2-methylpropane-1-sulfonamide

Step A: 2,6-Difluoro-3-(2-methylpropylsulfonamido)benzoic acid was prepared according to the general procedure for Example N (step A), substituting methyl 3-amino-2,6-difluorobenzoate for methyl 3-amino-2-chloro-6-fluorobenzoate.

Step B: N-(3-Amino-2,4-difluorophenyl)-2-methylpropane-1-sulfonamide was prepared according to the general procedure for Example M (step B), substituting 2,6-difluoro-3-(2-methylpropylsulfonamido)benzoic acid for 2-chloro-3-(cyclopropylmethylsulfonamido)-6-fluorobenzoic acid. ¹H NMR (400 MHz, DMSO-d₆) δ 9.36 (s, 1H), 6.86 (t, J=9.8 Hz, 1H), 6.55-6.47 (m, 1H), 5.32 (s, 2H), 2.93 (d, J=6.4 Hz, 2H), 2.21-2.10 (m, 1H), 1.01 (d, J=6.7 Hz, 6H); m/z (ES-MS) M+1=265.2.

### Example Q

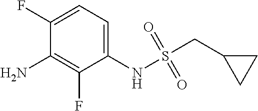

### N-(3-Amino-2,4-difluorophenyl)-1-cyclopropylmethanesulfonamide

Step A: 3-(Cyclopropylmethylsulfonamido)-2,6-difluorobenzoic acid was prepared according to the general procedure for Example M (step A), substituting methyl 3-amino-2,6-difluorobenzoate for methyl 3-amino-2-chloro-6-fluorobenzoate.

Step B: N-(3-Amino-2,4-difluorophenyl)-1-cyclopropylmethanesulfonamide was prepared according to the general procedure for Example M (step B), substituting 3-(cyclopropylmethylsulfonamido)-2,6-difluorobenzoic acid for 2-chloro-3-(cyclopropylmethylsulfonamido)-6-fluorobenzoic acid. ¹H NMR (400 MHz, DMSO-d₆) δ 9.37 (s, 1H), 6.84 (t, J=9.8 Hz, 1H), 6.57-6.50 (m, 1H), 5.30 (s, 2H), 3.01 (d, J=7.1 Hz, 2H), 1.11-0.98 (m, 1H), 0.59-0.51 (m, 2H), 0.35-0.27 (m, 2H); m/z (ES-MS) M+1=263.2.

### Example R

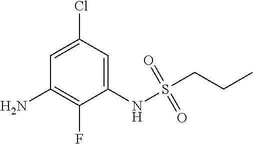

### N-(3-Amino-5-chloro-2-fluorophenyl)propane-1-sulfonamide

Step A: To a solution of methyl 5-chloro-2-fluorobenzoate (16.0 g, 84.8 mmol) in sulfuric acid (100 mL) at 0° C. was added fuming nitric acid (4.98 mL, 119 mmol). The reaction mixture was stirred at room temperature for 3 hours, poured into ice/water and the resulting precipitate was filtered. The obtained solid was purified by flash chromatography to afford methyl 5-chloro-2-fluoro-3-nitrobenzoate (6.78 g, 30%).

Step B: A round-bottom flask was charged with 5-chloro-2-fluoro-3-nitrobenzoate (6.78 g, 29.0 mmol), iron (16.2 g, 290 mmol), ammonium chloride (5.43 g, 102 mmol), ethanol (100 mL) and water (30 mL). The reaction mixture was stirred at 85° C. for 2 hours, then cooled to room temperature. The mixture was diluted with ethyl acetate and a saturated solution of NaHCO₃, and the layers were separated. The aqueous layer was extracted twice with ethyl acetate. The organic layers were combined, dried with sodium sulfate, filtered and concentrated in vacuo. The crude product was purified by flash chromatography to afford methyl 3-amino-5-chloro-2-fluorobenzoate (3.7 g, 63%).

Step C: To a solution of methyl 3-amino-5-chloro-2-fluorobenzoate (2.7097 g, 13.3 mmol) in THF (25 mL) and triethylamine (5.54 mL, 39.8 mmol) at 0° C. was added propane-1-sulfonyl chloride (3.12 mL, 27.8 mmol) dropwise. The reaction mixture was stirred at 0° C. for 90 minutes, after which 8N aqueous NaOH (16.6 mL, 130 mmol) was added. The reaction mixture was then warmed up at 40° C. and stirred for 16 hours. The volatiles were removed in vacuo, and the mixture was then acidified with concentrated HCl at 0° C. to pH 1. The acidified mixture was extracted with ethyl acetate twice. The organic layers were combined, dried with sodium sulfate, filtered and concentrated in vacuo to obtain crude 5-chloro-2-fluoro-3-(propylsulfonamido)benzoic acid, which was used in the next step without further purification.

Step D: N-(3-Amino-5-chloro-2-fluorophenyl)propane-1-sulfonamide was prepared according to the general procedure for Example M (step B), substituting 5-chloro-2-fluoro-3-(propylsulfonamido)benzoic acid for 2-chloro-3-(cyclopropylmethylsulfonamido)-6-fluorobenzoic acid. ¹H NMR (400 MHz, DMSO-d₆) δ 9.58 (s, 1H), 6.59 (dd, J=7.1 Hz, 2.6 Hz, 1H), 6.53 (dd, J=5.9 Hz, 2.6 Hz, 1H), 5.56 (s, 2H), 3.11-3.03 (m, 2H), 1.78-1.65 (m, 2H), 0.97 (t, J=7.4 Hz, 3H); m/z (ES-MS) M+1=267.0.

### Example S

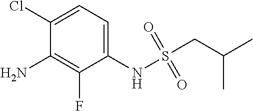

### N-(3-Amino-4-chloro-2-fluorophenyl)-2-methylpropane-1-sulfonamide

Step A: Benzyl 6-chloro-2-fluoro-3-(N-(isobutyl-sulfonyl)-2-methyl-propyl-□ulfonamide)benzoate was prepared according to the general procedure for Example F (step B), substituting 2-methylpropane-1-sulfonyl chloride for propane-1-sulfonyl chloride.

Step B: 6-Chloro-2-fluoro-3-(2-methylpropylsulfonamido)benzoic acid was prepared according to the general procedure for Example F (step C) substituting benzyl 6-chloro-2-fluoro-3-(N-(isobutylsulfonyl)-2-methylpropylsulfonamido)benzoate for benzyl 6-chloro-2-fluoro-3-(N-(propylsulfonyl)propylsulfonamido) benzoate.

Step C: N-(3-Amino-4-chloro-2-fluorophenyl)-2-methylpropane-1-sulfonamide was prepared according to the general procedure for Example M (step B), substituting 6-chloro-2-fluoro-3-(2-methylpropylsulfonamido)benzoic acid for 2-chloro-3-(cyclopropylmethylsulfonamido)-6-fluorobenzoic acid. ¹H NMR (400 MHz, DMSO-d₆) δ 9.50 (s, 1H), 7.02 (dd, J=8.8, 1.8 Hz, 1H), 6.62-6.54 (m, 1H), 5.45 (s, 2H), 2.97 (d, J=6.4 Hz, 2H), 2.21-2.10 (m, 1H), 1.01 (d, J=6.7 Hz, 6H); m/z (ES-MS) M+1=281.2.

### Example T

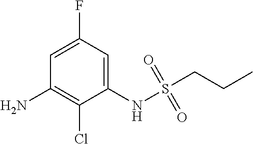

### N-(3-Amino-2-chloro-5-fluorophenyl)propane-1-sulfonamide

Step A: To a solution of 2-chloro-5-fluorobenzene-1,3-diamine (1.01 g, 6.29 mmol; 70% purity) (described in U.S. Pat. Publication No. 2006/0258888) in dichloromethane (30 mL) and triethylamine (1.93 mL, 13.8 mmol) was added propane-1-sulfonyl chloride (1.41 mL, 12.6 mmol) at 0° C. The reaction mixture was stirred at room temperature for 1 hour. An aqueous saturated solution of NaHCO₃ and ethyl acetate were added, and the layers were separated. The aqueous layer was extracted twice with ethyl acetate. The organic phases were combined, dried with sodium sulfate, filtered and concentrated in vacuo. The crude mixture was dissolved in tetrahydrofuran (15 mL) and methanol (5 mL), and 1.0 M of sodium hydroxide in water (6.3 mL) was added. The reaction mixture was stirred at room temperature for 30 minutes. An aqueous saturated solution of NaHCO₃ and ethyl acetate were added, and the layers were separated. The aqueous layer was extracted twice with ethyl acetate. The organic layers were combined, dried with sodium sulfate, filtered and concentrated in vacuo. The crude product was purified by flash chromatography to afford N-(3-amino-2-chloro-5-fluorophenyl)propane-1-sulfonamide (0.17 g, 7%). m/z (ES-MS) M+1=281.2.

### Example U

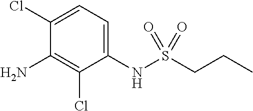

### N-(3-Amino-2,4-dichlorophenyl)propane-1-sulfonamide

Step A: 2,6-Dichloro-3-nitrobenzoic acid (2.13 g, 9.03 mmol) was dissolved in 2:1 THF:saturated aqueous NH₄Cl and cooled to 0° C. The mixture was treated with zinc (11.8 g, 181 mmol) and then allowed to warm to ambient temperature and stirred for 24 hours. The reaction mixture was filtered through GF/F paper while rinsing with THF. The mixture was acidified to a pH of 1 using 1.0 M HCl and extracted with 15% 2-propanol/dichloromethane (3×). The extracts were washed with water and brine, dried over sodium sulfate and concentrated to afford 3-amino-2,6-dichlorobenzoic acid (1.40 g, 6.82 mmol, 75.5% yield). MS (APCI-neg) m/z=203.6 (M−H).

Step B: 3-Amino-2,6-dichlorobenzoic acid (1.40 g, 6.82 mmol) was dissolved in dry dichloromethane (66.7 mL). Triethylamine (4.09 mL, 29.4 mmol) was added, and the mixture was chilled to 0° C. Propane-1-sulfonyl chloride (2.48 mL, 22 mmol) was then added using a syringe. When the addition was complete, the mixture was allowed to warm to ambient temperature and stirred for 1 hour. The mixture was concentrated in vacuo and diluted with diethyl ether. The mixture was washed with 0.25 M NaOH (80 mL) and the aqueous layer acidified to a pH of 1 using 1.0 M HCl. The aqueous layer was extracted with 15% 2-propanol:dichloromethane (2×300 mL). The organic layer was collected, dried over sodium sulfate, and concentrated to afford 2,6-dichloro-3-(propylsulfonamido)benzoic acid (1.55 g, 4.96 mmol, 74.4% yield). ¹H NMR (400 MHz, DMSO-d₆) δ 9.77-9.75 (s, 1H), 7.84-7.80 (d, 1H), 7.71-7.68 (d, 1H), 3.82-3.72 (m, 2H), 1.89-1.70 (m, 2H), 1.05-1.03 (m, 3H).

Step C: To a solution of 2,6-dichloro-3-(propylsulfonamido)benzoic acid (2.788 g, 8.93 mmol in THF (40 mL) was added triethylamine (2.863 mL, 20.5 mmol) and diphenylphosphonic azide (2.282 mL, 10.2 mmol). The reaction mixture was stirred for 6 hours at room temperature. Water (8 mL, 400 mmol) was added, and the reaction mixture was heated under reflux overnight. Ethyl acetate (300 mL) was added, followed by washing with saturated aqueous NaHCO₃ solution and brine. The solvent was removed under reduced pressure and the crude product purified via silica gel flash chromatography using ethyl acetate/hexane (1:1) as eluant to yield 834 mg (33%) of N-(3-amino-2,4-dichlorophenyl)propane-1-sulfonamide. ¹H NMR (500 MHz, DMSO-d₆) δ 9.24 (s, 1H), 7.20 (d, J=8.7 Hz, 1H), 6.71 (d, J=8.7 Hz, 1H), 5.55 (s, 2H), 3.13-2.92 (m, 2H), 1.73 (dd, J=15.2 Hz, 7.6 Hz, 2H), 0.96 (t, J=7.4 Hz, 3H). LC-MS [M+1]m/z 284.1.

### Example V

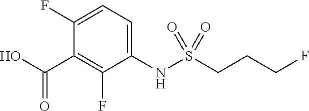

### 2,6-Difluoro-3-(3-fluoropropylsulfonamido)benzoic acid

Step A: Methyl 2,6-difluoro-3-(N-(3-fluoropropylsulfonyl)-3-fluoropropylsulfonamido)-benzoate was prepared according to the general procedure described in Example B, step C, substituting 3-fluoropropyl sulfonyl chloride for propane-1-sulfonyl chloride. ¹H NMR (400 MHz, DMSO-d₆) δ 8.05-7.99 (m, 1H), 7.44 (t, 1H), 4.62 (t, 2H), 4.50 (t, 2H), 3.93 (s, 3H), 3.89-3.74 (m, 4H), 2.26-2.11 (m, 4H).

Step B: 2,6-Difluoro-3-(3-fluoropropylsulfonamido)benzoic acid was prepared according to the general procedure in Example C, substituting methyl 2,6-difluoro-3-(N-(3-fluoropropylsulfonyl)-3-fluoropropylsulfonamido)benzoate for methyl 2,6-difluoro-3-(N-(propylsulfonyl)-propylsulfonamido)benzoate. ¹H NMR (500 MHz, DMSO-d₆) δ 14.05 (br s, 1H), 9.71 (s, 1H), 7.56-7.50 (m, 1H), 7.20 (t, 1H), 3.12-3.08 (m, 2H), 1.73-1.66 (m, 2H), 1.39 (sx, 2H), 0.87 (t, 3H). MS m/z 296.1 [M−1].

### Example W

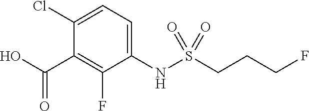

### 6-Chloro-2-fluoro-3-(3-fluoropropylsulfonamido)benzoic acid

Step A: Into a 5000-mL 4-necked round-bottom flask was placed a solution of benzyl 3-amino-6-chloro-2-fluorobenzoate (200 g, 714.29 mmol, 1.00 equiv) in dichloromethane (2000 mL) and triethylamine (216 g, 2.14 mol, 3.00 equiv) followed by the addition of a solution of 3-fluoropropane-1-sulfonyl chloride (227 g, 1.42 mol, 2.00 equiv) in dichloromethane (300 mL) dropwise with stirring at about 8° C. over 60 min. After stirring at room temperature for 3 hours, the resulting mixture was washed with 500 mL of 5N HCl and 2×500 mL of water. The organic layer was dried over anhydrous sodium sulfate and concentrated under vacuum to afford 360 g (91%) of benzyl 6-chloro-2-fluoro-3-(3-fluoro-N-(3-fluoropropylsulfonyl)propylsulfonamido)benzoate as a brown oil.

Step B: A solution of benzyl 6-chloro-2-fluoro-3-(3-fluoro-N-(3-fluoropropyl sulfonyl)propylsulfonamido)benzoate (360 g, 647.73 mmol, 1.00 equiv, 95%) in tetrahydrofuran (1800 mL) and KOH (2M, 1680 mL) was stirred at 50° C. for 12 hour. The resulting mixture was cooled and concentrated under vacuum to remove most of THF. The residual solution was washed with 3×500 mL of EtOAc. The aqueous layer was adjusted to pH 2-3 with HCl (6M). The resulting solution was extracted with 4×500 mL of ethyl acetate. The combined organic layers were dried over anhydrous sodium sulfate and concentrated under vacuum to afford 190 g (89%) of 6-chloro-2-fluoro-3-(3-fluoropropylsulfonamido)benzoic acid. ¹H NMR (400 MHz, DMSO-d₆) δ 9.65 (br s, 1H), 7.03 (m, 1H), 6.58 (m, 1H), 4.59 (m, 1H), 4.47 (m, 1H), 3.18 (m, 2H), 2.22-2.02 (m, 2H). MS m/z 312.1, 314.1 [M−1].

### Example X

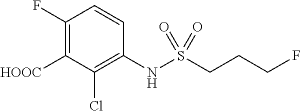

### 2-Chloro-6-fluoro-3-(3-fluoropropylsulfonamido)benzoic acid

Step A: Into a 2000-mL 3-necked round-bottom flask was placed a solution of methyl 3-amino-2-chloro-6-fluorobenzoate (50 g, 243.84 mmol, 1.00 equiv, 99%) in dichloromethane (900 mL) followed by the addition of triethylamine (75 g, 726.28 mmol, 3.00 equiv, 98%) dropwise with stirring at 0° C. To this was added a solution of 3-fluoropropane-1-sulfonyl chloride (55.6 g, 344.02 mmol, 1.30 equiv, 99%) in dichloromethane (100 mL) dropwise with stirring at −15° C. After stirring overnight at room temperature, the resulting solution was diluted with 500 mL of dichloromethane, washed with 2×500 mL of water and 5×500 mL of 4N HCl. The organic layer was washed with 2×500 mL of brine, dried over anhydrous sodium sulfate and concentrated under vacuum, to give 90 g crude mixture as a yellow oil which was used in the next step.

Step B: Into a 1000-mL round-bottom flask were placed tetrahydrofuran (250 mL) and a solution of potassium hydroxide (60 g, 1.05 mol, 3.00 equiv, 98%) in water (250 mL). The resulting solution was refluxed for 1 hour in an oil bath, cooled to room temperature with a water/ice bath, concentrated under vacuum, diluted with 100 mL of H₂O, and washed with 3×500 mL of ethyl acetate. The aqueous layer was adjusted to about pH 1 with HCl (2 mol/L). The resulting solution was extracted with 5×200 mL of ethyl acetate. The combined organic layers were washed with 1×500 mL of brine, dried over anhydrous sodium sulfate and concentrated under vacuum. The residue was washed with 1×200 mL of hexane and dried to afford 60 g (78%, two steps) of 2-chloro-6-fluoro-3-(3-fluoropropylsulfonamido)benzoic acid. ¹H NMR (400 MHz, MeOH-d₄) δ 7.63 (m, 1H), 7.19 (m, 1H), 4.56 (m, 1H), 4.44 (m, 1H), 3.21 (m, 2H), 2.25-2.12 (m, 2H). MS m/z 312.1, 314.1 [M−1].

### Example Y

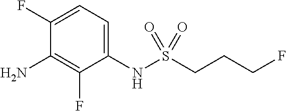

### N-(3-Amino-2,4-difluorophenyl)-3-fluoropropane-1-sulfonamide

Into a 3000-mL 4-necked round-bottom flask were placed a solution of 2,6-difluoro-3-(3-fluoropropylsulfonamido)benzoic acid (150 g, 479.80 mmol, 1.00 equiv, 95%) in N,N-dimethylformamide (1200 mL), TEA (153 g, 1.51 mol, 3.00 equiv) and DPPA (208.5 g, 758.18 mmol, 1.50 equiv). The resulting solution was stirred at 6° C. for 2 hours followed by the addition of water (364 mL, 40.00 equiv). The resulting solution was stirred at 80° C. in an oil bath for 1.5 hours, diluted with 3 L of H₂O, and extracted with 3×1 L of ethyl acetate. The combined organic layers were washed with 3×1 L of H₂O, dried over anhydrous sodium sulfate and concentrated under vacuum. The residue was applied onto a silica gel column eluted with ethyl acetate/petroleum ether (1:2) to afford 74.74 g (58%) of N-(3-amino-2,4-difluorophenyl)-3-fluoropropane-1-sulfonamide as a brown solid. ¹H NMR (400 MHz, CDCl₃) δ 2.265 (m, 2H), 3.252 (m, 2H), 3.805 (br s, 2H), 4.494 (t, 1H), 4.611 (t, 1H), 6.274 (s, 1H), 6.842 (m, 2H). LC-MS (ES, m/z): 268 [M+H]⁺.

### Example Z

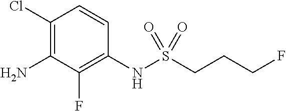

### N-(3-Amino-4-chloro-2-fluorophenyl)-3-fluoropropane-1-sulfonamide

Into a 3000-mL 3-necked round-bottom flask was placed a solution of 6-chloro-2-fluoro-3-(3-fluoropropylsulfonamido)benzoic acid (190 g, 574.84 mmol, 1.00 equiv, 95%) in N,N-dimethylformamide (1500 mL) and triethylamine (184 g, 1.82 mol, 3.00 equiv) followed by the addition of diphenylphosphoryl azide (“DPPA”) (250 g, 909.09 mmol, 1.50 equiv) dropwise with stirring at 5° C. over 10 min. After stirred at 5° C. for 2 hours, to the reaction mixture was added water (500 mL). The resulting solution was stirred at 80° C. in an oil bath for an additional 2 hours, cooled and diluted with 2000 mL of EtOAc. The organic layer was washed with 4×1000 mL of brine, dried over anhydrous sodium sulfate and concentrated under vacuum. The residue was applied onto a silica gel column eluted with ethyl acetate/petroleum ether (1:3) to afford 76 g (46%) of N-(3-amino-4-chloro-2-fluorophenyl)-3-fluoropropane-1-sulfonamide as a white solid. ¹H NMR (400 MHz, CDCl₃) δ 7.04-7.06 (m, 1H), 6.91-6.87 (t, 1H), 6.39 (s, 1H), 4.62-4.59 (t, 1H), 4.40-4.57 (t, 1H), 4.15 (br s, 1H), 3.27-3.24 (t, 2H), 2.30-2.16 (m, 2H). LC-MS (ES, m/z): 283 [M−H]⁻.

### Example AA

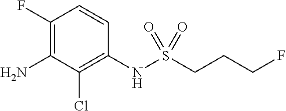

### N-(3-Amino-2-chloro-4-fluorophenyl)-3-fluoropropane-1-sulfonamide

Into three 1000-mL 3-necked round-bottom flask, purged and maintained with an inert atmosphere of nitrogen, was placed a solution of 2-chloro-6-fluoro-3-(3-fluoro-propylsulfonamido)benzoic acid (147 g, 422.68 mmol, 1.00 equiv, 90%) in N,N-dimethylformamide (1170 mL) followed by the addition of triethylamine (142 g, 1.38 mol, 3.00 equiv, 98%) dropwise with stirring at 0-5° C. To this was added diphenylphosphoryl azide (200 g, 712.73 mmol, 1.50 equiv, 98%) dropwise with stirring at 0° C. The resulting solution was stirred at 25° C. for 4 hours. The reaction mixture was diluted with water (340 mL). The resulting solution was stirred at 80° C. in an oil bath overnight, cooled to room temperature and concentrated under vacuum. The residual solution was diluted with 1500 mL of dichloromethane and washed with 4×1000 mL of saturated sodium bicarbonate solution and 1×1000 mL of brine. The organic layer was dried over anhydrous sodium sulfate and concentrated under vacuum. The residue was applied onto a silica gel column eluted with ethyl acetate/petroleum ether (1:4) to afford 50.3 g (41%) of N-(3-amino-2-chloro-4-fluorophenyl)-3-fluoropropane-1-sulfonamide as a white solid. ¹H NMR (400 MHz, DMSO-d₆) δ9.84 (s, 1H), 7.06-7.02 (d, 1H), 6.65-6.62 (d, 1H), 5.535 (s, 2H), 4.62-4.59 (m, 1H), 4.50-4.47 (m, 1H), 3.18-3.15 (m, 2H), 2.17-2.04 (m, 2H). LC-MS (ES, m/z): 285 [M+H]⁺.

### Example AB

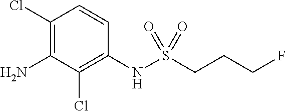

### N-(3-Amino-2,4-dichlorophenyl)-3-fluoropropane-1-sulfonamide

Step A: To 3-amino-2,6-dichlorobenzoic acid (8.00 g, 38.8 mmol) in tetrahydrofuran (200 mL) at 0° C. was added dropwise triethylamine (29.8 mL, 214 mmol) followed by 3-fluoropropane-1-sulfonyl chloride (15.1 mL, 136 mmol). The reaction mixture was stirred at 50° C. for 16 hours and then cooled to room temperature. Water and dichloromethane were added. The layers were separated, and the aqueous layer was extracted twice with dichloromethane. The organic layers were combined, dried with sodium sulfate, filtered and concentrated in vacuo. The obtained oil was dissolved in tetrahydrofuran (107 mL), and 8 M NaOH (49 mL) was added dropwise at room temperature. The reaction mixture was heated at 50° C. The volatiles were removed in vacuo and the reaction mixture acidified with concentrated HCl at 0° C. to pH 1. The aqueous phase was then extracted twice with ethyl acetate. The organic layers were combined, dried with sodium sulfate, filtered, and concentrated in vacuo. The crude product was purified by flash chromatography to afford 2,6-dichloro-3-(3-fluoropropylsulfonamido)benzoic acid (6.7 g, 44%).

Step B: To a solution of 2,6-dichloro-3-(3-fluoropropylsulfonamido)benzoic acid (6.7 g, 20.0 mmol) in 1,4-dioxane (50 mL) was added triethylamine (3.11 mL, 22.3 mmol), followed by diphenylphosphonic azide (4.73 mL, 21.3 mmol) at room temperature. The reaction mixture was stirred at room temperature for 1 hour, then at 50° C. for 7 hours. The reaction mixture was subsequently added dropwise, via an addition funnel, over 15 minutes in a round-bottom flask containing 1,4-dioxane (24 mL) and water (1.83 mL, 101 mmol) at 95° C. The reaction was stirred at this temperature for 16 hours. The reaction mixture was concentrated in vacuo to half its volume and then diluted with ethyl acetate and a saturated solution of NaHCO₃. The layers were separated, and the aqueous layer was extracted twice with ethyl acetate. The organic layers were combined, dried with sodium sulfate, filtered and concentrated in vacuo. The crude product was purified by flash chromatography to afford N-(3-amino-2,4-dichlorophenyl)-3-fluoropropane-1-sulfonamide (3.06 g, 50%). ¹H NMR (400 MHz, DMSO-d₆) δ 9.45 (s, 1H), 7.23 (d, J=8.6 Hz, 1H), 6.70 (d, J=8.6 Hz, 1H), 5.61 (s, 2H), 4.59 (t, J=5.8 Hz, 1H), 4.48 (t, J=5.8 Hz, 1H), 3.25-3.15 (m, 2H), 2.20-2.01 (m, 2H). m/z (ES-MS) 301.2 (100%) [M+1].

### Example AC

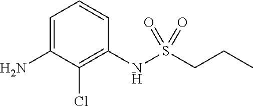

### N-(3-Amino-2-chlorophenyl)propane-1-sulfonamide

Step A: 2-Chloro-3-nitroaniline (Sienkowska, et. al., Tetrahedron 56 (2000) 165) (0.36 g, 2.086 mmol) was dissolved in dichloromethane (20 mL) and cooled to 0° C. Triethylamine (0.8723 mL, 6.258 mmol) was added followed by propane-1-sulfonyl chloride (0.5847 mL, 5.215 mmol) and the reaction was stirred at room temperature overnight. The reaction was quenched with 0.1 N HCl (10 mL), and the layers were separated. The organic layer was dried over Na₂SO₄, and concentrated to give N-(2-chloro-3-nitrophenyl)-N-(propylsulfonyl)propane-1-sulfonamide as an oil which was used directly in the next step.

Step B: N-(2-Chloro-3-nitrophenyl)-N-(propylsulfonyl)propane-1-sulfonamide (0.8028 g, 2.086 mmol) was dissolved in 3:1 THF/MeOH (4.0 mL). NaOH (2.0 M, 2.086 mL, 4.172 mmol) was added and the reaction was stirred for five minutes at room temperature. The reaction was quenched with 0.1N HCl (5 mL) and the volatiles were removed by rotary evaporation. EtOAc (10 mL) was added and the organic layer was washed with water and brine, dried with Na₂SO₄ and concentrated to give N-(2-chloro-3-nitro-phenyl)propane-1-sulfonamide as an oil which was used directly in the next step.

Step C: N-(2-Chloro-3-nitrophenyl)propane-1-sulfonamide (0.580 g, 2.08 mmol) was dissolved in 4:1 EtOH/water (10 mL). Fe(0) (1.16 g, 20.8 mmol) was added followed by a catalytic amount of NH₄Cl (5 mg) and the reaction was heated to 80° C. for 3 hours. The reaction mixture was cooled to room temperature, filtered through Celite®, concentrated, dissolved in EtOAc, washed with water, dried over Na₂SO₄ and concentrated. Purification by silica gel chromatography (10% to 90% EtOAc/Hex) gave N-(3-amino-2-chlorophenyl)propane-1-sulfonamide (259 mg, 1.04 mmol, 51%). ¹H NMR (400 MHz, DMSO-d₆) δ 9.06 (br s, 1H), 6.96-6.99 (d, 1H), 6.63-6.66 (m, 2H), 5.43 (bs, 1H), 3.03-3.07 (t, 1H), 1.71-1.77 (m, 2H), 0.94-0.98 (t, 3H); m/z (APCI-neg) M−1=247.1, 249.0.

### Example AD

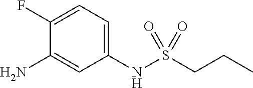

### N-(3-Amino-4-fluorophenyl)propane-1-sulfonamide

A solution of N-(3-amino-2-chloro-4-fluorophenyl)propane-1-sulfonamide (668 mg, 2.5 mmol) dissolved in methanol (100 mL) was passed through an H-Cube hydrogenator equipped with a Pd/C cartridge at 10 bar H₂ pressure at a flow rate of 1 mL/minute (reaction temperature: 50° C.). The solvent was concentrated under reduced pressure to afford 481 mg (83%) of N-(3-amino-4-fluorophenyl)propane-1-sulfonamide. ¹H NMR (500 MHz, DMSO-d₆) δ 9.37 (s, 1H), 6.89 (dd, J=11.2, 8.7, 1H), 6.67 (dd, J=8.1, 2.6, 1H), 6.49-6.24 (m, 1H), 5.19 (s, 2H), 3.09-2.86 (m, 2H), 1.67 (dq, J=15.0, 7.5, 2H), 0.93 (t, J=7.4, 3H). LC-MS [M+1]m/z 233.1.

### Example AE

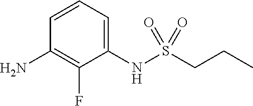

### N-(3-Amino-2-fluorophenyl)propane-1-sulfonamide

A solution of N-(3-Amino-4-chloro-2-fluorophenyl)propane-1-sulfonamide (477 mg, 1.8 mmol) dissolved in methanol (100 mL) was passed through an H-Cube hydrogenator equipped with a Pd/C cartridge at ambient temperature and pressure at a flow rate of 1 mL/minute. The solvent was concentrated under reduced pressure to afford 251 mg (60%) of N-(3-amino-2-fluorophenyl)propane-1-sulfonamide. ¹H NMR (500 MHz, DMSO-d₆) δ 9.29 (s, 1H), 6.79 (t, J=8.0, 1H), 6.58 (td, J=8.1, 1.4, 1H), 6.55-6.49 (m, 1H), 5.17 (s, 2H), 3.02 (dd, J=8.7, 6.7, 2H), 1.85-1.60 (m, 2H), 0.96 (t, J=7.4, 3H). LC-MS [M+1]m/z 233.1.

### Example AF

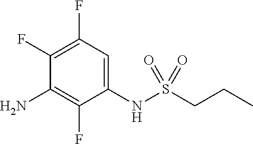

### N-(3-Amino-2,4,5-trifluorophenyl)propane-1-sulfonamide

2,4,5-Trifluorobenzene-1,3-diamine (1116 mg, 6.88 mmol) was dissolved in dichloromethane (27 mL, 420 mmol) and pyridine (557 ul, 6.88 mmol) was added. After cooling the mixture to 0° C., propane-1-sulfonyl chloride (772 ul, 6.88 mmol) was added drop-wise through a syringe. The ice bath was removed and the mixture was stirred at RT overnight. The solvent was removed under reduced pressure and the crude product purified via chromatography eluting with 1:1 ethyl acetate/hexane to afford N-(3-amino-2,4,5-trifluorophenyl)propane-sulfonamide (1847 mg, 83.6%). ¹H NMR (400 MHz, DMSO-d₆) δ 9.58 (s, 1H), 6.53 (dt, J=11.8, 7.5 Hz, 1H), 5.75 (s, 2H), 3.10-2.91 (m, 2H), 1.72 (dd, J=15.1, 7.5 Hz, 2H), 0.96 (t, J=7.4 Hz, 3H). LC-MS [M+1]m/z 269.0.

### Example AG

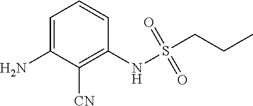

### N-(3-Amino-2-cyanophenyl)propane-1-sulfonamide

To propane-1-sulfonamide (0.950 g, 7.71 mmol) in 7 mL N-methylpyrrolidone in a vial was added 60% sodium hydride (0.194 g, 8.08 mmol). After gas evolution ceased, the mixture was stirred 30 minutes at 40° C., then 2-amino-6-fluorobenzonitrile (0.500 g, 3.67 mmol) was added and the sealed vial was heated at 120° C. overnight, then for 4 days at 150° C. The reaction mixture was partitioned between 0.5 M NaOH and EtOAc. The aqueous layer was acidified to pH 5 with concentrated HCl and extracted with EtOAc. The EtOAc extract was washed with twice with brine, dried over MgSO₄, filtered, and evaporated to yield 0.73 g. This material was dissolved in ether and washed with 3 portions water to remove NMP, dried over MgSO₄, filtered, and evaporated to yield N-(3-amino-2-cyanophenyl)propane-1-sulfonamide (0.34 g, 1.42 mmol, 38.7% yield) as an orange film. ¹H NMR (400 MHz, CDCl₃) δ 7.39 (d, 1H), 7.20-7.26 (m, 1H), 6.96 (d, 1H), 6.73 (br s, 1H), 4.92 (br s, 2H), 3.13-3.18 (m, 2H), 1.85-1.95 (m, 2H), 1.06 (t, 3H). m/z 272.1 (274.1 40%) (LC/MS negative ionization) [M−1].

### Example AH

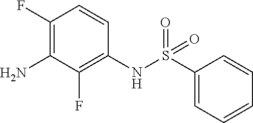

### N-(3-Amino-2,4-difluorophenyl)benzenesulfonamide

Step A: Methyl 3-amino-2,6-difluorobenzoate (1.14 g, 6.092 mmol) was dissolved in dichloromethane (30.5 mL) and treated sequentially with triethylamine (2.50 mL, 18.27 mmol) and benzenesulfonyl chloride (1.63 mL, 12.79 mmol). The reaction mixture was stirred at ambient temperature for 4 hours and then diluted with additional dichloromethane and washed with water (2×) and brine (1×). The organic phase was dried over Na₂SO₄ and concentrated to provide methyl 2,6-difluoro-3-(N-(phenylsulfonyl)phenylsulfonamido)benzoate (2.848 g, 6.092 mmol). The crude material was then immediately dissolved in 60.9 mL 4:1 THF:MeOH (0.1 M) and treated with 2.0 M KOH (15.23 mL, 30.46 mmol). The reaction mixture was stirred at ambient temperature for 2 hours. The organic solvent was removed under reduced pressure and the aqueous residue acidified to pH 3 using 1.0 M HCl. Extraction with EtOAc (2×) was followed by washing the combined organic extracts with water (2×). The crude product was then extracted as its carboxylate salt with 1.0 M NaOH (2×). The combined aqueous NaOH extracts were acidified to pH 3 using 6.0 M HCl and extracted with EtOAc (2×). The combined organic extracts were washed with water (2×) and brine (1×) and then dried over Na₂SO₄ and concentrated to afford 2,6-difluoro-3-(phenylsulfonamido)benzoic acid (1.53 g, 4.884 mmol, 80.17% yield). LC/MS: m/z 312.0 [M−1].

Step B: 2,6-Difluoro-3-(phenylsulfonamido)benzoic acid (1.53 g, 4.884 mmol) was dissolved in 25 mL DMF (25 mL) and treated sequentially with triethylamine (1.99 mL, 14.65 mmol) and then diphenylphosphoryl azide (1.633 mL, 7.326 mmol). The reaction mixture was stirred at ambient temperature for 1 hour and then treated with 10 mL water and heated to 80° C. for 16 hours. The reaction mixture was cooled to ambient temperature and diluted with water. Extraction with EtOAc (2×) and washing of the combined organic phases with water (4×) and brine (1×) was followed by drying over Na₂SO₄ and concentration under reduced pressure. Purification via flash chromatography eluting with a gradient of 10->70% EtOAc:hexanes afforded N-(3-amino-2,4-difluorophenyl)benzenesulfonamide (508.9 mg, 1.790 mmol, 35.65% yield). LC/MS: m/z 283.1 [M−1].

### Example AI

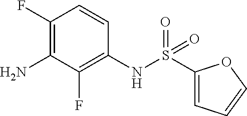

### N-(3-Amino-2,4-difluorophenyl)furan-2-sulfonamide

Step A: Methyl 3-amino-2,6-difluorobenzoate (652.8 mg, 3.488 mmol) was dissolved in 17.4 mL dichloromethane (0.2 M) and treated sequentially with triethylamine (1.42 mL, 10.46 mmol) and furan-2-sulfonyl chloride (1.162 g, 6.976 mmol). The reaction mixture was stirred at ambient temperature for 16 hours and then diluted with additional dichloromethane and washed with water (2×) and brine (1×). The organic phase was dried over Na₂SO₄ and concentrated to provide methyl 2,6-difluoro-3-(N-(furan-2-ylsulfonyl)furan-2-sulfonamido)benzoate (1.561 g, 3.489 mmol). The crude material was then immediately dissolved in 17.5 mL 4.1 THF:MeOH (0.2 M) and treated with 2.0 M KOH (8.7 mL, 17.45 mmol). The reaction mixture was stirred at ambient temperature for 2 hours. The organic solvent was removed under reduced pressure and the aqueous residue acidified to pH 3 using 1.0 M HCl. Extraction with EtOAc (2×) was followed by washing the combined organic extracts with water (2×). The crude product was then extracted as its carboxylate salt with 1.0 M NaOH (2×). The combined aqueous NaOH extracts were acidified to pH 3 using 6.0 M HCl and extracted with EtOAc (2×). The combined organic extracts were washed with water (2×) and brine (1×) and then dried over Na₂SO₄ and concentrated to afford 2,6-difluoro-3-(furan-2-sulfonamido)benzoic acid (475.0 mg, 1.566 mmol, 44.91% yield). LC/MS: m/z 302.0 [M−1].

Step B: 2,6-difluoro-3-(furan-2-sulfonamido)benzoic acid (475.0 mg, 1.566 mmol) was dissolved in DMF (15.7 mL) and treated sequentially with triethylamine (0.637 mL, 4.699 mmol) and then diphenylphosphoryl azide (0.524 mL, 2.350 mmol). The reaction mixture was stirred at ambient temperature for 1 hour and then treated with 5 mL water and heated to 80° C. for 16 hours. The reaction mixture was cooled to ambient temperature and diluted with water. Extraction with EtOAc (2×) and washing of the combined organic phases with water (4×) and brine (1×) was followed by drying over Na₂SO₄ and concentration under reduced pressure. Purification via flash chromatography eluting with a gradient of 5 to >60% EtOAc:hexanes afforded N-(3-amino-2,4-difluorophenyl)furan-2-sulfonamide (152.6 mg, 0.556 mmol, 35.52% yield). LC/MS: m/z 273.1 [M−1].

### Example AJ

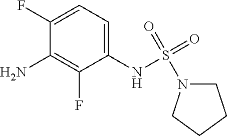

### N-(3-Amino-2,4-difluorophenyl)pyrrolidine-1-sulfonamide

To N-(3-amino-2,4-difluorophenyl)propane-1-sulfonamide (0.250 g, 0.999 mmol) in DMF (4.5 mL) was added potassium carbonate (0.414 g, 3.00 mmol) and pyrrolidine-1-sulfonyl chloride (0.196 mL, 1.50 mmol). The suspension was stirred at ambient temperature for 18 hours. To the suspension was then added 1 mL of 2M NaOH which stirred at ambient temperature for 1 hour. The resulting solution was diluted with water (20 mL) and brought to pH 9 with HCl followed by extraction with EtOAc (3×15 mL). The concentrated organics were purified via silica gel chromatography eluting with 1:1 Hexane-EtOAc to provide N-(3-amino-2,4-difluorophenyl)pyrrolidine-1-sulfonamide (184 mg, 66%). ¹H NMR (400 MHz, MeOD-d₄) δ 6.86-6.09 (m, 1H), 6.75-9.82 (m, 1H), 6.34 (br s, 1H), 3.78 (br s, 2H), 3.28-3.33 (m, 4H), 1.82-1.87 (m, 4H). LC/MS: m/z 276.1 [M−1].

### Example AK

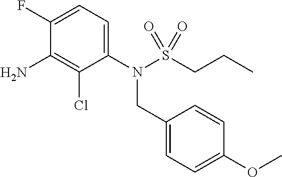

### N-(3-Amino-2-chloro-4-fluorophenyl)-N-(4-methoxybenzyl)propane-1-sulfonamide

N-(3-Amino-2-chloro-4-fluorophenyl)propane-1-sulfonamide (75 g, 280 mmol) was dissolved in N,N-dimethylformamide (200 mL, 2000 mmol). A 60% sodium hydride suspension in mineral oil (6:4, sodium hydride:mineral oil, 11.85 g, 296 mmol) was added in multiple portions over a period of 15 minutes. The reaction mixture was stirred at room temperature for 90 minutes and was then warmed to 40° C. for two hours. This homogeneous mixture was cooled to 0° C. and p-pethoxybenzyl chloride (40.03 mL, 295.25 mmol) was added over 5 minutes. The reaction was left to stir and warm to room temperature. After 14 hours, the reaction mixture was poured into a dilute ammonium chloride solution (1750 mL) and the water layer was decanted to leave an orange oil. This oil was triturated three times with water (2 L). The remaining product was transferred into a 1 L beaker, diluted with 800 mL water, sonicated for 30 minutes and then stirred at room temperature for 1 hour. The resulting light yellow solid was collected via filtration and dried by lyophilization to give 111.9 g (99%) of N-(3-amino-2-chloro-4-fluorophenyl)-N-(4-methoxybenzyl)propane-1-sulfonamide. ¹H NMR (500 MHz, DMSO-d₆) δ 7.11 (d, J=8.6 Hz, 2H), 6.96 (dd, J=10.6, 8.8 Hz, 1H), 6.81 (t, J=5.7 Hz, 2H), 6.51 (dd, J=8.7, 5.1 Hz, 1H), 5.42 (s, 2H), 4.71 (d, J=14.4 Hz, 1H), 4.57 (d, J=14.4 Hz, 1H), 3.70 (s, 3H), 3.21 (td, J=6.7, 1.4 Hz, 2H), 1.77 (dd, J=15.3, 7.5 Hz, 2H), 1.00 (t, J=7.4 Hz, 3H).

### Example AL

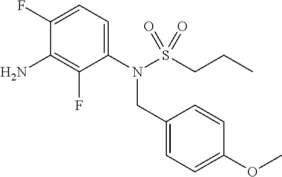

### N-(3-Amino-2,4-difluorophenyl)-N-(4-methoxybenzyl)propane-1-sulfonamide

The title compound was prepared using the procedure described in Example AK using N-(3-amino-2,4-difluorophenyl)propane-1-sulfonamide instead of N-(3-Amino-2-chloro-4-fluorophenyl)propane-1-sulfonamide as starting material. ¹H NMR (400 MHz, DMSO-d₆) δ 7.13 (m, 2H), 6.92-6.76 (m, 3H), 6.49 (td, J=8.5, 5.6 Hz, 1H), 5.25 (s, 2H), 4.64 (s, 2H), 3.70 (s, 3H), 3.25-3.16 (m, 2H), 1.85-1.69 (m, 2H), 1.00 (t, J=7.4 Hz, 3H). ES-MS [M+1]m/z 370.2.

### Example AM

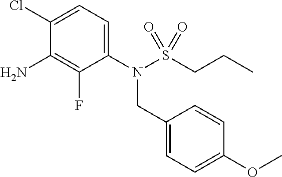

### N-(3-Amino-4-chloro-2-fluorophenyl)-N-(4-methoxybenzyl)propane-1-sulfonamide

The title compound was prepared using the procedure described in Example AK using N-(3-amino-4-chloro-2-fluorophenyl)propane-1-sulfonamide instead of N-(3-Amino-2-chloro-4-fluorophenyl)propane-1-sulfonamide as starting material. ¹H NMR (400 MHz, DMSO-d₆) δ 7.18-7.10 (m, 2H), 6.99 (dd, J=8.7, 1.7 Hz, 1H), 6.91-6.70 (m, 2H), 6.53 (dd, J=8.6, 7.7 Hz, 1H), 5.43 (s, 2H), 4.66 (s, 2H), 3.70 (s, 3H), 3.26-3.19 (m, 2H), 1.85-1.61 (m, 2H), 1.00 (t, J=7.4 Hz, 31-1). ES-MS [M+NH4+]m/z 404.2/406.2.

### Example AN

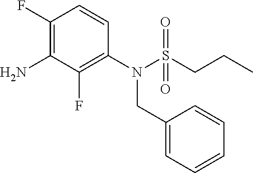

### N-(3-Amino-2,4-difluorophenyl)-N-benzylpropane-1-sulfonamide

N-(3-Amino-2,4-difluorophenyl)propanesulfonamide (5.6 g, 20 mmol) was dissolved in N,N-dimethylformamide (40 mL) and cooled to 0° C. Sodium hydride (0.88 g, 22 mmol) was added in small portions and the mixture was stirred at room temperature for 1 hour. The mixture was cooled to 0° C., benzyl bromide 2.6 mL, 22 mmol) was added, and the mixture was stirred at room temperature overnight. A dilute solution of ammonium chloride was added (200 mL), and the mixture was stirred at room temperature for 1 hour. The mixture was decanted, the oily residue was applied to a Varian Chemelut™ cartridge, and eluted with ethyl acetate. The crude product was purified using flash chromatography (gradient elution: 0-30% ethyl acetate in heptanes) to yield N-(3-amino-2,4-difluorophenyl)-N-benzylpropane-1-sulfonamide as an oil, which slowly solidified at room temperature (4.85 g, 71%). ¹H NMR (400 MHz, DMSO-d₆) δ 7.34-7.16 (m, 5H), 6.81 (t, J=9.2 Hz, 1H), 6.53 (td, J=8.5, 5.7 Hz, 1H), 5.26 (s, 2H), 4.72 (s, 2H), 3.27-3.16 (m, 6H), 1.86-1.67 (m, 2H), 1.01 (t, J=7.4 Hz, 3H).

### Example AO

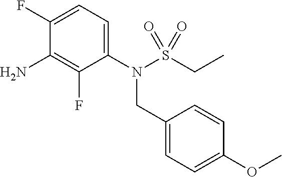

### N-(3-Amino-2,4-difluorophenyl)-N-(4-methoxybenzyl)ethanesulfonamide

N-(3-Amino-2,4-difluorophenyl)ethanesulfonamide (2.03 g, 8.57 mmol) was dissolved in N,N-dimethylformamide (8.4 mL), and the mixture was cooled over an ice bath. Sodium hydride (0.373 g, 9.31 mmol) was added, and the flask was removed from the ice water bath after vigorous bubbling subsided. The reaction mixture was stirred at room temperature for 1 hour and then cooled over an ice bath. p-Methoxybenzyl chloride (1.21 mL, 8.89 mmol) was added, followed by stirring overnight, gradually raising to room temperature. The reaction mixture was concentrated to remove the DMF, and then 50 mL of a solution of water and saturated aqueous ammonium chloride (v/v 50:50) was added. The reaction mixture was then stirred at room temperature overnight. The precipitated solid was collected by filtration and then purified by flash chromatography (120 g column, 0-50% ethyl acetate: heptane) to give N-(3-amino-2,4-difluorophenyl)-N-(4-methoxybenzyl)ethanesulfonamide as a solid (2.432 g, 79.6%). ¹H NMR (400 MHz, DMSO-d₆) δ 7.13 (d, J=8.6 Hz, 2H), 6.86-6.77 (m, 3H), 6.48 (td, J=8.5, 5.6 Hz, 1H), 5.28 (s, 2H), 4.64 (s, 2H), 3.70 (s, 3H), 3.24 (q, J=7.3, 2H), 1.29 (t, J=7.3 Hz, 3H).

### Example AP

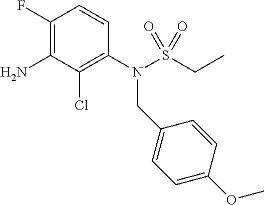

### N-(3-Amino-2-chloro-4-fluorophenyl)-N-(4-methoxybenzyl)ethanesulfonamide

The title compound was prepared using the procedure described in Example AO using N-(3-amino-2-chloro-4-fluorophenyl)ethanesulfonamide in place of N-(3-Amino-2,4-difluorophenyl)ethanesulfonamide as starting material. ¹H NMR (400 MHz, DMSO-d₆) δ 7.11 (d, J=8.6 Hz, 2H), 6.96 (dd, J=10.7, 8.8 Hz, 1H), 6.81 (d, J=8.7 Hz, 2H), 6.49 (dd, J=8.8, 5.1 Hz, 1H), 5.46 (s, 2H), 4.71 (d, J=14.3 Hz, 1H), 4.56 (d, J=14.4 Hz, 1H), 3.69 (s, 3H), 3.31-3.20 (m, 2H), 1.28 (q, J=7.2 Hz, 3H).

### Example AQ

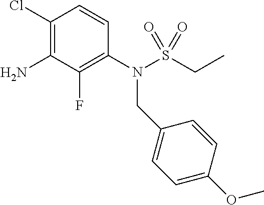

### N-(3-Amino-4-chloro-2-fluorophenyl)-N-(4-methoxybenzyl)ethane sulfonamide

The title compound was prepared using the procedure described in Example AO using N-(3-amino-4-chloro-2-fluorophenyl)ethanesulfonamide in place of N-(3-Amino-2,4-difluorophenyl)ethanesulfonamide as starting material. ¹H NMR (500 MHz, DMSO-d₆) δ 7.14 (d, J=8.6 Hz, 2H), 6.99 (dd, J=8.7, 1.5 Hz, 1H), 6.83 (t, J=5.8 Hz, 2H), 6.59-6.43 (m, 1H), 5.42 (s, 2H), 4.66 (s, 2H), 3.72 (s, 3H), 3.25 (q, J=7.4 Hz, 2H), 1.29 (t, J=7.3 Hz, 3H).

### Example AR

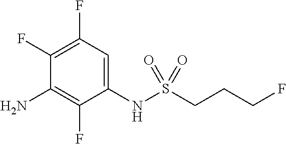

### N-(3-Amino-2,4,5-trifluorophenyl)-3-fluoropropane-1-sulfonamide

To a stirred solution of 2,4,5-trifluorobenzene-1,3-diamine (1116 mg, 6.88 mmol) in dichloromethane (27 mL, 420 mmol) was added pyridine (557 ul, 6.88 mmol). The reaction mixture was cooled to 0° C. and 3-fluoropropane-1-sulfonyl chloride (762 ul, 6.88 mmol) was added drop-wise. The ice bath was removed and the mixture was stirred at RT overnight. The organics were removed via reduced pressure and the crude product was purified through column chromatography eluted with 1:1 ethyl acetate/hexane to give N-(3-amino-2,4,5-trifluorophenyl)-3-fluoropropane-1-sulfonamide (628 mg, 32%). ¹H NMR (400 MHz, DMSO) δ 9.72 (s, 1H), 6.54 (dt, J=12.1, 7.4 Hz, 1H), 5.78 (s, 2H), 4.60 (t, J=5.9 Hz, 1H), 4.48 (t, J=5.9 Hz, 1H), 3.26-3.13 (m, 2H), 2.19-1.99 (m, 2H); LC-MS [M+1]m/z 287.0.

### Example AS

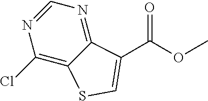

### Methyl 4-chlorothieno[3,2-d]pyrimidine-7-carboxylate

Step A: To a solution of 3H-thieno[3,2-d]□ulfona-4-one (25 g, 164 mmol) in acetic acid (200 mL) was added bromine (26 mL) dropwise. The reaction mixture was heated at 100° C. for 8 hours. The resulting suspension was cooled to room temperature, poured into water, and neutralized with solid sodium bicarbonate. The solid product was collected by vacuum filtration to yield 21.4 g of 7-bromo-3H-thieno[3,2-d]□ulfona-4-one (60% yield) as a solid. ¹H NMR (500 MHz, DMSO-d₆) δ 12.75 (s, 1H), 8.38 (s, 1H), 8.27 (s, 1H).

Step B: 7-Bromo-3H-thieno[3,2-d]LI ulfona-4-one (10.0 g, 40.7 mmol), [1,1′-bis-(diphenyl-phosphino)ferrocene]dichloropalladium(II) complex with dichloromethane (1:1) (830.5 mg, 1.017 mmol), triethylamine (28.35 mL, 203.4 mmol), and methanol (80 mL) were combined in an autoclave fitted with a large stir bar. The mixture was purged with nitrogen for five minutes. The vessel was placed under an atmosphere of carbon monoxide (300 psi) and heated to 120° C. for 3 hours. The vessel was cooled to room temperature and the reaction mixture was filtered. The collected solids were washed with methanol (250 mL). The solids were air-dried to give methyl 4-hydroxythieno[3,2-d]pyrimidine-7-carboxylate (6.8 g, 80%). ¹H NMR (400 MHz, DMSO-d₆) δ 12.74 (s, 1H), 8.90 (s, 1H), 8.27 (s, 1H), 3.89 (s, 3H).

Step C: Methyl 4-hydroxythieno[3,2-d]pyrimidine-7-carboxylate (6.8 g, 31 mmol) was dissolved in phosphoryl chloride (100 mL, 1000 mmol) and heated to reflux for 2 hours. The mixture was stirred at room temperature overnight. The phosphoryl chloride was distilled off, and the solids were neutralized with ice and sodium bicarbonate. The resulting suspension was filtered to give a solid, which was triturated with anhydrous ether. The resulting suspension was filtered to yield methyl 4-chlorothieno[3,2-d]pyrimidine-7-carboxylate as a solid (6.76 g, 96%). ¹H NMR (400 MHz, DMSO-d₆) δ 9.30 (s, 1H), 9.17 (s, 1H), 3.91 (s, 3H).

### Example AT

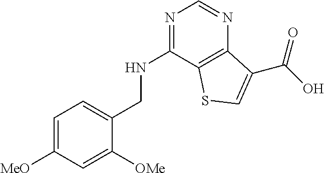

### 4-(2,4-Dimethoxybenzylamino)thieno[3,2-d]pyrimidine-7-carboxylic acid

Step A: Methyl 4-chlorothieno[3,2-d]pyrimidine-7-carboxylate (0.396 g, 1.73 mmol), DIEA (0.452 mL, 2.60 mmol), and (2,4-dimethoxyphenyl)methanamine (0.274 mL, 1.82 mmol) were dissolved in DMF and heated to 60° C. for 3 hours. The reaction was cooled to room temperature, partitioned between EtOAc and water and the layers separated. The organic layer was washed with water (3×), 0.1N HCl and brine, dried over Na₂SO₄ and concentrated to give methyl 4-(2,4-dimethoxybenzylamino)thieno[3,2-d]pyrimidine-7-carboxylate as a tan oil which was used directly in the next step.

Step B: Methyl 4-(2,4-dimethoxybenzylamino)thieno[3,2-d]pyrimidine-7-carboxylate (0.622 g, 1.73 mmol) was dissolved in 4:1 THF/MeOH (20 mL) NaOH (2.0 M, 2.60 mL, 5.19 mmol) was added and stirred at room temperature overnight. The solution was brought to pH 12 with 0.1N NaOH and diluted with EtOAc. The layers were separated and the aqueous layer was acidified with 1.0 N HCl to pH 3 and extracted with dichloromethane (3×). The combined organics were dried over Na₂SO₄ and concentrated to give 4-(2,4-dimethoxybenzylamino)thieno-[3,2-d]pyrimidine-7-carboxylic acid (365 mg, 1.06 mmol, 61%) as a tan solid. ¹H NMR (400 MHz, DMSO-d₆) δ 9.48 (br s, 1H), 8.99 (s, 1H), 8.63 (s, 1H), 7.13-7.15 (d, 1H), 6.59 (s, 1H), 6.46-6.49 (d, 1H), 4.69-4.70 (d, 2H), 3.81 (s, 3H), 3.74 (s, 3H); m/z (APCI-pos) M+1=346.1.

### Example AU

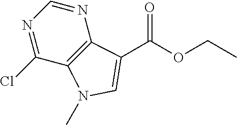

### Ethyl 4-chloro-5-methyl-5H-pyrrolo[3,2-d]pyrimidine-7-carboxylate

Step A: Diethyl 2-aminomalonate hydrochloride (20 g, 90 mmol) and ethyl 2-cyano-3-ethoxyacrylate (16 g, 94 mmol) were dissolved in ethanol (250 mL). Sodium ethoxide (21 g, 310 mmol) was added, and the mixture was heated to reflux for 14 hours. The mixture was neutralized with acetic acid (20 mL) and concentrated. The mixture was partitioned between dichloromethane and water. The aqueous layer was extracted twice with dichloromethane. The combined organic layers were washed with brine, dried over sodium sulfate, and concentrated. The resulting solid was triturated with a mixture of heptanes and dichloromethane (10:1) overnight. The suspension was filtered to yield 12.5 g of diethyl 3-amino-1H-pyrrole-2,4-dicarboxylate as a solid (80% yield). ¹H NMR (400 MHz, DMSO-d₆) δ 11.53 (s, 1H), 7.20 (d, J=4.0 Hz, 1H), 5.57 (s, 2H), 4.27-4.11 (m, 4H), 1.32-1.19 (m, 6H).

Step B: Diethyl 3-amino-1H-pyrrole-2,4-dicarboxylate (10.0 g, 44.2 mmol) was dissolved in ethanol (30 mL). Formamidine acetate (14.2 g, 136 mmol) was added, and the mixture was heated to reflux overnight. The mixture was filtered hot. The solids were triturated with hot methanol and filtered, followed by trituration with hot ethanol and methanol to yield 7.9 g of an 8:1 mixture of ethyl 4-hydroxy-5H-pyrrolo[3,2-d]pyrimidine-7-carboxylate and ethyl 4-hydroxy-6H-pyrrolo[3,4-d]pyrimidine-7-carboxylate as a solid. ¹H NMR (400 MHz, DMSO-d₆) δ 12.88 (s, 1H), 12.14 (s, 1H), 7.93 (s, 1H), 7.90 (s, 1H), 4.24 (q, J=7.1 Hz, 2H), 1.28 (t, J=7.1 Hz, 3H).

Step C: A 8:1 mixture of ethyl 4-hydroxy-5H-pyrrolo[3,2-d]pyrimidine-7-carboxylate and ethyl 4-hydroxy-6H-pyrrolo[3,4-d]pyrimidine-7-carboxylate (3.4 g) was suspended in phosphoryl chloride (30 mL, 300 mmol), and the mixture was heated to reflux overnight. The mixture was cooled, diluted with ether and filtered. The solid was suspended in dilute sodium bicarbonate solution, stirred for 2 hours and filtered to yield ethyl 4-chloro-5H-pyrrolo[3,2-d]pyrimidine-7-carboxylate as a single isomer (2.78 g, 94%). ¹H NMR (400 MHz, DMSO-d₆) δ 13.28 (s, 1H), 8.80 (s, 1H), 8.56 (d, J=3.2 Hz, 1H), 4.31 (q, J=7.1 Hz, 2H), 1.33 (t, J=7.1 Hz, 3H).

Step D: Ethyl 4-chloro-5H-pyrrolo[3,2-d]pyrimidine-7-carboxylate (500 mg, 2.22 mmol) was dissolved in N,N-dimethylformamide (4 mL, 50 mmol) and cooled to 0° C. Sodium hydride (60% in mineral oil, 115.2 mg) was added, and the mixture was stirred at 0° C. for 20 minutes. Methyl iodide (165.5 uL, 2.659 mmol) was added, and the mixture was slowly warmed to room temperature. The mixture was quenched with ammonium chloride solution and extracted with dichloromethane three times. The combined extracts were washed with brine, dried over sodium sulfate and concentrated. The crude product was purified using flash chromatography (gradient elution: 0-100% ethyl acetate+15% MeOH in heptanes) to yield ethyl 4-chloro-5-methyl-5H-pyrrolo[3,2-d]pyrimidine-7-carboxylate as a solid (380 mg, 72%). ¹H NMR (500 MHz, DMSO-d₆) δ 8.77 (s, 1H), 8.65 (s, 1H), 4.30 (q, J=7.1 Hz, 2H), 4.16 (s, 3H), 1.32 (t, J=7.1 Hz, 3H).

### Example AV

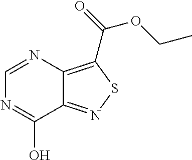

### Ethyl 7-hydroxyisothiazolo[4,3-d]pyrimidine-3-carboxylate

Step A: 2-Cyanoacetamide (10.0 g, 0.119 mol) and sodium nitrite (10.0 g, 0.145 mol) were dissolved in water (100 g, 5 mol) and cooled over an ice bath. Acetic acid (13.3 mL, 0.234 mol) was added by addition funnel over 30 minutes maintaining the temperature of the ice bath below 20° C. The reaction mixture was then stirred overnight, gradually warming to room temperature. After 16 hours the aqueous layer was extracted with 100 mL aliquots of ethyl acetate (2×). The combined organic layers were dried over magnesium sulfate, filtered and concentrated to give (E)-2-amino-N-hydroxy-2-oxoacetimidoyl cyanide (11.99 g, 89.1%). ¹H NMR (400 MHz, DMSO-d₆) δ 14.42 (s, 1H), 7.83 (d, J=27.0 Hz, 2H).

Step B: (E)-2-Amino-N-hydroxy-2-oxoacetimidoyl cyanide (5.032 g, 44.5 mmol) was suspended in pyridine (35.99 mL, 44.5 mmol) and cooled to 0° C. To this reaction mixture was added p-toluenesulfonyl chloride (8.48 g, 44.5 mmol) in four portions over 15 minutes, and the reaction was stirred over an ice bath for one hour and then diluted to 250 mL with ice water. The precipitated solid was collected by filtration and dried over vacuum to give (E)-2-amino-2-oxo-N-(tosyloxy)acetimidoyl cyanide (11.01 g, 92.6%). ¹H NMR (500 MHz, DMSO-d₆) δ 8.22 (s, 1H), 8.16 (s, 1H), 8.02 (d, J=8.4 Hz, 2H), 7.54 (d, J=8.1 Hz, 2H), 2.45 (s, 3H).

Step C: To a stirred suspension of (E)-2-Amino-2-oxo-N-(tosyloxy)acetimidoyl cyanide (11.01 g, 36.3 mmol) in ethanol (30 mL, 0.6 mol) cooled over an ice bath was added ethyl thioglycolate (4.77 mL, 43.5 mmol). Morpholine (4.75 mL, 54.5 mmol) dissolved in 6 mL ethanol was added via an addition funnel over fifteen minutes. After twenty minutes the reaction mixture was diluted with 150 mL ice water. The precipitated solid was collected by filtration and dried over vacuum to give ethyl 4-amino-3-carbamoylisothiazole-5-carboxylate (7.443 g, 95%). ¹H NMR (500 MHz, DMSO-d₆) δ 8.11 (s, 1H), 7.75 (s, 1H), 6.83 (s, 2H), 4.34-4.26 (m, 2H), 1.29 (t, J=7.1 Hz, 3H).

Step D: Ethyl 4-amino-3-carbamoylisothiazole-5-carboxylate (7.44 g, 34.6 mmol) was dissolved in a mixture of ethyl orthoformate (8.05 mL, 48.4 mmol) in acetic anhydride (34.58 mL, 0.3665 mol) and heated at 135° C. After 16 hours the reaction mixture was cooled to room temperature and the precipitated solid collected by filtration to give ethyl 7-hydroxyisothiazolo[4,3-d]pyrimidine-3-carboxylate (6.43 g, 83%). ¹H NMR (500 MHz, DMSO-d₆) δ 12.57 (s, 1H), 8.15 (s, 1H), 4.40 (q, J=7.1 Hz, 2H), 1.34 (t, J=7.1 Hz, 3H). MS m/z=226.0[M+1].

### Example AW

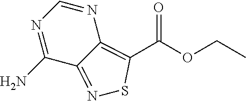

### Ethyl 7-aminoisothiazolo[4,3-d]pyrimidine-3-carboxylate

A mixture of ethyl 7-hydroxyisothiazolo[4,3-d]pyrimidine-3-carboxylate (0.509 g, 2.26 mmol) dissolved in acetonitrile (2.25 mL) and 2,6-lutidine (1.23 mL, 10.6 mmol) was heated to 50° C. Upon temperature stabilization, phosphoryl chloride (0.297 mL, 3.18 mmol) was added to the reaction mixture in a drop-wise fashion and heating continued for an additional 2 hours. The reaction mixture was then cooled to room temperature and 1H-1,2,4-triazole (1.56 g, 22.6 mmol) was added. The reaction mixture was allowed to stir at room temperature overnight. Ammonia gas was passed through the solution for 1 minute, and the reaction mixture was allowed to stir for an additional hour. The reaction mixture was concentrated under reduced pressure and purified by flash chromatography to give ethyl 7-aminoisothiazolo[4,3-d]pyrimidine-3-carboxylate (312 mg, 62%). ¹H NMR (400 MHz, DMSO-d₆) δ 8.47 (s, 1H), 8.36 (s, 2H), 4.41 (q, J=7.1 Hz, 2H), 1.35 (t, J=7.1 Hz, 3H). MS m/z=225.0 [M+1].

### Example AX

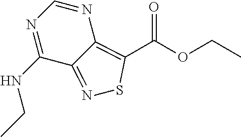

### Ethyl 7-(ethylamino)isothiazolo[4,3-d]pyrimidine-3-carboxylate

Step A: To a stirred mixture of ethyl 7-hydroxyisothiazolo[4,3-d]-pyrimidine-3-carboxylate (0.500 g, 0.00222 mol) in tetrahydrofuran (20 mL, 0.2 mol) was added benzotriazol-1-yloxytris(dimethylamino)phosphonium hexafluorophosphate (1500 mg, 0.0033 mol) followed by 1,8-diazabicyclo[5.4.0]undec-7-ene (432 uL, 0.00288 mol). After five minutes ethylamine gas was bubbled through the reaction mixture for 1 minute and the reaction mixture was allowed to stir at room temperature for one hour. The reaction mixture was concentrated under reduced pressure and purified by flash chromatography to give ethyl 7-(ethylamino)isothiazolo[4,3-d]pyrimidine-3-carboxylate (155 mg, 22% yield) as yellow foam. ¹H NMR (400 MHz, DMSO-d₆) δ 9.06 (s, 1H), 8.42 (s, 1H), 4.41 (q, J=7.1 Hz, 2H), 3.66-3.49 (m, 2H), 1.35 (t, J=7.1 Hz, 3H), 1.22 (t, J=7.2 Hz, 3H). MS m/z=253.0 [M+1].

### Example AY

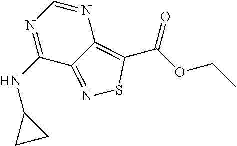

### Ethyl 7-(cyclopropylamino)isothiazolo[4,3-d]pyrimidine-3-carboxylate

The title compound was prepared using a similar procedure as described for Example AX using cyclopropanamine in place of ethylamine gas. ¹H NMR (400 MHz, DMSO-d₆) δ 9.17 (s, 1H), 8.47 (s, 1H), 4.41 (q, J=7.1 Hz, 2H), 3.16 (s, 1H), 1.35 (t, J=7.1 Hz, 3H), 0.89-0.66 (m, 4H). LC/MS m/z=265.1 [M+1].

### Example AZ

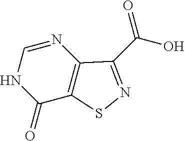

### 7-Oxo-6,7-dihydroisothiazolo[4,5-d]pyrimidine-3-carboxylic acid

Step A: To a well-mixed combination of ethyl 4-amino-3-carbamoylisothiazole-5-carboxylate (3.62 g, 16.82 mmol; Liebigs Ann. 1979, 1534-1546) and formamidine acetate (5.253 g, 50.46 mmol) was added formamide (13.36 mL, 336.4 mmol). The reaction mixture was heated at 180° C. The solution slowly turned dark and water was observed condensing. After 3.5 h heating, the cooled reaction mixture was diluted with water and filtered to afford 7-oxo-6,7-dihydroisothiazolo[4,5-d]pyrimidine-3-carboxamide (2.61 g, 13.30 mmol, 79.10% yield) as a brown solid. LC/MS: m/z 180.1 (100%), 197.1 [M+1](55%).

Step B: To 7-oxo-6,7-dihydroisothiazolo[4,5-d]pyrimidine-3-carboxamide (2.61 g, 13.3 mmol) was added 6 M hydrochloric acid (22.2 mL, 133 mmol). The mixture was heated at 100° C. for 2 h. The reaction mixture was cooled and poured onto 50 mL ice-water. The brown solid was collected by vacuum filtration to yield 7-oxo-6,7-dihydroisothiazolo[4,5-d]-pyrimidine-3-carboxylic acid (1.75 g, 8.88 mmol, 66.7% yield). ¹H NMR (400 MHz, DMSO-d₆) δ 8.30 (s, 1H). LC/MS: m/z 152.1 (100%), 195.1 [M+1](15%).

### Example BA

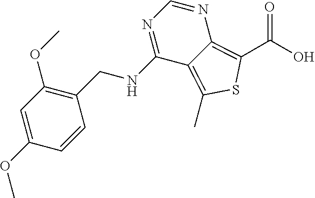

### 4-(2,4-Dimethoxybenzylamino)-5-methylthieno[3,4-d]pyrimidine-7-carboxylic acid

Step A: (Z)-2-Cyano-3-(dimethylamino)but-2-enamide (4.23 g, 0.0276 mol), ethyl thioglycolate (3.63 mL, 0.0331 mol) and potassium carbonate (0.267 g, 0.00193 mol) were dissolved in ethanol (30 mL, 0.5 mol) and heated at reflux overnight. Upon cooling to room temperature the reaction mixture was diluted with 100 mL water and the resulting solid collected by filtration and dried in vacuo to give ethyl 3-amino-4-carbamoyl-5-methylthiophene-2-carboxylate (92.82 g, 44.8%). ¹H NMR (500 MHz, DMSO-d₆) δ 7.52 (d, J=7.2 Hz, 2H), 6.51 (s, 2H), 4.32-4.03 (m, 2H), 1.34-1.14 (m, 3H). MS m/z=229.0

Step B: Ethyl 3-amino-4-carbamoyl-5-methylthiophene-2-carboxylate (2.82 g, 0.0123 mol), ethyl orthoformate (2.88 mL, 0.0172 mol) and acetic anhydride (12.4 mL, 0.131 mol) were combined and heated at 135° C. for 4 hours. The reaction mixture was cooled to room temperature and the precipitated material collected by filtration to give ethyl 4-hydroxy-5-methylthieno[3,4-d]pyrimidine-7-carboxylate (2.20 g, 75%). ¹H NMR (500 MHz, DMSO-d₆) δ11.92 (s, 1H), 7.94 (s, 1H), 4.43-4.20 (m, 2H), 2.89 (s, 3H), 1.29 (td, J=7.1, 5.1 Hz, 3H). MS m/z=239.0

Step C: Ethyl 4-hydroxy-5-methylthieno[3,4-d]pyrimidine-7-carboxylate (0.522 g, 2.19 mmol) and 2,6-lutidine (0.510 mL, 4.40 mmol) were dissolved in acetonitrile (2.22 mL, 42.5 mmol) and heated to 50° C. Upon temperature stabilization, phosphoryl chloride (0.408 mL, 4.38 mmol) was added to the reaction mixture in a drop-wise fashion and heating continued for an additional 4 hours. The reaction mixture was cooled to room temperature and quenched via drop-wise addition into a stirring, ice-cooled solution of N,N-diisopropylethylamine (7 mL, 40 mmol) and 2,4-dimethoxybenzylamine (0.383 mL, 2.55 mmol). After 30 minutes of stirring at room temperature, additional 2,4-dimethoxybenzylamine (0.4 mL, 2.6 mmol) was added to the reaction mixture. After an additional hour of stirring the reaction was concentrated and partitioned between ethyl acetate and 1M potassium bisulfate prior to purification by flash chromatography to give ethyl 4-(2,4-dimethoxybenzylamino)-5-methylthieno[3,4-d]pyrimidine-7-carboxylate (440 mg, 85% purity). This material was dissolved in tetrahydrofuran (20 mL, 0.2 mol) to which was added water (3 mL, 0.2 mol) and lithium hydroxide (34.5 mg, 0.00144 mol). The reaction mixture was stirred at room temperature for 1 hour and then concentrated to remove the solvents. The residue was then redissolved in 20 mL water and acidified with 2 drops of glacial acetic acid. The bright yellow precipitate was collected by filtration and dried under reduced pressure to give 4-(2,4-dimethoxybenzylamino)-5-methylthieno[3,4-d]pyrimidine-7-carboxylic acid (137 mg, 20%). ¹H NMR (400 MHz, DMSO-d₆) δ 7.22 (m, 1H), 6.59 (s, 1H), 6.46 (m, 3H), 4.72 (s, 2H), 3.84 (s, 3H), 3.73 (s, 6H). MS m/z=360.0 [M+1].

### Example BB

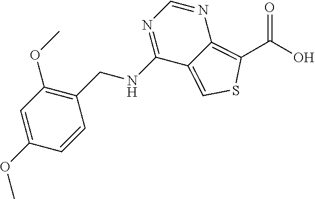

### 4-(2,4-Dimethoxybenzylamino)thieno[3,4-d]pyrimidine-7-carboxylic acid

Step A: Ethyl 4-aminothiophene-3-carboxylate hydrochloride (5 g, 20 mmol) and formamidine acetate (16.1 g, 155 mmol) were dissolved in ethanol (50 mL, 800 mmol) and heated to reflux overnight. The reaction mixture was cooled over an ice water bath and the precipitated solid collected by filtration. This solid was resuspended in 100 mL water and stirred for 5 minutes before being refiltered and dried over vacuum to give thieno[3,4-d]pyrimidin-4-ol as a tan solid (2.89 g, 70%). ¹H NMR (400 MHz, DMSO-d₆) δ 11.60 (s, 1H), 8.41 (s, 1H), 7.76 (d, J=13.9 Hz, 2H). MS m/z=158.2.

Step B: Thieno[3,4-d]pyrimidin-4-ol (7.529 g, 0.04948 mol) was suspended in a mixture of acetic acid (60 mL, 1 mol) and chloroform (60 mL, 0.8 mol). N-Bromosuccinimide (9.69 g, 0.0544 mol) was added, and the reaction was stirred at room temperature for 1 hour. The reaction mixture was diluted with ethyl ether and the precipitated solid collected by filtration to give 7-bromothieno[3,4-d]pyrimidin-4-ol (7.11 g, 62%). ¹H NMR (400 MHz, DMSO-d₆) δ 11.82 (s, 1H), 8.56 (s, 1H), 7.88 (d, J=2.8 Hz, 1H). MS m/z=232.9.

Step C: 7-Bromothieno[3,4-d]pyrimidin-4-ol (7.1112 g, 30.775 mmol), [1,1′-bis(diphenylphosphino)ferrocene]dichloropalladium(II), complex with dichloromethane (1:1) (1.508 g, 1.847 mmol), triethylamine (21.4 mL, 153.9 mmol), and methanol (37.4 mL, 923.2 mmol) were combined in an autoclave and the mixture was degassed with nitrogen for five minutes. The reaction was placed under an atmosphere of carbon monoxide at 300 psi, heated to 120° C. and stirred for 3 hours. After cooling, the precipitated solid was collected by filtration, rinsed with methanol, and dried in a vacuum oven overnight to give methyl 4-hydroxythieno[3,4-d]pyrimidine-7-carboxylate (5.72 g, 88%). ¹H NMR (400 MHz, DMSO-d₆) δ 12.06 (s, 1H), 8.81 (s, 1H), 8.02 (s, 1H), 3.93-3.77 (m, 3H). MS m/z=210.97.

Step D: Methyl 4-hydroxythieno[3,4-d]pyrimidine-7-carboxylate (1.02 g, 0.00485 mol) was dissolved in acetonitrile (50 mL, 0.9 mol) and benzotriazol-1-yloxytris(dimethylamino)phosphonium hexafluorophosphate (3220 mg, 0.00728 mol) and 1,8-diazabicyclo[5.4.0]undec-7-ene (943 uL, 0.00631 mol) were added to the solution. The reaction mixture was stirred for 5 minutes before 2,4-dimethoxybenzylamine (1090 uL, 7.28 mmol) was added. The reaction mixture was stirred at room temperature for 4 hours before it was diluted with ethyl acetate and washed with saturated sodium bicarbonate. The organic layer was concentrated to dryness and purified by flash chromatography. The product methyl 4-(2,4-dimethoxybenzylamino)thieno[3,4-d]pyrimidine-7-carboxylate was obtained as a tan oil (327 mg, 24%). ¹H NMR (400 MHz, DMSO-d₆) δ 10.36 (s, 1H), 9.24 (s, 1H), 8.53 (s, 1H), 7.23 (d, J=8.3 Hz, 1H), 6.62 (s, 1H), 6.51 (d, J=8.5 Hz, 1H), 4.78 (d, J=4.3 Hz, 2H), 3.92 (s, 3H), 3.81 (s, 3H), 3.76 (s, 3H). MS m/z=360.2.

Step E: Methyl 4-(2,4-dimethoxybenzylamino)thieno[3,4-d]pyrimidine-7-carboxylate (146 mg, 0.406 mmol) was dissolved in tetrahydrofuran (20 mL, 0.2 mol) and water (5 mL, 0.3 mol). Lithium hydroxide (24.3 mg, 1.02 mmol) was added, and the reaction mixture was stirred at 50° C. for 1 hour. The reaction mixture was concentrated to dryness, redissolved in 20 mL of water and acidified with 2 drops glacial acetic acid. The resulting tan precipitate was collected by filtration and rinsed with ether to give 4-(2,4-dimethoxybenzylamino)-thieno[3,4-d]pyrimidine-7-carboxylic acid (114 mg, 81%). ¹H NMR (400 MHz, DMSO-d₆) δ 9.54 (s, 1H), 9.05 (s, 1H), 8.40 (s, 1H), 7.19 (d, J=8.4 Hz, 1H), 6.60 (s, 1H), 6.49 (d, J=8.1 Hz, 1H), 4.70 (d, J=5.0 Hz, 2H), 3.81 (s, 3H), 3.75 (s, 3H). MS m/z=346.1.

### Example BC

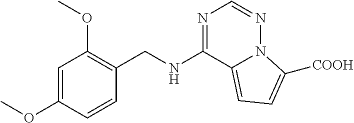

### 4-(2,4-Dimethoxybenzylamino)pyrrolo[1,2-f][1,2,4]triazine-7-carboxylic acid

Step A: To a solution of 4-chloropyrrolo[1,2-f][1,2,4]triazine (3.00 g, 19.5 mmol; Leadgen Labs) in DMF (35 mL) was added N-bromosuccinimide (3.51 g, 19.7 mmol) at 0° C., and the reaction mixture was stirred at 0° C. for 90 minutes. Subsequent dilution with ethyl acetate and addition of a saturated aqueous solution of NaHCO₃ is followed by separation of the layers. The organic layers were washed with water (3×), dried with sodium sulfate, filtered, and concentrated in vacuo. The crude product was purified by flash chromatography to afford 7-bromo-4-chloropyrrolo[1,2-f][1,2,4]triazine (3.75 g, 83%).

Step B: A microwave vial was charged with 7-bromo-4-chloropyrrolo[1,2-f]-[1,2,4]triazine (1.00 g, 4.3 mmol, 2,4-dimethoxybenzylamine (1.29 mL, 8.60 mmol), and THF (11 mL). The reaction mixture was subjected to microwave irradiation at 95° C. for 20 minutes. This reaction was repeated twice on 1 gram scale and twice on 0.85 gram scale of 7-bromo-4-chloropyrrolo[1,2-f][1,2,4]triazine. The reaction mixtures were combined and concentrated in vacuo. The crude was purified by flash chromatography to afford 7-bromo-N-(2,4-dimethoxybenzyl)pyrrolo[1,2-f][1,2,4]triazin-4-amine (2.69 g, 47%).

Step C: To a solution of 7-bromo-N-(2,4-dimethoxybenzyl)pyrrolo[1,2-f][1,2,4]triazin-4-amine (0.50 g, 1.38 mmol) in THF (13 mL) at −78° C. was added dropwise n-butyllithium (2.84 mL, 1.6 M in hexanes). The reaction mixture was stirred at −78° C. for 75 minutes, and a flow of carbon dioxide gas was passed through the reaction mixture for 30 minutes. The reaction was quenched with water at −78° C. and allowed to warm to room temperature. Removal of the solvent under reduced pressure and subsequent purification by flash chromatography afforded 4-(2,4-dimethoxybenzylamino)pyrrolo[1,2-f][1,2,4]triazine-7-carboxylic acid (0.19 g, 41%). ¹H NMR (500 MHz, DMSO-d₆) δ 12.59 (br s, 1H), 8.78 (s, 1H), 8.07 (s, 1H), 7.21 (m, 1H), 7.13 (d, J=8.3 Hz, 1H), 7.07-7.03 (m, 1H), 6.59 (s, 1H), 6.48 (d, J=8.5 Hz, 1H), 4.63 (d, J=5.3 Hz, 2H), 3.81 (s, 3H), 3.74 (s, 3H); m/z (ES-MS) 329.2 [M+1].

### Example 1

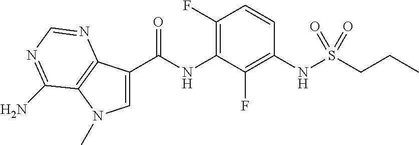

### 4-amino-5-methyl-pyrrolo[3,2-d]pyrimidine-7-carboxylic acid[2,6-difluoro-3-(propane-1-sulfonylamino)-phenyl]-amide

Step A: N-(3-Amino-2,4-difluorophenyl)-N-benzylpropane-1-sulfonamide (340 mg, 1.0 mmol) was dissolved in toluene (4 mL). A solution of trimethylaluminum in hexane (2 M; 0.5 mL) was added dropwise. The mixture was stirred at room temperature for 2 hours. A solution of ethyl 4-chloro-5-methyl-5H-pyrrolo[3,2-d]pyrimidine-7-carboxylate (200 mg, 0.8 mmol) was dissolved in toluene (2 mL) and 1,4-dioxane (4 mL) and was added to the reaction mixture. The mixture was stirred at 60° C. overnight. The mixture was quenched with methanol and 2N HCl. The mixture was applied to a Varian Chemelut cartridge, eluted with dichloromethane and ethyl acetate, and concentrated. The crude product was purified using flash chromatography (gradient elution: 0-100% ethyl acetate in heptanes) to yield N-(3-(N-benzylpropylsulfonamido)-2,6-difluorophenyl)-4-chloro-5-methyl-5H-pyrrolo[3,2-d]pyrimidine-7-carboxamide (330 mg, 70%). ¹H NMR (500 MHz, DMSO-d₆) δ 10.55 (s, 1H), 8.97 (s, 1H), 8.92 (s, 1H), 7.44-7.21 (m, 6H), 7.17 (t, J=9.1 Hz, 1H), 5.30 (s, 2H), 4.79 (s, 3H), 3.36-3.30 (m, 2H), 1.82-1.68 (m, 2H), 1.01 (t, J=7.4 Hz, 3H).

Step B: N-(3-(N-Benzylpropylsulfonamido)-2,6-difluorophenyl)-4-chloro-5-methyl-5H-pyrrolo[3,2-d]pyrimidine-7-carboxamide (244 mg, 0.457 mmol) was suspended in a solution of ammonia in isopropyl alcohol (2 M; 2.5 mL). The reaction was heated in a microwave reactor at 105° C. for 10 minutes. Ammonia gas was passed through the reaction mixture. The reaction was heated in a microwave reactor at 120° C. for 30 minutes. Purging with ammonia gas and heated in a microwave reactor at 120° C. for 30 minutes was repeated twice. The mixture was concentrated and loaded onto silica. The crude product was purified using flash chromatography (gradient elution using 0-100% ethyl acetate in heptanes) to yield 4-amino-N-(3-(N-benzylpropylsulfonamido)-2,6-difluorophenyl)-5-methyl-5H-pyrrolo[3,2-d]pyrimidine-7-carboxamide (204 mg, 74%). ¹H NMR (500 MHz, DMSO-d₆) δ 10.19 (s, 1H), 8.26 (s, 1H), 8.12 (s, 1H), 7.37-7.21 (m, 6H), 7.13 (t, J=9.1 Hz, 1H), 7.07 (s, 2H), 4.78 (s, 2H), 4.09 (s, 3H), 3.27 (s, 2H), 1.90-1.73 (m, 2H), 1.01 (t, J=7.4 Hz, 3H).

Step C: 4-Amino-N-(3-(N-benzylpropylsulfonamido)-2,6-difluorophenyl)-5-methyl-5H-pyrrolo[3,2-d]pyrimidine-7-carboxamide (200 mg, 0.4 mmol), palladium hydroxide on carbon 20% (55 mg), ammonium formate (500 mg, 8 mmol), and ethanol (20 mL) were combined in a vial. The mixture was stirred at 60° C. for 2 hours. The reaction mixture was concentrated under reduced pressure, and the resulting solids were dissolved in water. The mixture was filtered over Celite®, and the solids were washed with water. The solids were redissolved with ethyl acetate and methanol. The solvent was removed under reduced pressure to give a solid. The solid was triturated at 60° C. with ethyl acetate, filtered and dried in a vacuum oven to yield 4-ethyl-thieno[3,2-d]pyrimidine-7-carboxylic acid [2,6-difluoro-3-(propane-1-sulfonylamino)-phenyl]-amide as a solid (108 mg, 60%). ¹H NMR (400 MHz, DMSO-d₆) δ 10.23 (s, 1H), 8.27 (s, 1H), 8.14 (s, 1H), 8.13 (s, 1H), 7.34 (td, J=8.9, 5.7 Hz, 1H), 7.19 (td, J=9.3, 1.6 Hz, 1H), 7.08 (s, 2H), 4.09 (s, 3H), 3.12-3.03 (m, 2H), 1.83-1.68 (m, 2H), 0.98 (t, J=7.4 Hz, 3H). LC/MS: m/z 425.1 [M+1].

### Example 2

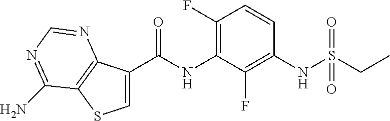

### 4-Amino-thieno pyrimidine-7-carboxylic acid (3-ethanesulfonylamino-2,6-difluoro-phenyl)-amide

Step A: N-(3-Amino-2,4-difluorophenyl)-N-(4-methoxybenzyl)ethanesulfonamide (428 mg, 1.20 mmol) was dissolved in toluene (5 mL, 50 mmol) and 2M trimethylaluminum in hexane (600 uL, 1 mmol) was slowly added. The reaction mixture was stirred at room temperature for 1 hour. Methyl 4-chlorothieno[3,2-d]pyrimidine-7-carboxylate (229 mg, 1.00 mmol) was added to this solution as a solid, and the resulting mixture was stirred at 80° C. overnight. The reaction mixture was cooled to room temperature and quenched with a solution of potassium sodium tartrate (1N, 5 mL). The reaction mixture was stirred at room temperature for 1 hour. The mixture was applied to a Varian Chemelut™ cartridge and eluted with ethyl acetate. The crude product was purified using flash chromatography (40 g column, 0-45% ethyl acetate: heptane) to give 4-chloro-N-(2,6-difluoro-3-(N-(4-methoxybenzyl)-ethylsulfonamido)phenyl)thieno[3,2-d]pyrimidine-7-carboxamide (345 mg, 62%). ¹H NMR (400 MHz, DMSO-d₆) δ 10.59 (s, 1H), 9.35 (s, 1H), 9.26 (s, 1H), 7.29 (dd, J=14.3, 8.6 Hz, 1H), 7.24-7.15 (m, 3H), 6.85 (d, J=8.7 Hz, 2H), 4.72 (s, 2H), 3.71 (s, 3H), 3.31-3.28 (m, 2H), 1.17 (t, J=7.1 Hz, 2H).

Step B: 4-Chloro-N-(2,6-difluoro-3-(N-(4-methoxybenzyl)ethylsulfonamido)phenyl)-thieno[3,2-d]pyrimidine-7-carboxamide (486.2 mg, 0.8792 mmol) was dissolved in 1,4-dioxane (5 mL, 60 mmol), and ammonia gas was passed through the resulting solution for 2 minutes. This mixture was heated in a microwave reactor at 120° C. for 40 minutes. The reaction mixture was concentrated under reduced pressure to remove the dioxane, and then 5 mL of water was added. The resulting solution was heated to boiling and then stirred for 30 minutes while cooling to room temperature. The resulting solid was collected by filtration, redissolved in methanol and then concentrated to give 4-amino-N-(2,6-difluoro-3-(N-(4-methoxybenzyl)ethylsulfonamido)phenyl)thieno[3,2-d]pyrimidine-7-carboxamide as an oil (435 mg, 93%). ¹H NMR (400 MHz, DMSO-d₆) δ 11.33 (s, 1H), 8.97 (s, 1H), 8.55 (s, 1H), 7.96 (s, 2H), 7.32-7.22 (m, 1H), 7.17 (t, J=9.0 Hz, 3H), 6.85 (d, J=8.7 Hz, 2H), 6.78-6.76 (m, 0H), 4.72 (s, 2H), 3.71 (s, 3H), 3.28 (s, 2H), 1.31 (t, J=7.3 Hz, 3H).

Step C: 4-Amino-N-(2,6-difluoro-3-(N-(4-methoxybenzyl)ethylsulfonamido)phenyl)-thieno[3,2-d]pyrimidine-7-carboxamide (435 mg, 0.815 mmol) was dissolved in dichloromethane (4 mL, 60 mmol) and trifluoroacetic acid (4 mL, 50 mmol) was added. The reaction mixture was stirred at room temperature for 4 hours and then concentrated to remove the dichloromethane and trifluoroacetic acid. The resulting oil was redissolved in ethyl acetate and washed once with water. The organic layer was dried over magnesium sulfate, filtered, and then purified by flash chromatography (40 g column, 0-50% (20% MeOH:dichloromethane): dichloromethane). The indicated fractions were concentrated to dryness, and the resulting oil was precipitated with methyl tert-butyl ether. The resulting solid was collected by filtration and dried in the vacuum oven overnight to yield 4-amino-thieno[3,2-d]pyrimidine-7-carboxylic acid (3-ethanesulfonylamino-2,6-difluoro-phenyl)-amide as a solid (167 mg, 50%). ¹H NMR (400 MHz, DMSO-d₆) δ 11.35 (s, 1H), 9.72 (s, 1H), 8.97 (s, 1H), 8.55 (s, 1H), 7.96 (s, 2H), 7.38 (td, J=8.9, 5.7 Hz, 1H), 7.23 (dd, J=9.1, 7.7 Hz, 1H), 3.11 (q, J=7.3 Hz, 2H), 1.27 (t, J=7.3 Hz, 3H). LC/MS: m/z 414.2 [M+1].

Examples 3-7 below in Table 1 were prepared according to the procedure described in Example 2 using appropriate starting materials.

TABLE 1

| Ex- | | | | |
|---|---|---|---|---|
| ample | | | | ¹H NMR δ |
| no. | Structure | Name | MS m/z | (400 MHz, DMSO-d6) |
| 3 | 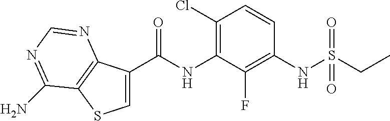 | 4-Amino-thieno[3,2-d] pyrimidine-7-carboxylic acid (6-chloro-3-ethane- sulfonylamino-2-fluoro- phenyl)-amide | 430.2 [M + 1] | 11.48 (s, 1H), 9.90 (s, 1H), 8.97 (s, 1H), 8.55 (s, 1H), 7.96 (s, 2H), 7.49-7.35 (m, 2H), 3.14 (q, J = 7.3 Hz, 2H), 1.26 (t, J = 7.3 Hz, 3H) |
| 4 | 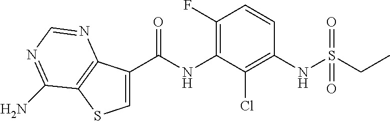 | 4-Amino-thieno[3,2-d] pyrimidine-7-carboxylic acid (2-chloro-3-ethane- sulfonylamino-6-fluoro- phenyl)-amide | 430.2 [M + 1] | 11.51 (s, 1H), 9.62 (s, 1H), 8.96 (s, 1H), 8.55 (s, 1H), 7.96 (s, 2H), 7.47 (dd, J = 9.1, 5.3 Hz, 1H), 7.38 (t, J = 9.1 Hz, 1H), 3.15 (q, J = 7.4 Hz, 2H), 1.29 (t, J = 7.3 Hz, 3H) |
| 5 | 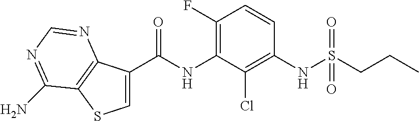 | 4-Amino-thieno[3,2-d] pyrimidine-7-carboxylic acid [2-chloro-6-fluoro-3- (propane-1-sulfonyl- amino)-phenyl]-amide | 444.0 [M + 1] | 11.49 (s, 1H), 9.62 (s, 1H), 8.98 (s, 1H), 8.56 (s, 1H), 7.97 (s, 2H), 7.47 (dd, J = 9.1, 5.3 Hz, 1H), 7.39 (t, J = 9.1 Hz, 1H), 3.18-3.09 (m, 2H), 1.77 (dd, J = 15.2, 7.5 Hz, 2H), 0.99 (t, J = 7.4 Hz, 3H) |
| 6 | 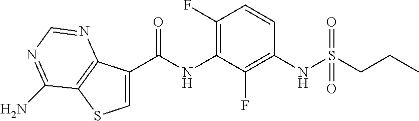 | 4-Amino-thieno[3,2-d] pyrimidine-7-carboxylic acid [2,6-difluoro-3-(propane- 1-sulfonylamino)- phenyl]-amide | 428.0 [M + 1] | 11.49 (s, 1H), 9.62 (s, 1H), 8.98 (s, 1H), 8.56 (s, 1H), 7.97 (s, 2H), 7.47 (dd, J = 9.1, 5.3 Hz, 1H), 7.39 (t, J = 9.1 Hz, 1H), 3.18-3.09 (m, 2H), 1.77 (dd, J = 15.2, 7.5 Hz, 2H), 0.99 (t, J = 7.4 Hz, 3H) |
| 7 | 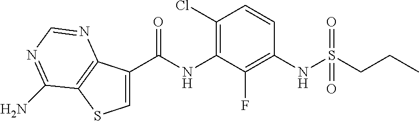 | 4-Amino-thieno[3,2-d] pyrimidine-7-carboxylic acid [6-chloro-2-fluoro-3- (propane-1-sulfonyl- amino)-phenyl]-amide | 444.0 [M + 1] | 11.47 (s, 1H), 9.90 (s, 1H), 8.98 (s, 1H), 8.55 (s, 1H), 7.97 (s, 2H), 7.49-7.36 (m, 2H), 3.18-3.07 (m, 2H), 1.81-1.67 (m, 2H), 0.97 (t, J = 7.4 Hz, 3H) |

### Example 8

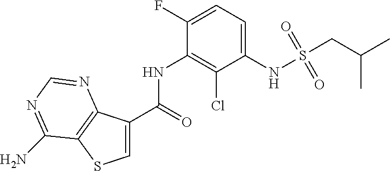

### 4-Amino-thieno[3,2-d]pyrimidine-7-carboxylic acid[2-chloro-6-fluoro-3-(2-methyl-propane-1-sulfonylamino)-phenyl]-amide

Step A: To a solution of methyl 4-chlorothieno[3,2-d]pyrimidine-7-carboxylate (2.00 g, 8.75 mmol) in tetrahydrofuran (60 mL) and water (20 mL) was added lithium hydroxide monohydrate (0.59 g, 14.0 mmol). The reaction mixture was stirred at room temperature for 2 hours, after which the volatiles were concentrated in vacuo. Water was added and a solid was obtain after filtration, which was rinsed with water and dried on a lyophilizer to afford 4-chlorothieno[3,2-d]pyrimidine-7-carboxylic acid (1.56 g, 81%).

Step B: To a solution of 4-chlorothieno[3,2-d]pyrimidine-7-carboxylic acid (0.099 g, 0.462 mmol) in tetrahydrofuran (5 mL) at 0° C. was added oxalyl chloride (137 uL, 1.62 mmol) followed by DMF (7.2 uL). The reaction mixture was stirred at room temperature for 1 hour and then concentrated in vacuo to give crude 4-chlorothieno[3,2-d]pyrimidine-7-carbonyl chloride as an oil which was used directly in the next step.

Step C: Crude 4-chlorothieno[3,2-d]pyrimidine-7-carbonyl chloride from step B was dissolved in tetrahydrofuran (40 mL) and N-(3-amino-2-chloro-4-fluorophenyl)-2-methylpropane-1-sulfonamide (1.00 g, 3.75 mmol) was added. The reaction mixture was stirred at 55° C. for 90 minutes, cooled to room temperature and diluted with dichloromethane and a saturated solution of NaHCO₃. The layers were separated and the aqueous layer extracted with dichloromethane (2×). The organics were combined, dried with sodium sulfate, filtered and concentrated in vacuo. The crude product was purified by flash chromatography to afford 4-chloro-N-(2-chloro-6-fluoro-3-(2-methylpropyl-sulfonamido)phenyl)thieno[3,2-d]pyrimidine-7-carboxamide (0.16 g, 72%).

Step D: A sealed tube was charged with 4-chloro-N-(2-chloro-6-fluoro-3-(2-methylpropyl-sulfonamido)phenyl)thieno[3,2-d]pyrimidine-7-carboxamide (0.14 g, 0.285 mmol), and a 2M ammonia solution in isopropanol (3.8 mL) was added. The reaction mixture was heated at 95° C. for 16 hours and then concentrated in vacuo. The crude product was purified by reverse phase HPLC to afford 4-amino-thieno[3,2-d]pyrimidine-7-carboxylic acid [2-chloro-6-fluoro-3-(2-methyl-propane-1-sulfonylamino)-phenyl]-amide (0.099 g, 76%). ¹H NMR (500 MHz, DMSO-d₆) δ 11.49 (s, 1H), 9.57 (s, 1H), 8.95 (s, 1H), 8.55 (s, 1H), 7.92 (s, 2H), 7.47 (dd, J=9.1, 5.4 Hz, 1H), 7.37 (t, J=9.1 Hz, 1H), 3.04 (d, J=6.5 Hz, 2H), 2.20 (dt, J=13.3, 6.7 Hz, 1H), 1.04 (d, J=6.7 Hz, 6H). m/z (ES-MS) 458.0 (100%) [M+1].

### Example 9

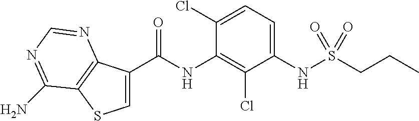

### 4-Amino-thieno[3,2-d]pyrimidine-7-carboxylic acid[2,6-dichloro-3-(propane-1-sulfonylamino)-phenyl]-amide

Step A: To a solution of 4-chlorothieno[3,2-d]pyrimidine-7-carboxylic acid (499 mg, 2.32 mmol, prepared as in Example 8, Step A) in THF (15 mL) at 0° C. was added oxalyl chloride (590 uL, 6.97 mmol) followed by DMF (18 uL, 0.23 mmol). The reaction mixture was stirred at room temperature for 1 hour and then concentrated in vacuo. The residue was dissolved in chloroform (15 mL), and N-(3-amino-2,4-dichlorophenyl)propane-1-sulfonamide (329 mg, 1.16 mmol) and pyridine (94 uL, 1.16 mmol) were added. The reaction was warmed to 55° C., stirred for 2 hours and then concentrated in vacuo. The crude product was purified by chromatography eluting with ethyl acetate/hexane (1:1) to obtain 4-chloro-N-(2,6-dichloro-3-(propylsulfonamido)phenyl)thieno[3,2-d]pyrimidine-7-carboxamide (557 mg, 94%).

Step B: 4-Chloro-N-(2,6-dichloro-3-(propylsulfonamido)phenyl)thieno[3,2-d]pyrimidine-7-carboxamide (170 mg, 0.35 mmol) was dissolved in isopropanol (8 mL). Ammonia gas was passed through the solution for 15 minutes. The mixture was heated in a microwave reactor at 120° C. for 2 hours, concentrated in vacuo, and then purified by preparative HPLC to obtain 4-amino-thieno[3,2-d]pyrimidine-7-carboxylic acid [2,6-dichloro-3-(propane-1-sulfonylamino)phenyl]-amide (37 mg, 23%). ¹H NMR (400 MHz, DMSO-d₆) δ 11.57 (s, 1H), 9.70 (s, 1H), 8.96 (s, 1H), 8.55 (s, 1H), 7.95 (s, 2H), 7.58 (s, 1H), 7.50 (s, 1H), 3.20-3.07 (m, 2H), 1.82-1.67 (m, 2H), 0.98 (t, J=7.4 Hz, 3H). LC-MS [M+1]m/z 460.0.

### Example 10

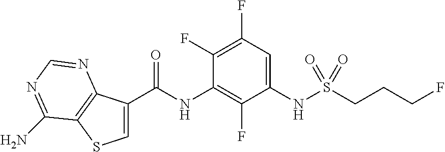

### 4-Amino-thieno[3,2-d]pyrimidine-7-carboxylic acid[2,3,6-trifluoro-5-(3-fluoro-propane-1-sulfonylamino)-phenyl]-amide

The title compound was prepared using a similar procedure as described in Example 9, using N-(3-amino-2,4-difluorphenyl)-3-fluoro-propane-1-sulfonamide in place of N-(3-amino-2,4-dichlorophenyl)propane-1-sulfonamide. ¹H NMR (400 MHz, DMSO-d₆) δ 11.46 (s, 1H), 9.96 (s, 2H), 8.99 (s, 1H), 8.55 (s, 1H), 7.97 (s, 2H), 7.44 (d, J=10.6 Hz, 1H), 6.54 (q, J=7.5 Hz, 1H), 5.77 (s, 2H), 4.60 (d, J=5.8 Hz, 2H), 4.49 (d, J=5.8 Hz, 2H), 3.26-3.12 (m, 4H), 2.19-1.97 (m, 4H). LC-MS [M+1]m/z 464.0.

### Example 11

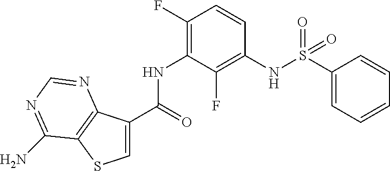

### 4-Amino-thieno[3,2-d]pyrimidine-7-carboxylic acid (3-benzenesulfonylamino-2,6-difluoro-phenyl)-amide

N-(3-Amino-2,4-difluorophenyl)benzenesulfonamide (230.0 mg, 0.809 mmol) was dissolved in 5.4 mL CHCl₃ (0.15 M) and treated with 4-chlorothieno[3,2-d]pyrimidine-7-carbonyl chloride (188.6 mg, 0.809 mmol, prepared as Example 38, Step B). The reaction mixture was heated to 60° C., stirred for 16 hours, and then cooled to ambient temperature and concentrated. The crude reaction mixture was dissolved in 1,4-dioxane (6 mL), and anhydrous ammonia gas was passed through the solution for 5 minutes. The vial was sealed, heated to 100° C. for 5 hours, and then cooled to ambient temperature and concentrated. Purification via flash chromatography eluting with a gradient of 10 to 80% acetone:hexanes afforded 4-amino-thieno[3,2-d]pyrimidine-7-carboxylic acid (3-benzenesulfonylamino-2,6-difluoro-phenyl)-amide (109.9 mg, 0.238 mmol, 29.4% yield). ¹H NMR (400 MHz, DMSO-d₆) δ 11.27 (s, 1H), 10.27 (s, 1H), 8.93 (s, 1H), 8.52 (s, 1H), 7.95 (s, 2H), 7.75-7.732 (d, 2H), 7.68-7.64 (t, 1H), 7.60-7.56 (t, 2H), 7.20-7.14 (m, 2H). LC/MS: m/z 460.1 [M−1].

### Example 12

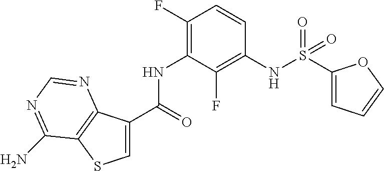

### 4-Amino-thieno[3,2-d]pyrimidine-7-carboxylic acid[2,6-difluoro-3-(furan-2-sulfonylamino)-phenyl]-amide

The title compound was made using a similar procedure as for example Example 11 using N-(3-Amino-2,4-difluorophenyl)furan-2-sulfonamide in place of N-(3-Amino-2,4-difluorophenyl)benzenesulfonamide. ¹H NMR (400 MHz, DMSO-d₆) δ 11.34 (s, 1H), 10.58 (s, 1H), 8.95 (s, 1H), 8.54 (s, 1H), 8.01 (s, 1H), 7.96 (s, 2H), 7.24-7.16 (m, 2H), 7.08-7.06 (d, 1H), 6.67-6.66 (m, 1H). LC/MS: m/z 450.0 [M−1].

### Example 13

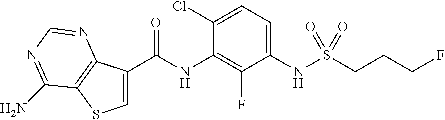

### 4-Amino-thieno[3,2-d]pyrimidine-7-carboxylic acid[6-chloro-2-fluoro-3-(3-fluoro-propane-1-sulfonylamino)-phenyl]-amide

Step A: To a solution of 4-chlorothieno[3,2-d]pyrimidine-7-carboxylic acid (0.050 g, 0.23 mmol, prepared as in Example 8, Step A) in THF (5 mL) at 0° C. was added 2.0 M oxalyl chloride in dichloromethane (0.23 mL, 0.27 mmol), followed by a drop of DMF. The reaction mixture was stirred at room temperature for 90 minutes and then concentrated. The residue was dissolved in THF (5.0 mL) and N-(3-amino-4-chloro-2-fluorophenyl)-3-fluoropropane-1-sulfonamide (0.053 g, 0.19 mmol) was added. The mixture was stirred at 55° C. for 2 hours and then concentrated. The crude product was purified via silica gel chromatography, eluting with hexanes/ethyl acetate (2:1) to give 4-chloro-N-(6-chloro-2-fluoro-3-(3-fluoropropylsulfonamido)phenyl)thieno[3,2-d]-pyrimidine-7-carboxamide (0.080 g, 71%) as a solid. LC/MS: m/z 479.0, 481.0 [M−1]

Step B: 4-Chloro-N-(6-chloro-2-fluoro-3-(3-fluoropropylsulfonamido)phenyl)-thieno[3,2-d]pyrimidine-7-carboxamide (0.080 g, 0.17 mmol) was suspended in 2 M ammonia (3.3 mL, 6.6 mmol) in i-PrOH. The reaction mixture was placed in a microwave reactor at 110° C. for 2 hours. The reaction mixture was concentrated and the crude product purified via silica gel chromatography, eluting with dichloromethane/ethyl acetate (3:2) to give 4-amino-thieno[3,2-d]pyrimidine-7-carboxylic acid [6-chloro-2-fluoro-3-(3-fluoro-propane-1-sulfonylamino)-phenyl]-amide (0.047 g, 61%) as a solid. ¹H NMR (400 MHz, DMSO-d₆) δ 11.49 (br s, 1H), 10.03 (br s, 1H), 8.97 (s, 1H), 8.55 (s, 1H), 7.96 (br s, 2H), 7.43 (m, 2H), 4.61 (m, 1H), 4.49 (m, 1H), 3.25 (m, 2H), 2.17-2.04 (m, 2H). LC/MS: m/z 462.1, 464.1 [M+1].

Examples 14-24 below in Table 2 were prepared according to the procedure described in Example 13 using appropriate starting materials.

TABLE 2

| Ex- | | | | |
|---|---|---|---|---|
| amp- | | | | ¹H NMR δ |
| le | | | MS | (400 MHz, DMSO-d6)* |
| no. | Structure | Name | m/z | (500 MHz DMSO-d6)** |
| 14 | 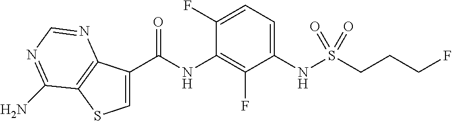 | 4-Amino-thieno[3,2-d] pyrimidine-7- carboxylic acid [2,6-difluoro-3-(3- fluoro-propane-1- sulfonylamino)- phenyl]-amide | 446.1 [M + 1] | *11.48 (s, 1H), 9.90 (s, 1H), 8.97 (s, 1H), 8.55 (s, 1H), 7.96 (s, 2H), 7.49-7.35 (m, 2H), 3.14 (q, J = 7.3 Hz, 2H), 1.26 (t, J = 7.3 Hz, 3H) |
| 15 | 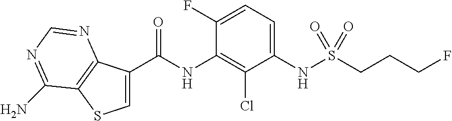 | 4-Amino-thieno[3,2-d] pyrimidine-7- carboxylic acid [2-chloro-6-fluoro-3- (3-fluoro-propane-1- sulfonylamino)- phenyl]-amide | 462.1, 464.1 [M + 1] | *11.51 (br s, 1H), 9.77 (br s, 1H), 8.97 (s, 1H), 8.55 (s, 1H), 7.96 (br s, 2H), 7.48 (m, 1H), 7.40 (m, 1H), 4.61 (m, 1H), 4.49 (m, 1H), 3.25 (m, 2H), 2.21-2.08 (m, 2H) |
| 16 | 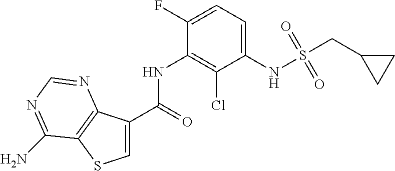 | 4-Amino-thieno[3,2-d] pyrimidine-7- carboxylic acid (2-chloro-3-cycloprop- ylmethanesulfonyl- amino-6-fluoro- phenyl)-amide | 456.0 [M + 1] | **11.50 (s, 1H), 9.56 (s, 1H), 8.96 (s, 1H), 8.55 (s, 1H), 7.95 (s, 2H), 7.49 (dd, J = 9.1, 5.2 Hz, 1H), 7.37 (t, J = 9.1 Hz, 1H), 3.13 (d, J = 7.1 Hz, 2H), 1.10 (ddd, J = 12.8, 8.0, 5.1 Hz, 1H), 0.61-0.54 (m, 2H), 0.39-0.34 (m, 2H) |
| 17 | 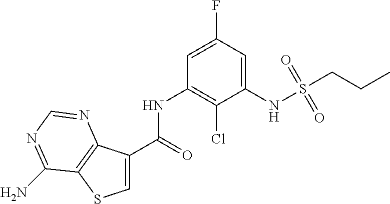 | 4-Amino-thieno[3,2-d] pyrimidine-7- carboxylic acid [2-chloro-5-fluoro-3- (propane-1-sulfonyl- amino)-phenyl]-amide | 444.0 [M + 1] | *12.53 (s, 1H), 9.78 (s, 1H), 9.02 (s, 1H), 8.62 (s, 1H), 8.34 (dd, J = 11.1, 2.9 Hz, 1H), 7.96 (s, 2H), 7.14 (dd, J = 9.7, 3.0 Hz, 1H), 3.24-3.13 (m, 2H), 1.83-1.70 (m, 2H), 0.98 (t, J = 7.4 Hz, 3H) |
| 18 | 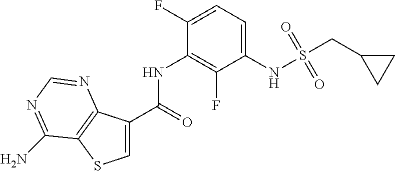 | 4-Amino-thieno[3,2-d] pyrimidine-7- carboxylic acid (3-cyclopropyl- methanesulfonyl- amino-2,6-di- fluoro-phenyl)-amide | 440.0 [M + 1] | *11.34 (s, 1H), 9.71 (s, 1H), 8.96 (s, 1H), 8.55 (s, 1H), 7.93 (s, 2H), 7.41 (td, J = 8.9, 5.6 Hz, 1H), 7.21 (t, J = 9.2 Hz, 1H), 3.10 (d, J = 7.1 Hz, 2H), 1.1-1.02 (m, 1H), 0.59- 0.53 (m, 2H), 0.34 (q, J = 4.6 Hz, 2H) |
| 19 | 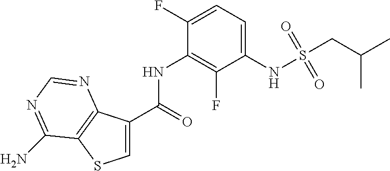 | 4-Amino-thieno[3,2-d] pyrimidine-7- carboxylic acid [2,6-difluoro-3-(2- methyl-propane-1- sulfonylamino)- phenyl]-amide | 442.0 [M + 1] | *11.35 (s, 1H), 9.70 (s, 1H), 8.96 (s, 1H), 8.55 (s, 1H), 7.93 (s, 2H), 7.43-7.32 (m, 1H), 7.22 (t,J = 8.9 Hz, 1H), 3.01 (d, J = 6.4 Hz, 2H), 2.18 (dt, J = 13.3, 6.5 Hz, 1H), 1.03 (d, J = 6.7 Hz, 6H) |
| 20 | 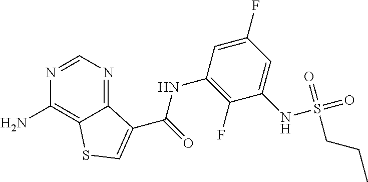 | 4-Amino-thieno[3,2-d] pyrimidine-7- carboxylic acid [2,5-difluoro-3- (propane-1-sulfonyl- amino)-phenyl]-amide | 428.0 [M + 1] | *12.30 (s, 1H), 10.01 (s, 1H), 9.00 (s, 1H), 8.55 (s, 1H), 8.16-8.08 (m, 1H), 7.96 (s, 2H), 7.02 (ddd, J = 9.4, 5.9, 3.2 Hz, 1H), 3.22- 3.13 (m, 2H), 1.81-1.68 (m, 2H), 0.99 (t, J = 7.4 Hz, 3H) |
| 21 | 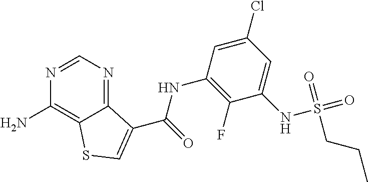 | 4-Amino-thieno[3,2-d] pyrimidine-7- carboxylic acid [5-chloro-2-fluoro-3- (propane-1-sulfonyl- amino)-phenyl]-amide | 444.0 [M + 1] | *12.28 (s, 1H), 10.00 (s, 1H), 9.01 (d, J = 7.8 Hz, 1H), 8.55 (s, 1H), 8.36 (dd, J = 5.8, 2.5 Hz, 1H), 7.96 (s, 2H), 7.22 (dd, J = 6.5, 2.6 Hz, 1H), 1H), 3.22-3.12 (m, 2H), 1.81- 1.67 (m, 2H), 0.99 (t, J = 7.4 Hz, 3H) |
| 22 | 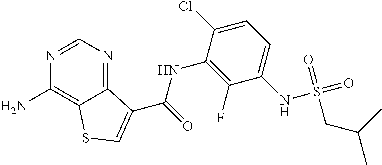 | 4-Amino-thieno[3,2-d] pyrimidine-7- carboxylic acid [6-chloro-2-fluoro-3- (2-methyl-propane-1- sulfonylamino)- phenyl]-amide | 458.0 [M + 1] | *11.48 (s, 1H), 9.91 (s, 1H), 8.97 (s, 1H), 8.55 (s, 1H), 7.96 (s, 2H), 7.47-7.37 (m, 2H), 3.04 (d, J = 6.4 Hz, 2H), 2.23-2.12 (m, 1H), 1.02 (d, J = 6.6 Hz, 6H) |
| 23 | 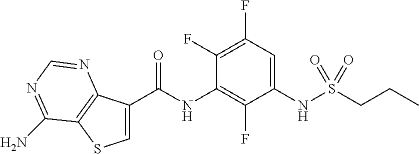 | 4-Amino-thieno[3,2-d] pyrimidine-7- carboxylic acid [2,3,6-trifluoro-5-(pro- pane-1-sulfonyl- amino)-phenyl]-amide | 445.9 [M + 1] | *11.48 (s, 1H), 9.95 (s, 1H), 8.99 (s, 1H), 8.55 (s, 1H), 7.95 (s, 2H), 7.45 (dt, J = 11.5, 7.7 Hz, 1H), 3.20-3.09 (m, 2H), 1.81-1.65 (m, 2H), 0.98 (t, J = 7.4 Hz, 3H) |
| 24 | 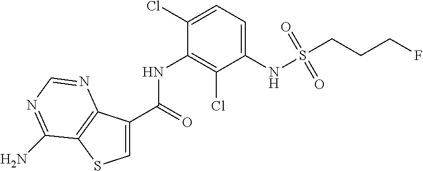 | 4-Amino-thieno[3,2-d] pyrimidine-7- carboxylic acid [2,6-dichloro-3-(3- fluoro-propane-1- sulfonylamino)- phenyl]-amide | 478.0 [M + 1] | *11.51 (s, 1H), 8.95 (s, 1H), 8.55 (s, 1H), 7.96 (s, 2H), 7.53-7.43 (m, 2H), 4.60 (t, J = 6.0 Hz, 1H), 4.48 (t, J = 6.0 Hz, 1H), 3.21-3.11 (m, 2H), 2.19-2.00 (m, 2H) |

### Example 25

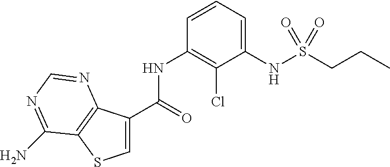

### 4-Amino-thieno[3,2-d]pyrimidine-7-carboxylic acid[2-chloro-3-(propane-1-sulfonylamino)-phenyl]-amide

Step A: 4-(2,4-Dimethoxybenzylamino)thieno[3,2-d]pyrimidine-7-carboxylic acid (0.0542 g, 0.157 mmol), N-(3-amino-2-chlorophenyl)propane-1-sulfonamide (0.030 g, 0.121 mmol), HATU (0.0596 g, 0.157 mmol) and DIEA (d 0.742) (0.0420 mL, 0.241 mmol) were dissolved in DMF and stirred at 55° C. overnight. The reaction mixture was cooled to room temperature and partitioned between EtOAc and water. The organic layer was washed with water (3×), 0.1 N HCl, saturated aqueous NaHCO₃ and brine, dried over Na₂SO₄ and concentrated to a tan oil. The oil was filtered through a plug of silica gel with the aid of 2:1 Hexanes/EtOAc to give N-(2-chloro-3-(propylsulfonamido)phenyl)-4-(2,4-dimethoxybenzyl-amino)thieno[3,2-d]pyrimidine-7-carboxamide as an oil which was used directly in the next step.

Step B: N-(2-chloro-3-(propylsulfonamido)phenyl)-4-(2,4-dimethoxybenzylamino)-thieno[3,2-d]pyrimidine-7-carboxamide (0.081 g, 0.14 mmol) was dissolved in trifluoroacetic acid (“TFA”) (5 mL) and heated at reflux for 3 hours. The reaction mixture was cooled to room temperature and concentrated to a red oil. The crude was dissolved in EtOAc, washed with saturated aqueous NaHCO₃ and brine, dried over Na₂SO₄ and concentrated to an oil. Methanol was added and a white precipitate formed which was collected by filtration, washed further with MeOH and dried under high vacuum to give 4-amino-thieno[3,2-d]pyrimidine-7-carboxylic acid [2-chloro-3-(propane-1-sulfonylamino)-phenyl]amide (9 mg, 0.021 mmol, 15%) as a white solid. ¹H NMR (400 MHz, DMSO-d₆) δ 12.30 (s, 1H), 9.55 (s, 1H), 8.99 (s, 1H), 8.61 (s, 1H), 8.41-8.43 (d, 1H), 7.93 (br s, 2H), 7.36-7.40 (t, 1H), 7.26-7.28 (d, 1H), 3.12-3.16 (t, 1H), 1.75-1.81 (m, 2H), 0.97-1.01 (t, 3H); m/z (APCI-pos) M+1=426.0, 428.0.

### Example 26

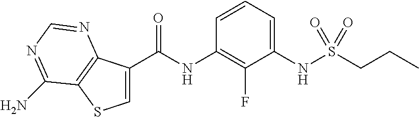

### 4-Amino-thieno[3,2-d]pyrimidine-7-carboxylic acid[2-fluoro-3-(propane-1-sulfonylamino)-phenyl]-amide

Step A: 4-(2,4-Dimethoxy-benzylamino)thieno[3,2-d]pyrimidine-7-carboxylic acid (69 mg, 0.2 mmol, prepared as in Example 8, Step A), N-(3-amino-2-fluorophenyl) propane-1-sulfonamide (57 mg, 0.2 mmol), HATU (84 mg, 0.22 mmol), and a catalytic amount of DMAP (2 mg, 0.02 mmol) were dissolved in DMF (2 mL). N,N-Diisopropyl-ethylamine (87 uL, 0.50 mmol) was added followed by stirring at room temperature for 1 hour. Ethyl acetate (50 mL) was added, and the mixture was washed with brine. Removal of the organics under reduced pressure gave 4-(2,4-dimethoxy-benzylamino)-N-(2-fluoro-3-(propyl-sulfonamido)-phenyl)thieno[3,2-d]pyrimidine-7-carboxamide (110 mg, 99%). LC-MS [M+1]m/z 560.1.

Step B: 4-(2,4-Dimethoxy-benzylamino)-N-(2-fluoro-3-(propylsulfonamido)phenyl)-thieno[3,2-d]pyrimidine-7-carboxamide (130 mg, 0.23 mmol) was taken up in TFA (4 mL). The reaction mixture was heated at reflux for 2 hours and the organics were removed under reduced pressure. Purification by preparative HPLC afforded 4-amino-thieno[3,2-d]pyrimidine-7-carboxylic acid [2-fluoro-3-(propane-1-sulfonylamino)-phenyl]-amide (23 mg, 23%) ¹H NMR (500 MHz, DMSO-d₆) δ 12.09 (s, 1H), 9.74 (br s, 1H), 8.97 (s, 1H), 8.56 (s, 1H), 8.26 (t, J=7.1 Hz, 1H), 7.92 (s, 2H), 7.22-7.14 (m, 2H), 3.11 (s, 2H), 1.86-1.62 (m, 2H), 0.99 (t, J=7.4 Hz, 3H). LC-MS [M+1]m/z 410.

### Example 27

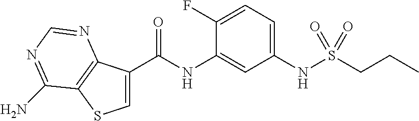

### 4-Amino-thieno[3,2-d]pyrimidine-7-carboxylic acid[2-fluoro-5-(propane-1-sulfonylamino)-phenyl]-amide

The title compound was prepared using a similar procedure described in Example 26, using N-(3-amino-4-fluorophenyl)propane-1-sulfonamide in place of N-(3-amino-2-fluorophenyl)propane-1-sulfonamide. ¹H NMR (500 MHz, DMSO-d₆) δ 12.11 (s, 1H), 9.79 (s, 1H), 8.97 (s, 1H), 8.55 (s, 1H), 8.43 (dd, J=6.9, 2.5 Hz, 1H), 7.91 (s, 1H), 7.29 (dd, J=10.6, 9.0 Hz, 1H), 7.02-6.92 (m, 1H), 3.11-2.92 (m, 2H), 1.80-1.56 (m, 2H), 0.95 (t, J=7.5 Hz, 2H). LC-MS [M+1]m/z 410.

### Example 28

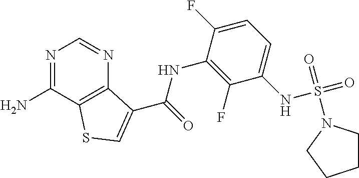

### 4-Amino-thieno[3,2-d]pyrimidine-7-carboxylic acid[2,6-difluoro-3-(pyrrolidine-1-sulfonylamino)-phenyl]-amide

The title compound was prepared using a similar procedure described in Example 25, using N-(3-amino-2,4-difluorophenyl)pyrrolidine-1-sulfonamide in place of N-(3-amino-2-chlorophenyl)propane-1-sulfonamide. ¹H NMR (400 MHz, MeOH-d₄) δ 8.88 (s, 1H), 8.54 (s, 1H), 7.52-7.59 (m, 1H), 7.06-7.12 (m, 1H), 3.26-3.30 (m, 4H), 1.85-1.90 (m, 4H); m/z (APCI-pos) M+1=455.1.

### Example 29

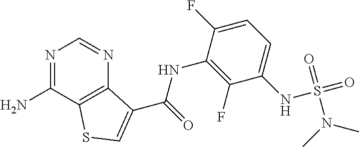

### 4-Amino-N-(3-(N,N-dimethylsulfamoylamino)-2,6-difluorophenyl)thieno[3,2-d]pyrimidine-7-carboxamide

Step A: To N-(3-amino-2,4-difluorophenyl)propane-1-sulfonamide (0.100 g, 0.400 mmol) in DMF (2 mL) was added potassium carbonate (0.166 g, 1.20 mmol) and dimethylsulfamoyl chloride (0.0599 mL, 0.559 mmol). The suspension was stirred at ambient temperature for 18 hours. To the suspension was then added 2 mL of 2M NaOH which was stirred at ambient temperature for 1 hour. The resulting solution was diluted with water (20 mL) and brought to pH 9 with HCl followed by extraction with EtOAc (3×15 mL). The concentrated organics were purified via silica gel chromatography eluting with hexane/EtOAc (1:1) to provide N-(3-amino-2,4-difluorophenyl)dimethylamino-1-sulfonamide (0.090 g, 90%).

Step B: To N-(3-amino-2,4-difluorophenyl)dimethylamino-1-sulfonamide (0.090 g, 0.19 mmol) in DMF (1 mL) was added 4-(2,4-dimethoxybenzylamino)thieno[3,2-d]pyrimidine-7-carboxylic acid (0.090 g, 0.26 mmol), Hunig's base (0.10 mL, 0.56 mmol) and HATU (0.100 g, 0.26 mmol). The solution was stirred at room temperature for 48 hours before dilution with EtOAc (15 mL) and washing with water and brine. The concentrated organics were purified via silica gel chromatography eluting with hexane/EtOAc (1:1) to provide 4-(2,4-dimethoxybenzylamino)-N-(3-(N,N-dimethylsulfamoylamino)-2,6-difluorophenyl)thieno[3,2-d]pyrimidine-7-carboxamide (0.016 g, 16%).

Step C: 4-(2,4-dimethoxybenzylamino)-N-(3-(N,N-dimethylsulfamoylamino)-2,6-difluorophenyl)thieno[3,2-d]pyrimidine-7-carboxamide (0.016 g, 0.028 mmol) was dissolved in TFA (0.5 mL) and warmed to 75° C. for 1 hour. The cooled solution was concentrated and the residue partitioned between EtOAc and saturated aqueous sodium bicarbonate solution. The organics were concentrated and the residue purified via trituration with dichloromethane to provide 4-amino-N-(3-(N,N-dimethylsulfamoylamino)-2,6-difluorophenyl)-thieno[3,2-d]pyrimidine-7-carboxamide (0.010 g, 84%). ¹H NMR (400 MHz, MeOH-d₄) δ 8.86 (s, 1H), 8.55 (s, 1H), 7.47-7.55 (m, 1H), 6.99-7.06 (m, 1H), 2.78 (s, 6H); m/z (APCI-pos) M+1=429.1.

### Example 30

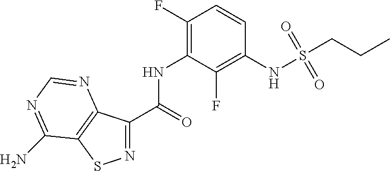

### 7-Amino-isothiazolo[4,5-d]pyrimidine-3-carboxylic acid [2,6-difluoro-3-(propane-1-sulfonylamino)-phenyl]-amide

Step A: To 7-oxo-6,7-dihydroisothiazolo[4,5-d]pyrimidine-3-carboxylic acid (0.100 g, 0.5072 mmol) was added thionyl chloride (3.693 mL, 50.72 mmol) and N,N-dimethylformamide (0.01964 mL, 0.2536 mmol). The mixture was refluxed for 2 h. The cooled reaction mixture was evaporated, chased with 3 mL CHCl₃, triturated with 3 mL hexane, and dried under high vacuum to afford 7-chloroisothiazolo[4,5-d]pyrimidine-3-carbonyl chloride as a brown solid.

Step B: To 7-chloroisothiazolo[4,5-d]pyrimidine-3-carbonyl chloride dissolved in 5 mL CHCl₃ was added N-(3-amino-2,4-difluorophenyl)propane-1-sulfonamide (0.118 g, 0.470 mmol). The reaction mixture was stirred at ambient temperature for 30 min. The solvent was evaporated, the residue was suspended in 5 mL dioxane, and a gentle stream of ammonia gas was bubbled in for 2 min. After 30 min the orange suspension was evaporated and the residue partitioned between water and EtOAc. The EtOAc was washed with brine, dried over MgSO₄, filtered, and evaporated to yield 0.17 g tan solid. This was chromatographed on a Biotage SNAP column with 10:10:1 dichloromethane:EtOAc:MeOH to afford 7-amino-isothiazolo[4,5-d]pyrimidine-3-carboxylic acid [2,6-difluoro-3-(propane-1-sulfonyl-amino)-phenyl]-amide (0.0076 g, 0.0177 mmol, 3.77% yield). ¹H NMR (400 MHz, CD₃OD) δ 8.58 (s, 1H), 7.50-7.56 (m, 1H), 7.11-7.16 (m, 1H), 3.08-3.12 (m, 2H), 1.82-1.91 (m, 2H), 1.05 (t, 3H). LC/MS: m/z 427.0 [M−1].

### Example 31

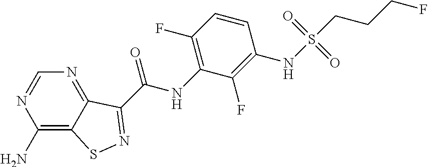

### 7-Amino-isothiazolo[4,5-d]pyrimidine-3-carboxylic acid [2,6-difluoro-3-(3-fluoro-propane-1-sulfonylamino)-phenyl]-amide

The title compound was prepared according to the general procedure as described in Example 30, substituting N-(3-amino-2,4-difluorophenyl)-3-fluoropropane-1-sulfonamide for N-(3-amino-2,4-difluorophenyl)propane-1-sulfonamide. ¹H NMR (400 MHz, CD₃OD) δ 8.58 (s, 1H), 7.50-7.56 (m, 1H), 7.12-7.17 (m, 1H), 4.62-4.46 (m, 2H), 3.22-3.27 (m, 2H), 2.16-2.28 (m, 2H). LC/MS: m/z 447.1 [M+1].

### Example 32

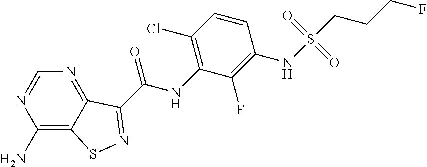

### 7-Amino-isothiazolo[4,5-d]pyrimidine-3-carboxylic acid [6-chloro-2-fluoro-3-(3-fluoro-propane-1-sulfonylamino)-phenyl]-amide

The title compound was prepared according to the general procedure as described in Example 30, substituting N-(3-amino-4-chloro-2-fluorophenyl)-3-fluoropropane-1-sulfon-amide for N-(3-amino-2,4-difluorophenyl)propane-1-sulfonamide. ¹H NMR (400 MHz, CD₃OD) δ 8.59 (s, 1H), 7.54-7.58 (m, 1H), 7.38-7.41 (m, 1H), 4.46-4.61 (m, 2H), 3.25-3.29 (m, 2H), 2.15-2.26 (m, 2H). LC/MS: m/z 463.1 [M+1].

### Example 33

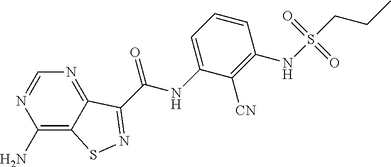

### 7-Amino-isothiazolo[4,5-d]pyrimidine-3-carboxylic acid [2-cyano-3-(propane-1-sulfonylamino)-phenyl]-amide

The title compound was prepared according to the general procedure as described in Example 30 substituting N-(3-Amino-2-cyanophenyl)propane-1-sulfonamide for N-(3-amino-2,4-difluorophenyl)propane-1-sulfonamide. ¹H NMR (400 MHz, DMSO-d₆) δ 12.62 (s, 1H), 10.18 (s, 1H), 8.54 (s, 1H), 8.48 (br s, 2H), 8.34 (d, 1H), 7.75 (dd, 1H), 7.32 (d, 1H), 3.19-3.23 (m, 2H), 1.76-1.86 (m, 2H), 1.01 (t, 3H). LC/MS: m/z 418.1 [M+1].

### Example 34

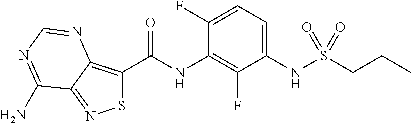

### 7-Amino-isothiazolo[4,3-d]pyrimidine-3-carboxylic acid [2,6-difluoro-3-(propane-1-sulfonylamino)-phenyl]-amide

Step A: N-(3-Amino-2,4-difluorophenyl)-N-(4-methoxybenzyl)propane-1-sulfonamide (424.9 mg, 1.147 mmol) was dissolved in toluene (5 mL, 40 mmol). 2 M of trimethylaluminum in hexane (1.912 mL) was added to this stirred solution and the reaction was stirred at room temperature for 1 hour. Ethyl 7-aminoisothiazolo[4,3-d]pyrimidine-3-carboxylate (200.0 mg, 0.8919 mmol) was added as a solid and the reaction was stirred at 80° C. overnight. After cooling to room temperature the reaction mixture was quenched with 5 mL of 1N Rochelle salt solution. The crude mixture was passed through a drying column, eluted with ethyl acetate, and purified by flash chromatography (0-100% ethyl acetate:heptane) and indicated fractions were concentrated to give 7-amino-N-(2,6-difluoro-3-(N-(4-methoxybenzyl)propylsulfonamido)phenyl)-isothiazolo[4,3-d]pyrimidine-3-carboxamide as a light yellow foam (352 mg, 68%). ¹H NMR (400 MHz, DMSO-d₆) δ 10.52 (s, 1H), 8.81 (s, 1H), 8.67 (s, 1H), 8.45 (s, 1H), 7.38-7.25 (m, 1H), 7.19 (t, J=10.7 Hz, 3H), 6.85 (d, J=8.6 Hz, 2H), 4.71 (s, 2H), 3.71 (s, 3H), 3.26 (s, 2H), 1.79 (dd, J=15.2, 7.6 Hz, 2H), 1.01 (t, J=7.4 Hz, 3H). MS m/z=549.3 [M+1].

Step B: 7-Amino-N-(2,6-difluoro-3-(N-(4-methoxybenzyl)propylsulfonamido)phenyl)-isothiazolo[4,3-d]pyrimidine-3-carboxamide (587 mg, 0.00107 mol) was dissolved in dichloromethane (5 mL, 0.08 mol). Ttrifluoroacetic acid (3 mL, 0.04 mol) was added and the reaction was allowed to stir at room temperature for two hours followed by heating to 40° C. for two hours and then 50° C. for thirty minutes. The reaction mixture was concentrated and dissolved in ethyl acetate. The precipitated solid was collected by filtration and dried in a vacuum oven to afford 7-amino-isothiazolo[4,3-d]pyrimidine-3-carboxylic acid [2,6-difluoro-3-(propane-1-sulfonylamino)-phenyl]-amide as a yellow solid (212 mg, 46%). ¹H NMR (400 MHz, DMSO-d₆) δ 10.54 (s, 1H), 9.73 (s, 1H), 8.80 (s, 1H), 8.67 (s, 1H), 8.46 (s, 1H), 7.42 (dd, J=14.5, 9.0 Hz, 1H), 7.25 (t, J=8.8 Hz, 1H), 3.17-3.01 (m, 2H), 1.76 (dt, J=15.1, 7.5 Hz, 2H), 0.98 (t, J=7.4 Hz, 3H). MS m/z=429.0 [M+1].

### Example 35

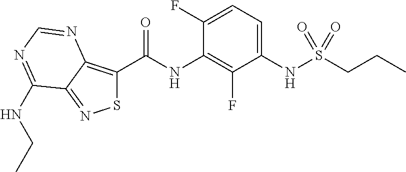

### 7-Ethylamino-isothiazolo[4,3-d]pyrimidine-3-carboxylic acid [2,6-difluoro-3-(propane-1-sulfonylamino)-phenyl]-amide

Step A: N-(3-Amino-2,4-difluorophenyl)-N-(4-methoxybenzyl)propane-1-sulfon-amide (143.9 mg, 0.388 mmol) was dissolved in toluene (2 mL, 20 mmol). 2 M of trimethylaluminum in hexane (0.5826 mL) was added to this stirred solution and the reaction was stirred at room temperature for 1 hour. Ethyl 7-(ethylamino)isothiazolo[4,3-d]pyrimidine-3-carboxylate (98 mg, 0.39 mmol) was added as a solid and the reaction was stirred at 80° C. overnight. After cooling to room temperature the reaction mixture was quenched with 5 mL of 1N Rochelle salt solution. The crude mixture was passed through a drying column, eluted with ethyl acetate, and purified by flash chromatography (0-100% ethyl acetate:heptane) to give N-(2,6-difluoro-3-(N-(4-methoxybenzyl)propylsulfonamido)phenyl)-7-(ethylamino)isothiazolo[4,3-d]pyrimidine-3-carboxamide as a yellow solid (111 mg, 50%). ¹H NMR (400 MHz, DMSO-d₆) δ 10.53 (s, 1H), 9.39 (s, 1H), 8.52 (s, 1H), 7.31 (d, J=6.1 Hz, 1H), 7.19 (t, J=9.7 Hz 3H), 6.85 (d, J=8.6 Hz, 2H), 4.71 (s, 2H), 3.71 (s, 3H), 3.68-3.60 (m, 2H), 3.26 (m, 3H), 1.79 (dd, J=15.2, 7.5 Hz, 2H), 1.23 (t, 3H), 1.01 (t, J=7.4 Hz, 3H). MS m/z=577.3 [M+1].

Step B: N-(2,6-Difluoro-3-(N-(4-methoxybenzyl)propylsulfonamido) phenyl)-7-(ethylamino)isothiazolo[4,3-d]pyrimidine-3-carboxamide (107 mg, 0.186 mmol) was dissolved in trifluoroacetic acid (1 mL, 10 mmol) and stirred at room temperature for three hours. The reaction mixture was concentrated and purified by flash chromatography (0-100% ethyl acetate:heptane) to give 7-ethylamino-isothiazolo[4,3-d]pyrimidine-3-carboxylic acid [2,6-difluoro-3-(propane-1-sulfonylamino)-phenyl]-amide (42 mg, 50%). ¹H NMR (400 MHz, DMSO-d₆) δ 10.56 (s, 1H), 9.76 (s, 1H), 9.43 (t, J=5.7 Hz, 1H), 8.53 (s, 1H), 7.46-7.38 (m, 1H), 7.26 (t, J=8.7 Hz, 1H), 3.69-3.57 (m, 2H), 3.12-3.05 (m, 2H), 1.83-1.67 (m, 2H), 1.25 (t, J=7.2 Hz, 3H), 0.98 (t, J=7.4 Hz, 3H). MS m/z=457.1 [M+1].

### Example 36

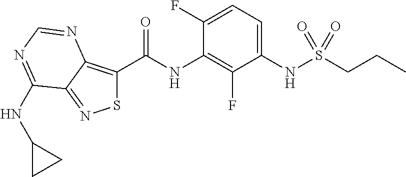

### 7-(Cyclopropylamino)-N-(2,6-difluoro-3-(propylsulfonamido)phenyl)isothiazolo[4,3-d]pyrimidine-3-carboxamide

The above example was prepared using a similar procedure as for Example 35 using ethyl 7-(cyclopropylamino)isothiazolo[4,3-d]pyrimidine-3-carboxylate in place of ethyl 7-(ethylamino)isothiazolo[4,3-d]pyrimidine-3-carboxylate. ¹H NMR (400 MHz, DMSO-d₆) δ 10.56 (s, 1H), 9.76 (s, 1H), 9.49 (d, J=4.6 Hz 1H), 8.57 (s, 1H), 7.42 (d, J=6.7 Hz, 1H), 7.26 (t, J=9.4 Hz, 1H), 3.26 (s, 1H), 3.15-3.02 (m, 2H), 1.75 (dd, J=15.0, 7.3 Hz, 2H), 0.98 (t, J=7.4 Hz, 3H), 0.85 (d, J=7.9 Hz, 4H). MS m/z=469.0 [M+1].

### Example 37

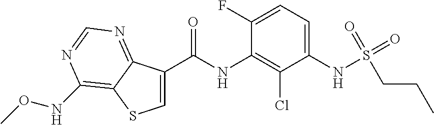

### 4-Methoxyamino-thieno[3,2-d]pyrimidine-7-carboxylic acid [2-chloro-6-fluoro-3-(propane-1-sulfonylamino)-phenyl]-amide

Step A: N-(3-Amino-2-chloro-4-fluorophenyl)-N-(4-methoxybenzyl)propane-1-sulfonamide (5.52 g, 14.26 mmol) was dissolved in toluene (75.9 mL, 712.9 mmol). A solution of 2 M trimethylaluminum in hexane (7.49 mL) was added over 20 minutes and the reaction was stirred at room temperature for 1 hour. Methyl 4-chlorothieno[3,2-d]pyrimidine-7-carboxylate (3.30 g, 14.42 mmol) was added and the reaction was stirred at 80° C. under nitrogen for 4 hours. LC/MS analysis indicated consumption of starting material and formation of two product peaks correlating to desired product and hydrolysis byproduct. The reaction mixture was cooled and quenched by slow addition of 70 mL 1N Rochelle salt solution and subsequent stirring at room temperature overnight. The aqueous solution was then extracted with 250 mL ethyl acetate (3×) and the combined organic layers dried over magnesium sulfate, filtered, and concentrated. The crude product was purified by chromatography to give a 1:1 mixture of 4-chloro-N-(2-chloro-6-fluoro-3-(N-(4-methoxybenzyl)propylsulfonamido)phenyl)thieno[3,2-d]pyrimidine-7-carboxamide (m/z=583.2 [M+1]) and N-(2-chloro-6-fluoro-3-(N-(4-methoxybenzyl)propylsulfonamido)phenyl)-4-methylthieno[3,2-d]pyrimidine-7-carboxamide (m/z=563.3 [M+1]). This mixture was carried forward without further purification.

Step B: Methoxyamine hydrochloride (180 mg, 0.00214 mmol) was suspended in 1 mL ethyl ether, stirred for 2 minutes, and allowed to settle to the bottom of the tube. The ether was decanted off and the procedure repeated with another 1 mL of ethyl ether. A 1:1 mixture of 4-chloro-N-(2-chloro-6-fluoro-3-(N-(4-methoxybenzyl)propylsulfonamido)phenyl)thieno[3,2-d]pyrimidine-7-carboxamide and N-(2-chloro-6-fluoro-3-(N-(4-methoxybenzyl)propylsulfonamido)phenyl)-4-methylthieno[3,2-d]pyrimidine-7-carboxamide (80 mg) was combined with the ether dried methoxyamine hydrochloride in DMSO (0.3 mL), and the reaction mixture was heated in a microwave reactor at 140° C. for 1 hour. LC-MS analysis indicated formation of N-(2-chloro-6-fluoro-3-(N-(4-methoxybenzyl)propylsulfonamido)-phenyl)-4-(methoxyamino)thieno[3,2-d]pyrimidine-7-carboxamide (m/z=594.3 [M+1]) and unreacted N-(2-chloro-6-fluoro-3-(N-(4-methoxybenzyl)propylsulfonamido)phenyl)-4-methylthieno[3,2-d]pyrimidine-7-carboxamide (m/z=563.3 [M+1]). This mixture was evaporated to dryness and used “as is” in subsequent reaction.

Step C: A mixture of N-(2-chloro-6-fluoro-3-(N-(4-methoxybenzyl)-propylsulfonamido)phenyl)-4-(methoxyamino)thieno[3,2-d]pyrimidine-7-carboxamide and N-(2-chloro-6-fluoro-3-(N-(4-methoxybenzyl)propylsulfonamido)phenyl)-4-methylthieno[3,2-d]pyrimidine-7-carboxamide in dichloromethane (5 mL, 80 mmol) and trifluoroacetic acid (5 mL, 60 mmol) was stirred for 1 hour. The reaction mixture was concentrated to dryness, redissolved in 1 mL DMF, filtered through a nylon disk and purified by reverse phase HPLC to afford 4-methoxyamino-thieno[3,2-d]pyrimidine-7-carboxylic acid [2-chloro-6-fluoro-3-(propane-1-sulfonylamino)-phenyl]-amide (10.7 mg, 32%). ¹H NMR (500 MHz, DMSO-d₆) δ 11.74 (s, 1H), 11.23 (s, 1H), 10.90 (s, 1H), 8.55 (s, 1H), 7.89 (s, 1H), 7.45 (dd, J=9.1, 5.2 Hz, 1H), 7.34 (t, J=9.0 Hz, 1H), 3.81 (s, 3H), 3.14-3.00 (m, 2H), 1.76 (dq, J=14.9, 7.4 Hz, 2H), 0.98 (t, J=7.4 Hz 3H). MS m/z=474.0 [M+1].

### Example 38

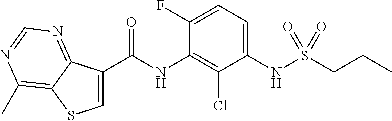

### 4-Methyl-thieno[3,2-d]pyrimidine-7-carboxylic acid [2-chloro-6-fluoro-3-(propane-1-sulfonylamino)-phenyl]-amide

The title compound was isolated as a side product from the preparation of Example 37 (10 mg, 30%). ¹H NMR (500 MHz, DMSO-d₆) δ 11.01 (s, 1H), 9.70 (br s, 1H), 9.27 (d, J=8.9 Hz, 2H), 7.48 (dd, J=9.1, 5.3 Hz, 1H), 7.36 (t, J=9.2 Hz, 1H), 3.13-3.05 (m, 2H), 2.86 (s, 3H), 1.77 (dq, J=15.0, 7.5 Hz, 2H), 0.98 (t, J=7.4 Hz, 3H). MS m/z=443.0 [M+1].

### Example 39

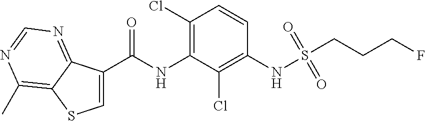

### 4-Methyl-thieno[3,2-d]pyrimidine-7-carboxylic acid [2,6-dichloro-3-(3-fluoro-propane-1-sulfonylamino)-phenyl]-amide

Step A: 4-Chloro-N-(2,6-dichloro-3-(3-fluoropropylsulfonamido)phenyl)thieno[3,2-d]-pyrimidine-7-carboxamide was prepared in a similar manner described in Example 13, Step A, using N-(3-amino-2,4-dichlorophenyl)-3-fluoropropane-1-sulfonamide in place of N-(3-amino-4-chloro-2-fluorophenyl)-3-fluoropropane-1-sulfonamide.

Step B: A microwave vial was charged with 4-chloro-N-(2,6-di-chloro-3-(3-fluoropropylsulfonamido)phenyl)thieno[3,2-d]pyrimidine-7-carboxamide (0.08 g, 0.16 mmol), trimethylaluminum (0.20 mL, 2M in heptane), tetrakis(triphenylphosphine)-palladium(0) (0.02 g, 0.02 mmol) and THF (1.6 mL). The reaction mixture was heated in a microwave reactor at 75° C. for 15 minutes. The salts were filtered off, and the filtrate was concentrated in vacuo, then purified by reverse phase HPLC to afford 4-methyl-thieno[3,2-d]pyrimidine-7-carboxylic acid [2,6-dichloro-3-(3-fluoro-propane-1-sulfonylamino)-phenyl]-amide (0.015 g, 20%). ¹H NMR (500 MHz, DMSO-d₆) δ 11.02 (s, 1H), 9.28 (s, 1H), 9.27 (s, 1H), 7.45 (s, 2H), 6.56 (s, 1H), 4.60 (t, J=6.0 Hz, 1H), 4.48 (t, J=6.0 Hz, 1H), 3.14-3.00 (m, 2H), 2.86 (s, 3H), 2.16-1.99 (m, 2H). m/z (ES-MS) 477.0 [M+1].

### Example 40

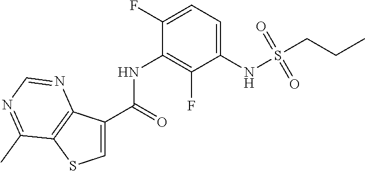

### 4-Methyl-thieno[3,2-d]pyrimidine-7-carboxylic acid [2,6-difluoro-3-(propane-1-sulfonylamino)-phenyl]-amide

Step A: 4-Chloro-N-(2,6-difluoro-3-(propylsulfonamido)phenyl)thieno[3,2-d]pyrimidine-7-carboxamide was prepared in a similar manner described in Example 13, Step A, using N-(3-amino-2,4-difluorophenyl)propane-1-sulfonamide in place of N-(3-amino-4-chloro-2-fluorophenyl)-3-fluoropropane-1-sulfonamide.

Step B: A microwave vial was charged with 4-chloro-N-(2,6-difluoro-3-(propyl-□ulfonamide)phenyl)thieno[3,2-d]pyrimidine-7-carboxamide (85 mg, 0.19 mmol), methylboronic acid (29 mg, 0.48 mmol), tetrakis(triphenylphosphine)palladium(0) (33 mg, 0.03 mmol), potassium phosphate (101 mg, 0.48 mmol) and 1,4-dioxane (1.9 mL). The reaction mixture was heated in a microwave reactor at 120° C. for 15 minutes. The salts were filtered off, and the filtrate was concentrated in vacuo, then purified by reverse phase HPLC to afford 4-methyl-thieno[3,2-d]pyrimidine-7-carboxylic acid [2,6-difluoro-3-(propane-1-sulfonylamino)-phenyl]-amide (24 mg, 30%). ¹H NMR (400 MHz, DMSO-d₆) δ 10.87 (s, 1H), 9.71 (s, 1H), 9.32-9.21 (m, 2H), 7.47-7.32 (m, 1H), 7.30-7.17 (m, 1H), 3.14-3.03 (m, 2H), 1.83-1.67 (m, 2H), 1.03-0.91 (m, 3H). m/z (ES-MS) 427.0 [M+1].

### Example 41

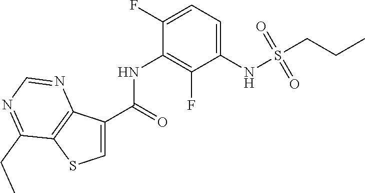

### 4-Ethyl-thieno[3,2-d]pyrimidine-7-carboxylic acid [2,6-difluoro-3-(propane-1-sulfonylamino)-phenyl]-amide

The title compound was prepared using a similar procedure as described in Example 40 using ethylboronic acid instead of methylboronic acid. ¹H NMR (400 MHz, DMSO-d₆) δ 10.87 (s, 1H), 9.72 (s, 1H), 9.31 (s, 1H), 9.28 (s, 1H), 7.40 (td, J=8.8, 5.7 Hz, 1H), 7.23 (t, J=9.1 Hz, 1H), 3.17 (q, J=7.5 Hz, 2H), 3.12-3.01 (m, 2H), 1.83-1.67 (m, 2H), 1.41 (t, J=7.5 Hz, 3H), 0.98 (t, J=7.4 Hz, 3H). m/z (ES-MS) 441.0 [M+1].

### Example 42

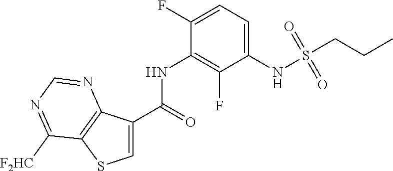

### 4-Difluoromethyl-thieno[3,2-d]pyrimidine-7-carboxylic acid [2,6-difluoro-3-(propane-1-sulfonylamino)-phenyl]-amide

Step A: 4-Chloro-N-(2,6-difluoro-3-(N-(4-methoxybenzyl)propylsulfonamido)phenyl)-thieno[3,2-d]pyrimidine-7-carboxamide was prepared in a similar manner as described in Example 2, Step A, using N-(3-amino-2,4-difluorophenyl)-N-(4-methoxybenzyl)propanesulfonamide instead of N-(3-amino-2,4-difluorophenyl)-N-(4-methoxybenzyl)ethanesulfonamide.

Step B: A microwave vial was charged with 4-chloro-N-(2,6-difluoro-3-(N-(4-methoxybenzyl)propylsulfonamido)phenyl)thieno[3,2-d]pyrimidine-7-carboxamide (0.54 g, 0.95 mmol), 4,4,5,5-tetramethyl-2-vinyl-1,3,2-dioxaborolane (0.24 mL, 1.43 mmol), bis(triphenylphosphine)palladium (II) chloride (0.067 g, 0.1 mmol), sodium carbonate (1.52 mL, 1M in water) and acetonitrile (4.3 mL). The reaction mixture was heated in a microwave reactor at 100° C. for 15 minutes. The reaction mixture was then diluted with ethyl acetate and water. The layers were separated and the aqueous layer extracted twice with ethyl acetate. The combined organic layers were dried with sodium sulfate, filtered and concentrated in vacuo. The crude mixture was purified by flash chromatography to afford N-(2,6-difluoro-3-(N-(4-methoxybenzyl)propylsulfonamido)phenyl)-4-vinylthieno[3,2-d]pyrimidine-7-carboxamide (0.13 g, 25%). m/z (ES-MS) 559.0 [M+1].

Step C: N-(2,6-difluoro-3-(N-(4-methoxybenzyl)propylsulfonamido)phenyl)-4-vinylthieno[3,2-d]pyrimidine-7-carboxamide (133 mg, 0.24 mmol) was dissolved in dichloromethane (2 mL). Then, at −78° C., a flow of ozone was passed through the solution for 5 minutes. The reaction mixture was allowed to warm to room temperature, and dimethyl sulfide (0.09 mL, 1.2 mmol) was added. The solution was diluted with dichloromethane and washed with a saturated aqueous solution of sodium thiosulfate. The organic layer was dried with sodium sulfate, filtered and concentrated in vacuo. The crude mixture was purified by flash chromatography to afford N-(2,6-difluoro-3-(N-(4-methoxybenzyl)-propylsulfonamido)phenyl)-4-(dihydroxymethyl)thieno[3,2-d]pyrimidine-7-carboxamide (47 mg, 34%). m/z (ES-MS) 579.0 [M+1].

Step D: To a solution of N-(2,6-difluoro-3-(N-(4-methoxybenzyl)propyl-□ulfonamide)phenyl)-4-(dihydroxymethyl)thieno[3,2-d]pyrimidine-7-carboxamide in dichloromethane (1.2 mL) was added bis(2-methoxyethyl)aminosulfur trifluoride (“Deoxo-Fluor”) (0.04 mL, 0.20 mmol) at −30° C. The reaction mixture was allowed to warm to room temperature and stirred for 16 hours, after which more bis(2-methoxyethyl)aminosulfur trifluoride (0.03 mL, 0.17 mmol) was added at −30° C. The reaction mixture was allowed again to warm up at room temperature and stirred for 8 more hours. The solution was diluted with dichloromethane and washed with a saturated aqueous solution of sodium bicarbonate. The organic layer was dried with sodium sulfate, filtered and concentrated in vacuo. The crude mixture was purified by flash chromatography to afford N-(2,6-difluoro-3-(N-(4-methoxybenzyl)propylsulfonamido)-phenyl)-4-(difluoromethyl)thieno[3,2-d]pyrimidine-7-carboxamide (23 mg, 61%). m/z (ES-MS) 583.0 [M+1].

Step E: To a solution of N-(2,6-difluoro-3-(N-(4-methoxybenzyl)propyl-□ulfonamide)phenyl)-4-(difluoromethyl)thieno[3,2-d]pyrimidine-7-carboxamide (23 mg, 0.04 mmol) in dichloromethane (0.45 mL) was added trifluoroacetic acid (0.15 mL) at 0° C., then reaction mixture was allowed to warm up at room temperature and stirred for 3 hours, after which more trifluoroacetic acid (0.05 mL) was added at 0° C. The reaction mixture was allowed again to warm up at room temperature and stirred for 8 more hours. The reaction mixture was then concentrated in vacuo, and purified by reverse phase HPLC to afford 4-difluoromethyl-thieno[3,2-d]pyrimidine-7-carboxylic acid [2,6-difluoro-3-(propane-1-sulfonylamino)-phenyl]-amide (13 mg, 71%). ¹H NMR (400 MHz, DMSO) δ 10.70 (s, 1H), 9.72 (s, 1H), 9.53 (s, 1H), 9.43 (s, 1H), 7.45-7.37 (m, 1H), 7.42 (t, J=54 Hz, 1H), 7.24 (t, J=8.9 Hz, 1H), 3.14-3.04 (m, 2H), 1.82-1.69 (m, 2H), 0.98 (t, J=7.4 Hz, 3H). m/z (ES-MS) 463.0 [M+1].

### Example 43

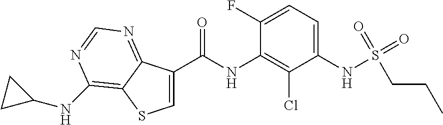

### 4-Cyclopropylamino-thieno[3,2-d]pyrimidine-7-carboxylic acid [2-chloro-6-fluoro-3-(propane-1-sulfonylamino)-phenyl]-amide

Step A: 4-Chloro-N-(2-chloro-6-fluoro-3-(N-(4-methoxybenzyl)propylsulfonamido)-phenyl)thieno[3,2-d]pyrimidine-7-carboxamide was prepared using a similar procedure described in Example 2, Step A, using N-(3-Amino-2-chloro-4-fluorophenyl)-N-(4-methoxybenzyl)propane-1-sulfonamide in place of N-(3-Amino-2,4-difluorophenyl)-N-(4-methoxybenzyl)ethane-sulfonamide.

Step B: 4-Chloro-N-(2-chloro-6-fluoro-3-(N-(4-methoxybenzyl)propylsulfonamido)-phenyl)thieno[3,2-d]pyrimidine-7-carboxamide (69.6 mg, 0.119 mmol) and cyclopropylamine (0.0827 mL, 1.193 mmol) were dissolved in 1,4-dioxane (0.5 mL, 6 mmol) and heated in a microwave reactor for 45 minutes at 120° C. The reaction mixture was concentrated to dryness to give N-(2-chloro-6-fluoro-3-(N-(4-methoxybenzyl)propylsulfonamido)-phenyl)-4-(cyclopropylamino)thieno[3,2-d]pyrimidine-7-carboxamide (72.1 mg, 0.119 mmol) which was dissolved in trifluoroacetic acid (1 mL, 10 mmol) and stirred at room temperature for 3 hours before being concentrated to dryness under reduced pressure. The resulting oil was redissovled in 1 mL dimethylformamide, filtered through a nylon disk and purified by reverse phase HPLC to give 4-cyclopropylamino-thieno[3,2-d]pyrimidine-7-carboxylic acid [2-chloro-6-fluoro-3-(propane-1-sulfonylamino)-phenyl]amide as a tan solid (27.9 mg, 48.3%). ¹H NMR (500 MHz, DMSO-d₆) δ 11.49 (s, 1H), 10.03-9.63 (m, 1H), 8.96 (s, 1H), 8.63 (s, 1H), 8.48 (s, 1H), 8.18 (s, OH), 7.45 (dd, J=9.1, 5.3 Hz, 1H), 7.32 (t, J=9.1 Hz, 1H), 3.06 (dd, J=8.6, 5.4 Hz, 3H), 1.76 (dq, J=14.9, 7.4 Hz, 2H), 0.98 (t, J=7.4 Hz, 3H), 0.85 (s, 2H), 0.71 (s, 2H). MS m/z=484.0 [M+1].

### Example 44

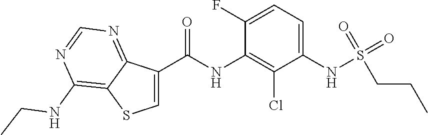

### 4-Ethylamino-thieno[3,2-d]pyrimidine-7-carboxylic acid [2-chloro-6-fluoro-3-(propane-1-sulfonylamino)-phenyl]-amide

The title compound was prepared using a similar procedure described in Example 43, using ethylamine instead of cyclopropylamine. ¹H NMR (500 MHz, DMSO-d₆) δ 11.49 (s, 1H), 9.59 (s, 2H), 8.92 (s, 1H), 8.62 (s, 1H), 8.37 (d, J=5.1 Hz, 1H), 7.45 (dd, J=9.2, 5.3 Hz, 1H), 7.34 (t, J=9.4 Hz, 1H), 3.59 (dt, J=14.3, 7.2 Hz, 2H), 3.14-3.01 (m, 2H), 1.76 (dq, J=15.3, 7.5 Hz, 2H), 1.24 (t, J=7.2 Hz, 3H), 0.98 (t, J=7.4 Hz, 3H). MS m/z=472.1 [M+1].

### Example 45

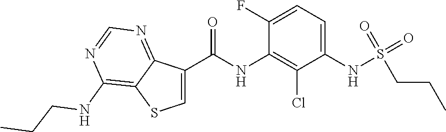

### 4-Propylamino-thieno[3,2-d]pyrimidine-7-carboxylic acid [2-chloro-6-fluoro-3-(propane-1-sulfonylamino)-phenyl]-amide

The title compound was prepared using a similar procedure described in Example 43, using propylamine instead of cyclopropylamine. ¹H NMR (500 MHz, DMSO-d₆) δ 11.51 (s, 1H), 9.57 (s, 1H), 8.92 (s, 1H), 8.61 (s, 1H), 8.38 (t, J=5.6 Hz, 1H), 7.46 (dd, J=9.1, 5.3 Hz, 1H), 7.36 (t, J=9.1 Hz, 1H), 3.52 (dd, J=13.5, 6.4 Hz, 2H), 3.18-3.05 (m, 2H), 1.86-1.73 (m, 2H), 1.73-1.59 (m, 2H), 0.99 (t, J=7.4 Hz, 3H), 0.94 (t, J=7.4 Hz, 3H). MS m/z=486.3 [M+1].

### Example 46

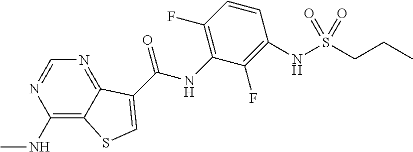

### 4-Methylamino-thieno[3,2-d]pyrimidine-7-carboxylic acid [2,6-difluoro-3-(propane-1-sulfonylamino)-phenyl]-amide

Step A: 4-Chloro-N-(2,6-difluoro-3-(N-(4-methoxybenzyl)propylsulfonamido)-phenyl)thieno[3,2-d]pyrimidine-7-carboxamide was prepared using a similar procedure described in for Example 2, Step A, using N-(3-amino-2,4-difluorophenyl)-N-(4-methoxy-benzyl)propane-1-sulfonamide in place of N-(3-Amino-2,4-difluorophenyl)-N-(4-methoxy-benzyl)ethane-sulfonamide.

Step B: 4-Chloro-N-(2,6-difluoro-3-(N-(4-methoxybenzyl)propylsulfonamido)-phenyl)thieno[3,2-d]pyrimidine-7-carboxamide (486.2 mg, 0.879 mmol) was dissolved in a solution of methylamine in ethanol (10 M, 4.0 mL). The reaction mixture was heated in a microwave reactor at 105° C. for 30 minutes. The mixture was concentrated under reduced pressure and loaded onto silica. The crude product was purified using flash chromatography (gradient elution: 0-100% (ethyl acetate+15% methanol) in heptanes) to yield N-(2,6-difluoro-3-(N-(4-methoxybenzyl)propylsulfonamido)phenyl)-4-(methylamino)thieno[3,2-d]pyrimidine-7-carboxamide as a foam (55 mg, 40%). ¹H NMR (500 MHz, DMSO-d₆) δ 11.33 (s, 1H), 8.93 (s, 1H), 8.63 (s, 1H), 8.40-8.30 (m, 1H), 7.33-7.22 (m, 1H), 7.21-7.11 (m, 3H), 6.85 (d, J=8.6 Hz, 2H), 4.71 (s, 2H), 3.71 (s, 3H), 3.28-3.23 (m, 2H), 3.04 (d, J=4.5 Hz, 3H), 1.85-1.73 (m, 2H), 1.01 (t, J=7.4 Hz, 3H).

Step C: N-(2,6-Difluoro-3-(N-(4-methoxybenzyl)propylsulfonamido)phenyl)-4-(methylamino)thieno[3,2-d]pyrimidine-7-carboxamide (55 mg, 0.098 mmol) was dissolved in a solution of hydrogen chloride in 1,4-dioxane (4 M, 8.0 mL). The reaction mixture was stirred at 45° C. for 3 days, and the resulting precipitate was filtered off. The solid was washed with ethyl acetate and dried in a vacuum oven overnight to yield 4-methylamino-thieno[3,2-d]pyrimidine-7-carboxylic acid [2,6-difluoro-3-(propane-1-sulfonylamino)-phenyl]-amide as the HCl salt (crystals, 25 mg, 53%). ¹H NMR (400 MHz, DMSO-d₆) δ 11.38-11.13 (m, 1H), 9.73 (s, 1H), 9.22-8.86 (m, 1H), 8.66 (s, 1H), 7.40 (td, J=8.9, 5.7 Hz, 1H), 7.24 (t, J=9.2 Hz, 1H), 3.15-2.99 (m, 5H), 1.83-1.67 (m, 2H), 0.98 (t, J=7.4 Hz, 3H). LC/MS: m/z 442.0 [M+1].

### Example 47

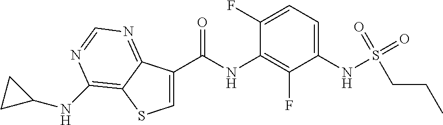

### 4-Cyclopropylamino-thieno[3,2-d]pyrimidine-7-carboxylic acid [2,6-difluoro-3-propane-1-sulfonylamino)-phenyl]-amide

4-Chloro-N-(2,6-difluoro-3-(propylsulfonamido)phenyl)thieno[3,2-d]pyrimidine-7-carboxamide (0.4 g, 0.9 mmol, as prepared in Example 40, Step A) and cyclopropylamine (0.186 mL, 2.68 mmol) were combined in 1,4-dioxane (0.5 mL) and heated in a microwave reactor at 120° C. for 20 minutes. The reaction mixture was concentrated under reduced pressure, redissolved in ethyl acetate and washed with saturated aqueous sodium bicarbonate. The organic layer was mixed with silica gel, concentrated to dryness and purified by silica gel chromatography (0-30% ethyl acetate:heptane) to give, after drying in a vacuum oven overnight, 4-cyclopropylamino-thieno[3,2-d]pyrimidine-7-carboxylic acid [2,6-difluoro-3-(propane-1-sulfonylamino)-phenyl]-amide (321 mg, 80%). ¹H NMR (400 MHz, DMSO-d₆) δ 11.39 (s, 1H), 9.67 (s, 1H), 8.97 (s, 1H), 8.63 (s, 1H), 8.51 (s, 1H), 7.38 (dd, J=14.0, 8.5 Hz, 1H), 7.22 (t, J=8.9 Hz, 1H), 3.18-2.96 (m, 3H), 1.76 (dd, J=14.9, 7.4 Hz, 2H), 0.98 (t, J=7.3 Hz, 3H), 0.85 (s, 2H), 0.71 (s, 2H). MS m/z=468.1 [M+1].

Examples 48-63 in Table 3 were prepared according to the procedure described in Example 47 using appropriate starting materials.

TABLE 3

| Ex- | | | | |
|---|---|---|---|---|
| ample | | | MS | ¹H NMR δ |
| no. | Structure | Name | m/z | (400 MHz, DMSO-d6) |
| 48 | 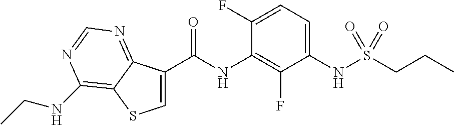 | 4-Ethylamino- thieno[3,2-d] pyrimidine-7- carboxylic acid [2,6-difluoro-3- (propane-1- sulfonylamino)- phenyl]-amide | 456.1 [M + 1] | 11.37 (s, 1H), 9.71 (s, 1H), 8.93 (s, 1H), 8.62 (s, 1H), 8.38 (t, J = 5.4 Hz, 1H), 7.38 (td, J = 8.9, 5.8 Hz, 1H), 7.22 (t, J = 9.1 Hz, 1H), 3.65-3.51 (m, 2H), 3.15- 3.02 (m, 2H), 1.82-1.66 (m, 2H), 1.33-1.13 (m, 3H), 0.98 (t, J = 7.4 Hz, 3H) |
| 49 | 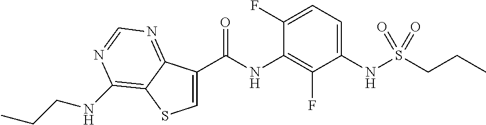 | 4-Propylamino- thieno[3,2-d] pyrimidine-7- carboxylic acid [2,6-difluoro-3- (propane-1- sulfonylamino)- phenyl]-amide | 470.1 [M + 1] | 11.33 (s, 1H), 10.33-9.63 (m, 1H), 8.92 (s, 1H), 8.61 (s, 1H), 8.39 (t, J = 5.5 Hz, 1H), 7.35 (dd, J = 14.6, 8.9 Hz, 1H), 7.16 (t, J = 9.1 Hz, 1H), 3.51 (dd, J = 13.4, 6.4 Hz, 2H), 3.10-2.95 (m, 2H), 1.74 (dt, J = 15.1, 7.5 Hz, 2H), 1.65 (dt, J = 14.6, 7.3 Hz, 2H), 0.95 (dt, J = 11.5, 7.4 Hz, 6H) |
| 50 | 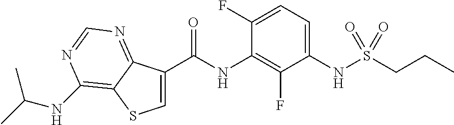 | 4-Isopropyl- amino-thieno [3,2-d] pyrimidine-7- carboxylic acid [2,6-difluoro-3- (propane-1- sulfonylamino)- phenyl]-amide | 470.1 [M + 1] | 11.38 (s, 1H), 9.71 (s, 1H), 8.93 (s, 1H), 8.61 (s, 1H), 8.19 (d, J = 7.5 Hz, 1H), 7.38 (td, J = 8.9, 5.8 Hz, 1H), 7.21 (t, J = 8.8 Hz, 1H), 4.51 (dq, J = 13.4, 6.6 Hz, 1H), 3.16-2.99 (m, 2H), 1.89-1.66 (m, 2H), 1.27 (d, J = 6.6 Hz, 6H), 0.98 (t, J = 7.4 Hz, 3H) |
| 51 | 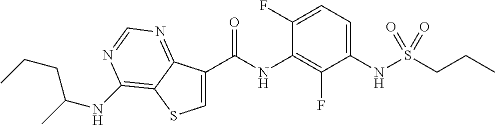 | 4-(1-Methyl- butylamino)- thieno[3,2-d] pyrimidine-7- carboxylic acid [2,6-difluoro- 3-(propane-1- sulfonylamino)- phenyl]-amide | 498.1 [M + 1] | 11.39 (s, 1H), 9.69 (s, 1H), 8.92 (s, 1H), 8.59 (s, 1H), 8.12 (d, J = 8.1 Hz, 1H), 7.38 (td, J = 8.8, 5.8 Hz, 1H), 7.22 (t, J = 9.2 Hz, 1H), 4.55-4.39 (m, 1H), 3.16-3.00 (m, 2H), 1.83-1.70 (m, 2H), 1.65 (dt, J = 13.7, 8.5 Hz, 1H), 1.58-1.46 (m, 1H), 1.36 (dt, J = 13.1, 6.8 Hz, 2H), 1.23 (d, J = 6.6 Hz, 3H), 0.98 (t, J = 7.4 Hz, 3H), 0.89 (t, J = 7.3 Hz, 3H) |
| 52 | 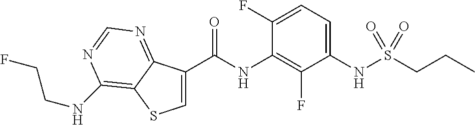 | 4-(2-Fluoro- ethylamino)- thieno[3,2-d] pyrimidine-7- carboxylic acid [2,6-difluoro- 3-(propane-1- sulfonylamino)- phenyl]-amide | 474.0 [M + 1] | 11.28 (s, 1H), 8.96 (s, 1H), 8.64 (m, J = 5.6 Hz, 2H), 7.35 (m, 1H), 7.25-7.11 (m, 1H), 4.71 (m, J = 5.1 Hz, 1H), 4.60 (m, J = 5.1 Hz, 1H), 3.97-3.80 (m, 2H), 3.02 (m, 2H), 1.74 (m, J = 7.9, 2H), 0.97 (t, J = 7.4 Hz, 3H) |
| 53 | 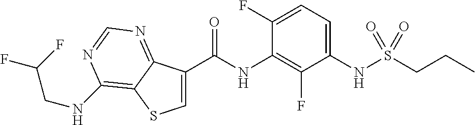 | 4-(2,2-Difluoro- ethylamino)- thieno[3,2-d] pyrimidine-7- carboxylic acid [2,6-difluoro- 3-(propane-1- sulfonylamino)- phenyl]-amide | 492.1 [M + 1] | 11.25 (s, 1H), 9.76 (s, 1H), 9.00 (s, 1H), 8.79 (t, J = 5.7 Hz, 1H), 8.70 (s, 1H), 7.47-7.29 (m, 1H), 7.20 (t, J = 9.4 Hz, 1H), 6.42- 6.09 (m, 1H), 4.00 (ddd, J = 16.4, 10.3, 4.9 Hz, 2H), 3.11- 2.99 (m, 2H), 1.85-1.63 (m, 2H), 0.98 (t, J = 7.4 Hz, 3H) |
| 54 | 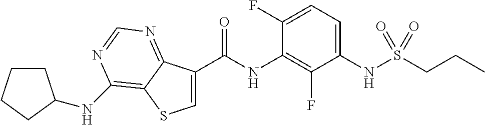 | 4-Cyclopentyl- amino-thieno [3,2-d] pyrimidine-7- carboxylic acid [2,6-difluoro- 3-(propane-1- sulfonylamino)- phenyl]-amide | 496.1 [M + 1] | 11.38 (s, 1H), 9.75 (s, 1H), 8.93 (s, 1H), 8.61 (s, 1H), 8.28 (d, J = 6.9 Hz, 1H), 7.37 (td, J = 8.8, 5.7 Hz, 1H), 7.21 (t, J = 9.1 Hz, 1H), 4.58 (dd, J = 13.8, 6.9 Hz, 1H), 3.14-3.00 (m, 2H), 2.04 (dd, J = 18.0, 6.0 Hz, 2H), 1.85- 1.50 (m, 8H), 0.98 (t, J = 7.4 Hz, 3H) |
| 55 | 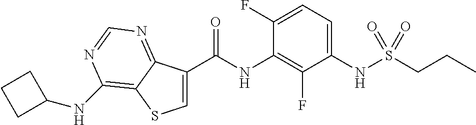 | 4-Cyclobutyl- amino-thieno [3,2-d] pyrimidine-7- carboxylic acid [2,6-difluoro- 3-(propane-1- sulfonylamino)- phenyl]-amide | 482.1 [M + 1] | 11.33 (s, 1H), 9.80 (s, 1H), 8.94 (s, 1H), 8.66-8.50 (m, 2H), 7.37 (dd, J = 14.5, 8.8 Hz, 1H), 7.19 (t, J = 9.1 Hz, 1H), 4.72 (dd, J = 15.5, 7.9 Hz, 1H), 3.10-2.99 (m, 2H), 2.33 (t, J = 8.1 Hz, 2H), 2.23-2.09 (m, 2H), 1.74 (dt, J = 17.8, 7.4 Hz, 4H), 0.97 (t, J = 7.4 Hz, 3H) |
| 56 | 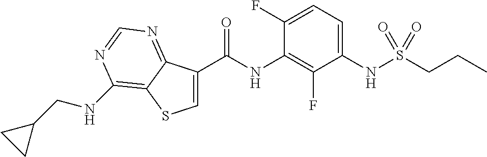 | 4-(Cyclopropyl- methylamino)- thieno[3,2-d] pyrimidine-7- carboxylic acid [2,6-difluoro- 3-(propane-1- sulfonylamino)- phenyl]-amide | 482.1 [M + 1] | 11.36 (s, 1H), 9.76 (s, 1H), 8.94 (s, 1H), 8.61 (s, 1H), 8.53 (t, J = 5.5 Hz, 1H), 7.37 (td, J = 8.9, 5.7 Hz, 1H), 7.20 (t, J = 8.7 Hz, 1H), 3.44 (t, J = 6.2 Hz, 2H), 3.14-2.97 (m, 2H), 1.83- 1.62 (m, 2H), 1.29-1.07 (m, 1H), 0.98 (t, J = 7.4 Hz, 3H), 0.57- 0.45 (m, 2H), 0.36-0.25 (m, 2H) |
| 57 | 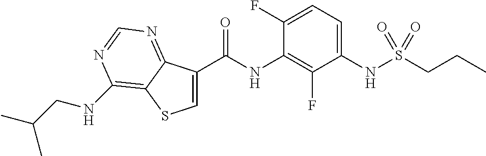 | 4-Isobutylamino- thieno[3,2-d] pyrimidine-7- carboxylic acid [2,6-difluoro- 3-(propane-1- sulfonylamino)- phenyl]-amide | 484.1 [M + 1] | 11.36 (s, 1H), 9.78 (s, 1H), 8.93 (s, 1H), 8.60 (s, 1H), 8.43 (t, J = 5.6 Hz, 1H), 7.37 (td, J = 8.9, 5.7 Hz, 1H), 7.20 (t, J = 9.1 Hz, 1H), 3.44-3.33 (m, 2H), 3.14-3.00 (m, 2H), 2.15-1.91 (m, 1H), 1.83-1.66 (m, 2H), 1.02-0.90 (m, 9H) |
| 58 | 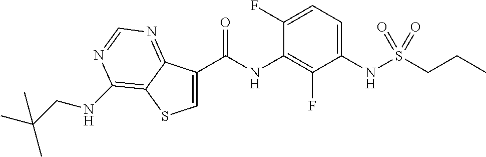 | 4-(2,2-Dimethyl- propylamino)- thieno[3,2-d] pyrimidine-7- carboxylic acid [2,6-difluoro- 3-(propane-1- sulfonylamino)- phenyl]-amide | 498.1 [M + 1] | 11.38 (s, 1H), 9.69 (s, 1H), 8.94 (s, 1H), 8.59 (s, 1H), 8.32 (t, J = 6.3 Hz, 1H), 7.38 (td, J = 8.7, 5.7 Hz, 1H), 7.22 (t, J = 9.3 Hz, 1H), 3.48 (d, J = 6.3 Hz, 2H), 3.14-3.03 (m, 2H), 1.85- 1.63 (m, 2H), 1.06-0.86 (m, 12H) |
| 59 | 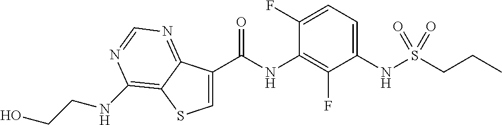 | 4-(2-Hydroxy- ethylamino)- thieno[3,2-d] pyrimidine-7- carboxylic acid [2,6-difluoro- 3-(propane-1- sulfonylamino)- phenyl]-amide | 472.1 [M + 1] | 11.33 (s, 1H), 10.26-9.60 (m, 1H), 8.93 (s, 1H), 8.61 (s, 1H), 8.41 (s, 1H), 7.47-7.27 (m, 1H), 7.17 (t, J = 8.6 Hz, 1H), 4.80 (s, 1H), 3.63 (s, 4H), 3.10- 2.96 (m, 2H), 1.81-1.65 (m, 2H), 0.97 (t, J = 7.4 Hz, 3H) |
| 60 | 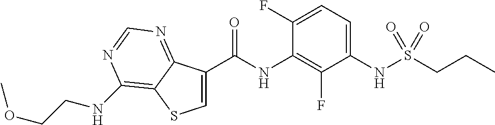 | 4-(2-Methoxy- ethylamino)- thieno[3,2-d] pyrimidine-7- carboxylic acid [2,6-difluoro- 3-(propane-1- sulfonylamino)- phenyl]-amide | 486.1 [M + 1] | 11.35 (s, 1H), 9.70 (s, 1H), 8.94 (s, 1H), 8.62 (s, 1H), 8.50 (t, J = 5.4 Hz, 1H), 7.38 (td, J = 8.8, 5.8 Hz, 1H), 7.22 (t, J = 8.8 Hz, 1H), 3.73 (q, J = 5.6 Hz, 2H), 3.58 (t, J = 5.7 Hz, 2H), 3.30 (s, 3H), 3.09 (dd, J = 14.6, 7.0 Hz, 2H), 1.81-1.67 (m, 2H), 0.98 (t, J = 7.4 Hz, 3H) |
| 61 | 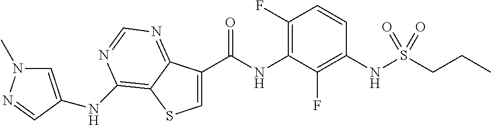 | 4-(1-Methyl- 1H-pyrazol- 4-ylamino)- thieno[3,2-d] pyrimidine-7- carboxylic acid [2,6-difluoro- 3-(propane-1- sulfonylamino)- phenyl]-amide | 506.0 [M − 1] | 11.31 (s, 1H), 10.30-10.50 (bs, 1H), 9.73 (s, 1H), 8.97-8.05 (bs, 1H), 8.75 (s, 1H), 8.15-8.21 (bs, 1H), 7.69 (s, 1H), 7.35-7.44 (m, 1H), 7.20-7.27 (m, 1H), 3.89 (s, 3H), 3.06-3.13 (m, 2H), 1.70- 1.81 (m, 2H), 0.95-1.01 (m, 3H) |
| 62 | 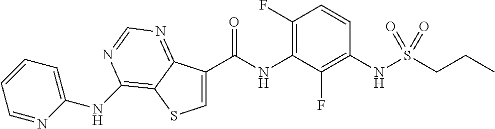 | 4-(Pyridin-2- ylamino)- thieno[3,2-d] pyrimidine-7- carboxylic acid [2,6-difluoro- 3-(propane-1- sulfonylamino)- phenyl]-amide | 503.2 [M − 1] | *** 11.39-11.41 (bs, 1H), 8.82- 8.89 (m, 2H), 8.30-8.40 (bs, 1H), 7.78-7.84 (m, 1H), 7.50-7.57 (m, 1H), 7.08-7.15 (m, 1H), 7.01- 7.08 (m, 1H), 6.60-6.75 (bs, 1H), 3.06-3.13 (m, 2H), 1.85-1.96 (m, 2H), 1.02-1.10 (m, 3H); |
| 63 | 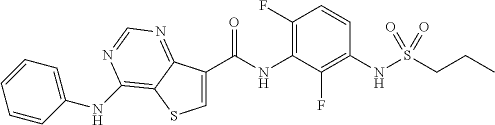 | 4-Phenylamino- thieno[3,2- d]pyrimidine-7- carboxylic acid [2,6-difluoro-3- (propane-1- sulfonylamino)- phenyl]-amide | 504.2 [M − 1] | *** 8.95 (s, 1H), 8.68 (s, 1H), 6.69-7.72 (m, 1H), 7.46-7.52 (m, 1H), 7.40-7.45 (m, 2H), 7.21- 7.26 (m, 1H), 7.09-7.15 (m, 1H), 3.07-3.14(m, 2H), 1.82-1.91 (m, 2H), 1.03-1.08 (m, 3H) |

The amine reagent was used as free base and 3 eq. DIPEA were used in the reaction
** The amine reagent was used as hydrochloride salt and 6 eq. DIPEA were used in the reaction
*** CDCl₃ was used as NMR solvent instead of DMSO-d6

### Example 64

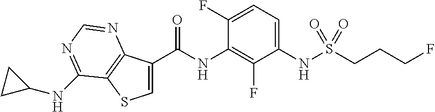

### 4-Cyclopropylamino-thieno[3,2-d]pyrimidine-7-carboxylic acid [2,6-difluoro-3-(3-fluoro-propane-1-sulfonylamino)-phenyl]amide

Step A: 4-Chloro-N-(2,6-difluoro-3-(3-fluoropropylsulfonamido)phenyl)thieno[3,2-d]-pyrimidine-7-carboxamide was prepared according to the general procedure in Example 13, Step A, using N-(3-amino-2,4-difluorophenyl)-3-fluoropropane-1-sulfonamide in place of N-(3-amino-4-chloro-2-fluorophenyl)-3-fluoropropane-1-sulfonamide.

Step B: The title compound was prepared according to the general procedure in Example 47, using 4-chloro-N-(2,6-difluoro-3-(3-fluoropropylsulfonamido)phenyl)thieno-[3,2-d]pyrimidine-7-carboxamide in place of 4-chloro-N-(2,6-difluoro-3-(propylsulfonamido)phenyl)thieno[3,2-d]pyrimidine-7-carboxamide. ¹H NMR (400 MHz, DMSO-d₆) δ 11.39 (s, 1H), 9.67 (s, 1H), 8.97 (s, 1H), 8.63 (s, 1H), 8.51 (s, 1H), 7.38 (dd, J=14.0, 8.5 Hz, 1H), 7.22 (t, J=8.9 Hz, 1H), 3.18-2.96 (m, 3H), 1.76 (dd, J=14.9, 7.4 Hz, 2H), 0.98 (t, J=7.3 Hz, 3H), 0.85 (s, 2H), 0.71 (s, 2H). MS [M+1]m/z 486.2.

### Example 65

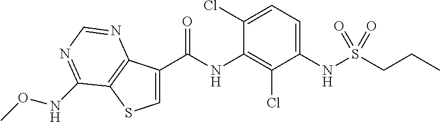

### 4-Methoxyamino-thieno[3,2-d]pyrimidine-7-carboxylic acid [2,6-dichloro-3-(propane-1-sulfonylamino)-phenyl]amide

4-Chloro-N-(2,6-dichloro-3-(propylsulfonamido)phenyl)thieno[3,2-d]pyrimidine-7-carboxamide (27 mg, 0.06 mmol, prepared as in Example 9, Step A) and methoxylamine hydrochloride (70 mg, 0.84 mmol) were taken up in isopropanol (1 mL). The addition of N,N-diisopropylethylamine (146 uL, 0.84 mmol) was followed by stirring at 60° C. for 1 hour. The reaction mixture was concentrated in vacuo, and the crude product was purified through preparative HPLC to obtain 4-methoxy-amino-thieno[3,2-d]pyrimidine-7-carboxylic acid [2,6-dichloro-3-(propane-1-sulfonylamino)-phenyl]amide (11 mg, 40%). ¹H NMR (400 MHz, DMSO-d₆) δ 11.74 (s, 1H), 10.97 (s, 1H), 9.66 (s, 1H), 8.54 (s, 1H), 7.89 (s, 1H), 7.56 (d, J=9.0 Hz, 1H), 7.48 (d, J=8.9 Hz, 1H), 3.82 (s, 3H), 3.18-3.07 (m, 2H), 1.85-1.69 (m, 2H), 0.98 (t, J=7.4 Hz, 3H). LC-MS [M+1]m/z 490.0.

### Example 66

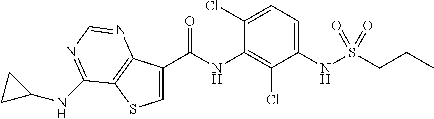

### 4-Cyclopropylamino-thieno[3,2-d]pyrimidine-7-carboxylic acid [2,6-dichloro-3-(propane-1-sulfonylamino)-phenyl]-amide

To a mixture of 4-chloro-N-(2,6-dichloro-3-(propylsulfonamido)phenyl)-thieno[3,2-d]pyrimidine-7-carboxamide (33.1 mg, 0.07 mmol, prepared as in Example 9, Step A) and cyclopropylamine (72 uL, 1.0 mmol) in isopropanol (1 mL) was added N,N-diisopropylethylamine (120 uL, 0.74 mmol). The reaction mixture was stirred at 60° C. for 1 hour. The reaction mixture was concentrated in vacuo and the crude product purified through preparative HPLC to obtain 4-cyclopropylamino-thieno[3,2-d]pyrimidine-7-carboxylic acid [2,6-dichloro-3-(propane-1-sulfonylamino)-phenyl]-amide (18 mg, 52%). ¹H NMR (400 MHz, DMSO-d₆) δ 11.60 (s, 1H), 9.66 (s, 1H), 8.97 (s, 1H), 8.63 (s, 1H), 8.50 (s, 1H), 7.59 (d, J=8.9 Hz, 1H), 7.49 (d, J=8.9 Hz, 1H), 3.20-3.11 (m, 2H), 3.05 (dt, J=10.4, 3.4 Hz, 1H), 1.85-1.69 (m, 2H), 0.98 (t, J=7.4 Hz, 3H), 0.86 (br d, J=5.2 Hz, 2H), 0.70 (br s, 2H). LC-MS [M+1]m/z 500.0.

Examples 67-98 in Table 4 were prepared according to the procedure described in Example 66 using appropriate starting materials.

TABLE 4

| Ex- | | | | |
|---|---|---|---|---|
| ample | | | MS | ¹H NMR δ |
| no. | Structure | Name | m/z | (400 MHz, DMSO-d6) |
| 67 | 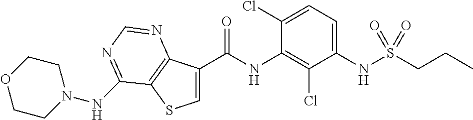 | 4-(Morpholin- 4-ylamino)- thieno[3,2-d] pyrimidine-7- carboxylic acid [2,6-dichloro- 3-(propane-1- sulfonyl- amino)- phenyl]-amide | 545.1 [M + 1] | 11.53 (s, 1H), 9.68 (s, 1H), 8.95 (s, 1H), 8.54 (s, 1H), 7.80 (m, 1H), 7.45 (d, J = 8.7, 2H), 3.89 (d, J = 10.5, 2H), 3.72 (t, J = 10.7, 2H), 3.12-2.96 (m, 4H), 2.87 (t, J = 9.8, 2H), 1.74 (dd, J = 15.0, 7.6, 2H), 0.97 (t, J = 7.5, 3H) |
| 68 | 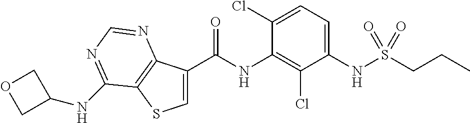 | 4-(Oxetan-3- ylamino)- thieno[3,2-d] pyrimidine-7- carboxylic acid [2,6-dichloro- 3-(propane-1- sulfonyl- amino)- phenyl]-amide | 516.0 [M + 1] | 11.48 (s, 1H), 9.66 (s, 1H), 9.02 (d, J = 5.4 Hz, 1H), 8.98 (s, 1H), 8.62 (s, 1H), 7.56 (d, J = 8.8 Hz, 1H), 7.48 (d, J = 8.9 Hz, 1H), 5.28- 5.17 (m, 1H), 4.88 (t, J = 6.9 Hz, 2H), 4.66 (t, J = 6.3 Hz, 2H), 3.15- 3.05 (m, 2H), 1.82-1.69 (m, 2H), 0.97 (t, J = 7.4 Hz, 3H) |
| 69 | 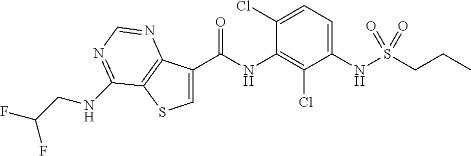 | 4-(2,2- Difluoro- ethylamino)- thieno[3,2-d] pyrimidine-7- carboxylic acid [2,6-dichloro- 3-(propane-1- sulfonyl- amino)- phenyl]-amide | 524.0 [M + 1] | 11.49 (s, 1H), 9.67 (s, 1H), 8.99 (s, 1H), 8.79 (t, J = 5.8 Hz, 1H), 8.69 (s, 1H), 7.59 (d, J = 8.9 Hz, 1H), 7.50 (d, J = 8.9 Hz, 1H), 6.26 (tt, J = 56.0, 3.9 Hz, 1H), 4.08-3.87 (m, 2H), 3.20-3.07 (m, 2H), 1.84- 1.67 (m, 2H), 0.98 (s, J = 7.4 Hz, 3H) |
| 70 | 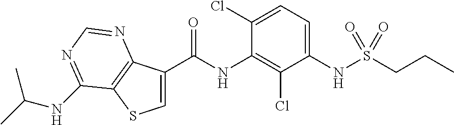 | 4-Isopropyl- amino-thieno [3,2-d] pyrimidine-7- carboxylic acid [2 6-dichloro- 3-(propane-1- sulfonyl- amino)- phenyl]-amide | 502.0 [M + 1] | 11.60 (s, 1H), 9.66 (s, 1H), 8.92 (s, 1H), 8.60 s, 1H), 8.18 (d, J = 7.6 Hz, 1H), 7.59 (d, J = 8.9 Hz, 1H), 7.49 (d, J = 8.9 Hz, 1H), 4.51 (dq, J = 13.3, 6.6 Hz, 1H), 3.22-3.05 (m, 2H), 1.85-1.66 (m, 2H), 1.27 (d, J = 6.6 Hz, 6H), 0.98 (s, 3H) |
| 71 | 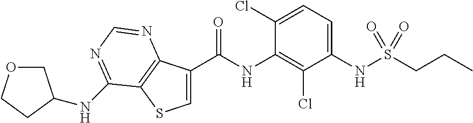 | 4-(Tetrahydro- furan-3- ylamino)- thieno[3,2-d] pyrimidine-7- carboxylic acid [2,6-dichloro- 3-(propane-1- sulfonyl- amino) phenyl]-amid | 530.1 [M + 1] | 11.54 (s, 1H), 10.01-9.17 (m, 1H), 8.94 (s, 1H), 8.64 (s, 1H), 8.51 (d, J = 6.2 Hz, 1H), 7.56 (d, J = 8.8 Hz, 1H), 7.48 (d, J = 8.9 Hz, 1H), 4.78 (d, J = 3.7 Hz, 1H), 4.00-3.82 (m, 2H), 3.82-3.63 (m, 3H), 3.13 (dd, J = 17.6, 10.0 Hz, 2H), 2.37-2.21 (m, 1H), 2.03 (td, J = 12.4, 6.6 Hz, 1H), 1.86-1.63 (m, 2H), 0.98 (s, 3H) |
| 72 | 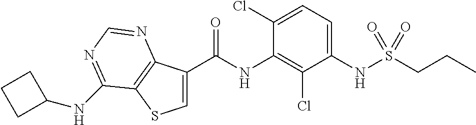 | 4-Cyclobutyl- amino-thieno [3,2-d] pyrimidine-7- carboxylic acid [2,6-dichloro- 3-(propane-1- sulfonyl- amino)- phenyl]-amide | 514.1 [M + 1] | 11.57 (s, 1H), 9.66 (s, 1H), 8.93 (s, 1H), 8.60 (s, 1H), 8.56 (d, J = 7.0 Hz, 1H), 7.59 (d, J = 8.8 Hz, 1H), 7.49 (d, J = 8.9 Hz, 1H), 4.78-4.65 (m, 1H), 3.17-3.11 (m, 2H), 2.39-2.29 (m, 2H), 2.21-2.06 (m, 2H), 1.84-1.67 (m, 4H), 0.98 (t, J = 7.4 Hz, 3H). |
| 73 | 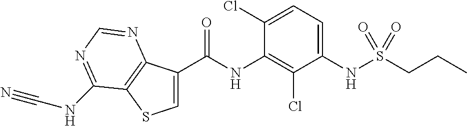 | 4-Cyano- amino- thieno[3,2-d] pyrimidine-7- carboxylic acid [2,6-dichloro- 3-(propane-1- sulfonyl- amino)- phenyl]amide | 485.0 [M + 1] | 10.96 (s, 1H), 9.69 (s, 1H), 9.05 (s, 1H), 8.51 (s, 1H), 7.61 (d, J = 8.8 Hz, 1H), 7.52 (d, J = 8.8 Hz, 1H), 3.20- 3.10 (m, 3H), 1.82-1.70 (m, 2H), 0.98 (t, J = 7.4, 3H) |
| 74 | 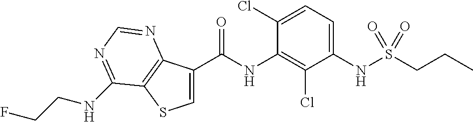 | 4-(2-Fluoro- ethylamino)- thieno[3,2-d] pyrimidine-7- carboxylic acid [2,6-dichloro- 3-(propane-1- sulfonyl- amino)- phenyl]amide | 506.0 [M + 1] | 11.53 (s, 1H), 9.66 (s, 1H), 8.96 (s, 1H), 8.64 (s, 2H), 7.58 (d, J = 8.9 Hz, 1H), 7.49 (d, J = 8.9 Hz, 1H), 4.71 (d, J = 5.0 Hz, 1H), 4.60 (d, J = 5.0 Hz, 1H), 3.88 (dd, J = 26.4, 5.2 Hz, 2H), 3.20-3.04 (m, 2H), 1.83-1.65 (m, 2H), 0.98 (t, J = 7.4 Hz, 3H) |
| 75 | 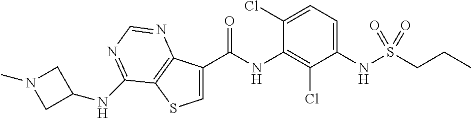 | 4-(1-Methyl- azetidin-3- ylamino)- thieno[3,2-d] pyrimidine-7- carboxylic acid [2,6-dichloro- 3-(propane-1- sulfonyl- amino)- phenyl]amide | 529.1 [M + 1] | 10.99 (s, 1H), 8.73 (s, 1H), 8.37 (s, 1H), 8.23 (s, 1H), 7.37 (d, J = 9.0 Hz, 1H), 7.28 (d, J = 9.0 Hz, 1H), 4.43 (d, J = 7.3 Hz , 2H), 3.99 (d, J = 7.3 Hz, 2H), 2.93 (dt, J = 10.4, 3.4 Hz, 1H), 2.86 (s, 3H), 2.47 (m, 2H), 1.75- 1.62 (m, 2H), 0.93 (t, J = 7.4 Hz, 3H) |
| 76 | 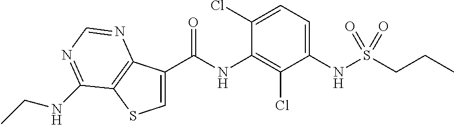 | 4-Ethylamino- thieno[3,2-d] pyrimidine-7- carboxylic acid [2,6-dichloro- 3-(propane-1- sulfonyl- amino)- phenyl]-amide | 488.0 [M + 1] | 11.44 (s, 1H), 8.94 (s, 1H), 8.62 (s, 1H), 8.39 (t, J = 5.4 Hz, 1H), 8.16 (s, 1H), 7.54-7.30 (m, 1H), 6.49 (s, 1H), 3.76-3.46 (m, 2H), 3.02 (s, 2H), 1.70 (dt, J = 15.1, 7.6 Hz, 2H), 1.24 (s, 3H), 0.96 (s, 3H) |
| 77 | 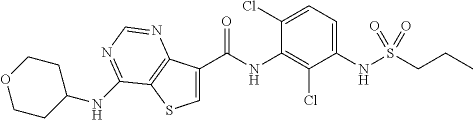 | 4-(Tetrahydro- pyran-4- ylamino)- thieno[3,2-d] pyrimidine-7- carboxylic acid [2,6-dichloro- 3-(propane-1- sulfonyl- amino)- phenyl]-amid | 544.0 [M + 1] | 11.45 (s, 1H), 8.92 (s, 1H), 8.62 (s, 1H), 8.26 (d, J = 7.2 Hz, 1H), 7.42 (s, 2H), 6.54 (s, 1H), 4.40 (s, 1H), 3.93 (d, J = 9.4 Hz, 2H), 3.44 (s, 4H), 2.98 (s, 2H), 1.91 (d, J = 13.4 Hz, 2H), 1.80-1.57 (m, 5H), 0.95 (t, J = 7.4 Hz, 3H) |
| 78 | 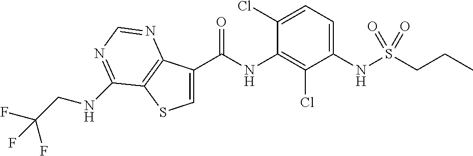 | 4-(2,2,2- Trifluoro- ethylamino)- thieno[3,2-d] pyrimidine-7- carboxylic acid [2,6-dichloro- 3-(propane-1- sulfonyl- amino)- phenyl]-amide | 542.0 [M + 1] | 11.42 (s, 1H), 9.72 (s, 1H), 9.03 (s, 2H), 8.74 (s, 1H), 7.55 (d, J = 8.8 Hz, 1H), 7.48 (d, J = 8.8 Hz, 1H), 4.57- 4.34 (m, 2H), 3.11 (m, 2H), 1.75 (m, 2H), 0.97 (t, J = 7.3 Hz, 3H) |
| 79 | 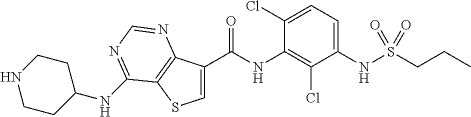 | 4-(Piperidin- 4-ylamino)- thieno[3,2-d] pyrimidine-7- carboxylic acid [2,6-dichloro- 3-(propane-1- sulfonyl- amino)- phenyl]amide | 543.0 [M + 1] | 11.47 (s, 1H), 9.04 (s, 1H), 8.69 (s, 1H), 8.24 (s, 1H), 7.37 (d, J = 8.9 Hz, 1H), 7.24 (d, J = 8.9 Hz, 1H), 6.61 (s, 1H), 4.71 (d, J = 13.9 Hz, 2H), 2.88- 2.76 (m, 4H), 2.05 (d, J = 11.2 Hz, 2H), 1.67 (dt, J = 14.4, 7.2 Hz, 2H), 1.59-1.42 (m, 2H), 0.93 (t, J = 7.4 Hz, 3H) |
| 80 | 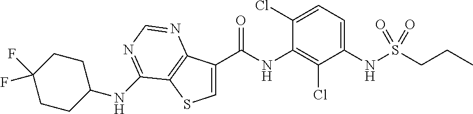 | 4-(4,4- Difluoro- cyclohexyl- amino)- thieno[3,2-d] pyrimidine-7- carboxylic acid [2,6-dichloro- 3-(propane-1- sulfonyl- amino)- phenyl]-amide | 578.0 [M + 1] | 11.54 (s, 1H), 9.71 (s, 1H), 8.94 (s, 1H), 8.64 (s, 1H), 8.25 (d, J = 7.7 Hz, 1H), 7.56 (d, J = 9.1 Hz, 1H), 7.48 (d, J = 8.9 Hz, 1H), 4.39 (s, 1H), 3.23- 2.95 (m, 2H), 2.00 (m, 4H), 1.86- 1.58 (m, 2H), 0.97 (t, J = 7.4 Hz, 3H) |
| 81 | 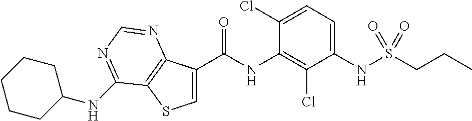 | 4-Cyclohexyl- amino-thieno [3,2-d] pyrimidine-7- carboxylic acid [2,6-dichloro- 3-(propane-1- sulfonyl- amino)- phenyl]-amide | 542.0 [M + 1] | 11.56 (s, 1H), 9.65 (s,1H), 8.91 (s, 1H), 8.59 (s, 1H), 8.19 (d, J = 7.7 Hz, 1H), 7.53 (d, J = 8.4 Hz, 1H), 7.46 (d, J = 8.6 Hz, 1H), 4.17 (s, 1H), 3.15- 3.00 (m, 2H), 1.96 (s, 2H), 1.84- 1.71 (m, 4H), 1.72-1.60 (m, 1H), 1.40 (m, 4H), 1.18 (s, 1H), 0.97 (t, J = 7.4 Hz, 3H) |
| 82 | 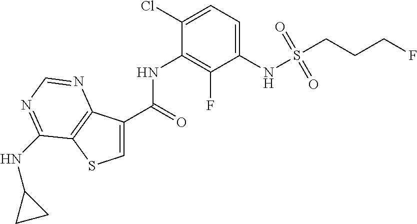 | 4-Cyclopropyl- amino-thieno [3,2-d] pyrimidine-7- carboxylic acid [2,6-dichloro- 3-(3-fluoro- propane-1- sulfonyl- amino)- phenyl]-amide | 518.0 [M + 1] | 11.62 (s, 1H), 9.89 (s, 1H), 8.97 (s, 1H), 8.63 (s, 1H), 8.55 (s, 1H), 7.62 (d, J = 8.9 Hz, 1H), 7.54-7.46 (m, 1H), 4.61 (t, J = 5.9 Hz, 1H), 4.49 (t, J = 6.0 Hz, 1H), 3.31-3.22 (m, 2H), 3.10-2.99 (m, 1H), 2.21-2.04 (m, 2H), 0.92-0.78 (m, 2H), 0.77-0.64 (m, 2H) |
| 83 | 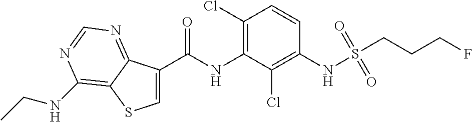 | 4-Ethylamino- thieno[3,2-d] pyrimidine-7- carboxylic acid [2,6-dichloro- 3-(3-fluoro- propane-1- sulfonyl- amino)- phenyl]-amide | 506.0 [M + 1] | 11.59 (s, 1H), 9.89 (s, 1H), 8.93 (s, 1H), 8.62 (s, 1H), 8.42 (t, J = 5.4 Hz, 1H), 7.59 (d, J = 8.8 Hz, 1H), 7.49 (d, J = 8.9 Hz, 1H), 4.61 (t, J = 5.9 Hz, 1H), 4.49 (t, J = 6.0 Hz, 1H), 3.63- 3.52 (m, 2H), 3.29-3.20 (m, 2H), 2.20-2.04 (m, 2H), 1.23 (t, J = 7.2 Hz, 3H) |
| 84 | 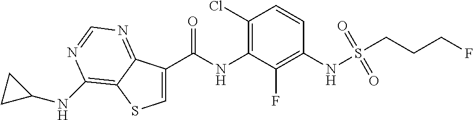 | 4-Cyclopropyl- amino-thieno [3,2-d] pyrimidine-7- carboxylic acid [6-chloro-2- fluoro-3-(3- fluoropropane- 1- sulfonyl- amino)- phenyl]-amide | 502.2 [M + 1] | 1.50 (s, 1H), 10.21 (br s, 1H), 8.98 (s, 1H), 8.64 (s, 1H), 8.55 (s, 1H), 7.45- 7.34 (m, 2H) 4.60 (t, J = 6.0 Hz, 1H), 4.48 (t, J = 6.0 Hz, 1H), 3.26- 3.14 (m, 2H), 3.09-3.01 (m, 1H), 2.18-2.00 (m, 2H), 0.92-0.80 (m, 2H), 0.77-0.63 (m, 2H) |
| 85 | 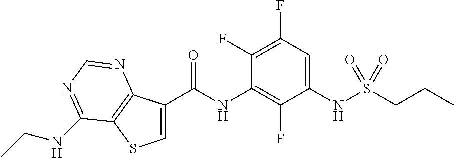 | 4-Ethylamino- thieno[3,2-d] pyrimidine-7- carboxylic acid [2,3,6- trifluoro- 5-(propane- 1-sulfonyl- amino)- phenyl]-amide | 474.1 [M + 1] | 1.44 (s, 1H), 8.94 (s, 1H), 8.62 (s, 1H), 8.39 (t, J = 5.4 Hz, 1H), 7.53- 7.29 (m, 1H), 6.49 (s, 1H), 3.58 (dt, J = 14.2, 7.1 Hz, 2H), 3.03 (s, 2H) 1.70 (dt, J = 14.8, 7.4 Hz, 2H), 1.24 (t, J = 7.2 Hz, 3H), 0.96 (t, J = 7.4 Hz, 3H) |
| 86 | 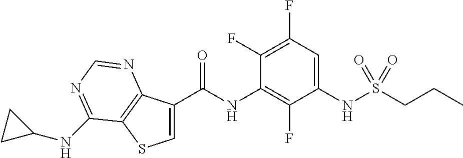 | 4-Cyclopropyl- amino-thieno [3,2-d] pyrimidine-7- carboxylic acid [2,3,6-trifluoro- 5-(propane-1- sulfonyl- amino)- phenyl]-amide | 486.1 [M + 1] | 11.53 (s, 1H), 9.94 (s, 1H), 9.00 (s, 1H), 8.64 (s, 1H), 8.52 (s, 1H), 7.46 (dt, J = 11.6, 7.7 Hz, 1H), 3.20-3.11 (m, 2H), 3.05 (dt, J = 10.4, 3.4 Hz, 1H), 1.81-1.68 (m, 2H), 0.98 (t, J = 7.4 Hz, 3H), 0.86 (d, J = 5.7 Hz, 2H), 0.70 (s, 2H) |
| 87 | 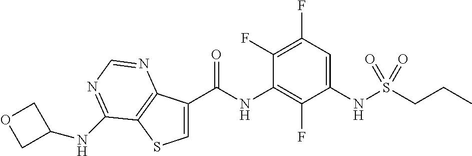 | 4-(Oxetan-3- ylamino)- thieno[3,2-d] pyrimidine-7- carboxylic acid [2,3,6-trifluoro- 5-(propane-1- sulfonyl- amino)- phenyl]amid | 502.1 [M + 1] | 11.38 (s, 1H), 10.59-9.68 (m, 1H), 9.01 (s, 2H), 8.63 (s, 1H), 7.42 (dd, J = 19.7, 7.8 Hz, 1H), 5.23 (dt, J = 13.2, 6.6 Hz, 1H), 4.88 (t, J = 6.9 Hz, 2H), 4.66 (t, J = 6.3 Hz, 2H), 3.16- 2.92 (m, 2H), 1.83-1.52 (m, 2H), 0.97 (s, 3H) |
| 88 | 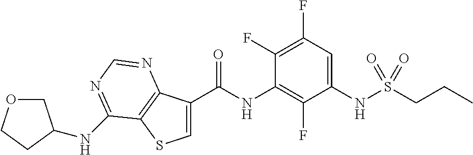 | 4-(Tetrahydro- furan-3- ylamino)- thieno[3,2-d] pyrimidine-7- carboxylic acid [2,3,6- trifluoro-5- (propane-1- sulfonyl- amino)- phenyl]-amide | 516.1 [M + 1] | 11.46 (s, 1H), 10.24 (s, 1H), 8.98 (s, 1H), 8.65 (s, 1H), 8.56 (d, J = 6.2 Hz, 1H), 7.43 (dt, J = 12.1, 8.1 Hz, 1H), 4.78 (s, 1H), 4.03-3.79 (m, 2H), 3.73 (ddd, J = 13.0, 11.6, 6.0 Hz, 2H), 3.08 (s, 2H), 2.27 (dt, J = 14.7, 7.7 Hz, 1H), 2.03 (dt, J = 12.3, 6.4 Hz, 1H), 1.72 (d, J = 7.6Hz, 2H), 0.97 (t, J = 7.4 Hz, 3H) |
| 89 | 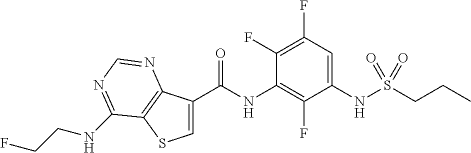 | 4-(2-Fluoro- ethylamino)- thieno[3,2-d] pyrimidine-7- carboxylic acid [2,3,6- trifluoro-5- (propane-1- sulfonyl- amino)e- phenyl]-amid | 492.1 [M + 1] | 11.43 (s, 1H), 10.21 (br s, 1H), 8.99 (s, 1H), 8.68 (t, J = 5.4 Hz, 1H), 8.65 (s, 1H), 7.43 (dt, J = 12.1, 7.8 Hz, 1H), 4.72 (t, J = 5.0 Hz, 1H), 4.61 (t, J = 5.0 Hz, 1H), 3.91 (q, 5.2 Hz, 1H), 3.84 (q, J = 5.2 Hz, 1H), 3.14-3.02 (m, 2H), 1.81-1.64 (m, 2H), 0.97 (t, J = 7.4 Hz, 3H) |
| 90 | 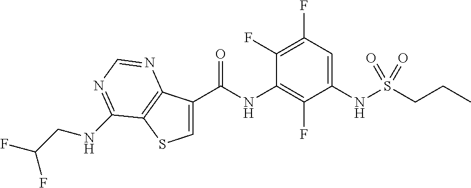 | 4-(2,2- Difluoro- ethylamino)- thieno[3,2-d] pyrimidine-7- carboxylic acid [2,3,6- trifluoro-5- (propane-1- sulfonyl- amino)- phenyl]-amide | 510.1 [M + 1] | 11.42 (s, 1H), 9.98 (s, 1H), 9.03 (s, 1H), 8.83 (t, J = 5.9 Hz, 1H), 8.70 (s, 1H), 7.47 (dt, J = 11.3, 7.6 Hz, 1H), 6.26 (tt, J = 56.0, 4.0 Hz, 1H), 4.09- 3.91 (m, 2H), 3.21-3.11 (m, 2H), 1.81-1.67 (m, 2H), 0.98 (t, J = 7.4 Hz, 3H) |
| 91 | 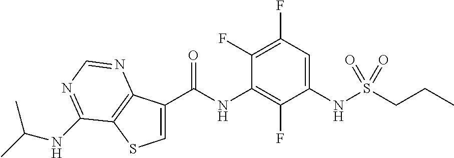 | 4-Isopropyl- amino-thieno [3,2-d] pyrimidine-7- carboxylic acid [2,3,6- trifluoro-5- (propane-1- sulfonyl- amino)- phenyl]-amide | 488.1 [M + 1] | 11.54 (s, 1H), 9.97 (s, 1H), 8.96 (s, 1H), 8.62 (s, 1H), 8.24 (s, 1H), 7.47 (d, J = 8.2 Hz, 1H), 4.52 (s, 1H), 3.16 (s, 2H), 1.74 (d, J = 7.1 Hz, 2H), 1.27 (d, J = 5.9 Hz, 6H), 0.99 (d, J = 6.9 Hz, 3H) |
| 92 | 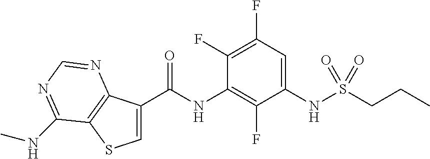 | 4-Methyl- amino- thieno[3,2-d] pyrimidine-7- carboxylic acid [2,3,6- trifluoro-5- (propane-1- sulfonyl- amino)- phenyl]-amide | 460.1 [M + 1] | 11.50 (s, 1H), 10.04 (s, 1H), 8.96 (s, 1H), 8.64 (s, 1H), 8.40 (s, 1H), 7.45 (d, J = 8.5 Hz, 1H), 3.12 (s, 2H), 3.04 (s, 3H), 1.73 (d, J = 7.5 Hz, 2H), 0.98 (t, J = 7.3 Hz, 3H) |
| 93 | 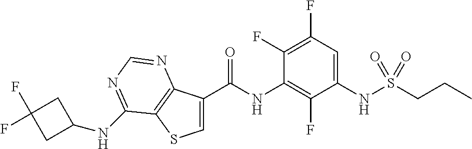 | 4-(3,3- Difluoro- cyclobutyl- amino)- thieno[3,2-d] pyrimidine-7- carboxylic acid [2,3,6- trifluoro-5- (propane-1- sulfonyl- amino)- phenyl]-amide | 536.1 [M + 1] | 11.42 (s, 1H), 9.97 (br s, 1H), 9.01 (s, 1H), 8.78 (d, J = 5.9 Hz, 1H), 8.68 (s, 1H), 7.50-7.41 (m, 1H), 4.54 (d, J = 6.0 Hz, 1H), 3.20-3.02 (m, 4H), 2.91-2.72 (m, 2H), 1.82-1.66 (m, 2H), 0.98 (t, J = 7.4 Hz, 3H) |
| 94 | 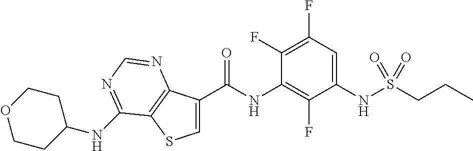 | 4-(Tetrahydro- pyran-4- ylamino)- thieno[3,2-d] pyrimidine-7- carboxylic acid [2,3,6- trifluoro-5- (propane-1- sulfonyl- amino)- phenyl]-amide | 530.1 [M + 1] | 11.51 (s, 1H), 9.95 (br s, 1H), 8.98 (s, 1H), 8.63 (s, 1H), 8.31 (d, J = 7.4 Hz, 1H), 7.50-7.41 (m, 1H), 4.47-4.35 (m, 1H), 3.93 (d, J = 8.4 Hz, 2H), 3.44 (t, J = 11.2 Hz, 2H), 3.20-3.08 (m, 2H), 1.90 (d, J = 12.2 Hz, 2H), 1.80-1.57 (m, 4H), 0.98 (t, J = 7.4 Hz, 3H) |
| 95 | 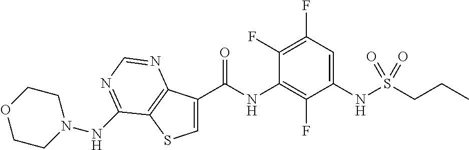 | 4-(Morpholin- 4-ylamino)- thieno[3,2-d] pyrimidine-7- carboxylic acid [2,3,6- trifluoro-5- (propane-1- sulfonyl- amino)- phenyl]-amide | 531.1 [M + 1] | 11.48 (s, 1H), 9.73 (s, 1H), 9.00 (s, 1H), 8.55 (s, 1H), 7.47-7.36 (m, 1H), 3.89 (d, J = 10.5 Hz, 2H), 3.72 (t, J = 11.0 Hz, 2H), 3.16-2.96 (m, 4H), 2.87 (t, J = 10.7 Hz, 2H), 1.78- 1.66 (m, 2H), 0.96 (t, J = 7.4 Hz, 3H) |
| 96 | 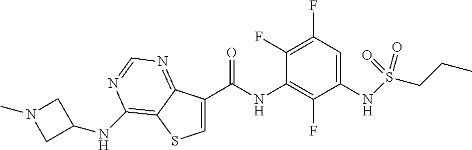 | 4-(1-Methyl- azetidin-3- ylamino)- thieno[3,2-d] pyrimidine-7- carboxylic acid [2,3,6- trifluoro-5- (propane-1- sulfonyl- amino)- phenyl]-amide | 515.1 [M + 1] | 11.13 (s, 1H), 9.42 (s, 1H), 9.11 (s, 1H), 8.14 (s, 2H), 3.17-3.02 (m, 4H), 1.81-1.68 (m, 2H), 1.24 (m, 6H), 0.98 (t, J = 7.4 Hz, 3H) |
| 97 | 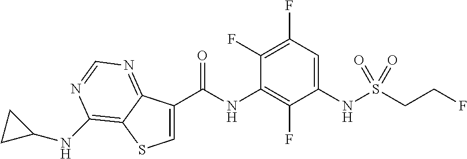 | 4-Cyclopropyl- amino-thieno [3,2-d] pyrimidine-7- carboxylic acid [2,3,6- trifluoro-5- (3-fluoro- propane-1- sulfonyl- amino)- phenyl]-amide | 504.1 [M + 1] | 11.45 (s, 1H), 8.99 (s, 1H), 8.63 (s, 1H), 8.53 (s, 1H), 7.41 (s, 1H), 4.59 (t, J = 5.9 Hz, 1H), 4.47 (t, J = 5.8 Hz, 1H), 3.17-3.01 (m, 3H), 2.18- 1.96 (m, 2H), 0.85 (br s, 2H), 0.70 (br s, 2H) |
| 98 | 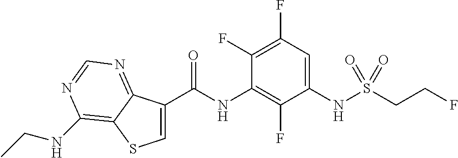 | 4-Ethylamino- thieno[3,2-d] pyrimidine-7- carboxylic acid [2,3,6- trifluoro-5- (3-fluoro- propane-1- sulfonyl- amino)- phenyl]-amide | 492.0 [M + 1] | 11.53 (s, 1H), 10.10 (s, 1H), 8.96 (s, 1H), 8.62 (s, 1H), 8.43 (s, 1H), 7.53- 7.42 (m, 1H), 4.60 (t, J = 5.8 Hz, 1H), 4.49 (t, J = 5.7 Hz, 114), 3.66- 3.52 (m, 2H), 3.26 (d, J = 7.5 Hz, 2H), 2.19-2.04 (m, 2H), 1.23 (t, J = 7.1 Hz, 3H) |

### Example 99

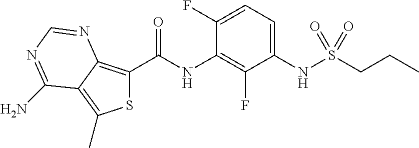

### 4-Amino-5-methyl-thieno[3,4-a]pyrimidine-7-carboxylic acid [2,6-difluoro-3-(propane-1-sulfonylamino)-phenyl]-amide

Step A: 4-(2,4-Dimethoxybenzylamino)-5-methylthieno[3,4-d]pyrimidine-7-carboxylic acid (176 mg, 0.49 mmol), N-(3-amino-2,4-difluorophenyl)-N-(4-methoxybenzyl)propane-1-sulfonamide (200 mg, 0.539 mmol), and HATU (279 mg, 0.74 mmol) were dissolved in N,N-dimethylformamide (4 mL) and N,N-diisopropylethylamine (0.26 mL, 1.47 mmol). The reaction was allowed to heat to 55° C. overnight. After cooling to room temperature the reaction mixture was diluted with ethyl acetate and washed with saturated sodium bicarbonate. The organic layer was concentrated and purified by flash chromatography (0-100% ethyl acetate:heptane) to give N-(2,6-difluoro-3-(N-(4-methoxybenzyl)propylsulfon-amido)phenyl)-4-(2,4-dimethoxybenzylamino)-5-methylthieno[3,4-d]pyrimidine-7-carboxamide (54 mg, 15%). ¹H NMR (400 MHz, DMSO-d₆) δ 10.85 (s, 1H), 8.30 (s, 1H), 7.86 (s, 1H), 7.23 (s, 1H), 7.16 (s, 4H), 6.83 (s, 2H), 6.60 (s, 1H), 6.47 (s, 1H), 4.70 (s, 2H), 3.86 (s, 3H), 3.74 (s, 3H), 3.70 (s, 4H), 3.27-3.22 (m, 2H), 1.78 (s, 2H), 1.01 (s, 3H). MS m/z=712.5 [M+H]⁺.

Step B: N-(2,6-Difluoro-3-(N-(4-methoxybenzyl)propylsulfonamido)phenyl)-4-(2,4-dimethoxybenzylamino)-5-methylthieno[3,4-d]pyrimidine-7-carboxamide (54 mg, 0.076 mmol) was dissolved in trifluoroacetic acid (3 mL) and heated to reflux for 3 hours before concentration to dryness. The residual oil was dissolved in ethyl acetate, washed subsequently with saturated sodium bicarbonate, water, and brine. The organic layer was purified by flash chromatography (0-100% ethyl acetate: heptane) to give 4-amino-5-methyl-thieno[3,4-d]pyrimidine-7-carboxylic acid [2,6-difluoro-3-(propane-1-sulfonylamino)-phenyl]amide (9 mg, 30%). ¹H NMR (400 MHz, DMSO-d₆) δ 10.89 (s, 1H), 9.67 (s, 1H), 8.26 (s, 1H), 7.36 (dd, J=14.5, 8.7 Hz, 1H), 7.20 (t, J=8.9 Hz, 1H), 3.14-3.04 (m, 2H), 3.01 (d, J=11.0 Hz, 3H), 1.83-1.69 (m, 2H), 1.02-0.95 (m, 3H). MS m/z=442.2 [M+H]⁺.

### Example 100

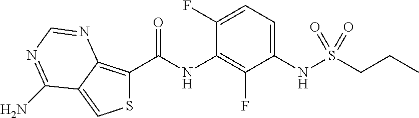

### 4-Amino-N-(2,6-difluoro-3-(propylsulfonamido)phenyl)thieno[3,4-d]pyrimidine-7-carboxamide

Step A: 4-(2,4-Dimethoxybenzylamino)thieno[3,4-d]pyrimidine-7-carboxylic acid (114 mg, 0.330 mmol) and benzotriazol-1-yloxytris(dimethylamino)phosphonium hexafluorophosphate (0.219 g, 0.495 mmol) were dissolved in DMF (3 mL, 0.04 mol). 1,8-Diazabicyclo[5.4.0]undec-7-ene (64.2 uL, 0.429 mmol) was added to this solution and the resulting mixture was stirred for 5 minutes before the addition of N-(3-amino-2,4-difluorophenyl)propane-1-sulfonamide (124 mg, 0.495 mmol). The reaction was heated to 55° C. overnight, cooled to room temperature, diluted with ethyl acetate and washed with saturated aqueous sodium bicarbonate. The organic layer was then purified via flash chromatography (0-100% ethyl acetate:heptane) to give N-(2,6-difluoro-3-(propylsulfonamido)phenyl)-4-(2,4-dimethoxybenzylamino)thieno[3,4-d]pyrimidine-7-carboxamide as a tan oil (44 mg, 24%). ¹H NMR (400 MHz, DMSO-d₆) δ 10.94 (s, 1H), 9.71 (s, 1H), 9.24 (s, 1H), 9.04 (s, 1H), 8.41 (s, 1H), 7.37 (d, J=5.8 Hz, 1H), 7.27-7.15 (m, 2H), 6.61 (s, 1H), 6.50 (d, J=8.7 Hz, 1H), 4.70 (d, J=4.4 Hz, 2H), 3.82 (s, 3H), 3.75 (s, 3H), 1.75 (dd, J=14.6, 7.0 Hz, 2H), 0.98 (t, J=7.3 Hz, 3H). MS m/z=578.3 [M+H]⁺.

Step B: N-(2,6-Difluoro-3-(propylsulfonamido)phenyl)-4-(2,4-dimethoxy benzylamino)thieno[3,4-d]pyrimidine-7-carboxamide (44.7 mg, 0.0774 mmol) was dissolved in trifluoroacetic acid (3 mL) and heated to 115° C. in a sealed vial for 2 hours. The reaction mixture was then concentrated to dryness, redissolved in ethyl acetate, washed with saturated sodium bicarbonate and purified by flash chromatography (0-100% ethyl acetate: heptane) to give 4-amino-N-(2,6-difluoro-3-(propylsulfonamido)phenyl)thieno[3,4-d]pyrimidine-7-carboxamide as tan solid (13 mg, 39%). ¹H NMR (400 MHz, DMSO-d₆) δ 10.90 (s, 1H), 9.72 (s, 1H), 8.95 (s, 1H), 8.57 (br s, 1H), 8.40 (br s, 1H), 8.34 (s, 1H), 7.43-7.32 (m, 1H), 7.23 (t, J=9.3 Hz, 1H), 3.13-3.04 (m, 2H), 1.75 (dd, J=15.0, 7.8 Hz, 2H), 0.98 (t, J=7.4 Hz, 3H). MS m/z=428.1 [M+H]⁺.

### Example 101

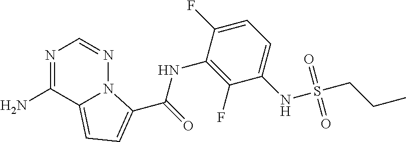

### 4-Amino-pyrrolo[2,1-f][1,2,4]triazine-7-carboxylic acid [2,6-difluoro-3-(propane-1-sulfonylamino)-phenyl]-amide

Step A: A vial was charged with 4-(2,4-dimethoxybenzylamino)-pyrrolo[1,2-f][1,2,4]triazine-7-carboxylic acid (166 mg, 0.51 mmol), N-(3-amino-2,4-difluoro-phenyl)propane-1-sulfonamide (152 mg, 0.61 mmol), HATU (202 mg, 0.53 mmol), N,N-diisopropylethylamine (0.18 mL, 1.01 mmol) and DMF (2.5 mL). Reaction mixture was stirred at 55° C. for 16 hours, after which it was diluted with ethyl acetate and water. The layers were separated and the aqueous layer extracted with ethyl acetate (2×). The organic layers were combined and dried with sodium sulfate, filtered and concentrated in vacuo. The crude product was purified by flash chromatography to afford N-(2,6-difluoro-3-(propyl-□ulfonamide)phenyl)-4-(2,4-dimethoxybenzylamino)pyrrolo[1,2-f][1,2,4]triazine-7-carboxamide (120 mg, 42%), which still contained numerous impurities. Material was still carried forward to the next step.

Step B: N-(2,6-difluoro-3-(propylsulfonamido)phenyl)-4-(2,4-dimethoxybenzyl-amino)pyrrolo[1,2-f][1,2,4]triazine-7-carboxamide (120 mg, 0.21 mmol) was dissolved in TFA (2 mL) and the reaction mixture stirred at 50° C. for 5 hours, after which it was concentrated in vacuo. The crude product was directly purified by SFC to afford 4-amino-pyrrolo[2,1-f][1,2,4]triazine-7-carboxylic acid [2,6-difluoro-3-(propane-1-sulfonyl-amino)-phenyl]-amide (12 mg, 14%). ¹H NMR (400 MHz, DMSO-d₆) δ 10.53 (s, 1H), 9.84 (br s, 1H), 8.30 (d, J=26.3 Hz, 2H), 8.16 (s, 1H), 7.41-7.28 (m, 2H), 7.21 (t, J=9.1 Hz, 1H), 7.06 (d, J=4.3 Hz, 1H), 3.11-3.01 (m, 2H), 1.81-1.68 (m, 2H), 0.98 (t, J=7.3 Hz, 3H). (ES-MS) [M+1] m/z 411.2 [M+H]⁺.

### Example 102

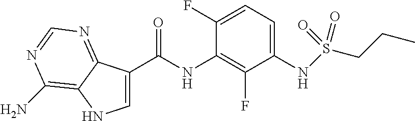

### 4-Amino-5H-pyrrolo[3,2-d]pyrimidine-7-carboxylic acid [2,6-difluoro-3-(propane-1-sulfonylamino)-phenyl]-amide

Step A: To a stirred solution of N-(3-Amino-2,4-difluorophenyl)-N-(4-methoxybenzyl)propane-1-sulfonamide (992 mg, 4.7 mmol) in toluene (20 mL) was added trimethyl-aluminum (2 M in hexane; 0.5 mL) dropwise. The mixture was stirred at room temperature for 2 hours. A solution of ethyl 4-chloro-5H-pyrrolo[3,2-d]pyrimidine-7-carboxylate (1737 mg, 4.7 mmol) dissolved in toluene (2 mL) and 1,4-dioxane (4 mL) and was added to the reaction mixture. The mixture was stirred at 60° C. overnight. The mixture was quenched with methanol and 2N HCl. The mixture was applied to a Varian Chemelut cartridge, eluted with dichloromethane and ethyl acetate, and concentrated. The crude product was purified using flash chromatography (gradient elution: 0-100% ethyl acetate in heptanes) to yield N-(3-(4-methoxybenzyl-propylsulfonamido)-2,6-difluorophenyl)-4-chloro-5H-pyrrolo[3,2-d]pyrimidine-7-carboxamide (1237 mg, 60%). LC/MS: m/z 550.1 [M+1]

Step B: N-[3-(4-Methoxybenzylpropylsulfonamido)-2,6-difluorophenyl]-4-chloro-5H-pyrrolo[3,2-d]pyrimidine-7-carboxamide (50 mg, 0.09 mmol) was suspended in a solution of ammonia in isopropyl alcohol (2 M; 2.5 mL). The reaction was heated in a microwave reactor at 105° C. for 10 minutes Ammonia gas was passed through the reaction mixture., and the reaction was heated in a microwave reactor at 120° C. for 30 minutes. Purging with ammonia gas and heating in a microwave reactor at 120° C. for 30 minutes was repeated twice. The mixture was concentrated and refluxed in trifluoracetic acid (2 mL) for 2 hours. The crude product was purified using flash chromatography (gradient elution using 0-100% ethyl acetate in heptanes) to yield 4-amino-5H-pyrrolo[3,2-d]pyrimidine-7-carboxylic acid [2,6-difluoro-3-(propane-1-sulfonylamino)-phenyl]-amide (16 mg, 40%). ¹H NMR ((400 MHz, DMSO-d₆) δ 12.26 (s, 1H), 10.29 (s, 1H), 9.68 (s, 1H), 8.42 (s, 2H), 7.38 (dd, J=14.3, 8.8 Hz, 1H), 7.22 (s, 1H), 3.21-3.00 (m, 2H), 1.76 (dd, J=15.1, 7.5 Hz, 2H), 0.99 (t, J=7.4 Hz, 3H). LC/MS: m/z 411.1 [M+1].

### Example 103

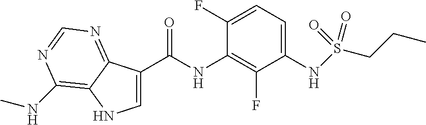

### 4-Methylamino-5H-pyrrolo[3,2-d]pyrimidine-7-carboxylic acid [2,6-difluoro-3-(propane-1-sulfonylamino)-phenyl]-amide

To N-(3-(4-methoxybenzyl-propylsulfonamido)-2,6-difluorophenyl)-4-chloro-5H-pyrrolo-[3,2-d]pyrimidine-7-carboxamide (50 mg, 0.09 mmol) and methylamine (60 ul, 1.36 mmol) in THF (1 mL) was added N,N-diisopropylethylamine (158 ul, 0.9 mmol). The reaction mixture was stirred at 100° C. for 1 hour and then concentrated in vacuo. The resultant crude material was refluxed in trifluoroacetic acid (2 mL) for 1 h and then evaporated in vacuo. Purification by prep HPLC gave 4-methylamino-5H-pyrrolo[3,2-d]pyrimidine-7-carboxylic acid [2,6-difluoro-3-(propane-1-sulfonylamino)-phenyl]-amide (17 mg, 52%). ¹H NMR (400 MHz, DMSO-d₆) δ 11.66 (s, 1H), 10.24 (s, 1H), 9.67 (s, 1H), 8.43 (s, 2H), 8.17 (s, 1H), 7.36 (d, J=5.9 Hz, 2H), 7.21 (t, J=9.2 Hz, 1H), 3.94-3.52 (m, 1H), 3.21-2.91 (m, 6H), 1.76 (d, J=7.6 Hz, 3H), 0.98 (t, J=7.4 Hz, 3H); LC-MS [M+1]m/z 425.1.

### Example 104

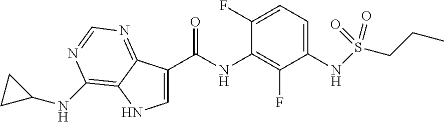

### 4-Cyclopropylamino-5H-pyrrolo[3,2-d]pyrimidine-7-carboxylic acid [2,6-difluoro-3-(propane-1-sulfonylamino)-phenyl]-amide

The title compound was prepared using a similar procedure as described for Example 103 using cyclopropylamine in place of methylamine in THF. ¹H NMR (400 MHz, DMSO-d₆) δ 12.68 (s, 1H), 11.59 (s, 1H), 10.25 (s, 1H), 9.67 (s, 1H), 8.46 (s, 2H), 8.13 (s, 1H), 7.36 (d, J=5.9 Hz, 1H), 7.21 (t, J=9.1 Hz, 1H), 3.58 (t, J=6.6 Hz, 1H), 3.19-3.00 (m, 3H), 2.10 (dt, J=15.0, 7.1 Hz, 1H), 1.76 (d, J=7.6 Hz, 3H), 0.98 (t, J=7.4 Hz, 6H), 0.74-0.44 (m, 3H), 0.36 (dt, J=6.8, 4.5 Hz, 1H). LC-MS [M+1]m/z 451.1.

### Example 105

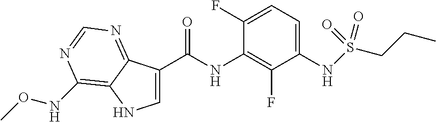

### 4-Methoxyamino-5H-pyrrolo[2-d]pyrimidine-7-carboxylic acid [2,6-difluoro-3-(propane-1-sulfonylamino)-phenyl]-amide

The title compound was prepared using a similar procedure as described for Example 103 using methoxyamine hydrochloride in place of methylamine in THF. ¹H NMR (400 MHz, DMSO-d₆) δ 12.41 (s, 1H), 11.34 (d, J=3.3 Hz, 1H), 9.76 (s, 1H), 9.65 (s, 1H), 7.67 (d, J=3.6 Hz, 1H), 7.59 (d, J=3.2 Hz, 1H), 7.34 (dd, J=14.5, 8.7 Hz, 1H), 7.18 (t, J=8.4 Hz, 1H), 6.48 (s, OH), 3.81 (s, 3H), 3.15-2.99 (m, 2H), 1.88-1.63 (m, 2H), 0.98 (s, 3H). LC-MS [M+1]m/z 441.1.

### Example 106

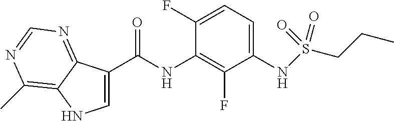

### 4-Methyl-5H-pyrrolo[3,2-d]pyrimidine-7-carboxylic acid [2,6-difluoro-3-(propane-1-sulfonylamino)-phenyl]-amide

The title compound was prepared using a similar procedure as described for Example 39 using 4-chloro-N-(2,6-difluoro-3-(4-methoxybenzyl)butylsulfonamido)phenyl)-5H-pyrrolo[3,2-d]pyrimidine-7-carboxamide in place of 4-chloro-N-(2,6-dichloro-3-(3-fluoropropylsulfonamido)phenyl)thieno[3,2-d]pyrimidine-7-carboxamide. ¹H NMR (400 MHz, DMSO-d₆) δ 2.91 (s, 1H), 9.97 (s, 1H), 9.68 (s, 1H), 8.90 (s, 1H), 8.56 (s, 1H), 7.37 (td, J=8.8, 5.7 Hz, 1H), 7.21 (dd, J=9.9, 8.4 Hz, 1H), 3.08 (dd, J=8.7, 6.7 Hz, 2H), 2.76 (s, 3H), 1.85-1.61 (m, 2H), 0.98 (t, J=7.4 Hz, 3H). LC-MS [M+1]m/z 410.1.

### Example 107

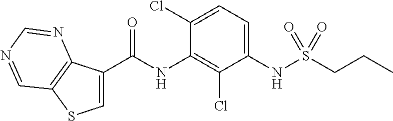

### Thieno[3,2-d]pyrimidine-7-carboxylic acid [2,6-dichloro-3-(propane-1-sulfonylamino)-phenyl]-amide

4-Chloro-N-(2,6-dichloro-3-(propylsulfonamido)phenyl)thieno[3,2-d]pyrimidine-7-carboxamide (27 mg, 0.06 mmol) and Zn (70 mg) were taken up in acetic acid (1 mL). The mixture was heated in a microwave reactor at 100° C. for 30 minutes. The reaction mixture was concentrated in vacuo and the crude product purified by preparative HPLC to obtain thieno[3,2-d]pyrimidine-7-carboxylic acid [2,6-dichloro-3-(propane-1-sulfonylamino)-phenyl]-amide (11 mg, 40%). ¹H NMR (400 MHz, DMSO-d₆) δ 10.81 (s, 1H), 9.86 (s, 2H), 9.45 (d, 2H), 7.57 (td, J=8.9, 5.8 Hz, 1H), 7.34 (dd, J=9.2, 7.9 Hz, 1H), 3.19-3.00 (m, 3H), 1.92-1.59 (m, 2H), 0.98 (t, J=7.4 Hz, 3H). LC-MS [M+1]m/z 445.0.

### Example 108

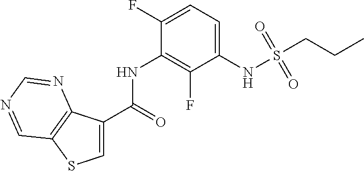

### Thieno[3,2-d]pyrimidine-7-carboxylic acid [2,6-difluoro-3-(propane-1-sulfonylamino)-phenyl]-amide

A microwave vial was charged with 4-chloro-N-(2,6-difluoro-3-(propylsulfonamido)-phenyl)thieno[3,2-d]pyrimidine-7-carboxamide (0.15 g, 0.336 mmol, as prepared from Example 40, Step A), tri-n-butyltin hydride (0.18 mL, 0.671 mmol), tetrakis(triphenylphosphine)palladium(0) (0.097 g, 0.084 mmol) and toluene (1.6 mL). The reaction mixture was heated in a microwave reactor at 150° C. for 15 minutes. The palladium residue was then filtered off and the filtrate concentrated in vacuo. The crude mixture was purified by reverse phase HPLC to afford thieno[3,2-d]pyrimidine-7-carboxylic acid [2,6-difluoro-3-(propane-1-sulfonylamino)-phenyl]-amide (0.07 g, 73%). ¹H NMR (400 MHz, DMSO-d₆) δ 10.79 (s, 1H), 9.75 (s, 1H), 9.71 (s, 1H), 9.38 (s, 1H), 9.32 (s, 1H), 7.41 (td, J=8.9, 5.8 Hz, 1H), 7.24 (dd, J=9.2, 7.9 Hz, 1H), 3.14-3.04 (m, 2H), 1.81-1.70 (m, 2H), 0.98 (t, J=7.4 Hz, 3H). LC-MS m/z (ES-MS) 413.0 [M+1].

Examples 109-113 below in Table 5 were prepared according to the procedure described in Example 108 using appropriate starting materials.

TABLE 5

| Ex- | | | | |
|---|---|---|---|---|
| ample | | | | ¹H NMR δ |
| no. | Structure | Name | MS m/z | (400 MHz, DMSO-d6) |
| 109 | 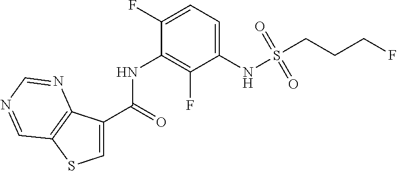 | Thieno[3,2-d]pyrimidine- 7-carboxylic acid [2,6-difluoro-3-(3-fluoro- propane-1-sulfonyl- amino)-phenyl]-amide | 431.0 [M + 1] | 10.79 (s, 1H), 9.80 (s, 1H), 9.75 (s, 2H), 9.38 (s, 1H), 9.32 (s, 1H), 7.41 (td, J = 8.8, 5.8 Hz, 1H), 7.25 (t, J = 9.2 Hz, 1H), 4.61 (t, J = 6.0 Hz, 1H), 4.49 (t, J = 6.0 Hz, 1H), 3.26-3.15 (m, 2H), 2.22-2.03 (m, 2H) |
| 110 | 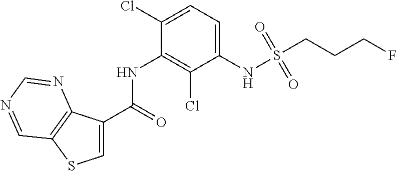 | Thieno[3,2-d]pyrimidine- 7-carboxylic acid [2,6-dichloro-3-(3-fluoro- propane-1-sulfonyl- amino)-phenyl]-amide | 463.0 [M + 1] | 11.02 (s, 1H), 9.88 (s, 1H), 9.76 (s, 1H), 9.38 (s, 1H), 9.33 (s, 1H), 7.60 (d, J = 8.7 Hz, 1H), 7.51 (d, J = 8.7 Hz, 1H), 4.61 (t, J = 5.7 Hz, 1H), 4.49 (t, J = 5.8 Hz, 1H), 3.28- 3.19 (m, 2H), 2.20-2.02 (m, 2H) |
| 111 | 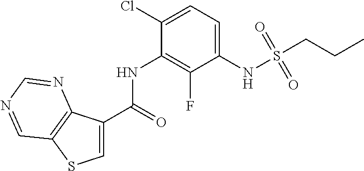 | Thieno[3,2-d]pyrimidine- 7-carboxylic acid [6-chloro-2-fluoro-3- (propane-1-sulfonylamino)- phenyl]-amide | 429.2 [M + 1] | 10.93 (s, 1H), 9.95 (s, 1H), 9.77 (s, 1H), 9.39 (s, 1H), 9.34 (s, 1H), 7.49-7.39 (m, 2H), 3.17-3.05 (m, 2H), 1.81-1.65 (m, 2H), 0.97 (t, J = 7.4 Hz, 3H) |
| 112 | 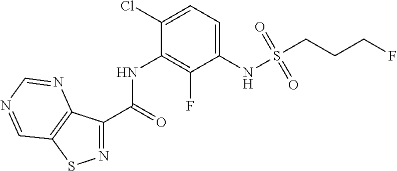 | Isothiazolo[4,5-d] pyrimidine-3-carboxylic acid [6-chloro-2-fluoro-3- (3-fluoro-propane-1- sulfonylamino)-phenyl]- amide | 448.0 [M + 1] | 10.86 (s, 1H), 10.00 (s, 1H), 9.48 (s, 1H), 7.47- 7.41 (m, 2H), 4.60 (t, J = 5.9 Hz, 1H), 4.49 (t, J = 5.9 Hz, 1H), 3.27-3.17 (m, 2H), 2.19-2.01 (m, 2H) |
| 113 | 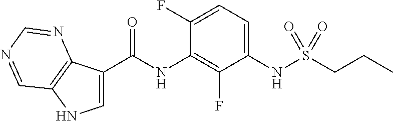 | 5H-Pyrrolo[3,2-d] pyrimidine-7-carboxylic acid [2,6-difluoro-3-(propane- 1-sulfonylamino)- phenyl]-amide | 396.1 [M + 1] | 9.89 (s, 1H), 9.10 (s, 1H), 9.06 (s, 1H), 8.58 (s, 1H), 7.37 (td, J = 8.9, 5.7 Hz, 1H), 7.21 (d, J = 1.5 Hz, 1H), 3.08 (dd, J = 8.7, 6.6 Hz, 2H), 1.82-1.67 (m, 2H), 0.98 (t, J = 7.4 Hz, 3H) |

Table 6 shows the activity of certain compounds of the invention tested in the above B-RAF V600E inhibition assay (Example A).

| Example | BRAF V600E IC₅₀ (μM) |
|---|---|
| 1 | 0.0184 |
| 2 | 0.01203 |
| 3 | 0.00389 |
| 4 | 0.00425 |
| 5 | 0.00086 |
| 6 | 0.00289 |
| 7 | 0.00101 |
| 8 | 0.0006 |
| 9 | 0.0001 |
| 10 | 0.00072 |
| 11 | 0.00089 |
| 12 | 0.00022 |
| 13 | 0.0001 |
| 14 | 0.0006 |
| 15 | 0.0002 |
| 16 | 0.0019 |
| 17 | 0.0014 |
| 18 | 0.0074 |
| 19 | 0.0026 |
| 20 | 0.0057 |
| 21 | 0.0471 |
| 22 | 0.002 |
| 23 | 0.0008 |
| 24 | 0.00011 |
| 25 | 0.0072 |
| 26 | 0.02 |
| 27 | 0.125 |
| 28 | 0.0116 |
| 29 | 0.088 |
| 30 | 0.0012 |
| 31 | 0.00082 |
| 32 | 0.00018 |
| 33 | 0.00038 |
| 34 | 0.005 |
| 35 | 0.105 |
| 36 | 0.194 |
| 37 | 0.0092 |
| 38 | 0.0465 |
| 39 | 0.00111 |
| 40 | 0.0855 |
| 41 | 0.112 |
| 42 | 0.026 |
| 43 | 0.0019 |
| 44 | 0.0087 |
| 45 | 0.0041 |
| 46 | 0.04957 |
| 47 | 0.0047 |
| 48 | 0.0114 |
| 49 | 0.0109 |
| 50 | 0.0365 |
| 51 | 0.221 |
| 52 | 0.0157 |
| 53 | 0.0265 |
| 54 | 0.416 |
| 55 | 0.0455 |
| 56 | 0.0751 |
| 57 | 0.0128 |
| 58 | 0.902 |
| 59 | 0.0571 |
| 60 | 0.177 |
| 61 | 0.0017 |
| 62 | 0.0271 |
| 63 | 0.0354 |
| 64 | 0.0011 |
| 65 | 0.0012 |
| 66 | 0.0002 |
| 67 | 0.0002 |
| 68 | 0.0018 |
| 69 | 0.0022 |
| 70 | 0.0034 |
| 71 | 0.0144 |
| 72 | 0.0024 |
| 73 | 0.014 |
| 74 | 0.0005 |
| 75 | >1.0 |
| 76 | 0.0006 |
| 77 | 0.0775 |
| 78 | 0.0117 |
| 79 | 0.159 |
| 80 | 0.419 |
| 81 | 0.11 |
| 82 | 0.00013 |
| 83 | 0.00032 |
| 84 | 0.00025 |
| 85 | 0.0051 |
| 86 | 0.0012 |
| 87 | 0.0151 |
| 88 | 0.12 |
| 89 | 0.0037 |
| 90 | 0.016 |
| 91 | 0.0279 |
| 92 | 0.0132 |
| 93 | 0.118 |
| 94 | 0.704 |
| 95 | 0.00509 |
| 96 | 1.00 |
| 97 | 0.00071 |
| 98 | 0.00216 |
| 99 | 0.003 |
| 100 | 0.0298 |
| 101 | 0.0025 |
| 102 | 0.02063 |
| 103 | 0.11538 |
| 104 | 0.04758 |
| 105 | 0.112 |
| 106 | 0.18908 |
| 107 | 0.0096 |
| 108 | 0.2884 |
| 109 | 0.0403 |
| 110 | 0.00269 |
| 111 | 0.0211 |
| 112 | 0.00729 |
| 113 | 0.35298 |

While the invention has been described in conjunction with the enumerated embodiments, it will be understood that they are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

Specific reference is made to U.S. Provisional Patent Appl. No. 61/238,109, filed Aug. 28, 2009, which is incorporated herein by reference in its entirety for all purposes.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. A compound selected from Formula I: 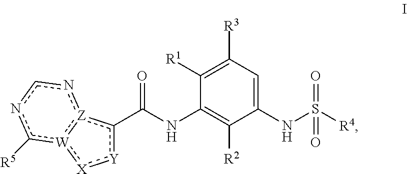
stereoisomers, tautomers, prodrugs and pharmaceutically acceptable salts thereof, wherein:
the dashed lines represent optional double bonds such that the bicycle containing the double bonds is aromatic;
W and Z are independently C or N;
X is O, S, NR⁶ or CR⁶, and Y is NR⁷ or CR⁷; or X is NR⁶ or CR⁶, and Y is O, S, NR⁷ or CR⁷; provided at least one of W, X, Y and Z is other than C, CR⁶ and CR⁷;
R¹ and R² are independently selected from hydrogen, halogen, CN, C₁-C₃ alkyl, C₁-C₃ alkoxy, C₁-C₃ alkenyl and C₁-C₃ alkynyl;
R³ is hydrogen, halogen or C₁-C₃ alkyl;
R⁴ is C₃-C₆ cycloalkyl, C₁-C₆ alkyl, C₂-C₆ alkenyl, C₂-C₆ alkynyl, phenyl, 3-6 membered heterocyclyl, a 5-6 membered heteroaryl, or NR⁸R⁹, wherein the cycloalkyl, alkyl, alkenyl, alkynyl, phenyl, heterocyclyl and heteroaryl are optionally substituted with OR²⁰, halogen, phenyl, C₃-C₆ cycloalkyl, or C₁-C₆ alkyl optionally substituted with halogen;
R⁵ is hydrogen, C₁-C₃ alkyl optionally substituted by halogen, or NR¹⁰R¹¹;
R⁶ is hydrogen, C₁-C₆ alkoxy or C₁-C₆ alkyl, wherein each C₁-C₆ alkoxy and C₁-C₆ alkyl is optionally substituted with halogen, OR²⁰, SR²⁰, NR¹⁴R¹⁵, C₃-C₆ cycloalkyl, 4-6 membered heterocyclyl, 5-6 membered heteroaryl or phenyl; provided when X is NR⁶ and is double bonded to an adjacent atom in formula I then R⁶ is absent;
R⁷ is hydrogen, C₁-C₆ alkoxy or C₁-C₆ alkyl, wherein each C₁-C₆ alkoxy and C₁-C₆ alkyl is optionally substituted with halogen, OR²⁰, SR²⁰, NR¹⁶R¹⁷, C₃-C₆ cycloalkyl, 4-6 membered heterocyclyl, 5-6 membered heteroaryl or phenyl; provided when Y is NR⁷ and is double bonded to an adjacent atom in formula I then R⁷ is absent;
R⁸ and R⁹ are each independently hydrogen or C₁-C₆ alkyl optionally substituted by halogen; or R⁸ and R⁹ are independently taken together with the atom to which they are attached to form a 3-6 membered heterocyclyl, optionally substituted by halogen, oxo or C₁-C₃ alkyl;
R¹⁰ is hydrogen;
R¹¹ is hydrogen, —(C₀-C₃ alkyl)CN, (C₀-C₃ alkyl)NR¹²R¹³, (C₀-C₃ alkyl)OR²⁰, (C₁-C₃ alkyl)SR²⁰, C₁-C₆ alkyl, C₂-C₆ alkenyl, C₂-C₆ alkynyl, (C₀-C₃ alkyl)C₃-C₆ cycloalkyl, (C₀-C₃ alkyl)phenyl, (C₀-C₃ alkyl) 3-6-membered heterocyclyl or (C₀-C₃ alkyl) 5-6-membered heteroaryl, wherein said alkyl, alkenyl, alkynyl, cycloalkyl, heterocyclyl, heteroaryl and phenyl are optionally substituted by halogen, oxo, OR²¹, NR¹⁸R¹⁹ or C₁-C₃ alkyl;
R¹² and R¹³ are independently hydrogen or C₁-C₆ alkyl optionally substituted by halogen; or R¹² and R¹³ are taken together with the atom to which they attached to form a 3-6 membered heterocyclyl, optionally substituted by halogen, oxo or C₁-C₃ alkyl;
R¹⁴ and R¹⁵ are independently hydrogen or C₁-C₆ alkyl optionally substituted by halogen; or R¹⁴ and R¹⁵ are taken together with the atom to which they attached to form a 3-6 membered heterocyclyl optionally substituted by halogen, oxo or C₁-C₃ alkyl;
R¹⁶ and R¹⁷ are independently hydrogen or C₁-C₆ alkyl optionally substituted by halogen; or R¹⁶ and R¹⁷ are taken together with the atom to which they attached to form a 3-6 membered heterocyclyl optionally substituted by halogen, oxo or C₁-C₃ alkyl;
R¹⁸ and R¹⁹ are independently hydrogen or C₁-C₆ alkyl optionally substituted by halogen; or R¹⁸ and R¹⁹ are taken together with the atom to which they attached to form a 3-6 membered heterocyclyl optionally substituted by halogen, oxo or C₁-C₃ alkyl;
each R²⁰ is independently hydrogen or C₁-C₆ alkyl optionally substituted by halogen; and
each R²¹ is independently hydrogen or C₁-C₆ alkyl optionally substituted by halogen.

2. A compound of claim 1 selected from:

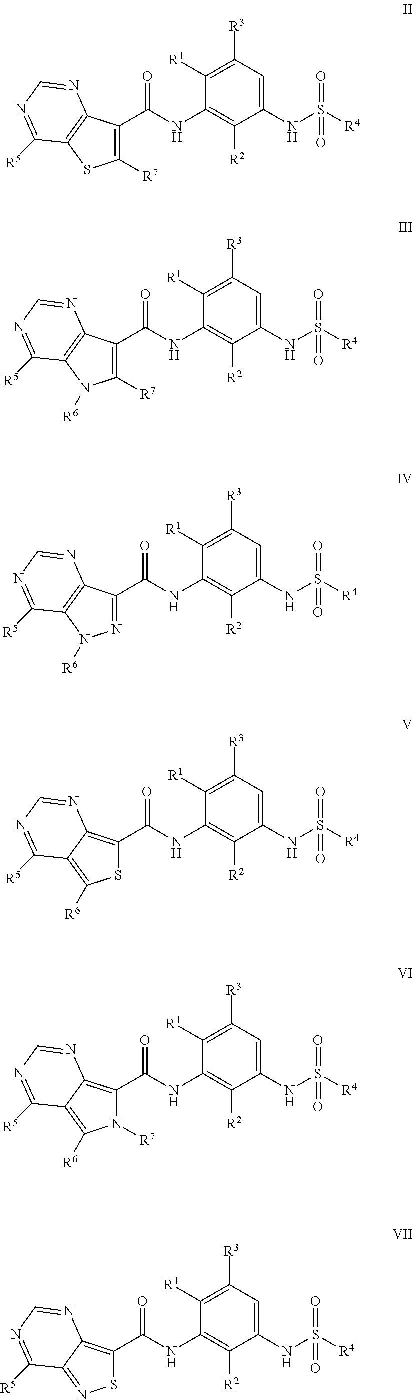

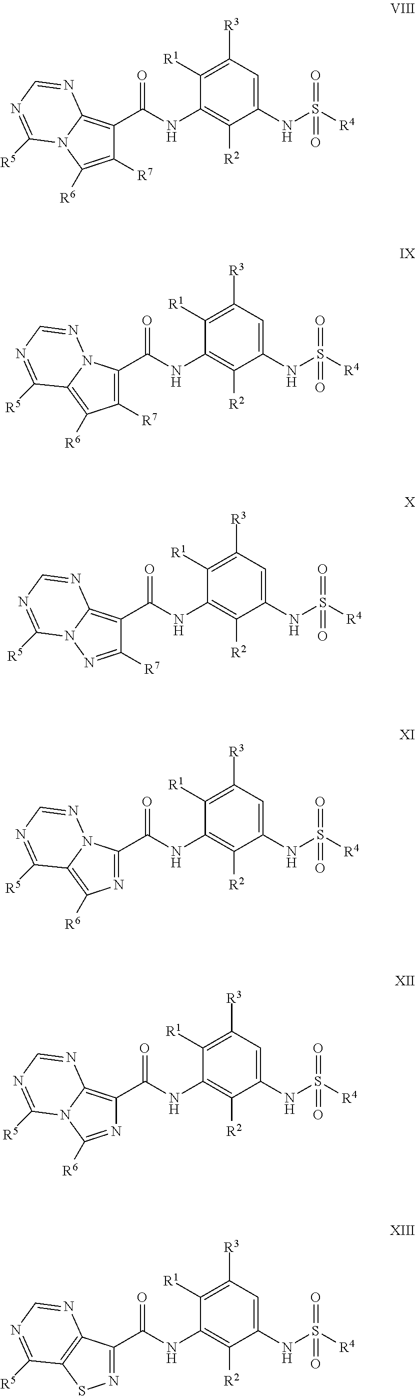

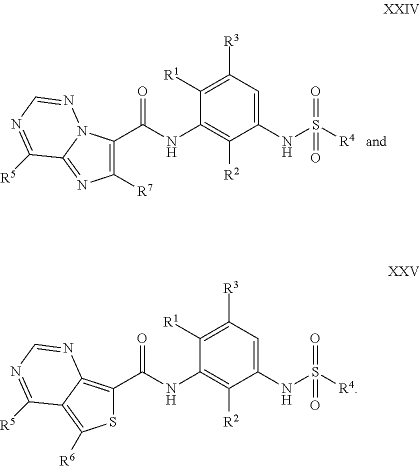

3. A compound of claim 1, wherein W and Z are C, X is S and Y is CR⁷.

4. A compound of claim 1, wherein W and Z are C, X is NR⁶ and Y is CR⁷.

5. A compound of claim 1, wherein W and Z are C, X is NR⁶ and Y is N.

6. A compound of claim 1, wherein W and Z are C, X is CR⁶ and Y is S.

7. A compound of claim 1, wherein W and Z are C, X is CR⁶ and Y is NR⁷.

8. A compound of claim 1, wherein W and Z are C, X is N and Y is S.

9. A compound of claim 1, wherein W is N, Z is C, X is CR⁶ and Y is CR⁷.

10. A compound of claim 1, wherein W is C, Z is N, X is CR⁶ and Y is CR⁷.

11. A compound of claim 1, wherein W is N, Z is C, X is N and Y is CR⁷.

12. A compound of claim 1, wherein W is C, Z is N, X is CR⁶ and Y is N.

13. A compound of claim 1, wherein W is N, Z is C, X is CR⁶ and Y is N.

14. A compound of claim 1, wherein W and Z are C, X is S and Y is N.

15. A compound of claim 1, wherein W is C, Z is N, X is N and Y is CR⁷.

16. A compound of claim 1, wherein W and Z are C, X is CR⁶ and Y is S.

17. A compound of any one of claims 1-16, wherein R¹, R² and R³ are independently selected from hydrogen, halogen or C₁-C₃ alkyl.

18. A compound of any one of claims 1-17, wherein R¹ and R² are F or Cl and R³ is hydrogen.

19. A compound of any one of claims 1-18, wherein R¹ is Cl, and R² is F and R³ is hydrogen.

20. A compound of any one of claims 1-19, wherein R⁴ is C₁-C₃ alkyl optionally substituted by halogen.

21. A compound of any one of claims 1-20, wherein R⁴ is ethyl, propyl or —CH₂CH₂CH₂F.

22. A compound of any one of claims 1-21, wherein R⁵ is NR¹⁰R¹¹.

23. A compound of any one of claims 1-21, wherein R⁵ is NH₂.

24. A compound named in any one of Examples 1-113.

25. A pharmaceutical composition, comprising a compound of any one of claims 1-24 and 30 and a pharmaceutically acceptable carrier or excipient.

26. A method of preventing or treating a disease or disorder modulated by b-Raf, comprising administering to a mammal in need of such treatment an effective amount of a compound of any one of claims 1-24 and 30.

27. A compound of any one of claims 1-24 and 30 for use in therapy.

28. Use of a compound of any one of claims 1-24 and 30 in the manufacture of a medicament for the treatment of cancer.

29. A pharmaceutical composition comprising a compound of any one of claims 1-24 and 30 for use in the treatment of cancer.

30. A compound of claim 1, wherein:
W and Z are independently C or N;
X is O, S, NR⁶ or CR⁶, and Y is NR⁷ or CR⁷; or X is NR⁶ or CR⁶, and Y is O, S, NR⁷ or CR⁷; provided at least one of W, X, Y and Z is other than C, CR⁶ and CR⁷;
R¹ and R² are independently selected from hydrogen, halogen, CN, C₁-C₃ alkyl, C₁-C₃ alkoxy, C₁-C₃ alkenyl and C₁-C₃ alkynyl;
R³ is hydrogen, halogen or C₁-C₃ alkyl;
R⁴ is C₃-C₅ cycloalkyl, C₁-C₆ alkyl, C₂-C₆ alkenyl, C₂-C₆ alkynyl, phenyl, a 5-6 membered heteroaryl, or NR⁸R⁹, wherein the cycloalkyl, alkyl, alkenyl, alkynyl, phenyl and heteroaryl are optionally substituted with OR²⁰, halogen, phenyl, C₃-C₄ cycloalkyl, or C₁-C₄ alkyl optionally substituted with halogen;
R⁵ is hydrogen, C₁-C₃ alkyl, or NR10R11;
R⁶ is hydrogen, C₁-C₆ alkoxy or C₁-C₆ alkyl, wherein each C₁-C₆ alkoxy and C₁-C₆ alkyl is optionally substituted with halogen, OR²⁰, SR²⁰, NR¹⁴R¹⁵, C₃-C₆ cycloalkyl, 4-6 membered heterocyclyl, 5-6 membered heteroaryl or phenyl; provided when X is NR⁶ and is double bonded to an adjacent atom in formula I then R⁶ is absent;
R⁷ is hydrogen, C₁-C₆ alkoxy or C₁-C₆ alkyl, wherein each C₁-C₆ alkoxy and C₁-C₆ alkyl is optionally substituted with halogen, OR²⁰, SR²⁰, NR¹⁶R¹⁷, C₃-C₆ cycloalkyl, 4-6 membered heterocyclyl, 5-6 membered heteroaryl or phenyl; provided when Y is NR⁷ and is double bonded to an adjacent atom in formula I then R⁷ is absent;
R⁸ and R⁹ are each independently hydrogen or C₁-C₆ alkyl optionally substituted by halogen; or R⁸ and R⁹ are independently taken together with the atom to which they are attached to form a 3-6 membered heterocyclyl, optionally substituted by halogen, oxo or C₁-C₃ alkyl;
R¹⁰ is hydrogen;
R¹¹ is hydrogen, —(C₀-C₃ alkyl)CN, (C₀-C₃ alkyl)NR¹²R¹³, (C₀-C₃ alkyl)OR²⁰, (C₁-C₃ alkyl)SR²⁰, C₁-C₆ alkyl, C₂-C₆ alkenyl, C₂-C₆ alkynyl, (C₀-C₃ alkyl)C₃-C₆ cycloalkyl, (C₀-C₃ alkyl)phenyl, (C₀-C₃ alkyl) 3-6-membered heterocyclyl or (C₀-C₃ alkyl) 5-6-membered heteroaryl, wherein said alkyl, alkenyl, alkynyl, cycloalkyl, heterocyclyl, heteroaryl and phenyl are optionally substituted by halogen, oxo, OR²¹, NR¹⁸R¹⁹ or C₁-C₃ alkyl;
R¹² and R¹³ are independently hydrogen or C₁-C₆ alkyl optionally substituted by halogen; or R¹² and R¹³ are taken together with the atom to which they attached to form a 3-6 membered heterocyclyl, optionally substituted by halogen, oxo or C₁-C₃ alkyl;
R¹⁴ and R¹⁵ are independently hydrogen or C₁-C₆ alkyl optionally substituted by halogen; or R¹⁴ and R¹⁵ are taken together with the atom to which they attached to form a 3-6 membered heterocyclyl optionally substituted by halogen, oxo or C₁-C₃ alkyl;
R¹⁶ and R¹⁷ are independently hydrogen or C₁-C₆ alkyl optionally substituted by halogen; or R¹⁶ and R¹⁷ are taken together with the atom to which they attached to form a 3-6 membered heterocyclyl optionally substituted by halogen, oxo or C₁-C₃ alkyl;
R¹⁸ and R¹⁹ are independently hydrogen or C₁-C₆ alkyl optionally substituted by halogen; or R¹⁸ and R¹⁹ are taken together with the atom to which they attached to form a 3-6 membered heterocyclyl optionally substituted by halogen, oxo or C₁-C₃ alkyl;
each R²⁰ is independently hydrogen or C₁-C₆ alkyl optionally substituted by halogen; and
each R²¹ is independently hydrogen or C₁-C₆ alkyl optionally substituted by halogen.
